(19) United States
(12) Patent Application Publication
(10) Pub. No.: US 2025/0302998 A1
(43) Pub. Date: Oct. 2, 2025

(54) VECTORS AND METHODS FOR IN VIVO ANTIBODY PRODUCTION

(71) Applicant: Regeneron Pharmaceuticals, Inc., Tarrytown, NY (US)

(72) Inventors: Leah SABIN, Goldens Bridge, NY (US); Christos KYRATSOUS, Irvington, NY (US); Kurt EDELMAN, Yorktown Heights, NY (US)

(73) Assignee: Regeneron Pharmaceuticals, Inc., Tarrytown, NY (US)

(21) Appl. No.: 18/863,922

(22) PCT Filed: May 9, 2023
(86) PCT No.: PCT/US2023/066790

§ 371 Date: Nov. 7, 2024

Related U.S. Application Data
(60) Provisional application No. 63/339,665, filed on May 9, 2022.

(51) Int. Cl. A61K 48/00; C07K 16/28; C12N 5/00; C12N 5/0781; C12N 9/22; C12N 15/11; C12N 15/86; C12N 15/90

(52) U.S. Cl. CPC A61K 48/0058; A61K 48/0075; A61K 48/0083; C07K 16/2878; C07K 16/2896; C12N 5/0018; C12N 5/0635; C12N 9/226; C12N 15/111; C12N 15/86; C12N 15/902; C12N 2310/20; C12N 2501/52; C12N 2501/599; C12N 2510/00; C12N 2740/15043; C12N 2750/14143; C12N 2750/14145; C12N 2800/40

(57) ABSTRACT

The invention relates to compositions and methods that can target B cells and/or hematopoietic stem cells (HSCs) in order to engineer those cells to express specific antibodies ex vivo or in vivo and become part of the host's long-lived immune repertoire.

### CROSS-REFERENCE TO RELATED APPLICATIONS

This patent application claims priority to U.S. Provisional Application No. 63/339,665, filed May 9, 2022, the disclosure of which is herein incorporated by reference in its entirety.

### FIELD OF THE INVENTION

The invention relates to compositions and methods that can target B cells and/or hematopoietic stem cells (HSCs) in order to engineer those cells to express specific antibodies ex vivo or in vivo and become part of the host's long-lived immune repertoire.

### BACKGROUND OF THE INVENTION

In general, the success of immunization depends on the ability of the host to respond to a given immunogen and generate the appropriate response. In certain populations (e.g., young children, elderly, immunocompromised etc.) vaccines sometimes fail to properly elicit the desired response. For several infectious agents, design of an immunogen to generate a sufficiently broad and potent immune response has not been successful even in normal healthy populations. Additionally, for some pathogens (e.g., Dengue), vaccination may actually result in enhancement of infection (ADE) rather than protection depending on individual vaccine responses such as the isotype of antibodies elicited. Monoclonal antibodies can either be selected or designed to overcome many of these issues, but compared to vaccines, passively delivered antibodies are short lived, and life-long immunity would require frequent re-administration. Over the past few years, approaches to express monoclonal antibodies in vivo have been pursued.

### SUMMARY OF THE INVENTION

As specified in the Background section, there is a great need in the art to express monoclonal antibodies in vivo. The present invention addresses this and other needs by providing compositions and methods that can target B cells and/or hematopoietic stem cells (HSCs) in order to engineer those cells to express specific antibodies ex vivo or in vivo and become part of the host's long-lived immune repertoire.

In one aspect, the present disclosure provides a system for producing an antibody or an antigen-binding fragment thereof in a subject, comprising:
a) a first component comprising a polynucleotide molecule, wherein the polynucleotide molecule comprises a sequence encoding the antibody or antigen-binding fragment thereof, and
b) a second component comprising a gene editing molecule or a polynucleotide molecule comprising a sequence encoding said gene editing molecule.

In some embodiments, administration of the first and second components to the subject results in an integration of the sequence encoding the antibody or the antigen-binding fragment thereof into the DNA of a B cell and/or a hematopoietic stem cell (HSC) of the subject, causing the production of the antibody or the antigen-binding fragment in the subject.

In some embodiments, administration of the first and second components to a B cell and/or a hematopoietic stem cell (HSC) ex vivo results in an integration of the sequence encoding the antibody or antigen-binding fragment thereof into the DNA of the cell to produce a modified B cell or a modified HSC, causing the production of the antibody or antigen-binding fragment thereof in the subject upon administration of the modified B cell or HSC to the subject.

In some embodiments, the B cell is a B1 B cell. In some embodiments, the B cell is a B2 B cell.

In some embodiments, the first component and/or the second component are independently selected from a viral vector, a virus-like particle (VLP), a liposome, a lipid nanoparticle (LNP), and a ribonuclear protein (RNP) complex.

In some embodiments, the first component and the second component are both viral vectors. In some embodiments, the viral vectors are derived from the same viral species. In other embodiments, the viral vectors are derived from different viral species.

In some embodiments, the first or second component further comprises a guide RNA (gRNA) molecule or a sequence encoding the gRNA molecule.

In some embodiments, the first component comprises the polynucleotide molecule comprising the sequence encoding the antibody or antigen-binding fragment thereof and the sequence encoding the gRNA.

In some embodiments, the first component comprises (i) a first polynucleotide molecule comprising the sequence encoding the antibody or antigen-binding fragment thereof, and (ii) a second polynucleotide molecule comprising the sequence encoding the gRNA.

In some embodiments, the first component comprises (i) a first polynucleotide molecule comprising the sequence encoding the antibody or antigen-binding fragment thereof; and (ii) the gRNA molecule.

In some embodiments, the second component comprises the gRNA molecule or the sequence encoding said gRNA molecule.

In one aspect, the disclosure provides a vector system for generation of a cell population capable of producing an antibody or an antigen-binding fragment thereof in vivo, comprising: a first viral vector comprising a sequence encoding the target antibody or a fragment thereof and a sequence encoding a guide RNA (gRNA), a second viral vector comprising a sequence encoding a gene editing molecule, wherein, the vector system integrates the sequence encoding the target antibody or an antigen-binding fragment thereof into the DNA of the cell, causing the cell to produce the antibody or an antigen-binding fragment thereof.

In some embodiments, the cell population is a human cell population. In some embodiments, the cell population is a B cell population (e.g., comprising B1 B cells and/or comprising B2 B cells). In some embodiments, the cell population is a hematopoietic stem cell (HSC) population.

In some embodiments, one or both viral vectors used in the system of the present disclosure are adeno-associated virus (AAV) vectors. In some embodiments, the AAV vector is derived from AAV1, AAV2, AAV6, AAV9, or AAV9.PHP. In some embodiments, the AAV vector capsid comprises one or more mutations, wherein the one or more mutations abolish a natural tropism of the AAV vector. In some embodiments, the AAV vector capsid is derived from AAV1 or AAV6 and comprises mutation Y445F and/or V473D. In some embodiments, the AAV vector capsid is derived from AAV9 and comprises mutation W503A.

In some embodiments, one or both viral vectors used in the system of the present disclosure are retroviral vectors, such as lentiviral vectors.

In some embodiments, the viral vector used in the system of the present disclosure further comprises a targeting moiety. In some embodiments, the targeting moiety is expressed on the outer surface of the virus capsid. In some embodiments, the targeting moiety is attached to the outer surface of the virus capsid by a linker.

In some embodiments, the viral vector is an AAV vector and the targeting moiety is inserted into a protein forming the viral capsid or is covalently or non-covalently attached to the protein forming the viral capsid. In some embodiments, the targeting moiety is attached to the viral capsid via a first member and a second member of a binding pair. The first member and the second member may form an isopeptide bond.

In some embodiments, the viral vector is a lentiviral vector and the targeting moiety is covalently or non-covalently attached to a fusogen.

In some embodiments, the targeting moiety is attached to the outer surface of the virus capsid by a SpyTag:SpyCatcher system. In some embodiments, the targeting moiety is a targeting antibody or an antigen-binding fragment thereof. Non-limiting examples of useful antibodies include, e.g., anti-CD19, anti-CD20, anti-CD34, anti-CD38, anti-CD40, anti-CD117, anti-CD22, anti-CD79, anti-CD180, anti-CD5, anti-B cell receptor (BCR) (e.g., IgM, IgD, IgG), anti-B-cell activating factor (BAFF) and anti-Sca-1 antibodies, or antigen-binding fragments thereof.

In some embodiments, the gene editing molecule is a Cas nuclease, such as a Cas9 nuclease.

In various embodiments, the gRNA is complimentary to a sequence at the IgH locus, J Chain locus, or Ig Kappa locus. In some embodiments, the gRNA is complimentary to a sequence at the J Chain locus. In one embodiment, the gRNA is complimentary to a sequence in the 4th exon of the J Chain locus. In one embodiment, the gRNA is complimentary to a sequence in the 1st intron of the J Chain locus.

In some embodiments, the gRNA-encoding sequence encodes a gRNA that is complimentary to a sequence that encodes a V13 region of an antibody. In some embodiments, the gRNA is selected from gRNA1, gRNA2, gRNA3, gRNA4, gRNA5, gRNA6, gRNA7, gRNA8, gRNA9, gRNA10, and gRNA12.

In some embodiments, the sequence encoding the antibody or antigen-binding fragment thereof comprises a sequence encoding the light chain variable region and optionally the light chain constant region of the antibody. In some embodiments, the sequence encoding the antibody or a fragment thereof comprises a sequence encoding the heavy chain variable sequence of the antibody.

In some embodiments, the sequence encoding the antibody or antigen-binding fragment thereof is integrated at the IgH locus in the genomic region downstream of the final J gene but upstream of the Ep enhancer.

In some embodiments, the integration of the sequence encoding the antibody or antigen-binding fragment thereof into the DNA of the B cell or HSC results in the disruption of the Kappa light chain constant region.

In some embodiments, the polynucleotide molecule comprising the sequence encoding the antibody or antigen-binding fragment thereof comprises from 5′ to 3′ a 5′ IgH homology region, splice acceptor, a 2A sequence with 5′ furin cleavage sequence, a sequence encoding the light chain variable region of the antibody, a sequence encoding the light chain constant region of the antibody, a 2A sequence with 5′ furin cleavage sequence, a sequence encoding the heavy chain variable region of the antibody, splice donor sequence, and 3′ IgH homology region.

In some embodiments, the polynucleotide molecule comprising the sequence encoding the antibody or antigen-binding fragment thereof comprises from 5′ to 3′ 5′ J Chain exon 4 homology region, a 2A sequence with 5′ furin cleavage sequence, a sequence encoding the light chain variable region of the antibody, a sequence encoding the light chain constant region of the antibody, a 2A sequence with 5′ furin cleavage sequence, a sequence encoding the heavy chain variable region of the antibody, a sequence encoding the heavy chain constant region of said antibody, 3′ J Chain exon 4 homology region, wherein the heavy chain and light chain sequences can be placed in either order.

In some embodiments, the polynucleotide molecule comprising the sequence encoding the antibody or antigen-binding fragment thereof comprises from 5′ to 3′ a sequence encoding a guide RNA (gRNA) sequence, a splice acceptor sequence, a 2A sequence, a sequence encoding a light chain of the target antibody, a 2A sequence, a sequence encoding a heavy chain variable sequence of the target antibody, and a splice donor sequence.

In some embodiments, the sequence encoding the antibody or antigen-binding fragment thereof does not comprise a promoter sequence. Upon integration into the DNA of the B cell or HSC, the sequence encoding the antibody or antigen-binding fragment thereof may be under the transcriptional control of an endogenous promoter. In one embodiment, upon integration of the sequence encoding the antibody or antigen-binding fragment thereof into the DNA of the B cell or HSC, the sequence is under the transcriptional control of an endogenous heavy chain promoter in the B cell or HSC. In one embodiment, upon integration of the sequence encoding the antibody or antigen-binding fragment thereof into the DNA of the B cell or HSC, the sequence is under the transcriptional control of an endogenous J Chain promoter in the B cell or HSC.

In some embodiments, the sequence encoding the antibody or antigen-binding fragment thereof comprises a promoter sequence. In some embodiments, the promoter is a B cell specific promoter or HSC specific promoter. Non-limiting examples of B cell specific promoter or HSC specific promoter include Hg38-mCP promoter, and spleen focus forming virus (SFFV) promoter, or a fragment thereof.

In some embodiments, the antibody or antigen-binding fragment thereof binds an antigen associated with a disease or disorder. The diseases or disorders can include, but are not limited to, an infection, cancer, autoimmune disease, cardiovascular disease, musculoskeletal disorder, or neurodegenerative disease. In some embodiments, the infection is a viral infection, bacterial infection, fungal infection, or a parasite infection. In some embodiments, the antigen is a viral antigen, a bacterial antigen, a fungal antigen, a parasite antigen, or a tumor-associated antigen (TAA).

In various embodiments of the system described herein, the subject is human.

In various embodiments of the system described herein, the subject is an experimental animal, such as a mouse or rat.

In a related aspect, provided herein is a modified B cell or a modified hematopoietic stem cell (HSC) comprising the system of any one of the embodiments described herein.

In another aspect, provided herein is a pharmaceutical composition comprising the system of any one of the embodiments described herein and a pharmaceutically acceptable carrier or excipient.

In another aspect, provided herein is a kit comprising (i) the system of any one of embodiments described herein and optionally (ii) a container and/or instructions for use.

In another aspect, provided herein a method for generating a modified B cell or a modified hematopoietic stem cell (HSC) producing an antibody or antigen-binding fragment thereof. The method may comprise transducing ex vivo a B cell or HSC with an effective amount of the system of any one of the embodiments described herein, wherein the first component and the second component of the system are administered to the cell either simultaneously or sequentially in any order, and wherein the administration of the first and second components results in an integration of the sequence encoding the antibody or antigen-binding fragment thereof into the DNA of the cell, wherein said cell becomes a modified cell.

In some embodiments of the above ex vivo method, the first component and the second component of the system are administered to the cell simultaneously as two separate compositions.

In some embodiments of the above ex vivo method, the first component and the second component of the system are administered to the cell simultaneously as one composition.

In various embodiments of the above ex vivo method, wherein the B cell or HSC is present in a heterogeneous cell population during the transduction.

In various embodiments of the above ex vivo method, the B cell has been isolated from spleen, peritoneum, or peripheral blood.

In some embodiments, the B cell is a primary B cell.

In various embodiments of the above ex vivo method, the B cell is a B2 B cell.

In various embodiments of the above ex vivo method, the B cell is a B1 B cell. In some embodiments, the B cell is B1a B cell (CD19+/CD5+/CD23−) or B1b B cell (CD19+/CD5−/CD23−).

In various embodiments of the above ex vivo method, the B cell is cultured under stimulation conditions prior to and/or after the transduction.

In some embodiments, the stimulation conditions promote B cell activation without differentiation

In various embodiments of the above ex vivo method, the B cell is cultured in the presence of a CD40 agonist and/or a CD180 agonist prior to and/or after the transduction. In some embodiments, the B cell is cultured in the presence of a CD40 agonist and a CD180 agonist prior to and/or after the transduction. In some embodiments, the CD40 agonist is CD40 ligand (CD40L) or an anti-CD40 antibody or antigen-binding fragment thereof. In some embodiments, the CD180 agonist is an anti-CD180 antibody or antigen-binding fragment thereof.

In some embodiments, the B cell is cultured in the presence of about 20 ng/ml or less of a CD40 agonist (e.g., CD40L) and/or about 100 ng/ml or less of a CD180 agonist (e.g., anti-CD180 antibody) prior to and/or after the transduction. In some embodiments, the CD40 agonist (e.g., CD40L) used in the B cell culture is about 0.5, about 1, about 2, about 3, about 4, about 5, about 6, about 7, about 8, about 9, about 10, about 11, about 12, about 13, about 14, about 15, about 16, about 17, about 18, about 19, about 20 ng/ml, or about 0-5 ng/ml, about 4-8 ng/ml, about 5-10 ng/ml, about 6-12 ng/ml, about 8-15 ng/ml, about 10-15 ng/ml, about 12-18 ng/ml, or about 15-20 ng/ml. In some embodiments, the CD180 agonist (e.g., anti-CD180 antibody) used in the B cell culture is about 0.5, about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 85, about 90, about 95, about 100, or about 0-5 ng/ml, about 4-8 ng/ml, about 5-10 ng/ml, about 6-12 ng/ml, about 8-15 ng/ml, about 10-15 ng/ml, about 12-18 ng/ml, about 15-20 ng/ml, about 20-25 ng/ml, about 20-30 ng/ml, about 25-40 ng/ml, about 30-50 ng/ml, about 40-60 ng/ml, about 50-75 ng/ml, about 60-80 ng/ml, about 70-90 ng/ml or about 80-100 ng/ml. In some embodiments, the B cell is cultured in the presence of about 20 ng/ml of a CD40 agonist (e.g., CD40L) and about 20 ng/ml of a CD180 agonist (e.g., anti-CD180 antibody) prior to and/or after the transduction. In some embodiments, the B cell is cultured in the presence of a CD40 agonist (e.g., CD40L) and/or a CD180 agonist (e.g., anti-CD180 antibody) for about 4 days or less prior to the transduction. In some embodiments, the B cell is cultured in the presence of a CD40 agonist (e.g., CD40L) and/or a CD180 agonist (e.g., anti-CD180 antibody) for about 4 hours, about 8 hours, about 12 hours, about 16 hours, about 20 hours, about 1 day, about 36 hours, about 2 days, about 60 hours, about 3 days, about 84 hours, or about 4 days prior to the transduction. In some embodiments, the B cell is cultured in the presence of about a CD40 agonist (e.g., CD40L) and/or a CD180 agonist (e.g., anti-CD180 antibody) for about 2 days prior to the transduction. In some embodiments, the B cell is cultured in the presence of a CD40 agonist (e.g., CD40L) and/or a CD180 agonist (e.g., anti-CD180 antibody) for about 2 days after the transduction.

In some embodiments of the above ex vivo method, the method further comprises culturing the modified B cell or modified HSC under differentiating conditions to promote differentiation of the modified B cell or modified HSC into a modified plasma cell.

In various embodiments of the above ex vivo method, the method further comprises introducing the modified B cell or the modified HSC or the modified plasma cell into a subject. In some embodiments, the modified B cell or HSC or the modified plasma cell is introduced into the subject intraperitoneally. In some embodiments, the subject has been depleted of CD20+ cells prior to introducing the modified B cell or HSC. In some embodiments, after introducing the modified B cell or HSC or plasma cell into the subject, the modified B cell or HSC or plasma cell is expanded in vivo by administering to the subject an antigen that is recognized by the antibody or antigen-binding fragment thereof which is produced by the modified B cell or HSC or plasma cell. In some embodiments, the subject is autologous to the modified B cell or HSC or plasma cell. In some embodiments, the subject is allogeneic to the modified B cell or HSC or plasma cell. In some embodiments, the subject is human. In some embodiments, the subject is an experimental animal.

In a related aspect, provided herein is a modified B cell or modified HSC, or a population thereof, produced by the method of any one of the embodiments described above. In a related aspect, provided herein is a modified plasma cell produced by the method described above.

In another aspect, the disclosure provides a method for producing an antibody or antigen-binding fragment thereof in vivo in a subject in need thereof, comprising transducing ex vivo B cells and/or hematopoietic stem cells (HSCs) isolated from the subject or a donor with an effective amount of any of the above systems, wherein the first component (e.g., viral vector) and the second component (e.g., viral vector) are administered either simultaneously or sequentially in any order, and then re-introducing the transduced cells into the subject. In some embodiments, B cells comprise B1 B cells. In some embodiments, B cells comprise B2 B cells. In some embodiments, the B cells comprise primary B cells.

In a related aspect, the disclosure provides a method for producing an antibody or antigen-binding fragment thereof in vivo in a subject in need thereof, comprising administering to the subject an effective amount of any of the above systems, wherein the first component (e.g., viral vector) and the second component (e.g., viral vector) are administered either simultaneously or sequentially in any order. In some embodiments, the administration of the first and second components to the subject results in an integration of the sequence encoding the antibody or antigen-binding fragment thereof into the DNA of B cells and/or hematopoietic stem cells (HSCs) of the subject, causing a production of the antibody or antigen-binding fragment thereof in the subject.

In some embodiments of the above in vivo method, the first component and the second component of the system are administered to the subject simultaneously as two separate compositions.

In some embodiments of the above in vivo method, the first component and the second component of the system are administered to the subject simultaneously as one composition.

In some embodiments of the above in vivo method, the first component and/or the second component of the system is administered to the subject intraperitoneally.

In some embodiments of the above in vivo method, the method further comprises administering to the subject an effective amount of a CD180 agonist and/or a CD40 agonist prior to the administration of the system to the subject. In some embodiments, the CD40 agonist is CD40 ligand (CD40L) or an anti-CD40 antibody or antigen-binding fragment thereof. In some embodiments, the CD180 agonist is an anti-CD180 antibody or antigen-binding fragment thereof.

In some embodiments, the method comprises administering to the subject an effective amount of a CD180 agonist (e.g., anti-CD180 antibody) and a CD40 agonist (e.g., anti-CD40 antibody) prior to the administration of the system to the subject. In some embodiments, the method comprises administering to the subject about 250 μg or less of a CD180 agonist (e.g., anti-CD180 antibody) and/or about 50 μg or less of a CD40 agonist (e.g., anti-CD40 antibody) prior to the administration of the system to the subject. In some embodiments, the method comprises administering to the subject about 0.5, about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, about 55, about 60, about 65, about 70, about 75, about 85, about 90, about 95, about 100, about 110, about 120, about 130, about 140, about 150, about 160, about 170, about 180, about 190, about 200, about 210, about 220, about 230, about 240, about 250, or about 0-5, about 4-8, about 5-10, about 6-12, about 8-15, about 10-15, about 12-18, about 15-20, about 20-25, about 20-30, about 25-40, about 30-50, about 40-60, about 50-75, about 60-80, about 70-90, about 80-100, about 100-120, about 120-150, about 140-160, about 150-180, about 175-200, about 200-225, about 225-250 μg of a CD180 agonist (e.g., anti-CD180 antibody) prior to the administration of the system to the subject. In some embodiments, the method comprises administering to the subject about 0.5, about 1, about 2, about 3, about 4, about 5, about 6, about 7, about 8, about 9, about 10, about 11, about 12, about 13, about 14, about 15, about 16, about 17, about 18, about 19, about 20, about 25, about 30, about 35, about 40, about 45, about 50, or about 0-5, about 4-8, about 5-10, about 6-12, about 8-15, about 10-15, about 12-18, about 15-20, about 20-25, about 20-30, about 25-35, about 30-40, about 40-50 μg of a CD40 agonist (e.g., anti-CD40 antibody) prior to the administration of the system to the subject. In one embodiment, the method comprises administering to the subject about 12.5 μg of a CD180 agonist (e.g., anti-CD180 antibody) and no CD40 agonist (e.g., anti-CD40 antibody). In one embodiment, the method comprises administering to the subject no CD180 agonist (e.g., anti-CD180 antibody) and about 12.5 μg of a CD40 agonist (e.g., anti-CD40 antibody).

In some embodiments, the method comprises administering to the subject an effective amount of a CD180 agonist (e.g., anti-CD180 antibody) and a CD40 agonist (e.g., anti-CD40 antibody) prior to the administration of the system to the subject. In some embodiments, the method comprises administering to the subject about 8.5 mg/kg of body weight or less of a CD180 agonist (e.g., anti-CD180 antibody) and/or about 1.8 mg/kg of body weight or less of a CD40 agonist (e.g., anti-CD40 antibody) prior to the administration of the system to the subject. In some embodiments, the method comprises administering to the subject about 0.1, about 0.2, about 0.3, about 0.4, about 0.5, about 0.6, about 0.7, about 0.8, about 0.9, about 1, about 1.5, about 2, about 2.5, about 3, about 3.5, about 4, about 4.5, about 5, about 5.5, about 6, about 6.5, about 7, about 7.5, about 8, about 8.33, about 8.5, or about 0-0.5, about 0.4-0.8, about 0.5-1, about 1-2, about 1.5-2.5, about 2-4, about 3-5, about 4-6, about 5-7, about 6-8, or about 7.5-8.5 mg/kg of body weight of a CD180 agonist (e.g., anti-CD180 antibody) prior to the administration of the system to the subject. In some embodiments, the method comprises administering to the subject about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, or about 0-0.5, about 0.2-0.6, about 0.4-0.8, about 0.5-1, about 0.6-1.2, about 0.8-1.5, about 1-1.5, about 1.2-1.6, about 1.5-1.8 mg/kg of body weight of a CD40 agonist (e.g., anti-CD40 antibody) prior to the administration of the system to the subject. In one embodiment, the method comprises administering to the subject about 0.4 mg/kg of body weight of a CD180 agonist (e.g., anti-CD180 antibody). In one embodiment, the method comprises administering to the subject about 0.4 mg/kg of body weight of a CD40 agonist (e.g., anti-CD40 antibody). In some embodiments, the method comprises administering to the subject a CD180 agonist without a CD40 agonist. In some embodiments, the method comprises administering to the subject a CD40 agonist without a CD180 agonist.

In some embodiments, the method comprises administering to the subject a CD180 agonist (e.g., anti-CD180 antibody) and/or a CD40 agonist (e.g., anti-CD40 antibody) about 7 days or less prior to the administration of the system to the subject. In some embodiments, the method comprises administering to the subject a CD180 agonist (e.g., anti-CD180 antibody) and/or a CD40 agonist (e.g., anti-CD40 antibody) about 4 hours, about 8 hours, about 12 hours, about 16 hours, about 20 hours, about 1 day, about 36 hours, about 2 days, about 60 hours, about 3 days, about 84 hours, about 4 days, about 5 days, about 6 days, or about 7 days prior to the administration of the system to the subject. In some embodiments, the method comprises administering to the subject a CD180 agonist (e.g., anti-CD180 antibody) and/or a CD40 agonist (e.g., anti-CD40 antibody) about 2-3 days prior to the administration of the system to the subject.

In some embodiments of the above in vivo method, the method further comprises administering to the subject an effective amount of an antigen which is recognized by the antibody or antigen-binding fragment thereof, wherein the antigen is administered before and/or after administering the first and/or second component of the system. In some embodiments, the antigen has a low affinity for the antibody or antigen-binding fragment thereof. In some embodiments, the antigen has a high affinity (e.g., picomolar range) for the antibody or antigen-binding fragment thereof. In some embodiments, the method comprises administering the antigen which has a low affinity for the antibody or antigen-binding fragment thereof prior to administering the first and second components of the system, and administering the antigen which has a high affinity (e.g., picomolar range) for the antibody or antigen-binding fragment thereof after administering the first and second components of the system.

In some embodiments of the above in vivo method, the method comprises administering to the subject an effective amount of a first antigen, wherein the first antigen has a low affinity for the antibody or antigen-binding fragment thereof and wherein said first antigen is administered prior to administering the first and second components of the system, and administering to the subject an effective amount of a second antigen, wherein the second antigen has a high affinity for the antibody or antigen-binding fragment thereof and wherein the second antigen is administered after administering the first and second components of the system.

In some embodiments of the above in vivo method, the subject is human.

In some embodiments of the above in vivo method, the subject is an experimental animal.

In a further aspect, the disclosure provides a method for treating or reducing the likelihood of a disease or disorder in a subject in need thereof, comprising performing the ex vivo method of any one of the embodiments described above or the in vivo method of any one of the embodiments described above, wherein the method results in a production in the subject of an effective amount of the antibody or antigen-binding fragment thereof.

In yet another aspect, the disclosure provides a method for treating or reducing the likelihood of a disease or disorder in a subject in need thereof, comprising administering to the subject an effective amount of any of the above systems, wherein the first component (e.g., viral vector) and the second component (e.g., viral vector) are administered either simultaneously or sequentially in any order, wherein the administration of the system results in production of an effective amount of the an antibody or antigen-binding fragment thereof in vivo in the subject. The produced antibody or antigen-binding fragment thereof binds an antigen associated with a disease or disorder.

In yet another aspect, the disclosure provides a method for treating a disease in a subject in need thereof, comprising transducing ex vivo B cells and/or hematopoietic stem cells (HSCs) isolated from the subject or a donor with an effective amount of any of the above systems, wherein the first component (e.g., viral vector) and the second component (e.g., viral vector) are administered either simultaneously or sequentially in any order, and then re-introducing the transduced cells into the subject, wherein the administration of the system results in production of an effective amount of an antibody or antigen-binding fragment thereof in vivo in the subject. The produced antibody or antigen-binding fragment thereof binds an antigen associated with a disease or disorder. In some embodiments, B cells comprise primary B cells. In some embodiments, B cells comprise B1 B cells. In some embodiments, B cells comprise B2 B cells.

In some embodiments of any of the above treatment methods, the disease is an infection, a cancer, autoimmune disease, cardiovascular disease, musculoskeletal disorder, or neurodegenerative disease. In some embodiments, the infection is a viral infection, a bacterial infection, a fungal infection, or a parasite infection.

In some embodiments of any of the above methods, the first component (e.g., viral vector) and the second component (e.g., viral vector) are administered in the same composition.

In some embodiments of any of the above methods, the subject is human.

In some embodiments of any of the above methods, the subject is an experimental animal, such as a mouse or rat.

These and other aspects described herein will be apparent to those of ordinary skill in the art in the following description, claims and drawings.

### BRIEF DESCRIPTION OF THE DRAWINGS

FIG. 1 depicts an overview of an AAV-mediated delivery of transgene to a mouse for episome expression.

FIG. 2 depicts an overview of an AAV plus Cas9/guide RNA (gRNA) mediated insertion of a transgene into a genomic locus of a mouse liver.

FIG. 3 depicts results showing in vitro neutralization with episomal and liver-inserted anti-PcrV monoclonal antibodies (mAbs) from mouse serum is within 2-5-fold of CHO-purified mAb.

FIG. 4 depicts results showing that episomal and liver-inserted anti-PcrV mAbs gives protection from lethal infection in an in vivo challenge model with P. aeruginosa.

FIG. 5A depicts an overview of an ex vivo strategy for adaptive antibody vaccination in mice. FIG. 5B depicts an exemplary AAV vector carrying antibody heavy chain and light chain genes.

FIG. 6 depicts results showing the percentage of B cells that express the introduced B-cell receptor (BCR) of interest for mock controls, RNP controls, and RNP+AAV1.

FIGS. 7A-7C depict an overview of engineering B-cell specificity by insertion of antibody genes into the heavy chain locus of peripheral B cells. FIG. 7A depicts the removal of mouse B-cells, their modification by Cas9/gRNA via AAV delivery. FIG. 7B depicts the VI3 heavy chain, the gRNA cut site, and the BCR insert. FIG. 7C depicts the BCR insert spliced into the VI3 heavy chain.

FIGS. 8A-8B depict ULC-pairing and full length BCR insertion. FIG. 8A depicts the two BCR variants used, ULC-pairing anti-BCMA (top) and the anti-PcrV (bottom). FIG. 8B depicts the results for both variants, in AAV only and RNP only controls, plus the AAV+RNP experimental condition. Antigen binding is given as a percentage for each condition.

FIGS. 9A-9B depict insertion of mCherry constructs to test template designs. FIG. 9A depicts the two mCherry variants, T2A-mCherry (top) and pVh3-23-mCherry (bottom). FIG. 9B depicts the results for both variants, with no virus and no RNP controls. Results were analyzed 3 days post-infection. Positive mCherry expression in both variants with 150 pmol Cas9 and 400 pmol gRNA3.

FIGS. 10A-10B depict the use of multiple gRNA targeting sites for VI3 insertion. FIG. 10A depicts the insertion location of the eight different gRNAs used. FIG. 10B depicts the results of T2A-mCherry expression using each of the eight different gRNAs.

FIGS. 11A-11B depict multiple gRNA targeting sites available in the Ig Kappa locus to disrupt expression of the endogenous light chain and support a full-length antibody insertion. FIG. 11A depicts the seven different gRNA that were used for comparison: gRNA4, gRNA6, gRNA7, gRNA8, gRNA9, gRNA10, and gRNA4+6. FIG. 11B depicts results that show gRNA7, which cuts at the splice acceptor site and doesn't require recoding of Kappa constant in the AAV template, has a low mlg Lambda and mlg Kappa expression at 91.7%, along with gRNA10 (92.2) and gRNA4+6 (92.0). Other results: mock control (5.42), gRNA4 (82.4), gRNA6 (85.7), gRNA8 (37.3), and gRNA9 (81.7).

FIGS. 12A-12B depict results of mouse splenic B cells cultured with the following growth factors: 1) CD40L-HA, Anti-HA, and IL-4 (as previous), 2) anti-CD180, 3) CD40L-HA, anti-HA, and BAFF, 4) anti-CD180 and BAFF, and 5) CD40L-HA, anti-HA, anti-CD180, and BAFF. 3 million cells were nucleofected at 24 hrs with 150 pmol Cas9, 400 pmol gRNA, and AAV6-V13-gRNA1-T2A-mCherry (FIG. 12A). 500,000 cells were infected with AAV6 at 2.5e5 vg/cell and analyzed 3 days post-infection. mCherry expression was strongest in conditions 1 (19.5) and 5 (10.8) as shown in FIG. 12B.

FIGS. 13A-13B depict results of mouse splenic B cells cultured with either 1) CD40L-HA, anti-HA, and IL-4, or 2) CD40L-HA, anti-HA, and anti-CD180. 3 million cells were nucleofected with 150 pmol Cas9, 400 pmol total gRNA (BCR-gRNA1 and mlgK-gRNA7), and a full-length HIH29338 antibody (FIG. 13A). 500,000 cells were infected with AAV1 at 2e5 vg/cell and analyzed 2 days post-infection. Both conditions worked, with condition 1 at 8.24 and condition 2 at 3.64% respectively as shown in FIG. 13B.

FIGS. 14A-14B depict the results of transfer and immunization experiment with anti-PcrV edited B cells. B cells from a CHC WT mouse were grown in CD40L-HA, anti-HA, and anti-CD180 before RNP nucleofection and AAV1 infection with h1h29339 anti-PcrV full length antibody (FIG. 14A) after 24 hours. Compared to the standard non-insertion VI3/ULC, the antibodies from the CHC WT littermates performed well (13.7 to 9.42 respectively, FIG. 14B). Full results of PerV-binding for 1) CHC WT littermate: mock control (0.28), RNP only (0.13), RNP+AAV1 (13.7); and 2) VI3/ULC: mock control (0.25), RNP only (0.54), RNP+AAV1 (9.42).

FIGS. 15A-15B depict results of B cells edited to express anti-PcrV BCR can mature to produce anti-PcrV antibodies both in vitro and in vivo after the adoptive transfer and immunization into mice. In vitro, supernatant analysis for PcrV antibodies from B cells edited for PcrV BCR and cultured in LPS for 7 days showed antibody production (FIG. 15A). In vivo, B cells were edited for PcrV BCR and transferred into Flu-CHC mice as previously described, with the serum analysis about one week post immunization, with the mice serum again producing antibodies (FIG. 15B).

FIG. 16 depicts an adoptive transfer of donor B cells from HA antigen immune mice into a CD20 cell-depleted naïve mouse recipient, the donor in vitro activated B cells appear greatly expanded one week post transfer but fail to persist 1 month later.

FIG. 17A depicts the SFFV promoter with indicated subsequences 1,2,3,4, ‘B cell core’ and the putative core promoter (predicted based on the location of a TATA box). FIG. 17B depicts expression and cell type specificity for enriched transcription factors. The expression data is used for choosing B cell specific transcription factors. FIG. 17C depicts consensus sites which can be engineered into enhancer backbones to enhance expression.

FIGS. 18A-18B depict an overview and results of the generation of reporter constructs. FIG. 18A depicts an overview of a representative reporter construct within an AAV context. SFFV subsequences were paired with MLP and cloned upstream of an eGFP coding sequence. The overview only depicts the sequence between AAV ITRs, which would be paired with bacterial sequences (such as an Ampicillin resistance) for propagation in Stbl2 cells. FIG. 18B depicts GFP expression (x-axis) after infection of primary murine B cells with AAV encoding the 5 SFFV subsequences as promoter constructs. All subsequences are paired with the adenovirus major late promoter.

FIG. 19 depicts results of full length SFFV-eGFP and variants, including SFFV-core-mCP-GFP, SFFV1-mCP-eGFP, SFFV2-mCP-eGFP, SFFV3-mCP-eGFP, and SFFV4-mCP-eGFP, that were transfected into Ramos and HEK293-HZ cells. SFFV4 shows activity in Ramos and HEK cells at 121 bp in length.

FIG. 20 depicts results showing HS-B is a 180 bp B cell specific Pax5 enhancer as shown in luciferase expression in mouse B cell line. Note top row GFP right shift for Pro-B, Pre-B, Immature B and Mature B cells compared to controls.

FIG. 21A depicts results of AAV-GFP tests of 120-170 base pair promoters in primary B cells and HEK293-HZ cells, three promoters were used with mCP-eGFP: 1) HS-B, 2) hg38HS-B, and 3) SFFV4. The cells were cultured and transfected with 5e5 vg per cell of AAV6 crude viral prep and CD40L-HA, anti-HA, and IL-4. FIG. 21B depicts the ranking of HS-B based on B cell enrichment in Corces et al. ATAC-seq dataset: #286/589,844 (#5344 if ranked by B cell signal).

FIG. 22 depicts a graphic representation of SpyTag:SpyCatcher used to attach mAb to the surface of the AAV capsid for retargeting purposes.

FIG. 23A shows that the AAV2 and AAV6 can be conjugated to hCD20 mAb at similar levels as the ASGR1 control antibody. FIG. 23B depicts results showing AAV2 (top) and AAV6 (bottom) can be targeted to CD20-expressing HEK293 cells. AAV2 or AA6 with attached anti-hCD20 mAb via SpyTag:SpyCatcher accurately targets HEK293-hCD20 cells.

FIG. 24 depicts results showing AAV2 (top row) and AAV6 (bottom row) can be targeted to CD20-expressing Ramos cells. AAV2 or AA6 with attached anti-hCD20 mAb via SpyTag:SpyCatcher accurately targets Ramos3-hCD20 cells. Graph of results (bottom) shows slight off-target with AAV6 but none with AAV2.

FIG. 25A depicts human B cells cultured under various stimulation conditions. CD19+ B cells were isolated from human peripheral blood, and cultured under various stimulation conditions: 1) IL-4 only, 2) IL-4, CD40L-HA, and anti-HA mAb, and 3) IL-4 and anti-CD40 mAb. The cells were infected with AAV2/CD20 or AAV6/CD20, and virus-delivered eGFP was measured by flow cytometry on day 4 post-infection. FIG. 25B depicts results showing AAV2 (top row) and AAV6 (bottom row) can be targeted to CD20-expressing human B cells. The results showed that while both AAV2 and AAV6 can be targeted to primary human B cells via CD20, AAV6/CD20 demonstrates a dramatic enhancement in transduction.

FIG. 26 depicts AAV1 WT, AAV1 detargeted mutant, and AAV1-hCD20, all packed with SSF-eGFP, retargeted against HEK 293 CD20(−), HEK 293 CD20(+), Jurkat T cell, and Duadi B cell lines. The results show that the AAV1 detargeted mutant still transduces and antibody conjugation slightly decreases off-target transduction, while the retargeted virus is comparable to WT in the Daudi cell line.

FIG. 27 depicts AAV2 WT, AAV2 detargeted mutant, and AAV2-hCD20, all packed with SSF-eGFP, retargeted against HEK 293 CD20(−), HEK 293 CD20(+), Jurkat T cell, and Duadi B cell lines. The results show that AAV2-CD20 shows a gain-of-function on the Daudi cell line.

FIG. 28 depicts AAV6 WT, AAV6 detargeted mutant, and AAV6-hCD20, all packed with SSF-eGFP, retargeted against HEK 293 CD20(−), HEK 293 CD20(+), Jurkat T cell, and Duadi B cell lines. The results show that the AAV6 retargeted mutant is not completely detargeted, non-binding mAb decreases off-target transduction, and AAV6-CD20 shows a gain-of-function in the 293 hCD20(+) cell line.

FIG. 29 depicts AAV9 WT, AAV9 detargeted mutant, and AAV9-hCD20, all packed with SSF-eGFP, retargeted against HEK 293 CD20(−), HEK 293 CD20(+), Jurkat T cell, and Duadi B cell lines. The results show that AAV9-CD20 sees a gain-of-function in the hCD20(+) cell line and low off-target transduction (FIG. 29).

FIG. 30 depicts graphic overview of method to translate the ex vivo B cell targeting and editing technology to in vivo application by delivering the viral vectors in vivo to mediate BCR insertion.

FIG. 31 depicts graphic showing that human stem cells are upstream of immune cells and represent a target for transduction by a range of viruses, including AAV and lentivirus.

FIG. 32 depicts resulting showing that in AAV6, attached to an anti-hCD34 (My10) antibody via the SpyTag:SpyCatcher system, was used to infect human cord blood cells and primary mouse B cells, with different promotors attached to GFP. Results showed that SFFV was the preferred promoter over CAG and EF1.

FIG. 33 depicts AAV2-hCD34, packed with SFFV-eGFP, retargeted to HSCs. Results indicate that natural tropism overrides retargeting antibody on human cord blood cells, while non-binding mAb decreases off-target transduction and anti-CD34 mAb can retarget AAV2 HBM mutant in 293/hCD34 and human cord blood cells.

FIG. 34 depicts AAV9-hCD34, packed with SFFV-eGFP, retargeted to HSCs. Results show a gain of function on 293 hCD34+ cell line in presence of CD34 antibody; low off-target transduction; and poor transduction of human cord blood cells with AAV9+/−anti-hCD34 antibody.

FIG. 35 depicts AAV6-hCD34, packed with SFFV-eGFP, retargeted to HSCs. Results show that natural tropism again overrides retargeting antibody on human cord blood cells, but anti-CD34 mAbs can robustly retarget AAV6 HBM mutants in 293/hCD34 cells and moderately retarget in human cord blood cells.

FIG. 36 depicts results showing that lentiviral vectors conjugated to anti-CD34 comparator mAbs are specifically retargeted to CD34-expressing cells, with a mAb-dependent transduction efficacy. 10,000 cells were seeded per plate (96-well plate), and 2E+08 VG of LV-SINmuZZ EF1a-FLuc was mixed with 2-fold serial diluted CHOt supe in DMEM (starting at 100 ul). After 30 min Incubation at 37 C, LV-CHOt mix is added to cells and incubated at 37 C. Fluc readout was performed 4 days after transduction. Results shown with 9 conditions: 1) 9C5 (CD34)-SpyC, 2) My1C (CD34)-SpyC, 3) 563 (CD34)-SpyC, 4) CD20-SpyC, 5) 9C5, 6) CD20, 7) BSTpro MOCK, 8) VLP only, and 9) NT. The experiment was repeated for HEK293 cells, 293-hCD20 cells, and 293-hCD34 cells.

FIG. 37 depicts results showing that SpyTagged AAV2 conjugated to anti-CD34-SpyCatcher comparator mAbs can also be specifically retargeted to CD34-expressing cells, with a mAb-dependent transduction efficacy. Results are shown with 9 conditions: 1) 9C5 (CD34)-SpyC, 2) My1C (CD34)-SpyC, 3) 563 (CD34)-SpyC, 4) CD20-SpyC, 5) 9C5, 6) CD20, 7) BSTpro MOCK, 8) VLP only, and 9) NT. The experiment was repeated for HEK293 cells, 293-hCD20 cells, and 293-hCD34 cells.

FIG. 38 depicts results showing that the optimization of mosaicism ratio reveals that AAV2 HBM-mixer 14 leads to higher transduction in HEK293T/hCD34 cell line. Screening for platform gene delivery against CD34 begins by seeding 10,000 cells per well in 96-well black wall clear bottom plate in with three cell types (293, 293-hCD20, 293-hCD34). Next, mix 5E+09 VG of AAV2 ⅛ SpyTag/HBM SFFV-Fluc with 2-fold serial diluted CHOt supe in DMEM (starting at 100 l) and incubate at 37° C. for 1.5 hr. Then, add AAV2-CHOt mix to cells and incubate at 37° C. In three days, collect the cells for flow analysis. The results shown in FIG. 34 are for different types of HBM mixers. AAV2 HBM-mixer ¼ has the higher transduction in the 293-CD34 cells, followed by AAV2 HBM-mixer ½.

FIGS. 39A-39C depict results showing lentiviral vectors retargeted with anti-CD 117 and anti-Sca-1 transduce cell lines expressing those respective target antigen receptors in vitro. 1E+04 cells were seeded in a 96-well black well clear bottom plate in 100 ul DCM with 4 ug/ml polybrene. The cells were transduced with 2E+04 VG per cell in 1001 DCM aliquots with 4 ug/ml polybrene. After 2 days, fluorescent imaging (FIG. 39A) and analysis of GFP by flow cytometry (FIGS. 39B and 39C). Results show that the vectors successfully retargeted the cell lines expressing their respective target antigen via imaging and flow cytometry.

FIG. 40 depicts results showing surface expression of CD117 and Sca-1 are detected on mouse HSPCs. At day zero, mouse HSPCs are isolated from collected bone marrow. Cells are cultured in a progenitors medium of SFEM+SCF (100 ng/mL), TPO (100 ng/mL), Flt3L (100 ng/mL), IL-6 (50 ng/mL), and IL-3 (30 ng/mL). Two days post isolation, the cells are stared for CD117 and Sca-1, then transduced with pseudoparticles with a SFFV-GFP reporter. Two days after transduction (day 4 post isolation) the readout of GFP expression is performed via FACS. The results show surface expression of CD 117 and Sca-1 on the murine HSPCs.

FIG. 41 depicts results showing that mouse HSPCs are transduced with Lentiviral Vector pseudotyped with anti-mouse CD 117 mAb and SINmu. Conditions were no additives, vectofusin-1, or lentiboost (from left to right). Cells were either non-transduced, or transduced with LV-VSVg, LV-ahASGR1+SINmu, or LV-amCD117+SINmu (top to bottom). LV pseudotyped with α-CD 117+SINmu can transduce expanded mouse primary HSPCs with very low efficiency. This is an entry issue as LV pseudotyped with VSVg is able to transduce expanded mouse HSPCs efficiently.

FIGS. 42A-42B depict results showing that SpyTagged AAV2 are efficiently retargeted to cell lines expressing CD 117 or Sca-1 in vitro. Retargeted AAV2-HBM ⅛ mosaic with either CD117, Sca-1, hCD34, of hCD20 successfully retargeted to HEK293 cell lines expressing those markers at 5E+05 VG per cell as shown by fluorescent imaging (FIG. 42A) and analysis of GFP by flow cytometry (FIG. 42B). FIGS. 42C-42D depict the rational for the design of a SpyTagged AAV2 with an anti-Sca1 antibody.

FIG. 43 shows proposed strategies for B1 B cell antibody engineering.

FIG. 44 shows proposed strategies for ectopic engineered antibody expression in B1 B cells.

FIG. 45 shows that B1a B cells activated with CD40L/aCD180 and transferred intraperitoneally have enhanced recovery at 14 and 32 days.

FIG. 46 shows that CD180 stimulation of B1a cells causes proliferation without differentiation to plasmablasts/Plasma Cells (PCs).

FIG. 47 demonstrates that transduction efficiency differs between B1 and B2 peritoneal cavity (PerC) B cell subsets.

FIG. 48 shows that Pan B cells from peritoneum can be edited but less efficiently than B2 splenocytes.

FIGS. 49A-49B depict results showing in vitro culture conditions that favor re-engraftment of ex vivo cultured mouse B cells. In vitro B cell culturing with low levels of aCD40 and/or aCD180 promoted re-engraftment of cells into mice and high B cell activation was not conducive to long term re-engraftment (FIG. 49A). 3-day ex vivo cultured CD45.1 B cells adoptively transferred into CD45.2 mice (FIG. 49B).

FIGS. 50A-50D depicts results showing ex vivo AAV transduction/editing and transfer of cultured non-differentiated Cas9 mouse B cells into SRG mice. FIG. 50A depicts an overview of an ex vivo AAV transduction/editing and transfer of cultured non-differentiated Cas9 mouse B cells into SRG mice. FIG. 50B depicts J chain locus Exon 4 insertion. FIG. 50C depicts ROSA insertion. FIG. 50D depicts luciferase signal measured in vivo over time using IVIS technology.

FIGS. 51A-51B depict exemplary editing strategies into different murine loci for different modalities. FIG. 51A depicts an exemplary “immune repertoire enhancement” modality. FIG. 51B depicts an exemplary “protein factory” modality.

FIGS. 52A-52B depict exemplary mouse J Chain locus insertion strategies to highly express proteins of interest in plasma cells. FIG. 51A depicts an exemplary strategy that uses endogenous J Chain promoter and preserves J chain. FIG. 52B depicts an exemplary strategy that uses endogenous J Chain promoter and eliminates J chain.

FIGS. 53A-53B show that generation of memory B cells is key to success of in vivo B cell editing for both adaptive antibody and protein factory modalities. FIG. 53A depicts an exemplary scheme from expansion of BCR edited B cell via new BCR. FIG. 53B depicts an exemplary scheme from expansion on non-BCR edited B cells via “linked specificity” to priming Ag.

FIGS. 54A-54D depict results showing that modulation of “Pan B-cell” stimulation of Cas9 mice enables AAV editing of B cells and Ab production. FIG. 54A depicts the VI3 heavy chain, the gRNA cut site, and the BCR insert. FIG. 54B depicts results showing synergistic effect of aCD40 and aCD180 in vivo stimulation on B cell editing and antibody expression. FIG. 54C depicts results showing that Ab1 Ab expression is short-lived in “pan-B” primed/edited mice but shows evidence of Ab recall after subsequent Ag challenge. FIG. 54D depicts results showing that in vivo B cell editing of “Pan B” stimulated mice results in edited B cells that can be recruited into subsequent response to Ag challenge.

FIGS. 55A-55D depict results showing that prime and boost with suboptimal BCR:Ag interaction encourages Ab1 memory B cells over Ab producing PC. FIG. 55A shows that suboptimal BCR: Ag interactions (“low affinity”) is predicted to skew edited B cells into memory compartment. “High affinity” BCRs result high B activation and increase Tfh interactions drives cells to become Ab producing plasma cells. B cells with “Low affinity” BCRs have lower activation and fail to adequately engage Tfh cells. This neglect skews towards memory B cells. FIG. 55B depicts results showing that Ab1 has 90-fold reduced neutralization of F490L Spike. FIG. 55C depicts results showing that prime and boosting with F490L Spike Ag fails to elicit Ab1 Ab production from Ab1 edited B cells. FIG. 55D depicts results showing that editing Ab1 BCR into mice primed with “low affinity” Ag results in reproducible induction of Ab expression following d28 boost with WT “high affinity” Ag.

FIGS. 56A-56C depict results showing that addition of aCD180 to Ag prime increases number of edited B cells able to be recalled one month and 3 months post editing. FIGS. 56A-56B depict an exemplary experimental workflow. FIG. 56C shows that Ag prime only group results in lower Ab levels post “high affinity” Ag boost compared to aCD180 treated groups and that increased Ab titers in aCD180 groups upon Ag boosting is evidence that more edited B cells were initially generated.

FIGS. 57A-57C shows results demonstrating long-term persistence of in vivo edited B cells (non-IgH locus) in Ag-primed mice. FIG. 57A depicts an exemplary workflow for B cell specific promoter driven luciferase editing into Rosa locus of Cas9 mice primed with Ag. FIG. 57B depicts IVIS imaging results of B-cell specific luciferase expression in edited mice. FIG. 57C depicts longitudinal analysis of luciferase signal indicating durability of in vivo edited B cells in mice.

FIGS. 58A-58B depict results showing that AAV “Nluc-Ab1” editing into IgH locus enables in vivo tracking of BCR edited cells over time. FIG. 58A depicts an exemplary workflow. FIG. 58B depicts results showing tracking of BCR edited cells over time.

FIGS. 59A-59C depict results showing peritoneal cavity B cell editing achieved via TP delivery of AAV into unprimed Cas9Ready mice. FIG. 59A depicts an exemplary workflow. FIG. 59B depicts that luciferase signal was readily observed in all the draining lymph nodes of the peritoneal cavity of Cas9Ready mice edited with B cell specific luciferase. FIG. 59C depicts that nLuc positive signal is predominantly observed in B1b and B1a B cells of the peritoneal cavity.

### DETAILED DESCRIPTION

### Definitions

Unless defined otherwise, all technical and scientific terms used herein have the same meaning as commonly understood by one of ordinary skill in the art to which this invention belongs.

Singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “protein,” “polypeptide,” and “peptide,” used interchangeably herein, include polymeric forms of amino acids of any length, including coded and non-coded amino acids and chemically or biochemically modified or derivatized amino acids. The terms also include polymers that have been modified, such as polypeptides having modified peptide backbones.

The terms “nucleic acid” and “polynucleotide,” used interchangeably herein, include polymeric forms of nucleotides of any length, including ribonucleotides, deoxyribonucleotides, or analogs or modified versions thereof. They include single-, double-, and multi-stranded DNA or RNA, genomic DNA, cDNA, DNA-RNA hybrids, and polymers comprising purine bases, pyrimidine bases, or other natural, chemically modified, biochemically modified, non-natural, or derivatized nucleotide bases.

Nucleic acids are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. An end of an oligonucleotide is referred to as the “5′ end” if its 5′ phosphate is not linked to the 3′ oxygen of a mononucleotide pentose ring. An end of an oligonucleotide is referred to as the “3′ end” if its 3′ oxygen is not linked to a 5′ phosphate of another mononucleotide pentose ring. A nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements.

The term “wild type” includes entities having a structure and/or activity as found in a normal (as contrasted with mutant, diseased, altered, or so forth) state or context. Wild type genes and polypeptides often exist in multiple different forms (e.g., alleles).

The term “B cell” as used herein refers to a cell of B cell lineage. In some embodiments, B cells used in the compositions and methods of the present disclosure include, but are not limited to, B1 B cells, B2 B cells, memory B cells, plasmablasts, or plasma cells, or a combination thereof. In some embodiments, the B cells used in the compositions and methods of the present disclosure may be primary B cells.

The term “antibody” includes immunoglobulin molecules comprising four polypeptide chains, two heavy (H) chains and two light (L) chains inter-connected by disulfide bonds. Each heavy chain comprises a heavy chain variable domain (V_{H}) and a heavy chain constant region (C_{H}). The heavy chain constant region comprises at least three domains, C_{H}1, C_{H}2, C_{H}3 and optionally CH₄. Each light chain comprises a light chain variable domain (C_{H}) and a light chain constant region (CL). The heavy chain and light chain variable domains can be further subdivided into regions of hypervariability, termed complementarity determining regions (CDR), interspersed with regions that are more conserved, termed framework regions (FR). Each heavy and light chain variable domain comprises three CDRs and four FRs, arranged from amino-terminus to carboxy-terminus in the following order: FR1, CDR1, FR2, CDR2, FR3, CDR3, FR4 (heavy chain CDRs may be abbreviated as HCDR1, HCDR2 and HCDR3; light chain CDRs may be abbreviated as LCDR1, LCDR2 and LCDR3. Typical tetrameric antibody structures comprise two identical antigen-binding domains, each of which formed by association of the V_{H} and V_{L} domains, and each of which together with respective C_{H} and C_{L} domains form the antibody Fv region. Single domain antibodies comprise a single antigen-binding domain, e.g., a V_{H} or a V_{L}. As used herein, the term “antibody” encompasses, among others, B cell receptors (BCRs) and secreted antibodies. The term “antibody” also encompasses monoclonal antibodies, multispecific (e.g., bispecific) antibodies, human antibodies, humanized antibodies, chimeric antibodies, single-chain Fvs (scFv), single chain antibodies, single domain antibodies, Fab fragments, F(ab′) fragments, disulfide-linked Fvs (sdFv), intrabodies, minibodies, diabodies and anti-idiotypic (anti-Id) antibodies (including, e.g., anti-Id antibodies to antigen-specific TCR), and epitope-binding fragments of any of the above. The terms “antibody” and “antibodies” also refer to covalent diabodies such as those disclosed in U.S. Pat. Appl. Pub. 2007/0004909 and Ig-DARTS such as those disclosed in U.S. Pat. Appl. Pub. 2009/0060910. Antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The antigen-binding domain of an antibody, e.g., the part of an antibody that recognizes and binds to the epitope of an antigen, is also referred to as a “paratope.” It is a small region (of 5 to 10 amino acids) of an antibody's Fv region, part of the fragment antigen-binding (Fab region), and may contains parts of the antibody's heavy and/or light chains. A paratope specifically binds an epitope when the paratope binds the epitope with a high affinity. The term “high affinity” antibody refers to an antibody that has a K_{D} with respect to its target epitope about of 10⁻⁹ M or lower (e.g., about 1×10⁻⁹ M, 1×10⁻¹⁰ M, 1×10⁻¹¹ M, or about 1×10⁻¹² M). In one embodiment, K_{D} is measured by surface plasmon resonance, e.g., BIACORE™; in another embodiment, K_{D} is measured by ELISA.

The phrase “complementarity determining region,” or the term “CDR,” includes an amino acid sequence encoded by a nucleic acid sequence of an organism's immunoglobulin genes that normally (i.e., in a wild-type animal) appears between two framework regions in a variable region of a light or a heavy chain of an immunoglobulin molecule (e.g., an antibody or a T cell receptor). A CDR can be encoded by, for example, a germ line sequence or a rearranged or unrearranged sequence, and, for example, by a naïve or a mature B cell or a T cell. A CDR can be somatically mutated (e.g., vary from a sequence encoded in an animal's germ line), humanized, and/or modified with amino acid substitutions, additions, or deletions. In some circumstances (e.g., for a CDR3), CDRs can be encoded by two or more sequences (e.g., germ line sequences) that are not contiguous (e.g., in an unrearranged nucleic acid sequence) but are contiguous in a B cell nucleic acid sequence, e.g., as the result of splicing or connecting the sequences (e.g., V-D-J recombination to form a heavy chain CDR3).

An “epitope” is the part of a macromolecule that is recognized by the immune system, specifically by antibodies, B cells, or cytotoxic T cells. Although epitopes are usually thought to be derived from nonself proteins, sequences derived from the host that can be recognized are also classified as epitopes. Epitopes have a length of at least 4 amino acids, preferably 4 to 30 amino acids, more preferably 5 to 20 amino acids, especially 5 to 15 amino acids. Epitopes can be linear or three-dimensional formed typically by amino acids that are distant from each other in the primary protein structure but become closely related in a secondary and/or tertiary structure. Epitopes that are specifically recognized by B cells are referred to as B-cell epitopes.

The phrase “light chain” includes an immunoglobulin light chain sequence from any organism, and unless otherwise specified includes human κ and λ light chains and a VpreB, as well as surrogate light chains. Light chain variable domains typically include three light chain CDRs and four framework (FR) regions, unless otherwise specified. Generally, a full-length light chain includes, from amino terminus to carboxyl terminus, a variable domain that includes FR1-CDR1-FR2-CDR2-FR3-CDR3-FR4, and a light chain constant region. A light chain variable domain is encoded by a light chain variable region gene sequence, which generally comprises V_{L} and J_{L} segments, derived from a repertoire of V and J segments present in the germ line. Sequences, locations and nomenclature for V and J light chain segments for various organisms can be found in IMGT database, imgt.org. Light chains include those, e.g., that do not selectively bind either a first or a second epitope selectively bound by the epitope-binding protein in which they appear. Light chains also include those that bind and recognize, or assist the heavy chain or another light chain with binding and recognizing, one or more epitopes selectively bound by the epitope-binding protein in which they appear. Common or universal light chains include those derived from a human Vκ1-39Jκ gene or a human Vκ3-20Jκ gene, and include somatically mutated (e.g., affinity matured) versions of the same. Exemplary human V_{L} segments include a human Vκ1-39 gene segment, a human Vκ3-20 gene segment, a human Vκ1-40 gene segment, a human Vκ1-44 gene segment, a human Vκ2-8 gene segment, a human Vκ2-14 gene segment, and human Vκ3-21 gene segment, and include somatically mutated (e.g., affinity matured) versions of the same. Light chains can be made that comprise a variable domain from one organism (e.g., human or rodent, e.g., rat or mouse; or bird, e.g., chicken) and a constant region from the same or a different organism (e.g., human or rodent, e.g., rat or mouse; or bird, e.g., chicken).

The phrase “heavy chain,” or “immunoglobulin heavy chain” includes an immunoglobulin heavy chain sequence, including immunoglobulin heavy chain constant region sequence, from any organism. Heavy chain variable domains include three heavy chain CDRs and four FR regions, unless otherwise specified. Fragments of heavy chains include CDRs, CDRs and FRs, and combinations thereof. A typical heavy chain has, following the variable domain (from N-terminal to C-terminal), a C_{H}1 domain, a hinge, a C_{H}2 domain, and a C_{H}3 domain. A functional fragment of a heavy chain includes a fragment that is capable of specifically recognizing an epitope (e.g., recognizing the epitope with a K_{D} in the micromolar, nanomolar, or picomolar range), that is capable of expressing and secreting from a cell, and that comprises at least one CDR. heavy chain variable domains are encoded by variable region nucleotide sequence, which generally comprises V_{H}, D_{H}, and J_{H} segments derived from a repertoire of V_{H}, D_{H}, and J_{H} segments present in the germline. Sequences, locations and nomenclature for V, D, and J heavy chain segments for various organisms can be found in IMGT database, which is accessible via the internet on the world wide web (www) at the URL “imgt.org.”

The term “heavy chain only antibody,” “heavy chain only antigen binding protein,” “single domain antigen binding protein,” “single domain binding protein” or the like refers to a monomeric or homodimeric immunoglobulin molecule comprising an immunoglobulin-like chain comprising a variable domain operably linked to a heavy chain constant region, that is unable to associate with a light chain because the heavy chain constant region typically lacks a functional C_{H}1 domain. Accordingly, the term “heavy chain only antibody,” “heavy chain only antigen binding protein,” “single domain antigen binding protein,” “single domain binding protein” or the like encompasses a both (i) a monomeric single domain antigen binding protein comprising one of the immunoglobulin-like chain comprising a variable domain operably linked to a heavy chain constant region lacking a functional C_{H}1 domain, or (ii) a homodimeric single domain antigen binding protein comprising two immunoglobulin-like chains, each of which comprising a variable domain operably linked to a heavy chain constant region lacking a functional C_{H}1 domain. In various aspects, a homodimeric single domain antigen binding protein comprises two identical immunoglobulin-like chains, each of which comprising an identical variable domain operably linked to an identical heavy chain constant region lacking a functional C_{H}1 domain. Additionally, each immunoglobulin-like chain of a single domain antigen binding protein comprises a variable domain, which may be derived from heavy chain variable region gene segments (e.g., V_{H}, D_{H}, J_{H}), light chain gene segments (e.g., V_{L}, J_{L}), or a combination thereof, linked to a heavy chain constant region (C_{H}) gene sequence comprising a deletion or inactivating mutation in a C_{H}1 encoding sequence (and, optionally, a hinge region) of a heavy chain constant region gene, e.g., IgG, IgA, IgE, IgD, or a combination thereof. A single domain antigen binding protein comprising a variable domain derived from heavy chain gene segments may be referred to as a “V_{H}-single domain antibody” or “V_{H}-single domain antigen binding protein”, see, e.g., U.S. Pat. No. 8,754,287; U.S. Patent Publication Nos. 20140289876; 20150197553; 20150197554; 20150197555; 20150196015; 20150197556 and 20150197557, each of which is incorporated in its entirety by reference. A single domain antigen binding protein comprising a variable domain derived from light chain gene segments may be referred to as a “V_{L}-single domain antibody” or “V_{L}-single domain antigen binding protein,” see, e.g., U.S. Publication No. 20150289489, incorporated in its entirety by reference.

The term “about” or “approximately” includes being within a statistically meaningful range of a value. Such a range can be within an order of magnitude, preferably within 50%, more preferably within 20%, still more preferably within 10%, and even more preferably within 5% of a given value or range. The allowable variation encompassed by the term “about” or “approximately” depends on the particular system under study, and can be readily appreciated by one of ordinary skill in the art.

The term “affinity tag” includes a polypeptide sequence that is a member of a specific binding pair, e.g., that specifically binds with another polypeptide sequence, e.g., an antibody paratope, with high affinity. Exemplary and non-limiting affinity tags include hexahistidine tag, FLAG tag, Strep II tag, streptavidin-binding peptide (SBP) tag, calmodulin-binding peptide (CBP), glutathione S-transferase (GST), maltose-binding protein (MBP), S-tag, HA tag, and c-Myc tag. (Reviewed in Zhao et al. (2013) J. Analytical Meth. Chem. 1-8; incorporated herein by reference).

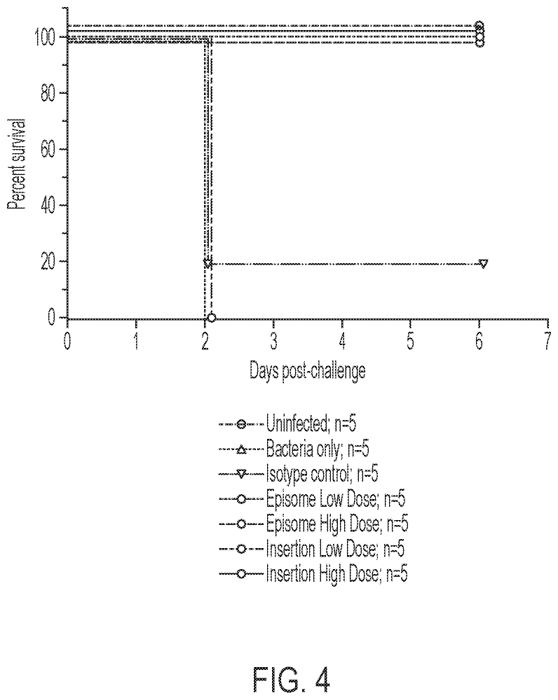

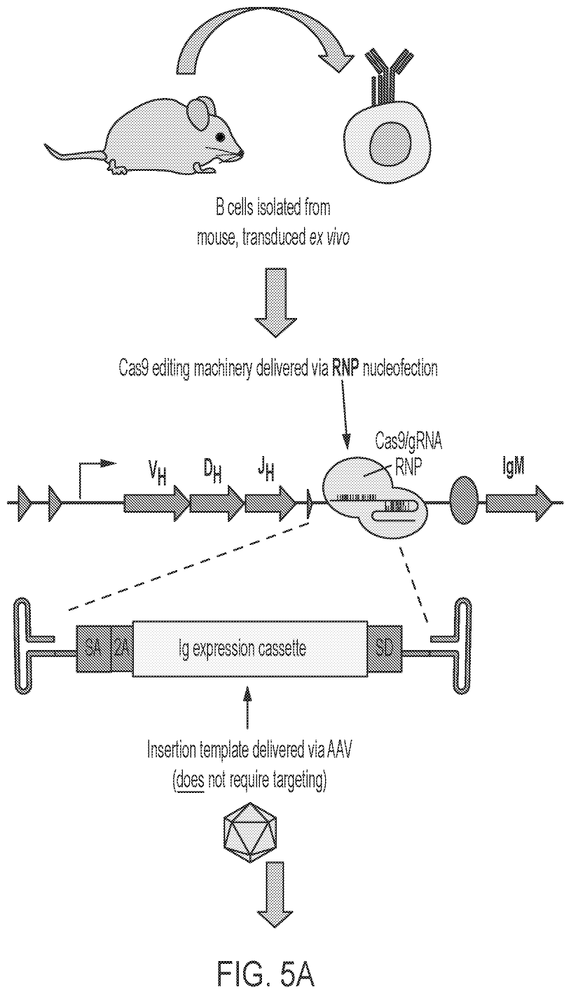

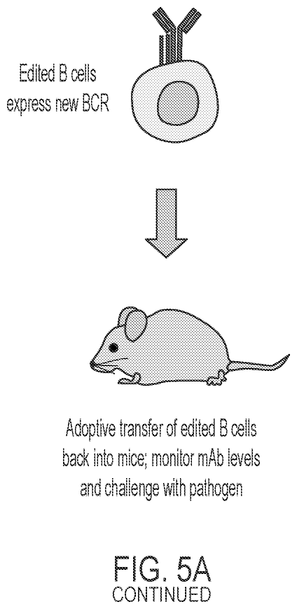

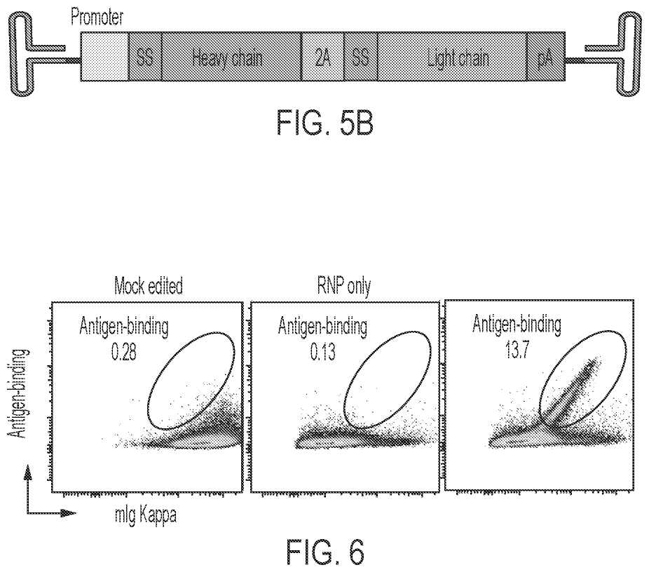

The term “capsid protein” includes a protein that is part of the capsid of the virus. For adeno-associated viruses (AAV), the capsid proteins are generally referred to as VP1, VP2 and/or VP3, and each are encoded by a single cap gene. For AAV, the three AAV capsid proteins are produced in an overlapping fashion from the cap open reading frame (ORF) via alternative mRNA splicing and/or alternative translational start codon usage, although all three proteins use a common stop codon. Warrington et al. (2004) J. Virol. 78:6595, incorporated herein by reference in its entirety. VP1 of AAV2 is generally translated from an ATG start codon (amino acid M1) on a 2.4-kb mRNA, while VP2 and VP3 of AAV2 arise from a smaller 2.3-kb mRNA, using a weaker ACG start codon for VP2 production (amino acid T138) and readthrough translation to the next available ATG codon (amino acid M203) for the production of the most abundant capsid protein, VP3. Warrington, supra; Rutledge et al. (1998) J. Virol. 72:309-19, incorporated herein by reference in its entirety. The amino acid sequences of capsid proteins of adeno-associated viruses are well-known in the art and generally conserved, particularly upon the dependoparvoviruses. See, Rutledge et al., supra. For example, Rutledge et al. (1998), supra, provides at FIG. 4B amino acid sequence alignments for VP1, VP2, and VP3 capsid proteins of AAV2, AAV3, AAV4 and AAV6, wherein the start sites for each of the VP1, VP2, and VP3 capsid proteins are indicated by arrows and the variable domains are boxed. Accordingly, although amino acid positions provided herein may be provided in relation to the VP1 capsid protein of the AAV, a skilled artisan would be able to respectively and readily determine the position of that same amino acid within the VP2 and/or VP3 capsid protein of the AAV, and the corresponding position of amino acids among different serotypes. Additionally, a skilled artisan would be able to swap domains between capsid proteins of a different AAV serotypes for the formation of a “chimeric capsid protein.”

Domain swapping between two AAV capsid protein constructs for the generation of a “chimeric AAV capsid protein” has been described, see, e.g., Shen et al. (2007) Mol. Therapy 15(11):1955-1962, incorporated herein in its entirety by reference. A “chimeric AAV capsid protein” includes an AAV capsid protein that comprises amino acid sequences, e.g., domains, from two or more different AAV serotypes and that is capable of forming and/or forms an AAV-like viral capsid/viral particle. A chimeric AAV capsid protein is encoded by a chimeric AAV capsid gene, e.g., a nucleotide comprising a plurality, e.g., at least two, nucleic acid sequences, each of which plurality is identical to a portion of a capsid gene encoding a capsid protein of distinct AAV serotypes, and which plurality together encodes a functional chimeric AAV capsid protein. Reference to a chimeric capsid protein in relation to a specific AAV serotype indicates that the capsid protein comprises one or more domains from a capsid protein of that serotype and one or more domains from a capsid protein of a different serotype. For example, an AAV2 chimeric capsid protein includes a capsid protein comprising one or more domains of an AAV2 VP1, VP2, and/or VP3 capsid protein and one or more domains of a VP1, VP2, and/or VP3 capsid protein of a different AAV.

A “mosaic capsid” comprises at least two sets of VP1, VP2, and/or VP3 proteins, each set of which is encoded by a different cap gene.

In some embodiments, a mosaic capsid described herein comprises recombinant VP1, VP2, and/or VP3 proteins encoded by a cap gene genetically modified with an insertion of a nucleic acid sequence encoding a heterologous epitope, and further comprises VP1, VP2, and/or VP3 proteins encoded by a reference cap gene, e.g., a wildtype reference cap gene encoding the wildtype VP1, VP2, and/or VP3 proteins of the same AAV serotype as the recombinant VP1, VP2, and/or VP3 proteins, a control reference cap gene encoding VP1, VP2, and/or VP3 proteins identical to the recombinant VP1, VP2, and VP3 proteins but for the absence of the heterologous epitope, a mutated wildtype reference cap gene encoding substantially wildtype VP1, VP2, and/or VP3 proteins of the same AAV serotype as the recombinant VP1, VP2, and/or VP3 proteins but for a mutation (e.g., insertion, substitution, deletion), which mutation preferably reduces the tropism of the wildtype VP1, VP2, and VP3 proteins. In some embodiments, the reference capsid protein is a chimeric reference protein comprising at least one domain of VP1, VP2, and/or VP3 proteins of the same AAV serotype as the recombinant VP1, VP2, and/or VP3 proteins. In some embodiments, the reference cap gene encodes a chimeric VP1, VP2, and/or VP3 protein.

The term “recombinant capsid protein” includes a capsid protein that has at least one mutation in comparison to the corresponding capsid protein of the wild-type virus, which may be a reference and/or control virus for comparative study. A recombinant capsid protein includes a capsid protein that comprises a heterologous epitope, which may be inserted into and/or displayed by the capsid protein. “Heterologous” in this context means heterologous as compared to the virus, from which the capsid protein is derived. The inserted amino acids can simply be inserted between two given amino acids of the capsid protein. An insertion of amino acids can also go along with a deletion of given amino acids of the capsid protein at the site of insertion, e.g., 1 or more capsid protein amino acids are substituted by 5 or more heterologous amino acids.

“Retargeting” or “redirecting” may include a scenario in which the wildtype vector targets several cells within a tissue and/or several organs within an organism, which general targeting of the tissue or organs is reduced to abolished by insertion of the heterologous epitope, and which retargeting to more a specific cell in the tissue or a specific organ in the organism is achieved with the targeting ligand that binds a marker expressed by the specific cell. Such retargeting or redirecting may also include a scenario in which the wildtype vector targets a tissue, which targeting of the tissue is reduced to abolished by insertion of the heterologous epitope, and which retargeting to a completely different tissue is achieved with the targeting ligand.

The phrase “Inverted terminal repeat” or “ITR” includes symmetrical nucleic acid sequences in the genome of adeno-associated viruses (AAV) required for efficient replication. ITR sequences are located at each end of the AAV DNA genome. The ITRs serve as the origins of replication for viral DNA synthesis and are essential cis components for generating AAV integrating vectors.

“Codon optimization” takes advantage of the degeneracy of codons, as exhibited by the multiplicity of three-base pair codon combinations that specify an amino acid, and generally includes a process of modifying a nucleic acid sequence for enhanced expression in particular host cells (e.g., packaging cells) and/or target cells by replacing at least one codon of the native sequence with a codon that is more frequently or most frequently used in the genes of the host cells and/or target cells while maintaining the native amino acid sequence. For example, a nucleic acid encoding a Cas9 protein can be modified to substitute codons having a higher frequency of usage in a given prokaryotic or eukaryotic cell, including a bacterial cell, a yeast cell, a human cell, a non-human cell, a mammalian cell, a rodent cell, a mouse cell, a rat cell, a hamster cell, or any other host and/or target cell, as compared to the naturally occurring nucleic acid sequence. Codon usage tables are readily available, for example, at the “Codon Usage Database.” These tables can be adapted in a number of ways. See Nakamura et al. (2000) Nucleic Acids Research 28:292, herein incorporated by reference in its entirety for all purposes. Computer algorithms for codon optimization of a particular sequence for expression in a particular host and/or target are also available (see, e.g., Gene Forge).

A “promoter” is a regulatory region of DNA usually comprising a TATA box capable of directing RNA polymerase II to initiate RNA synthesis at the appropriate transcription initiation site for a particular polynucleotide sequence. A promoter may additionally comprise other regions which influence the transcription initiation rate. As used herein, the term “promoter” encompasses enhancers. The promoter sequences disclosed herein modulate transcription of an operably linked polynucleotide. A promoter can be active in one or more of the cell types disclosed herein (e.g., a eukaryotic cell, a non-human mammalian cell, a human cell, a rodent cell, a pluripotent cell, a one-cell stage embryo, a differentiated cell, or a combination thereof). A promoter can be, for example, a constitutively active promoter, a conditional promoter, an inducible promoter, a temporally restricted promoter (e.g., a developmentally regulated promoter), or a spatially restricted promoter (e.g., a cell-specific or tissue-specific promoter). RNA Pol III promoters are frequently used to express small RNAs, such as small interfering RNA (siRNA)/short hairpin RNA (shRNA) and guide RNA sequences used in CRISPR-Cas9 systems. Examples of RNA Pol III promoters that can be used in the invention include, but are not limited to, the human U6 promoter, a rat U6 polymerase III promoter, or a mouse U6 polymerase III promoter, and the HI promoter, which are described in, for example Goomer and Kunkel, Nucl. Acids Res., 20 (18): 4903-4912 (1992), and Myslinski et al., Nucleic Acids Res., 29(12): 2502-9 (2001). Examples of promoters can be found, for example, in WO 2013/176772, herein incorporated by reference in its entirety for all purposes.

Examples of inducible promoters include, for example, chemically regulated promoters and physically-regulated promoters. Chemically regulated promoters include, for example, alcohol-regulated promoters (e.g., an alcohol dehydrogenase (alcA) gene promoter), tetracycline-regulated promoters (e.g., a tetracycline-responsive promoter, a tetracycline operator sequence (tetO), a tet-On promoter, or a tet-Off promoter), steroid regulated promoters (e.g., a rat glucocorticoid receptor, a promoter of an estrogen receptor, or a promoter of an ecdysone receptor), or metal-regulated promoters (e.g., a metalloprotein promoter). Physically regulated promoters include, for example temperature-regulated promoters (e.g., a heat shock promoter) and light-regulated promoters (e.g., a light-inducible promoter or a light-repressible promoter).

Tissue-specific promoters can be, for example, neuron-specific promoters, glia-specific promoters, muscle cell-specific promoters, heart cell-specific promoters, kidney cell-specific promoters, bone cell-specific promoters, endothelial cell-specific promoters, or immune cell-specific promoters (e.g., a B cell promoter or a T cell promoter).

Developmentally regulated promoters include, for example, promoters active only during an embryonic stage of development, or only in an adult cell.

A “self-cleaving peptide” or a “self-cleaving sequence” encoding a self-cleaving domain is a peptide or coding sequence, respectively, that induces ribosomal skipping during protein translation, resulting in a break. Suitable protease cleavages sites and self-cleaving peptides are known to the skilled person (see, e.g., in Ryan et al. (1997) J. Gener. Virol. 78, 699-722; Scymczak et al. (2004) Nature Biotech. 5, 589-594). Examples of protease cleavage sites are the cleavage sites of potyvirus NIa proteases (e.g. tobacco etch virus protease), potyvirus HC proteases, potyvirus P1 (P35) proteases, byovirus NIa proteases, byovirus RNA-2-encoded proteases, aphthovirus L proteases, enterovirus 2A proteases, rhinovirus 2A proteases, picorna 3C proteases, comovirus 24K proteases, nepovirus 24K proteases, RTSV (rice tungro spherical virus) 3C-like protease, PYVF (parsnip yellow fleck virus) 3C-like protease, thrombin, factor Xa and enterokinase. Due to its high cleavage stringency, TEV (tobacco etch virus) protease cleavage sites are particularly preferred. In some embodiments, the isolated nucleic acid includes a self-cleaving peptidyl sequence encoding a self-cleaving peptidyl domain between the heavy chain sequence and the light chain sequence. Preferred self-cleaving peptides (also called “cis-acting hydrolytic elements”, CHYSEL; see deFelipe (2002) Curr. Gene Ther. 2, 355-378) are derived from potyvirus and cardiovirus 2A peptides. Especially preferred self-cleaving peptides are selected from 2A peptides derived from FMDV (foot-and-mouth disease virus), equine rhinitis A virus, Thosea asigna virus and porcine teschovirus.

In some embodiments, self-cleaving peptidyl linker sequences used herein is a 2A sequence. In some embodiments, the self-cleaving peptidyl linker sequence is a T2A sequence, a P2A sequence, E2A sequence, or F2A sequence. In some embodiments, the self-cleaving peptidyl linker sequence is a foot-and-mouth disease virus sequence. In some embodiments, the self-cleaving peptidyl linker sequence is PVKQLLNFDLLKLAGDVESNPGP (SEQ ID NO: 6). In some embodiments, the self-cleaving peptidyl linker sequence is an equine rhinitis A virus sequence. In some embodiments, the self-cleaving peptidyl linker sequence is QCTNYALLKLAGDVESNPGP (SEQ ID NO: 7). In embodiments, the self-cleaving peptidyl linker sequence is a porcine teschovirus 1 sequence. In embodiments, the self-cleaving peptidyl linker sequence is ATNFSLLKQAGDVEENPGP (SEQ ID NO: 8). In some embodiments, the self-cleaving peptidyl linker sequence is Thosea asigna virus sequence. In some embodiments, the self-cleaving peptidyl linker sequence is EGRGSLLTCGDVESNPGP (SEQ ID NO: 9). In some embodiments, the light chain sequence is 3′ to the heavy chain sequence. In some embodiments, the light chain sequence is 5′ to the heavy chain sequence.

The phrase “operably linked”, as used herein, includes a physical juxtaposition (e.g., in three-dimensional space) of components or elements that interact, directly or indirectly with one another, or otherwise coordinate with each other to participate in a biological event, which juxtaposition achieves or permits such interaction and/or coordination. To give but one example, a control sequence (e.g., an expression control sequence) in a nucleic acid is said to be “operably linked” to a coding sequence when it is located relative to the coding sequence such that its presence or absence impacts expression and/or activity of the coding sequence. In many embodiments, “operable linkage” involves covalent linkage of relevant components or elements with one another. Those skilled in the art will readily appreciate, however, that in some embodiments, covalent linkage is not required to achieve effective operable linkage. For example, in some embodiments, nucleic acid control sequences that are operably linked with coding sequences that they control are contiguous with the nucleotide of interest. Alternatively or additionally, in some embodiments, one or more such control sequences acts in trans or at a distance to control a coding sequence of interest. In some embodiments, the term “expression control sequence” as used herein refers to polynucleotide sequences which are necessary and/or sufficient to effect the expression and processing of coding sequences to which they are ligated. In some embodiments, expression control sequences may be or comprise appropriate transcription initiation, termination, promoter and/or enhancer sequences; efficient RNA processing signals such as splicing and polyadenylation signals; sequences that stabilize cytoplasmic mRNA; sequences that enhance translation efficiency (e.g., Kozak consensus sequence); sequences that enhance protein stability; and/or, in some embodiments, sequences that enhance protein secretion. In some embodiments, one or more control sequences are preferentially or exclusively active in a particular host cell or organism, or type thereof. To give but one example, in prokaryotes, control sequences typically include promoter, ribosomal binding site, and transcription termination sequence; in eukaryotes, in many embodiments, control sequences typically include promoters, enhancers, and/or transcription termination sequences. Those of ordinary skill in the art will appreciate from context that, in many embodiments, the term “control sequences” refers to components whose presence is essential for expression and processing, and in some embodiments, includes components whose presence is advantageous for expression (including, for example, leader sequences, targeting sequences, and/or fusion partner sequences).

“Specific binding pair,” “protein:protein binding pair” and the like includes two proteins (e.g., a first member (e.g., a first polypeptide) and a second cognate member (e.g., a second polypeptide)) that interact to form a covalent isopeptide bond under conditions that enable or facilitate isopeptide bond formation, wherein the term “cognate” refers to components that function together, i.e. to react together to form an isopeptide bond. Thus, two proteins that react together efficiently to form an isopeptide bond under conditions that enable or facilitate isopeptide bond formation can also be referred to as being a “complementary” pair of peptide linkers. Specific binding pairs capable of interacting to form a covalent isopeptide bond are reviewed in Veggiani et al. (2014) Trends Biotechnol. 32:506, and include peptide:peptide binding pairs such as SpyTag:SpyCatcher; SpyTag002:SpyCatcher002; SpyTag003: SpyCatcher003; SpyTag:KTag; isopeptag:pilin C; SnoopTag:SnoopCatcher; SnoopTagJr:DogTag, etc. Generally, a peptide tag refers to member of a protein:protein binding pair, which is generally less than 30 amino acids in length, and which forms a covalent isopeptide bond with the second cognate protein, wherein the second cognate protein is generally larger, but may also be less than 30 amino acids in length such as in the SpyTag:KTag system.

The term “isopeptide bond” refers to an amide bond between a carboxyl or carboxamide group and an amino group at least one of which is not derived from a protein main chain or alternatively viewed is not part of the protein backbone. An isopeptide bond may form within a single protein or may occur between two peptides or a peptide and a protein. Thus, an isopeptide bond may form intramolecularly within a single protein or intermolecularly i.e. between two peptide/protein molecules, e.g. between two peptide linkers. Typically, an isopeptide bond may occur between a lysine residue and an asparagine, aspartic acid, glutamine, or glutamic acid residue or the terminal carboxyl group of the protein or peptide chain or may occur between the alpha-amino terminus of the protein or peptide chain and an asparagine, aspartic acid, glutamine or glutamic acid. Each residue of the pair involved in the isopeptide bond is referred to herein as a reactive residue. In preferred embodiments of the invention, an isopeptide bond may form between a lysine residue and an asparagine residue or between a lysine residue and an aspartic acid residue. Particularly, isopeptide bonds can occur between the side chain amine of lysine and carboxamide group of asparagine or carboxyl group of an aspartate.

The SpyTag:SpyCatcher system is described in U.S. Pat. No. 9,547,003 Zakeri et al. (2012) PNAS 109:E690-E697, and WO2019006046, each of which is incorporated herein in its entirety by reference, and is derived from the CnaB2 domain of the Streptococcus pyogenes fibronecting-binding protein FbaB. By splitting the domain, Zakeri et al. obtained a peptide “SpyTag” having the sequence AHIVMVDAYKPTK (SEQ ID NO: 13) which forms an amide bond to its cognate protein “SpyCatcher”. (Zakeri (2012), supra). An additional specific binding pair derived from CnaB2 domain is SpyTag:KTag, which forms an isopeptide bond in the presence of SpyLigase. (Fierer (2014) PNAS 111:E1176-1181) SpyLigase was engineered by excising the (3 strand from SpyCatcher that contains a reactive lysine, resulting in KTag, 10-residue peptide tag having the amino acid sequence ATHIKFSKRD (SEQ ID NO: 14). The SpyTag002:SpyCatcher002 system is described in Keeble et al (2017) Angew Chem Int Ed Engl 56:16521-25, incorporated herein in its entirety by reference. SpyTag002 has the amino acid sequence VPTIVMVDAYKRYK (SEQ ID NO: 15), and binds SpyCatcher002.

The SnoopTag:SnoopCatcher system is described in Veggiani (2016) PNAS 113:1202-07. The D4 Ig-like domain of RrgA, an adhesion from Streptococcus pneumoniae, was split to form SnoopTag (residues 734-745) and SnoopCatcher (residues 749-860). Incubation of SnoopTag and SnoopCatcher results in a spontaneous isopeptide bond that is specific between the complementary proteins. Veggiani (2016)), supra.

The isopeptag:pilin-C specific binding pair was derived from the major pilin protein Spy0128 from Streptococcus pyogenes. (Zakeir and Howarth (2010) J. Am. Chem. Soc. 132:4526-27). Isopeptag has the amino acid sequence TDKDMTITFTNKKDAE (SEQ ID NO: 16), and binds pilin-C (residues 18-299 of Spy0128). Incubation of SnoopTag and SnoopCatcher results in a spontaneous isopeptide bond that is specific between the complementary proteins. Zakeir and Howarth (2010), supra.

The term “peptide tag” includes polypeptides that are (1) heterologous to the protein which is tagged with the peptide tag, (2) a member of a specific protein:protein binding pair capable of forming an isopeptide bond, and (3) no more than 50 amino acids in length.

The term “detectable label” includes a polypeptide sequence that is a member of a specific binding pair, e.g., that specifically binds via a non-covalent bond with another polypeptide sequence, e.g., an antibody paratope, with high affinity. Exemplary and non-limiting detectable labels include hexahistidine tag, FLAG tag, Strep II tag, streptavidin-binding peptide (SBP) tag, calmodulin-binding peptide (CBP), glutathione S-transferase (GST), maltose-binding protein (MBP), S-tag, HA tag, and c-myc. (Reviewed in Zhao et al. (2013) J. Analytical Meth. Chem. 1-8; incorporated herein by reference). A common detectable label for primate AAV is the B1 epitope. Non-primate AAV capsid proteins of the invention, which do not naturally comprise the B1 epitope, may be modified herein to comprise a B1 epitope. Generally, non-primate AAV capsid proteins may comprise a sequence with substantial homology to the B1 epitope within the last 10 amino acids of the capsid protein. Accordingly, in some embodiments, a non-primate AAV capsid protein of the invention may be modified with one but less than five point mutations within the last 10 amino acids of the capsid protein such that the AAV capsid protein comprises a B1 epitope.

In various embodiments, Fc domains may be modified to have altered Fc receptor binding, which in turn affects effector function. In some embodiments, an engineered heavy chain constant region (CH), which includes the Fc domain, is chimeric. As such, a chimeric CH region combines CH domains derived from more than one immunoglobulin isotype. For example, a chimeric CH region comprises part or all of a CH2 domain derived from a human IgG1, human IgG2 or human IgG4 molecule, combined with part or all of a CH3 domain derived from a human IgG1, human IgG2 or human IgG4 molecule. In some embodiments, a chimeric CH region contain a chimeric hinge region. For example, a chimeric hinge may comprise an “upper hinge” amino acid sequence (amino acid residues from positions 216 to 227 according to EU numbering; amino acid residues from positions 226 to 240 according to Kabat numbering) derived from a human IgG1, a human IgG2 or a human IgG4 hinge region, combined with a “lower hinge” sequence (amino acid residues from positions 228 to 236 according to EU numbering; amino acid positions from positions 241 to 249 according to Kabat numbering) derived from a human IgG1, a human IgG2 or a human IgG4 hinge region. In some embodiments, the chimeric hinge region comprises amino acid residues derived from a human IgG1 or a human IgG4 upper hinge and amino acid residues derived from a human IgG2 lower hinge.

In some embodiments, the Fc domain may be engineered to activate all, some, or none of the normal Fc effector functions, without affecting the Fc-containing protein's (e.g. antibody's) desired pharmacokinetic properties. For examples of proteins comprising chimeric CH regions and having altered effector functions, see WO2014022540, which is herein incorporated in its entirety.

The term “transduction”, “transfection”, or “infection” or the like used herein interchangeably, refers to the introduction of a nucleic acid into a target cell, for example, by a viral vector. The term efficiency in relation to transduction or the like, e.g., “transduction efficiency” refers to the fraction (e.g., percentage) of cells expressing a nucleotide of interest after incubation with a set number of viral vectors comprising the nucleotide of interest. Well-known methods of determining transduction efficiency include fluorescence activated cell sorting of cells transduced with a fluorescent reporter gene, PCR for expression of the nucleotide of interest, etc.

The term “wild-type”, as used herein, includes an entity having a structure and/or activity as found in nature in a “normal” (as contrasted with mutant, diseased, altered, etc.) state or context. Those of ordinary skill in the art will appreciate that wildtype viral vectors, e.g., AAV vectors comprising wild-type capsid proteins, may be used as reference viral vector in comparative studies. Generally, a reference viral capsid protein/capsid/vector are identical to the test viral capsid protein/capsid/vector but for the change for which the effect is to be tested. For example, to determine the effect, e.g., on transduction efficiency, of inserting a heterologous epitope into a test viral vector, the transduction efficiencies of the test viral vector (in the absence or presence of an appropriate binding molecule) can be compared to the transduction efficiencies of a reference viral vector (in the absence or presence of an appropriate binding molecule if necessary) which is identical to the test viral vector in every instance (e.g., additional mutations, nucleotide of interest, numbers of viral vectors and target cells, etc.) except for the presence of a heterologous epitope.

“Complementarity” or “complementary” of nucleic acids means that a nucleotide sequence in one strand of nucleic acid, due to orientation of its nucleobase groups, forms hydrogen bonds with another sequence on an opposing nucleic acid strand. The complementary bases in DNA are typically A with T and C with G. In RNA, they are typically C with G and U with A. Complementarity can be perfect or substantial/sufficient. Perfect complementarity between two nucleic acids means that the two nucleic acids can form a duplex in which every base in the duplex is bonded to a complementary base by Watson-Crick pairing. “Substantial” or “sufficient” complementary means that a sequence in one strand is not completely and/or perfectly complementary to a sequence in an opposing strand, but that sufficient bonding occurs between bases on the two strands to form a stable hybrid complex in set of hybridization conditions (e.g., salt concentration and temperature). Such conditions can be predicted by using the sequences and standard mathematical calculations to predict the Tm (melting temperature) of hybridized strands, or by empirical determination of Tm by using routine methods. Tm includes the temperature at which a population of hybridization complexes formed between two nucleic acid strands are 50% denatured (i.e., a population of double-stranded nucleic acid molecules becomes half dissociated into single strands). At a temperature below the Tm, formation of a hybridization complex is favored, whereas at a temperature above the Tm, melting or separation of the strands in the hybridization complex is favored. Tm may be estimated for a nucleic acid having a known G+C content in an aqueous 1 M NaCl solution by using, e.g., Tm=81.5+0.41(% G+C), although other known Tm computations take into account nucleic acid structural characteristics.

“Hybridization condition” includes the cumulative environment in which one nucleic acid strand bonds to a second nucleic acid strand by complementary strand interactions and hydrogen bonding to produce a hybridization complex. Such conditions include the chemical components and their concentrations (e.g., salts, chelating agents, formamide) of an aqueous or organic solution containing the nucleic acids, and the temperature of the mixture. Other factors, such as the length of incubation time or reaction chamber dimensions may contribute to the environment. See, e.g., Sambrook et al., Molecular Cloning, A Laboratory Manual, 2.sup.nd ed., pp. 1.90-1.91, 9.47-9.51, 11.47-11.57 (Cold Spring Harbor Laboratory Press, Cold Spring Harbor, N.Y., 1989), herein incorporated by reference in its entirety for all purposes.

Hybridization requires that the two nucleic acids contain complementary sequences, although mismatches between bases are possible. The conditions appropriate for hybridization between two nucleic acids depend on the length of the nucleic acids and the degree of complementation, variables which are well known. The greater the degree of complementation between two nucleotide sequences, the greater the value of the melting temperature (Tm) for hybrids of nucleic acids having those sequences. For hybridizations between nucleic acids with short stretches of complementarity (e.g. complementarity over 35 or fewer, 30 or fewer, 25 or fewer, 22 or fewer, 20 or fewer, or 18 or fewer nucleotides) the position of mismatches becomes important (see Sambrook et al., supra, 11.7-11.8). Typically, the length for a hybridizable nucleic acid is at least about 10 nucleotides. Illustrative minimum lengths for a hybridizable nucleic acid include at least about 15 nucleotides, at least about 20 nucleotides, at least about 22 nucleotides, at least about 25 nucleotides, and at least about 30 nucleotides. Furthermore, the temperature and wash solution salt concentration may be adjusted as necessary according to factors such as length of the region of complementation and the degree of complementation.

The sequence of polynucleotide need not be 100% complementary to that of its target nucleic acid/target locus to be specifically hybridizable. Moreover, a polynucleotide may hybridize over one or more segments such that intervening or adjacent segments are not involved in the hybridization event (e.g., a loop structure or hairpin structure). A polynucleotide (e.g., gRNA) can comprise at least 70%, at least 80%, at least 90%, at least 95%, at least 99%, or 100% sequence complementarity to a target region within the target nucleic acid/target locus sequence to which they are targeted. For example, a gRNA in which 18 of 20 nucleotides are complementary to a target region, and would therefore specifically hybridize, would represent 90% complementarity. In this example, the remaining noncomplementary nucleotides may be clustered or interspersed with complementary nucleotides and need not be contiguous to each other or to complementary nucleotides.

Percent complementarity between particular stretches of nucleic acid sequences within nucleic acids can be determined routinely using BLAST programs (basic local alignment search tools) and PowerBLAST programs (Altschul et al. (1990) J. Mol. Biol. 215:403-410; Zhang and Madden (1997) Genome Res. 7:649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

“Sequence identity” or “identity” in the context of two polynucleotides or polypeptide sequences makes reference to the residues in the two sequences that are the same when aligned for maximum correspondence over a specified comparison window. When percentage of sequence identity is used in reference to proteins, residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

“Percentage of sequence identity” includes the value determined by comparing two optimally aligned sequences (greatest number of perfectly matched residues) over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity. Unless otherwise specified (e.g., the shorter sequence includes a linked heterologous sequence), the comparison window is the full length of the shorter of the two sequences being compared.

Unless otherwise stated, sequence identity/similarity values include the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. “Equivalent program” includes any sequence comparison program that, for any two sequences in question, generates an alignment having identical nucleotide or amino acid residue matches and an identical percent sequence identity when compared to the corresponding alignment generated by GAP Version 10.

The term “conservative amino acid substitution” refers to the substitution of an amino acid that is normally present in the sequence with a different amino acid of similar size, charge, or polarity. Examples of conservative substitutions include the substitution of a non-polar (hydrophobic) residue such as isoleucine, valine, or leucine for another non-polar residue. Likewise, examples of conservative substitutions include the substitution of one polar (hydrophilic) residue for another such as between arginine and lysine, between glutamine and asparagine, or between glycine and serine. Additionally, the substitution of a basic residue such as lysine, arginine, or histidine for another, or the substitution of one acidic residue such as aspartic acid or glutamic acid for another acidic residue are additional examples of conservative substitutions. Examples of non-conservative substitutions include the substitution of a non-polar (hydrophobic) amino acid residue such as isoleucine, valine, leucine, alanine, or methionine for a polar (hydrophilic) residue such as cysteine, glutamine, glutamic acid or lysine and/or a polar residue for a non-polar residue. Typical amino acid categorizations are summarized below.

| | | | | | |
|---|---|---|---|---|---|
| Alanine | Ala | A | Nonpolar | Neutral | 1.8 |
| Arginine | Arg | R | Polar | Positive | −4.5 |
| Asparagine | Asn | N | Polar | Neutral | −3.5 |
| Aspartic acid | Asp | D | Polar | Negative | −3.5 |
| Cysteine | Cys | C | Nonpolar | Neutral | 2.5 |
| Glutamic acid | Glu | E | Polar | Negative | −3.5 |
| Glutamine | Gln | Q | Polar | Neutral | −3.5 |
| Glycine | Gly | G | Nonpolar | Neutral | −0.4 |
| Histidine | His | H | Polar | Positive | −3.2 |
| Isoleucine | Ile | I | Nonpolar | Neutral | 4.5 |
| Leucine | Leu | L | Nonpolar | Neutral | 3.8 |
| Lysine | Lys | K | Polar | Positive | −3.9 |
| Methionine | Met | M | Nonpolar | Neutral | 1.9 |
| Phenylalanine | Phe | F | Nonpolar | Neutral | 2.8 |
| Proline | Pro | P | Nonpolar | Neutral | −1.6 |
| Serine | Ser | S | Polar | Neutral | −0.8 |
| Threonine | Thr | T | Polar | Neutral | −0.7 |
| Tryptophan | Trp | W | Nonpolar | Neutral | −0.9 |
| Tyrosine | Tyr | Y | Polar | Neutral | −1.3 |
| Valine | Val | V | Nonpolar | Neutral | 4.2 |

The term “in vitro” includes artificial environments and to processes or reactions that occur within an artificial environment (e.g., a test tube). The term “in vivo” includes natural environments (e.g., a cell or organism or body) and to processes or reactions that occur within a natural environment. The term “ex vivo” includes cells that have been removed from the body of an individual and to processes or reactions that occur within such cells.

Compositions or methods “comprising” or “including” one or more recited elements may include other elements not specifically recited. For example, a composition that “comprises” or “includes” a protein may contain the protein alone or in combination with other ingredients. The transitional phrase “consisting essentially of” means that the scope of a claim is to be interpreted to encompass the specified elements recited in the claim and those that do not materially affect the basic and novel characteristic(s) of the claimed invention. Thus, the term “consisting essentially of” when used in a claim of this invention is not intended to be interpreted to be equivalent to “comprising.”

An “individual” or “subject” or “animal” refers to humans, veterinary animals (e.g., cats, dogs, cows, horses, sheep, pigs, etc.) and experimental animal models of diseases (e.g., mice, rats). In some embodiments, the subject is a human.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition, but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable”, as used in connection with compositions described herein, refers to molecular entities and other ingredients of such compositions that are physiologically tolerable and do not typically produce untoward reactions when administered to a mammal (e.g., a human). Preferably, the term “pharmaceutically acceptable” means approved by a regulatory agency of the Federal or a state government or listed in the U.S. Pharmacopeia or other generally recognized pharmacopeia for use in mammals, and more particularly in humans.

In accordance with the disclosure herein, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition. Cold Spring Harbor, NY: Cold Spring Harbor Laboratory Press, 1989 (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); Ausubel, F. M. et al. (eds.). Current Protocols in Molecular Biology. John Wiley & Sons, Inc., 1994. These techniques include site directed mutagenesis as described in Kunkel, Proc. Natl. Acad. Sci. USA 82: 488-492 (1985), U.S. Pat. No. 5,071,743, Fukuoka et al., Biochem. Biophys. Res. Commun. 263: 357-360 (1999); Kim and Maas, BioTech. 28: 196-198 (2000); Parikh and Guengerich, BioTech. 24: 4 28-431 (1998); Ray and Nickoloff, BioTech. 13: 342-346 (1992); Wang et al., BioTech. 19: 556-559 (1995); Wang and Malcolm, BioTech. 26: 680-682 (1999); Xu and Gong, BioTech. 26: 639-641 (1999), U.S. Pat. Nos. 5,789,166 and 5,932,419, Hogrefe, Strategies 14. 3: 74-75 (2001), U.S. Pat. Nos. 5,702,931, 5,780,270, and 6,242,222, Angag and Schutz, Biotech. 30: 486-488 (2001), Wang and Wilkinson, Biotech. 29: 976-978 (2000), Kang et al., Biotech. 20: 44-46 (1996), Ogel and McPherson, Protein Engineer. 5: 467-468 (1992), Kirsch and Joly, Nucl. Acids. Res. 26: 1848-1850 (1998), Rhem and Hancock, J. Bacteriol. 178: 3346-3349 (1996), Boles and Miogsa, Curr. Genet. 28: 197-198 (1995), Barrenttino et al., Nuc. Acids. Res. 22: 541-542 (1993), Tessier and Thomas, Meths. Molec. Biol. 57: 229-237, and Pons et al., Meth. Molec. Biol. 67: 209-218.

“Optional” or “optionally” means that the subsequently described event or circumstance may or may not occur and that the description includes instances in which the event or circumstance occurs and instances in which it does not.

The present disclosure provides, among other things, a system for producing an antibody or an antigen-binding fragment thereof in a subject, comprising: a) a first component comprising a polynucleotide molecule, wherein the polynucleotide molecule comprises a sequence encoding the antibody or antigen-binding fragment thereof, and b) a second component comprising a gene editing molecule or a polynucleotide molecule comprising a sequence encoding said gene editing molecule.

In some embodiments, administration of the first and second components to the subject results in an integration of the sequence encoding the antibody or the antigen-binding fragment thereof into the DNA of a B cell and/or a hematopoietic stem cell (HSC) of the subject, causing the production of the antibody or the antigen-binding fragment in the subject.

In some embodiments, administration of the first and second components to a B cell and/or a hematopoietic stem cell (HSC) ex vivo isolated from the subject results in an integration of the sequence encoding the antibody or antigen-binding fragment thereof into the DNA of said cell to produce a modified B cell or a modified HSC, causing the production of the antibody or antigen-binding fragment thereof in the subject upon administration of the modified B cell or HSC to the subject.

In some embodiments, the first component and/or the second component are independently selected from a viral vector, a virus-like particle (VLP), a lipid nanoparticle (LNP), a liposome, and a ribonuclear protein (RNP) complex.

### Recombinant Virus Capsid Proteins, Viral Vectors and Nucleic Acids

In some embodiments, the first component and the second component are both viral vectors. In some embodiments, the viral vectors are derived from the same viral species. In other embodiments, the viral vectors are derived from different viral species.

Viral vectors that can be used in the compositions and methods of present application include, but are not limited to, an adenoviral vector, an adeno-associated viral (AAV) vector, a retrovirus (e.g., lentivirus), a baculoviral vector, a herpes viral vector, a cytomegalovirus (CMV), an Epstein-Barr virus (EBV), a mouse mammary tumor virus (MMTV), a human polyomavirus 2 (JC virus or John Cunningham virus), a hepatitis C virus (HCV), a hepatitis B virus (HBV), a human immunodeficiency virus 1 (HIV-1), an influenza virus, a norovirus, a measles virus, a polyoma virus, a rhabdovirus (e.g., vesicular stomatitis virus), or a variant thereof.

In some embodiments, one or both viral vectors used in the system of the present disclosure are adeno-associated virus (AAV) vectors. “AAV” is an abbreviation for adeno-associated virus and may be used to refer to the virus itself or derivatives thereof. AAVs are small, non-enveloped, single-stranded DNA viruses. Generally, a wildtype AAV genome is 4.7 kb and is characterized by two inverted terminal repeats (ITR) and two open reading frames (ORFs), rep and cap. The wildtype rep reading frame encodes four proteins of molecular weight 78 kD (“Rep78”), 68 kD (“Rep68”), 52 kD (“Rep52”) and 40 kD (“Rep 40”). Rep78 and Rep68 are transcribed from the p5 promoter, and Rep52 and Rep40 are transcribed from the p19 promoter. These proteins function mainly in regulating the transcription and replication of the AAV genome. The wildtype cap reading frame encodes three structural (capsid) viral proteins (VPs) having molecular weights of 83-85 kD (VP1), 72-73 kD (VP2) and 61-62 kD (VP3). More than 80% of total proteins in an AAV virion (capsid) comprise VP3; in mature virions VP1, VP2 and VP3 are found at relative abundance of approximately 1:1:10, although ratios of 1:1:8 have been reported. Padron et al. (2005) J. Virology 79:5047-58.

The genomic sequences of various serotypes of AAV, as well as the sequences of the native inverted terminal repeats (ITRs), Rep proteins, and capsid subunits are known in the art. Such sequences may be found in the literature or in public databases such as GenBank. See, e.g., GenBank Accession Numbers NC_002077 (AAV1), AF063497 (AAV1), NC001401 (AAV-2), AF043303 (AAV2), NC_001729 (AAV3), NC_001829 (AAV4), U89790 (AAV4), NC_006152 (AAV5), AF513851 (AAV7), AF513852 (AAV8), and NC_006261 (AAV8); the disclosures of which are incorporated by reference herein for teaching AAV nucleic acid and amino acid sequences. See also, e.g., Srivistava et al. (1983) J. Virology 45:555; Chiorini et al. (1998) J. Virology 71:6823; Chiorini et al. (1999) J. Virology 73: 1309; Bantel-Schaal et al. (1999) J. Virology 73:939; Xiao et al. (1999) J. Virology 73:3994; Muramatsu et al. (1996) Virology 221:208; Shade et al., (1986) J. Virol. 58:921; Gao et al. (2002) Proc. Nat. Acad. Sci. USA 99: 11854; Moris et al. (2004) Virology 33:375-383; US Patent Publication 20170130245; international patent publications WO 00/28061, WO 99/61601, WO 98/11244; and U.S. Pat. No. 6,156,303, each of which is incorporated by reference in its entirety by reference. Table 2 herein provides sequences of various non-primate AAV.

“AAV” encompasses all subtypes and both naturally occurring and modified forms that are well-known in the art. AAV includes primate AAV (e.g., AAV type 1 (AAV1), primate AAV type 2 (AAV2), primate AAV type 3 (AAV3B), primate AAV type 4 (AAV4), primate AAV type 5 (AAVS), primate AAV type 6 (AAV6), primate AAV type 7 (AAV7), primate AAV type 8 (AAV8), primate AAV type 9 (AAV9), AAV10, AAV11, AAV12, AAV13, AAVDJ, Anc80L65, AAV2G9, AAV-LK03, primate AAV type rh10 (AAV rh10), AAV type h10 (AAV h10), AAV type hu11 (AAV hu11), AAV type rh32.33 (AAV rh32.33), AAV retro (AAV retro), AAV PHP.B, AAV PHP.eB, AAV PITP.S, AAV2/8, etc., non-primate animal AAV (e.g., avian AAV (AAAV)) and other non-primate animal AAV such as mammalian AAV (e.g., bat AAV, sea lion AAV, bovine AAV, canine AAV, equine AAV, caprine AAV, and ovine AAV etc.), squamate AAV (e.g., snake AAV, bearded dragon AAV), etc. “Primate AAV” refers to AAV generally isolated from primates. Similarly, “non-primate animal AAV” refers to AAV isolated from non-primate animals.

In some embodiments, the AAV vector is derived from AAV1, AAV2, AAV6, AAV9, or AAV9.PHP.

Also included herein are recombinant viral particles that are genetically modified to display a heterologous amino acid sequence comprising a first member of a specific binding pair, wherein the amino acid sequence is less than 50 amino acids in length, and wherein the recombinant viral capsid/particle protein exhibits reduced to abolished natural tropism. In some embodiments, the viral particle further comprises a second cognate member of the specific binding pair, wherein the first and second members are covalently bonded, and wherein the second member is fused to a targeting ligand.

In some embodiments, the heterologous amino acid sequence comprises a first member of a specific binding pair and one or more linkers. In some embodiments, the heterologous amino acid sequence comprises a first member of a specific binding pair flanked by a linker, e.g., the heterologous amino acid sequence comprises from N-terminus to C-terminus a first linker, a first member of a specific binding pair, and a second linker. In some embodiments, the first and second linkers are each independently at least one amino acid in length. In some embodiments, the first and second linkers are identical.

Generally, a heterologous amino acid sequence as described herein, e.g., comprising a first member of a specific binding pair by itself or in combination with one or more linkers, is between about 5 amino acids to about 50 amino acids in length. In some embodiments, the heterologous amino acid sequence is at least 5 amino acids in length. In some embodiments, the heterologous amino acid sequence is 6 amino acids in length. In some embodiments, the heterologous amino acid sequence is 7 amino acids in length. In some embodiments, the heterologous amino acid sequence is 8 amino acids in length. In some embodiments, the heterologous amino acid sequence is 9 amino acids in length. In some embodiments, the heterologous amino acid sequence is 10 amino acids in length. In some embodiments, the heterologous amino acid sequence is 11 amino acids in length. In some embodiments, the heterologous amino acid sequence is 12 amino acids in length. In some embodiments, the heterologous amino acid sequence is 13 amino acids in length. In some embodiments, the heterologous amino acid sequence is 14 amino acids in length. In some embodiments, the heterologous amino acid sequence is 15 amino acids in length. In some embodiments, the heterologous amino acid sequence is 16 amino acids in length. In some embodiments, the heterologous amino acid sequence is 17 amino acids in length. In some embodiments, the heterologous amino acid sequence is 18 amino acids in length. In some embodiments, the heterologous amino acid sequence is 19 amino acids in length. In some embodiments, the heterologous amino acid sequence is 20 amino acids in length. In some embodiments, the heterologous amino acid sequence is 21 amino acids in length. In some embodiments, the heterologous amino acid sequence is 22 amino acids in length. In some embodiments, the heterologous amino acid sequence is 23 amino acids in length. In some embodiments, the heterologous amino acid sequence is 24 amino acids in length. In some embodiments, the heterologous amino acid sequence is 25 amino acids in length. In some embodiments, the heterologous amino acid sequence is 26 amino acids in length. In some embodiments, the heterologous amino acid sequence is 27 amino acids in length. In some embodiments, the heterologous amino acid sequence is 28 amino acids in length. In some embodiments, the heterologous amino acid sequence is 29 amino acids in length. In some embodiments, the heterologous amino acid sequence is 30 amino acids in length. In some embodiments, the heterologous amino acid sequence is 31 amino acids in length. In some embodiments, the heterologous amino acid sequence is 32 amino acids in length. In some embodiments, the heterologous amino acid sequence is 33 amino acids in length. In some embodiments, the heterologous amino acid sequence is 34 amino acids in length. In some embodiments, the heterologous amino acid sequence is 35 amino acids in length. In some embodiments, the heterologous amino acid sequence is 36 amino acids in length. In some embodiments, the heterologous amino acid sequence is 37 amino acids in length. In some embodiments, the heterologous amino acid sequence is 38 amino acids in length. In some embodiments, the heterologous amino acid sequence is 39 amino acids in length. In some embodiments, the heterologous amino acid sequence is 40 amino acids in length. In some embodiments, the heterologous amino acid sequence is 41 amino acids in length. In some embodiments, the heterologous amino acid sequence is 42 amino acids in length. In some embodiments, the heterologous amino acid sequence is 43 amino acids in length. In some embodiments, the heterologous amino acid sequence is 44 amino acids in length. In some embodiments, the heterologous amino acid sequence is 45 amino acids in length. In some embodiments, the heterologous amino acid sequence is 46 amino acids in length. In some embodiments, the heterologous amino acid sequence is 47 amino acids in length. In some embodiments, the heterologous amino acid sequence is 48 amino acids in length. In some embodiments, the heterologous amino acid sequence is 49 amino acids in length. In some embodiments, the heterologous amino acid sequence is 50 amino acids in length.

In some embodiments, the specific binding pair is a SpyTag:SpyCatcher binding pair, wherein the first member is SpyTag, and wherein the second cognate member is SpyCatcher. In some embodiments, the specific binding pair is SpyTag:KTag, wherein the first member is SpyTag and wherein the second cognate member is KTag. In some embodiments, the specific binding pair is SpyTag:KTag, wherein the first member is KTag and wherein the second cognate member is SpyTag. In some embodiments, the specific binding pair is isopeptag:pilin-C, wherein the first member is isopeptag, and wherein the second cognate member is pilin-C, or a portion thereof. In some embodiments, the specific binding pair is SnoopTag:SnoopCatcher, and the first member is SnoopTag, and the second cognate member is SnoopCatcher.

In some embodiments, a recombinant viral capsid protein described herein is derived from an adeno-associated virus (AAV) capsid gene, e.g., is a genetically modified capsid protein of an AAV serotype selected from AAV1, AAV2, AAV3, AAV4, AAV5, AAV6, AAV7, AAV8 and AAV9. In some embodiments, the recombinant viral capsid protein is derived from an AAV2 capsid gene, an AAV6 capsid gene, an AAV1 capsid gene, or an AAV9 capsid gene. In some embodiments, the recombinant viral capsid protein is derived from an AAV2 capsid gene, e.g., is a genetically modified AAV2 VP1 capsid protein. In some embodiments, the recombinant viral capsid protein is derived from an AAV1 capsid gene, e.g., is a genetically modified AAV1 VP1 capsid protein. In some embodiments the recombinant viral capsid protein is derived from an AAV9 capsid gene, e.g., is a genetically modified AAV9 VP1 capsid protein. In some embodiments, the recombinant viral capsid protein is derived from an AAV6 capsid gene, e.g., is a genetically modified VP1 capsid protein of AAV6. In some embodiments, a heterologous epitope is inserted into I-453 of an AAV9 capsid protein.

Generally, a recombinant viral capsid protein as described herein comprises a heterologous epitope inserted into and/or displayed by the capsid protein such that the heterologous epitope reduces and/or abolishes the natural tropism of the capsid protein or capsid comprising same. In some embodiments, the heterologous epitope is inserted into a region of the capsid protein involved with the natural tropism of the wildtype reference capsid protein, e.g., a region of the capsid protein involved with cell receptor. In some embodiments, the heterologous epitope is inserted into and/or displayed by a knob domain of an Ad fiber protein. In some embodiments, the heterologous epitope is inserted into and/or displayed by the HI loop of an Ad fiber protein. In some embodiments, the heterologous epitope is inserted after an amino acid position selected from the group consisting of G453 of AAV2 capsid protein VP1, N587 of AAV2 capsid protein VP1, Q585 of AAV6 capsid protein VP1, G453 of AAV9 capsid protein VP1, and A589 of AAV9 capsid protein VP1. In some embodiments, the heterologous epitope is inserted and/or displayed between amino acids N587 and R588 of an AAV2 VP1 capsid. Additional suitable insertion sites identified by using AAV2 are well known in the art (Wu et al. (2000). Virol. 74:8635-8647) and include I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713 and I-716. A recombinant virus capsid protein as described herein may be an AAV2 capsid protein comprising a heterologous epitope inserted into a position selected from the group consisting of I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713, I-716, and a combination thereof. Additional suitable insertion sites identified by using additional AAV serotypes are well-known and include I-587 (AAV1), I-589 (AAV1), I-585 (AAV3), 1-585 (AAV4), and I-585 (AAV5). In some embodiments, a recombinant virus capsid protein as described herein may be an AAV2 capsid protein comprising a heterologous epitope inserted into a position selected from the group consisting of I-587 (AAV1), I-589 (AAV1), I-585 (AAV3), I-585 (AAV4), I-585 (AAV5), and a combination thereof.

The used nomenclature I-### herein refers to the insertion site with ### naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s) N- or C-terminal of the given amino acid. Additionally, the positions referred to herein are relative to the VP1 protein encoded by an AAV capsid gene, and corresponding positions (and mutations thereof) may be easily identified for the VP2 and VP3 capsid proteins encoding by the capsid gene by performing a sequence alignment of the VP1, VP2 and VP3 proteins encoding by the reference AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site I-587. Therefore, the present invention encompasses structural genes of AAV with corresponding insertions in the VP1, VP2 and/or VP3 proteins.

Additionally, due to the high conservation of at least large stretches and the large member of closely related family member, the corresponding insertion sites for AAV other than the enumerated AAV can be identified by performing an amino acid alignment or by comparison of the capsid structures. See, e.g., Rutledge et al. (1998) J. Virol. 72:309-19 and U.S. Pat. No. 9,624,274 for exemplary alignments of different AAV capsid proteins, each of which reference is incorporated herein by reference in its entirety.

In some compositions disclosed herein comprising the recombinant viral capsid, the recombinant viral capsid protein is an AAV2 capsid protein VP1 with a heterologous epitope is inserted at an 1587 site, wherein the heterologous epitope does not comprise an Arg-Gly-Asp (RGD) motif, an NGR motif, or c-myc. In some compositions disclosed herein comprising the recombinant viral capsid, the recombinant viral capsid protein is a VP1 capsid protein with a heterologous epitope is inserted between T448 and N449, wherein the heterologous epitope does not comprise c-myc. In some compositions disclosed herein comprising the recombinant viral capsid, the recombinant viral capsid protein is a VP1 capsid protein with a heterologous epitope is inserted at an I-447 site, wherein the heterologous epitope does not comprise L14 or HA.

In some compositions comprising the recombinant viral capsid, the recombinant viral capsid protein is a VP1 capsid protein with a heterologous epitope is inserted at an 1587 site, wherein the heterologous epitope comprises an Arg-Gly-Asp (RGD) motif, an NGR motif, or c-myc. In some compositions disclosed herein comprising the recombinant viral capsid, the viral capsid is a VP1 capsid, the heterologous epitope comprises c-myc, and the heterologous epitope is inserted between T448 and N449, or between N587 and R588. In some compositions disclosed herein comprising the recombinant viral capsid, the recombinant viral capsid protein is a VP1 capsid protein with a heterologous epitope is inserted at an I-447 site, wherein the heterologous epitope comprises L14 or HA. In some compositions disclosed herein comprising the recombinant viral capsid, the recombinant viral capsid protein is a VP1 capsid protein with a heterologous epitope is inserted between T448 and N449, wherein the heterologous epitope comprises c-myc. U.S. Pat. No. 9,624,274 describes I-453 of an AAV capsid protein as a suitable insertion site for a heterologous epitope.

In some embodiments, insertion (display) of the heterologous epitope abolishes the natural tropism of the viral vector, e.g., transduction of a cell naturally permissive to infection by wildtype reference viral vectors and/or a target cell is undetectable in the absence of an appropriate binding molecule. In some embodiments, insertion (display) of the heterologous epitope reduces the natural tropism of the viral vector, e.g., compared to transduction of a cell naturally permissive to infection by wildtype reference viral vectors. In some embodiments, the insertion (display) of the heterologous epitope reduces the natural tropism of the viral vector by at least 5%. In some embodiments, the insertion (display) of the heterologous epitope reduces the natural tropism of the viral vector by at least 5%. In some embodiments, the insertion (display) of the heterologous epitope reduces the natural tropism of the viral vector by at least 10%. In some embodiments, the insertion (display) of the heterologous epitope reduces the natural tropism of the viral vector by at least 20%. In some embodiments, the insertion (display) of the heterologous epitope reduces the natural tropism of the viral vector by at least 30%. In some embodiments, the insertion (display) of the heterologous epitope reduces the natural tropism of the viral vector by at least 40%. In some embodiments, the insertion (display) of the heterologous epitope reduces the natural tropism of the viral vector by at least 50%. In some embodiments, the insertion (display) of the heterologous epitope reduces the natural tropism of the viral vector by at least 60%. In some embodiments, the insertion (display) of the heterologous epitope reduces the natural tropism of the viral vector by at least 70%. In some embodiments, the insertion (display) of the heterologous epitope reduces the natural tropism of the viral vector by at least 80%. In some embodiments, the insertion (display) of the heterologous epitope reduces the natural tropism of the viral vector by at least 90%. In some embodiments, the insertion (display) of the heterologous epitope reduces the natural tropism of the viral vector by at least 95%. In some embodiments, the insertion (display) of the heterologous epitope reduces the natural tropism of the viral vector by at least 90%. In these embodiments, wherein the insertion (display) of the heterologous epitope does not abolish the natural tropism of the recombinant viral capsids, the natural tropism of such recombinant viral capsids may be abolished by a second and different mutation. For example, in one embodiment, a recombinant viral capsid protein as described herein may be derived from an AAV9 capsid gene, comprise a heterologous epitope, and may further comprise a mutation, e.g., a W503A mutation. Other non-limiting examples of a second mutation include, e.g., Y445F and V473D for AAV1 or AAV6 capsid.

This detargeting of the virus from its natural host cell is important especially if systemic versus local or loco-regional administration of the viral vectors is intended, as uptake of the viral vectors by the natural host cells limits the effective dose of the viral vectors. In case of AAV2 and AAV6 HSPG is reported to be the primary receptor for viral uptake in a large number of cells, especially liver cells. For AAV2 HSPG-binding activity is dependent on a group of 5 basic amino acids, R484, R487, R585, R588 and K532 (Kern et al., (2003) J Virol. 77(20):11072-81). Recently it was reported that the lysine-to-glutamate amino acid substitution K531E leads to the suppression of AAV6's ability to bind heparin or HSPG ((Wu et al., 2006) J. of Virology 80(22):11393-11397). Accordingly, preferred point mutations are those that reduce the transducing activity of the viral vector for a given target cell mediated by the natural receptor by at least 50%, preferably at least 80%, especially at least 95%, in case of HSPG as primary receptor the binding of the viral vectors to HSPG.

Consequently, further mutations preferred for HSPG-binding viral vectors are those mutations that deplete or replace a basic amino acid such as R, K or H, preferably R or K which is involved in HSPG binding of the respective virus, by a non-basic amino acid such as A, D, G, Q, S and T, preferably A or an amino acid that is present at the corresponding position of a different but highly conserved AAV serotype lacking such basic amino acid at this position. Consequently, preferred amino acid substitutions are R484A, R487A, R487G, K532A, K532D, R585A, R585S, R585Q, R585A or R588T, especially R585A and/or R588A for AAV2, and K531A or K531E for AAV6. One especially preferred embodiment of the invention are such capsid protein mutants of AAV2 that additionally contain the two point mutations R585A and R588A as these two point mutations are sufficient to ablate HSPG binding activity to a large extent. These point mutations enable an efficient detargeting from HSPG-expressing cells which—for targeting purposes—increases specificity of the respective mutant virus for its new target cell.

One embodiment of the present invention is a multimeric structure comprising a recombinant viral capsid protein of the present invention. A multimeric structure comprises at least 5, preferably at least 10, more preferably at least 30, most preferably at least 60 recombinant viral capsid proteins comprising a heterologous epitope as described herein. They can form regular viral capsids (empty viral particles) or viral vectors (capsids encapsulating a nucleotide of interest). The formation of viral vectors capable of packaging a viral genome is a highly preferred feature for use of the recombinant viral capsids described herein as viral vectors.

In some embodiments, a targeting ligand may be associated with (e.g., displayed by, operably linked to, bound to) a modified AAV capsid protein and resulting AAV capsids according to indirect recombinatorial approaches, wherein the AAV capsid protein is modified to comprise a first member of a binding pair (e.g., a heterologous scaffold), and optionally wherein the first member of the binding pair is linked to (e.g., covalently or non-covalently bound to) a second cognate member of the binding pair (e.g., an adaptor), further optionally wherein the second cognate member of the binding pair is fused to the targeting ligand. Non-limiting and exemplary binding pairs are listed in Buning and Srivastava (2019) Mol. Ther. Methods Cin Dev 12:248-265.

Accordingly, in some embodiments, modifications of a capsid protein as described herein include those that generally result from modifications at the genetic level, e.g., via modification of a cap gene, such as modifications that insert first member of a binding pair (e.g., a protein:protein binding pair, a protein:nucleic acid binding pair), a detectable label, etc., for display by the Cap protein.

In some embodiments, the first member forms a binding pair with an immunoglobulin constant domain. In some embodiments, the first member forms a binding pair with a metal ion, e.g., Ni²⁺, Co²⁺, Cu²⁺, Zn²⁺, Fe³⁺, etc. In some embodiments, the first member is selected from the group consisting of Streptavidin, Strep II, HA, L14, 4C-RGD, LH, and Protein A.

In some embodiments, the binding pair comprises an enzyme:nucleic acid binding pair. In some embodiments, the first member comprises a HUH-endonuclease or HUB-tag and the second member comprises a nucleic acid binding domain. In some embodiments, the first member comprises a HUH tag. See, e.g., U.S. 2021/0180082, incorporated herein in its entirety by reference.

In some embodiments, a capsid protein of the invention comprises at least a first member of a peptide:peptide binding pair.

In some embodiments, each of a first member and a second member of a peptide:peptide binding pair comprises an intein. See, e.g., Wagner et al., (2021) Adv. Sci. 8: 2004018 (1 of 22); Muik et al. (2017) Biomaterials 144: 84, each of which is incorporated herein in its entirety by reference.

In some embodiments, a first member is a B cell epitope, e.g., is between about 1 amino acid and about 35 amino acids in length, and forms a binding pair with an antibody paratope, e.g., an immunoglobulin variable domain.

In some embodiments, a capsid protein of the invention comprises a first member of a protein:protein binding pair comprising a detectable label, which may also be used for the detection and/or isolation of the Cap protein and/or as a first member of a protein:protein binding pair. In some embodiments, a detectable label acts as a first member of a protein:protein binding pair for the binding of a targeting ligand comprising a multispecific binding protein that may bind both the detectable label and a target expressed by a cell of interest. In some embodiments, a Cap protein of the invention comprises a first member of a protein:protein binding pair comprising c-myc, FLAG, or HA. Use of a detectable label as a first member of a protein:protein binding pair is described in, e.g., WO2019006043.

In some embodiments, a capsid protein comprises a first member of a protein:protein binding pair, wherein the protein:protein binding pair forms a covalent isopeptide bond. In some embodiments, the first member of a peptide:peptide binding pair is covalently bound via an isopeptide bond to a cognate second member of the peptide:peptide binding pair, and optionally wherein the cognate second member of the peptide:peptide binding pair is fused with a targeting ligand, which targeting ligand binds a target expressed by a cell of interest. In some embodiments, the protein:protein binding pair may be selected from SpyTag:SpyCatcher, SpyTag002:SpyCatcher002, SpyTag003:SpyCatcher003, SpyTag:KTag, Isopeptag:pilin-C, and SnoopTag:SnoopCatcher. In some embodiments, wherein the first member is SpyTag (or a biologically active portion or variant thereof) and the protein (second cognate member) is SpyCatcher (or a biologically active portion or variant thereof). In some embodiments, wherein the first member is SpyTag (or a biologically active portion or variant thereof) and the protein (second cognate member) is KTag (or a biologically active portion or variant thereof). In some embodiments, wherein the first member is KTag (or a biologically active portion or variant thereof) and the protein (second cognate member) is SpyTag (or a biologically active portion or variant thereof). In some embodiments, wherein the first member is SnoopTag (or a biologically active portion or variant thereof) and the protein (second cognate member) is SnoopCatcher (or a biologically active portion or variant thereof). In some embodiments, wherein the first member is Isopeptag (or a biologically active portion or variant thereof) and the protein (second cognate member) is Pilin-C (or a biologically active portion or variant thereof). In some embodiments, wherein the first member is SpyTag002 (or a biologically active portion or variant thereof) and the protein (second cognate member) is SpyCatcher002 (or a biologically active portion or variant thereof). In some embodiments, wherein the first member is SpyTag003 (or a biologically active portion or variant thereof) and the protein (second cognate member) is SpyCatcher003 (or a biologically active portion or variant thereof). In some embodiments, a Cap protein of the invention comprises a SpyTag, or a biologically active portion or variant thereof. Use of a first member of a protein:protein binding pair is described in WO2019006046, incorporated herein in its entirety.

In some embodiments a viral capsid comprising a modified viral capsid protein as described herein is a mosaic capsid, e.g., comprises at least two sets of VP1, VP2, and/or VP3 proteins, each set of which is encoded by a different cap gene. A mosaic capsid herein generally refers to a mosaic of a first viral capsid protein modified to comprise a first member of a binding pair and a second corresponding viral capsid protein lacking the first member of a binding pair. In relation to a mosaic capsid, the second viral capsid protein lacking the first member of a binding pair may be referred to as a reference capsid protein encoded by a reference cap gene. In some mosaic capsid embodiments, preferably when the VP1, VP2, and/or VP3 capsid proteins modified with a first member of protein:protein pair is not a chimeric capsid protein, a VP1, VP2, and/or VP3 reference capsid protein may comprise an amino acid sequence identical to that of the viral VP1, VP2, and/or VP3 capsid protein modified with a first member of a binding pair, except that the reference capsid protein lacks the first member of a binding pair. In some mosaic capsid embodiments, a VP1, VP2, and/or VP3 reference capsid protein corresponds to the viral VP1, VP2, and/or VP3 capsid protein modified with a first member of a binding pair, except that the reference capsid protein lacks the first member of a binding pair. In some embodiments, a VP1 reference capsid protein corresponds to the viral VP1 capsid protein modified with a first member of a binding pair, except that the reference capsid protein lacks the first member of a binding pair. In some embodiments, a VP2 reference capsid protein corresponds to the viral VP2 capsid protein modified with a first member of a binding pair, except that the reference capsid protein lacks the first member of a binding pair. In some embodiments, a VP3 reference capsid protein corresponds to the viral VP3 capsid protein modified with a first member of a binding pair, except that the reference capsid protein lacks the first member of a binding pair. In some mosaic capsid embodiments comprising a chimeric VP1, VP2, and/or VP3 capsid protein further modified to comprise a first member of a binding pair, a reference protein may be a corresponding capsid protein from which portions thereof form part of the chimeric capsid protein. As a non-limiting example in some embodiments, mosaic capsid comprising a chimeric AAV2/AAAV VP1 capsid protein modified to comprise a first member of a binding pair may further comprise as a reference capsid protein: an AAV2 VP1 capsid protein lacking the first member, an AAAV VP1 capsid protein lacking the first member, a chimeric AAV2/AAAV VP1 capsid protein lacking the first member. Similarly, in some embodiments, a mosaic capsid comprising a chimeric AAV2/AAAV VP2 capsid protein modified to comprise a first member of a binding pair may further comprise as a reference capsid protein: an AAV2 VP2 capsid protein lacking the first member, an AAAV VP1 capsid protein lacking the first member, a chimeric AAV2/AAAV VP2 capsid protein lacking the first member. In some embodiments, a mosaic capsid comprising a chimeric AAV2/AAAV VP3 capsid protein modified to comprise a first member of a binding pair may further comprise as a reference capsid protein: an AAV2 VP2 capsid protein lacking the first member, an AAAV VP1 capsid protein lacking the first member, a chimeric AAV2/AAAV VP3 capsid protein lacking the first member. In some mosaic capsid embodiments, a reference capsid protein may be any capsid protein so long as it that lacks the first member of the binding pair and is able to form a capsid with the first capsid protein modified with the first member of a binding pair.

Generally mosaic particles may be generated by transfecting mixtures of the modified and reference Cap genes into production cells at the indicated ratios. The protein subunit ratios, e.g., modified VP protein:unmodified VP protein ratios, in the particle may, but do not necessarily, stoichiometrically reflect the ratios of the at least two species of the cap gene encoding the first capsid protein modified with a first member of a binding pair and the one or more reference cap genes, e.g., modified cap gene:reference cap gene(s) transfected into packaging cells. In some embodiments, the protein subunit ratios in the particle do not stoichiometrically reflect the modified cap gene:reference cap gene(s) ratio transfected into packaging cells.

In some mosaic viral particle embodiments, the protein subunit ratio ranges from about 1:59 to about 59:1.

In some non-mosaic viral particle embodiments, the protein subunit ratio may be 1:0 wherein each capsid protein of the non-mosaic viral particle is modified with a first member of a binding pair. In some non-mosaic viral particle embodiments, the protein subunit ratio may be 0:1 wherein each capsid protein of the non-mosaic viral particle is not modified with a first member of a binding pair.

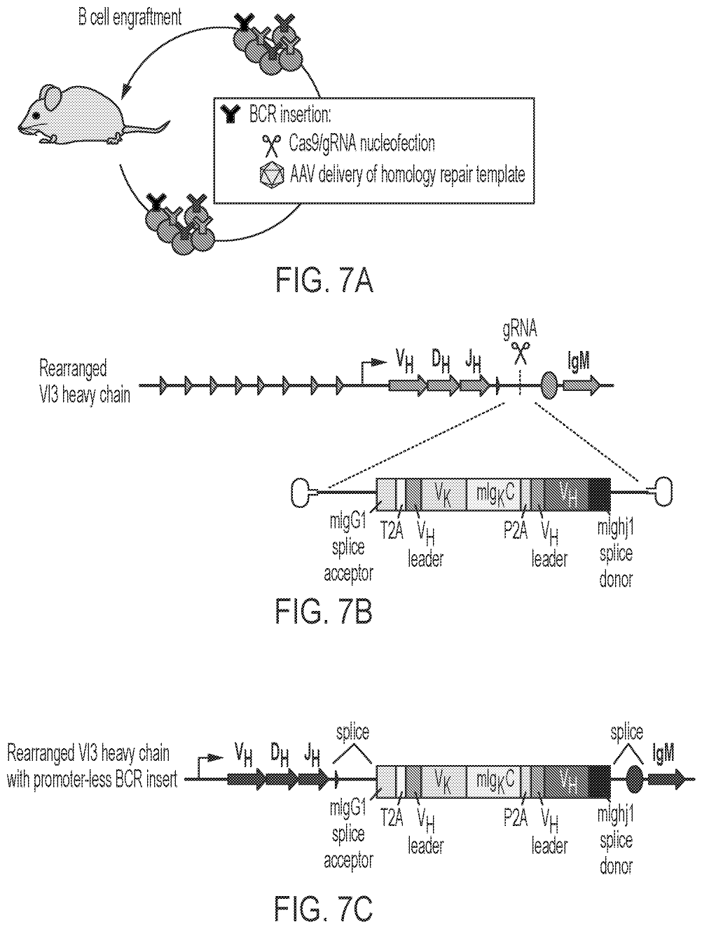

Due to the high conservation of at least large stretches and the large member of closely related family members, the corresponding insertion sites for AAV other than the enumerated AAV can be identified by performing an amino acid alignment or by comparison of the capsid structures. See, e.g., Rutledge et al. (1998) J. Virol. 72:309-19; Mietzsch et al. (2019) Viruses 11, 362, 1-34, and U.S. Pat. No. 9,624,274 for exemplary alignments of different AAV capsid proteins, each of which is incorporated herein by reference in its entirety. For example, Mietzcsh et al. (2019) provide an overlay of ribbons from different dependoparvovirus at FIG. 7, depicting the variable regions VR I to VR IX. Using such structural analysis as described therein, and sequence analysis, a skilled artisan may determine which amino acids within the variable region correspond to amino acid sequence of AAV that can accommodate the insertion of, e.g., a targeting ligand as described herein, a first member of a binding pair and/or detectable label.

Generally, the targeting ligand, first member of a binding pair, and/or detectable label may be inserted into a variable region or variable loop of an AAV capsid protein, a GH loop of an AAV capsid protein, etc.

In some embodiments, the first member of a binding pair and/or detectable label is inserted in a VP1 capsid protein of a non-primate animal AAV after an amino acid position corresponding with an amino acid position selected from the group consisting of G453 of AAV2 capsid protein VP1, N587 of AAV2 capsid protein VP1, G453 of AAV9 capsid protein VP1, and A589 of AAV9 capsid protein VP1. In some embodiments, the first member of a binding pair and/or detectable label is inserted in a VP1 capsid protein of a non-primate animal AAV between amino acids that correspond with N587 and R588 of an AAV2 VP1 capsid. Additional suitable insertion sites of a non-primate animal VP1 capsid protein include those corresponding to I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713 and I-716 of the VP1 capsid protein of AAV2 (Wu et al. (2000) J. Virol. 74:8635-8647). A modified virus capsid protein as described herein may be a non-primate animal capsid protein comprising a first member of a binding pair and/or detectable label inserted into a position corresponding with a position of an AAV2 capsid protein selected from the group consisting of I-1, I-34, I-138, I-139, I-161, I-261, I-266, I-381, I-447, I-448, I-459, I-471, I-520, I-534, I-570, I-573, I-584, I-587, I-588, I-591, I-657, I-664, I-713, I-716, and a combination thereof. Additional suitable insertion sites of a non-primate animal AAV that include those corresponding to I-587 or 1-590 of AAV1, I-589 of AAV1, I-585 of AAV3, I-584 or I-585 of AAV4, and I-575 or 1-585 of AAV5. In some embodiments, a modified virus capsid protein as described herein may be a non-primate animal capsid protein comprising a targeting ligand, first member of a binding pair and/or detectable label inserted into a position corresponding with a position selected from the group consisting of I-587 (AAV1), I-589 (AAV1), I-585 (AAV3), I-585 (AAV4), I-585 (AAV5), and a combination thereof.

In some embodiments, the first member of a binding pair and/or detectable label is inserted in a VP1 capsid protein of a non-primate animal AAV after an amino acid position corresponding with an amino acid position selected from the group consisting of I444 of an avian AAV capsid protein VP1, I580 of an avian AAV capsid protein VP1, I573 of a bearded dragon AAV capsid protein VP1, I436 of a bearded dragon AAV capsid protein VP1, I429 of a sea lion AAV capsid protein VP1, I430 of a sea lion AAV capsid protein VP1, I431 of a sea lion AAV capsid protein VP1, I432 of a sea lion AAV capsid protein VP1, I433 of a sea lion AAV capsid protein VP1, I434 of a sea lion AAV capsid protein VP1, I436 of a sea lion AAV capsid protein VP1, I437 of a sea lion AAV capsid protein VP1, and I565 of a sea lion AAV capsid protein VP1.

The nomenclature I-###, I# or the like herein refers to the insertion site (I) with ### naming the amino acid number relative to the VP1 protein of an AAV capsid protein, however such the insertion may be located directly N- or C-terminal, preferably C-terminal of one amino acid in the sequence of 5 amino acids N- or C-terminal of the given amino acid, preferably 3, more preferably 2, especially 1 amino acid(s) N- or C-terminal of the given amino acid. Additionally, the positions referred to herein are relative to the VP1 protein encoded by an AAV capsid gene, and corresponding positions (and point mutations thereof) may be easily identified for the VP2 and VP3 capsid proteins encoding by the capsid gene by performing a sequence alignment of the VP1, VP2 and VP3 proteins encoded by the appropriate AAV capsid gene.

Accordingly, an insertion into the corresponding position of the coding nucleic acid of one of these sites of the cap gene leads to an insertion into VP1, VP2 and/or VP3, as the capsid proteins are encoded by overlapping reading frames of the same gene with staggered start codons. Therefore, for AAV2, for example, according to this nomenclature insertions between amino acids 1 and 138 are only inserted into VP1, insertions between 138 and 203 are inserted into VP1 and VP2, and insertions between 203 and the C-terminus are inserted into VP1, VP2 and VP3, which is of course also the case for the insertion site I-587. Therefore, the present invention encompasses structural genes of AAV with corresponding insertions in the VP1, VP2 and/or VP3 proteins.

Also provided herein are nucleic acids that encode a VP3 capsid protein described herein. AAV capsid proteins may be, but are not necessarily, encoded by overlapping reading frames of the same gene with staggered start codons. In some embodiments, a nucleic acid that encodes a VP3 capsid protein described herein does not also encode a VP2 capsid protein or VP1 capsid protein of the invention. In some embodiments, a nucleic acid that encodes a VP3 capsid protein described herein may also encode a VP2 capsid protein described herein but does not also encode a VP1 capsid of the invention. In some embodiments, a nucleic acid that encodes a VP3 capsid protein described herein may also encode a VP2 capsid protein described herein and a VP1 capsid described herein.

In some embodiments, a viral capsid comprising the modified viral capsid protein comprising the first and second members of a binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a binding pair, e.g., comprises a control capsid protein. In some embodiments, a viral capsid comprising a modified viral capsid protein as described herein bound to the first and second members of a binding pair linked to a targeting ligand exhibits a detectable transduction efficiency compared to the undetectable transduction efficiency of a control viral capsid.

In some embodiments, a viral capsid comprising the modified viral capsid protein comprising the first and second members of a binding pair (e.g., wherein the second member is operably linked to a targeting ligand, comprises a multispecific binding protein, etc.) is able to infect a specific cell, e.g., has an enhanced capacity to target and bind a specific cell compared to that of a control viral capsid that is identical to the modified viral capsid protein except that it lacks either or both the first and second members of a binding pair, e.g., comprises a control capsid protein. In some embodiments, a viral particle of the invention comprising a viral capsid protein comprising an amino acid sequence of a capsid protein of a non-primate animal AAV, a remote AAV, or a combination thereof, and optionally comprising a first and second members of a binding pair (e.g., wherein the second member is operably linked to a targeting ligand, comprises a multispecific binding protein, etc.) is better able to evade neutralization by pre-existing antibodies in serum isolated from a human patient compared to an appropriate control viral particle (e.g., comprising a viral capsid of an AAV serotype from which a portion is included in the viral capsid of the invention, e.g., as part of the viral capsid protein comprising an amino acid sequence of a capsid protein of a non-primate animal AAV, a remote AAV, or a combination thereof), which also optionally comprises a first and second members of a binding pair (e.g., wherein the second member is operably linked to a targeting ligand, comprises a multispecific binding protein, etc.). In some embodiments, a viral particle of the invention comprising a viral capsid protein comprising an amino acid sequence of a capsid protein of a non-primate animal AAV, a remote AAV, or a combination thereof requires at least 2-fold more total IVIG or IgG for neutralization (e.g., 50% or more infection inhibition) compared to an appropriate control viral particle, e.g., (e.g., a viral particle of the invention has an IC₅₀ value that is at least 2-fold that of a control virus particle).

In some embodiments of the invention comprising a detectable label, a targeting ligand comprises a multispecific binding molecule comprising (i) an antibody paratope that specifically binds the detectable label and (ii) a second binding domain that specifically binds a receptor, which may be conjugated to the surface of a bead (e.g., for purification) or expressed by a target cell. Accordingly, a multispecific binding molecule comprising (i) an antibody paratope that specifically binds the detectable label and (ii) a second binding domain that specifically binds a receptor targets the viral particle. Such “targeting” or “directing” may include a scenario in which the wildtype viral particle targets several cells within a tissue and/or several organs within an organism, which broad targeting of the tissue or organs is reduced to abolished by insertion of the detectable label, and which retargeting to more specific cells in the tissue or more specific organ in the organism is achieved with the multispecific binding molecule. Such retargeting or redirecting may also include a scenario in which the wildtype viral particle targets a tissue, which targeting of the tissue is reduced to abolished by insertion of the detectable label, and which retargeting to a completely different tissue is achieved with the multispecific binding molecule. An antibody paratope as described herein generally comprises at a minimum a complementarity determining region (CDR) that specifically recognizes the detectable label, e.g., a CDR3 region of a heavy and/or light chain variable domain. In some embodiments, a multispecific binding molecule comprises an antibody (or portion thereof) that comprises the antibody paratope that specifically binds the detectable label. For example, a multispecific binding molecule may comprise a single domain heavy chain variable region or a single domain light chain variable region, wherein the single domain heavy chain variable region or single domain light chain variable region comprises an antibody paratope that specifically binds the detectable label. In some embodiments, a multispecific binding molecule may comprise an Fv region, e.g., a multispecific binding molecule may comprise an scFv, that comprises an antibody paratope that specifically binds the detectable label. In some embodiments, a multispecific binding molecule as described herein comprises an antibody paratope that specifically binds c-myc.

A further embodiment of the present invention is the use of at least one modified viral capsid protein and/or a nucleic acid encoding same, preferably at least one multimeric structure (e.g., viral particle) for the manufacture of and use in transfer of a nucleotide of interest to a target cell.

In some embodiments, the viral particle described herein comprises components, e.g., capsomers, glycoproteins, etc., from a virus selected from the group consisting of Human Immunodeficiency Virus (HIV), Bovine Immunodeficiency Virus (BIV), Feline Immunodeficiency Virus (FIV), Simian Immunodeficiency Virus (SIV), Equine Infectious Anemia Virus (EIAV), Murine Stem Cell Virus (MSCV), or Murine Leukemia Virus (MLV). In some embodiments, a viral particle described herein comprises an HIV capsomer, a plurality of HIV capsomers, and/or an HIV capsid, e.g., is a HIV viral particle and/or is derived from HIV.

In some embodiments, a viral particle as described herein displays, in addition to a B cell or HSC targeting moiety, a fusogen. In some embodiments, the fusogen is a protein; e.g., a viral protein (e.g., a vesiculovirus protein [e.g., vesicular stomatitis virus G glycoprotein (VSVG)], an alphavirus protein [e.g., a Sindbis virus glycoprotein], an orthomyxovirus protein [e.g., an influenza HA protein], a paramyxovirus protein [e.g., a Nipah virus F protein or a measles virus F protein]); or a fragment, mutant or derivative thereof. In one specific embodiment, the fusogen is heterologous to the reference wild-type virus from which the particle is derived. In some embodiments, the fusogen is a mutated protein which does not bind its natural ligand.

In some embodiments, the targeting moiety and the fusogen are comprised within a fusion protein.

In some embodiments described herein, the viral particles comprise a fusogen. Many different protein and non-protein fusogens can be used. In some embodiments, the fusogen is a protein. In one specific embodiment, the fusogen is a viral protein. Non-limiting examples of useful viral fusogens include, e.g., vesiculovirus fusogens (e.g., vesicular stomatitis virus G glycoprotein (VSVG)), alphavirus fusogens (e.g., a Sindbis virus glycoprotein), orthomyxovirus fusogens (e.g., influenza HA protein), paramyxovirus fusogens (e.g., a Nipah virus F protein or a measles virus F protein) as well as fusogens from Dengue virus (DV), Lassa fever virus, tick-borne encephalitis virus, Dengue virus, Hepatitis B virus, Rabies virus, Semliki Forest virus, Ross River virus, Aura virus, Borna disease virus, Hantaan virus, SARS-CoV virus, and various fragments, mutants and derivatives thereof. Other exemplary fusogenic molecules and related methods are described, for example, in U.S. Pat. Appl. Pub. 2005/0238626 and 2007/0020238.

In one specific embodiment, the fusogen is heterologous to the virus from which the particle is derived.

There are two recognized classes of viral fusogens and both can be used as targeting moieties (D. S. Dimitrov, Nature Rev. Microbio. 2, 109 (2004)). The class I fusogens trigger membrane fusion using helical coiled-coil structures, whereas the class II fusogens trigger fusion with 13 barrels. In some embodiments, class I fusogens are used. In other embodiments, class II fusogens are used. In still other embodiments, both class I and class II fusogens are used. See, e.g., Skehel and Wiley, Annu. Rev. Biochem. 69, 531-569 (2000); Smit, J. et al. J. Virol. 73, 8476-8484 (1999), Morizono et al. J. Virol. 75, 8016-8020 (2005), Mukhopadhyay et al. (2005) Rev. Microbiol. 3, 13-22.

In some specific embodiments, a form of hemagglutinin (HA) from influenza A/fowl plague virus/Rostock/34 (FPV), a class I fusogen, is used (Hatziioannou et al., J. Virol. 72, 5313 (1998)). In some specific embodiments, a form of FPV HA is used (Lin et al., Hum. Gene. Ther. 12, 323 (2001)). HA-mediated fusion is generally considered to be independent of receptor binding (Lavillette et al., Cosset, Curr. Opin. Biotech. 12, 461 (2001)).

In other embodiments, the Sindbis virus glycoprotein (a class II fusogen) from the alphavirus family is used (Wang et al., J. Virol. 66, 4992 (1992); Mukhopadhyay et al., Nature Rev. Microbio. 3, 13 (2005), Morizono et al., Nature Med. 11, 346 (2005)).

In some embodiments, mutant fusogens are used which maintain their fusogenic ability but have a decreased or eliminated binding ability or specificity. Functional properties of mutant fusogens can be tested, e.g., in cell culture or by determining their ability to stimulate an immune response without causing undesired side effects in vivo.

To select most effective and non-toxic combinations of targeting moieties and fusogens (either wild-type or mutant), viral particles bearing these molecules can be tested for their selectivity and/or their ability to facilitate penetration of the target cell membrane.

In certain embodiments, the fusogenic molecule is a Sindbis virus envelope protein (SIN). The SINdbis virus transfers its RNA into the cell by low pH mediated membrane fusion. SIN contains five structural proteins, E1, E2, E3, 6K and capsid. E2 contains the receptor binding sequence that allows the wild-type SIN to bind, while E1 is known to contain the properties necessary for membrane fusion (Konoochik et al., Virology Journal 2011, 8:304). E1, E2, and E3 are encoded by a polyprotein, the amino acid sequence of which is provided, e.g., by Accession No. VHWVB, VHWVB2, and P03316: the nucleic acid sequence is provided, e.g., by Accession No. SVU90536 and V01403 (see also Rice & Strauss, Proc. Nat'l Acad. Sci USA 78:2062-2066 (1981); and Strauss et al., Virology 133:92-110 (1984)).

In certain embodiments, the Sindbis virus envelope protein is mutated (SINmu). In certain embodiments, the mutation reduces the natural tropism of the Sindbis virus. In certain embodiments, a SINmu comprising SIN proteins E1, E2, and E3, wherein at least one of E1, E2, or E3 is mutated as compared to a wild-type sequence. For example, one or more of the E1, E2, or E3 proteins can be mutated at one or more amino acid positions. In addition, combinations of mutations in E1, E2, and E3 are encompassed by fusogen as described herein, e.g., mutations in E1 and E2, or in E2 and E3, or E3 and E1, or E1, E2, and E3. In certain embodiments, at least E2 is mutated.

In certain embodiments, the SINmu comprises the following envelope protein mutations in comparison to wild-type Sindbis virus envelope proteins: (i) deletion of E3 amino acids 61-64; (ii) E2 KE159-160AA; and (iii) E2 SLKQ68-71AAAA (“SLKQ” and “AAAA” disclosed as SEQ ID NOs 10-11, respectively). In a further embodiment, the SINmu additionally comprises the envelope protein mutation E1 AK226-227SG. Examples of SINmu may be found in, for example, in U.S. Pat. No. 9,163,248; WO2011011584; Cronin et. al., Curr Gene Ther. 2005 August; 5(4): 387-398.

Other Togaviridae family envelopes, e.g., from the Alphavirus genus, e.g., Semliki Forest Virus, Ross River Virus, and equine encephalitis virus, can also be used to pseudotype the vectors described herein. The envelope protein sequences for such Alphaviruses are known in the art.

In certain embodiments, the fusogen is a vesicular stomatitis virus (VSV) envelope protein. In certain embodiments, the fusogen is the G protein of VSV (VSV-G; Burns et al., Proc. Natl. Acad. Sci. U.S.A. 1993, vol. 90, no. 17, p. 1833-7) or a fragment, mutant, derivative or homolog thereof. VSV-G interacts with a phospholipid component of the cell (e.g., T cell) membrane to mediate viral entry by membrane fusion (Mastromarino et al., J Gen Virol. 1998, vol. 68, no. 9, p. 2359-69; Marsh et al., Adv Virus Res. 1989, vol. 107, no. 36, p. 107-51. Examples of VSV-G may be found in, for example, WO2008058752.

One embodiment of the present disclosure is a nucleic acid encoding a capsid protein as described above. The nucleic acid is preferably a vector comprising the claimed nucleic acid sequence. Nucleic acids, especially vectors are necessary to recombinantly express the capsid proteins of this disclosure.

A further embodiment of the present disclosure is the use of at least one recombinant viral capsid protein and/or a nucleic acid encoding same, preferably at least one multimeric structure (e.g., viral vector) for the manufacture of and use as a gene transfer vector.

### Heterologous Epitopes

Generally, a recombinant viral capsid protein and/or a viral vector comprising the recombinant viral capsid comprises a heterologous epitope, which enables the retargeting of the viral vector, e.g., via a binding molecule (e.g., an antibody). In some embodiments, a heterologous epitope is a B cell epitope, e.g., which is between about 1 amino acid and about 35 amino acids in length, and forms a binding pair with an antibody paratope, e.g., an immunoglobulin variable domain. In some embodiments, the heterologous epitope comprises an affinity tag.

A large number of tags are known in the art. (See, e.g.: Nilsson et al. (1997) “Affinity fusion strategies for detection, purification, and immobilization of recombinant proteins” Protein Expression and Purification 11: 1-16, Terpe et al. (2003) “Overview of tag protein fusions: From molecular and biochemical fundamentals to commercial systems” Applied Microbiology and Biotechnology 60:523-533, and references therein). Affinity tags include, but are not limited to, a polyhistidine tag (e.g., a His-6, His-8, or His-10 tag) that binds immobilized divalent cations (e.g., Ni²⁺), a biotin moiety (e.g., on an in vivo biotinylated polypeptide sequence) that binds immobilized avidin, a GST (glutathione S-transferase) sequence that binds immobilized glutathione, an S tag that binds immobilized S protein, an antigen that binds an immobilized antibody or domain or fragment thereof (including, e.g., T7, myc, FLAG, and B tags that bind corresponding antibodies), a FLASH Tag (a high affinity tag that couples to specific arsenic based moieties), a receptor or receptor domain that binds an immobilized ligand (or vice versa), protein A or a derivative thereof (e.g., Z) that binds immobilized IgG, maltose-binding protein (MBP) that binds immobilized amylose, an albumin-binding protein that binds immobilized albumin, a chitin binding domain that binds immobilized chitin, a calmodulin binding peptide that binds immobilized calmodulin, and a cellulose binding domain that binds immobilized cellulose. Another exemplary tag is a SNAP-tag, commercially available from Covalys (www.covalys.com). In some embodiments, a heterologous epitope disclosed herein comprises an affinity tag recognized only by an antibody paratope. In some embodiments, a heterologous epitope disclosed herein comprises an affinity tag recognized by an antibody paratope and other specific binding pairs.

In some embodiments, the heterologous epitope and/or affinity tag does not form a binding pair with an immunoglobulin constant domain. In some embodiments, the heterologous epitope and/or affinity tag does not form a binding pair with a metal ion, e.g., Ni²⁺, Co²⁺, Cu²⁺, Zn²⁻, Fe³⁺, etc. In some embodiments, the heterologous epitope is not a polypeptide selected from the group consisting of Streptavidin, Strep II, HA, L14, 4C-RGD, LH, and Protein A.

In some embodiments, the affinity tag is selected from the group consisting of FLAG, HA and c-myc (EQKLISEEDL (SEQ ID NO: 12)). In some embodiments, the heterologous epitope is c-myc.

In some embodiments, a recombinant viral capsid as described herein comprises the amino acid sequence EQKLISEEDL (SEQ ID NO: 12) flanked by and/or operably linked to at least 5 contiguous amino acids of an AAV VP1 capsid protein. In some embodiments, a recombinant vital capsid as described herein comprises the amino acid sequence EQKLISEEDL (SEQ ID NO: 12) flanked by and/or operably linked to at least 5 contiguous amino acids of an AAV2 VP1 capsid protein. In some embodiments, a recombinant viral capsid as described herein comprises EQKLISEEDL (SEQ ID NO: 12) inserted between N587 and R588 of an AAV2 VP1 capsid protein.

In some embodiments, the heterologous epitope comprises an affinity tag and one or more linkers. In some embodiments, the heterologous epitope comprises an affinity tag flanked by a linker, e.g., the heterologous epitope comprises from N-terminus to C-terminus a first linker, an affinity tag, and a second linker. In some embodiments, the first and second linkers are each independently at least one amino acid in length. In some embodiments, the first and second linkers are identical.

Generally, a heterologous epitope as described herein, e.g., an affinity tag by itself or in combination with one or more linkers, is between about 5 amino acids to about 35 amino acids in length. In some embodiments, the heterologous epitope (by itself or in combination with one or more linkers) is at least 5 amino acids in length. In some embodiments, the heterologous epitope (by itself or in combination with one or more linkers) is 6 amino acids in length. In some embodiments, the heterologous epitope (by itself or in combination with one or more linkers) is 7 amino acids in length. In some embodiments, the heterologous epitope (by itself or in combination with one or more linkers) is 8 amino acids in length. In some embodiments, the heterologous epitope (by itself or in combination with one or more linkers) is 9 amino acids in length. In some embodiments, the heterologous epitope (by itself or in combination with one or more linkers) is 10 amino acids in length. In some embodiments, the heterologous epitope (by itself or in combination with one or more linkers) is 11 amino acids in length. In some embodiments, the heterologous epitope (by itself or in combination with one or more linkers) is 12 amino acids in length. In some embodiments, the heterologous epitope (by itself or in combination with one or more linkers) is 13 amino acids in length. In some embodiments, the heterologous epitope (by itself or in combination with one or more linkers) is 14 amino acids in length. In some embodiments, the heterologous epitope (by itself or in combination with one or more linkers) is 15 amino acids in length. In some embodiments, the heterologous epitope (by itself or in combination with one or more linkers) is 16 amino acids in length. In some embodiments, the heterologous epitope (by itself or in combination with one or more linkers) is 17 amino acids in length. In some embodiments, the heterologous epitope (by itself or in combination with one or more linkers) is 18 amino acids in length. In some embodiments, the heterologous epitope (by itself or in combination with one or more linkers) is 19 amino acids in length. In some embodiments, the heterologous epitope (by itself or in combination with one or more linkers) is 20 amino acids in length. In some embodiments, the heterologous epitope (by itself or in combination with one or more linkers) is 21 amino acids in length. In some embodiments, the heterologous epitope (by itself or in combination with one or more linkers) is 22 amino acids in length. In some embodiments, the heterologous epitope (by itself or in combination with one or more linkers) is 23 amino acids in length. In some embodiments, the heterologous epitope (by itself or in combination with one or more linkers) is 24 amino acids in length. In some embodiments, the heterologous epitope (by itself or in combination with one or more linkers) is 25 amino acids in length. In some embodiments, the heterologous epitope (by itself or in combination with one or more linkers) is 26 amino acids in length. In some embodiments, the heterologous epitope (by itself or in combination with one or more linkers) is 27 amino acids in length. In some embodiments, the heterologous epitope (by itself or in combination with one or more linkers) is 28 amino acids in length. In some embodiments, the heterologous epitope (by itself or in combination with one or more linkers) is 29 amino acids in length. In some embodiments, the heterologous epitope (by itself or in combination with one or more linkers) is 30 amino acids in length. In some embodiments, the heterologous epitope (by itself or in combination with one or more linkers) is 31 amino acids in length. In some embodiments, the heterologous epitope (by itself or in combination with one or more linkers) is 32 amino acids in length. In some embodiments, the heterologous epitope (by itself or in combination with one or more linkers) is 33 amino acids in length. In some embodiments, the heterologous epitope (by itself or in combination with one or more linkers) is 34 amino acids in length. In some embodiments, the heterologous epitope (by itself or in combination with one or more linkers) is 35 amino acids in length.

### Retargeting Moieties

A viral vector as described herein has reduced to abolished transduction capabilities in the absence of a binding molecule, specifically a binding molecule that specifically binds a surface molecule expressed by a target cell (e.g., a B cell or a hematopoietic stem cell). In some embodiments, a binding molecule comprises an antibody (or a fragment thereof) that comprises the antibody paratope that specifically binds the heterologous epitope. For example, a binding molecule may comprise a single domain heavy chain variable region or a single domain light chain variable region, wherein the single domain heavy chain variable region or single domain light chain variable region comprises an antibody paratope that specifically binds the heterologous epitope. In some embodiments, a binding molecule may comprise an Fv region, e.g., a binding molecule may comprise an scFv, that comprises an antibody paratope that specifically binds the heterologous epitope. In some embodiments, a binding molecule as described herein comprises an antibody paratope that specifically binds c-myc.

Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

In some embodiments, the binding molecule binds a protein expressed on the surface of a cell, e.g., a cell surface protein on a hematopoietic cell, e.g., a B cell or a hematopoietic stem cell (HSC). There are a large number of cell surface proteins, e.g., cell surface receptors, suitable which may be targeted by a retargeting ligand, and for which a retargeting ligand, e.g., antibodies or portions thereof, are already available. Such structures include, but are not limited to B cell receptors and associated proteins (e.g., CD19, CD20, CD22, CD34, CD38, CD40, CD22, CD79, CD180, B-cell activating factor (BAFF), ASGR1, CD 117, Sca1, etc.) and HSC receptors and associated proteins (e.g., CD34, etc.). A recombinant viral capsid described herein allows for the specific infection of a cell type by employing a binding molecule comprising a retargeting ligand that binds differentiation cell surface antigens as targets for the viral vector complex.

A viral particle described herein may further comprise a second member of the specific binding pair that specifically forms a covalent bond with the first member of the specific binding pair that is inserted into/displayed by a recombinant viral capsid protein, wherein the second member is fused to a binding molecule.

In certain exemplary embodiments, the binding molecule is a bispecific antibody. Each antigen-binding domain of a bispecific antibody comprises a heavy chain variable domain (HCVR) and a light chain variable domain (LCVR). In the context of a bispecific antigen-binding molecule comprising a first and a second antigen-binding domain (e.g., a bispecific antibody), the CDRs of the first antigen-binding domain may be designated with the prefix “A1” and the CDRs of the second antigen-binding domain may be designated with the prefix “A2”. Thus, the CDRs of the first antigen-binding domain may be referred to herein as A1-HCDR1, A1-HCDR2, and A1-HCDR3; and the CDRs of the second antigen-binding domain may be referred to herein as A2-HCDR1, A2-HCDR2, and A2-HCDR3.

The first antigen-binding domain and the second antigen-binding domain may be directly or indirectly connected to one another to form a bispecific antigen-binding molecule of the present invention. Alternatively, the first antigen-binding domain and the second antigen-binding domain may each be connected to a separate multimerizing domain. The association of one multimerizing domain with another multimerizing domain facilitates the association between the two antigen-binding domains, thereby forming a bispecific antigen-binding molecule. As used herein, a “multimerizing domain” is any macromolecule, protein, polypeptide, peptide, or amino acid that has the ability to associate with a second multimerizing domain of the same or similar structure or constitution. For example, a multimerizing domain may be a polypeptide comprising an immunoglobulin CH3 domain. A non-limiting example of a multimerizing component is an Fc portion of an immunoglobulin (comprising a CH2-CH3 domain), e.g., an Fc domain of an IgG selected from the isotypes IgG1, IgG2, IgG3, and IgG4, as well as any allotype within each isotype group.

Bispecific antigen-binding molecules of the present invention will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

In certain embodiments, the multimerizing domain is an Fc fragment or an amino acid sequence of 1 to about 200 amino acids in length containing at least one cysteine residues. In other embodiments, the multimerizing domain is a cysteine residue, or a short cysteine-containing peptide. Other multimerizing domains include peptides or polypeptides comprising or consisting of a leucine zipper, a helix-loop motif, or a coiled-coil motif.

Any bispecific antibody format or technology may be used to make the bispecific antigen-binding molecules of the present invention. For example, an antibody or fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats; see also Brinkmann and Konterman (2017) mAbs 9:182-212; each of which is incorporated by reference in its entirety).

The present invention also includes bispecific antigen-binding molecules comprising a first CH3 domain and a second Ig CH3 domain, wherein the first and second Ig CH3 domains differ from one another by at least one amino acid, and wherein at least one amino acid difference reduces binding of the bispecific antibody to Protein A as compared to a bi-specific antibody lacking the amino acid difference. In one embodiment, the first Ig CH3 domain binds Protein A and the second Ig CH3 domain contains a mutation that reduces or abolishes Protein A binding such as an H95R modification (by IMGT exon numbering; H435R by EU numbering). The second CH3 may further comprise a Y96F modification (by IMGT; Y436F by EU). Further modifications that may be found within the second CH3 include: D16E, L18M, N44S, K52N, V57M, and V82I (by IMGT; D356E, L358M, N384S, K392N, V397M, and V422I by EU) in the case of IgG1 antibodies; N44S, K52N, and V82I (IMGT; N384S, K392N, and V422I by EU) in the case of IgG2 antibodies; and Q15R, N44S, K52N, V57M, R69K, E79Q, and V82I (by IMGT; Q355R, N384S, K392N, V397M, R409K, E419Q, and V422I by EU) in the case of IgG4 antibodies; see, e.g., WO 2010/151792.

In certain embodiments, the Fc domain may be chimeric, combining Fc sequences derived from more than one immunoglobulin isotype. For example, a chimeric Fc domain can comprise part or all of a CH2 sequence derived from a human IgG1, human IgG2 or human IgG4 CH2 region, and part or all of a CH3 sequence derived from a human IgG1, human IgG2 or human IgG4. A chimeric Fc domain can also contain a chimeric hinge region. For example, a chimeric hinge may comprise an “upper hinge” sequence, derived from a human IgG1, a human IgG2 or a human IgG4 hinge region, combined with a “lower hinge” sequence, derived from a human IgG1, a human IgG2 or a human IgG4 hinge region. A particular example of a chimeric Fc domain that can be included in any of the antigen-binding molecules set forth herein comprises, from N- to C-terminus: [IgG4 CH1]-[IgG4 upper hinge]-[IgG2 lower hinge]-[IgG4 CH2]-[IgG4 CH3]. Another example of a chimeric Fc domain that can be included in any of the antigen-binding molecules set forth herein comprises, from N- to C-terminus: [IgG1 CH1]-[IgG1 upper hinge]-[IgG2 lower hinge]-[IgG4 CH2]-[IgG1 CH3]. These and other examples of chimeric Fe domains that can be included in any of the antigen-binding molecules of the present invention are described in PCT Application No. WO2014/022540, incorporated by reference in its entirety. Chimeric Fe domains having these general structural arrangements, and variants thereof, can have altered Fc receptor binding, which in turn affects Fe effector function.

### Liposomes, Lipid Nanoparticles and Other Carriers

In some embodiments, the first component and/or the second component of a system described herein may be a lipid-based carrier, such as a lipid nanoparticle (LNP), a liposome, a lipidoid, or a lipoplex.

In some embodiments, the first component and/or the second component of a system described herein may comprise a liposome or LNP. Liposomes and LNPs are vesicles including one or more lipid bilayers. In some embodiments, a liposome or LNP includes two or more concentric bilayers separated by aqueous compartments. Lipid bilayers can be functionalized and/or crosslinked to one another. Lipid bilayers can include one or more proteins, polysaccharides or other molecules.

Lipid formulations can protect biological molecules from degradation while improving their cellular uptake. Liposomes or LNPs are particles comprising a plurality of lipid molecules physically associated with each other by intermolecular forces. These include microspheres (including unilamellar and multilamellar vesicles, e.g., liposomes), a dispersed phase in an emulsion, micelles, or an internal phase in a suspension. Such liposomes or LNPs can be used to encapsulate one or more nucleic acids or proteins for delivery. Formulations which contain cationic lipids are useful for delivering polyanions such as nucleic acids. Other lipids that can be included are neutral lipids (i.e., uncharged or zwitterionic lipids), anionic lipids, helper lipids that enhance transfection, and stealth lipids that increase the length of time for which nanoparticles can exist in vivo. Examples of suitable cationic lipids, neutral lipids, anionic lipids, helper lipids, and stealth lipids can be found in WO 2016/010840 A1 and WO 2017/173054 A1, each of which is herein incorporated by reference in its entirety for all purposes. An exemplary lipid nanoparticle can comprise a cationic lipid and one or more other components. In one example, the other component can comprise a helper lipid such as cholesterol. In another example, the other components can comprise a helper lipid such as cholesterol and a neutral lipid such as distearoylphosphatidylcholine (DSPC). In another example, the other components can comprise a helper lipid such as cholesterol, an optional neutral lipid such as DSPC, and a stealth lipid such as S010, S024, S027, S031, or S033.

Liposomes are amphiphilic lipids which can form bilayers in an aqueous environment to encapsulate an aqueous core. The polypeptide (e.g., Cas protein) or polynucleotide (e.g., guide RNA) may be incorporated into the aqueous core. These lipids can have an anionic, cationic or zwitterionic hydrophilic head group. Liposomes can be formed from a single lipid or from a mixture of lipids. A mixture may comprise (1) a mixture of anionic lipids; (2) a mixture of cationic lipids; (3) a mixture of zwitterionic lipids; (4) a mixture of anionic lipids and cationic lipids; (5) a mixture of anionic lipids and zwitterionic lipids; (6) a mixture of zwitterionic lipids and cationic lipids; or (7) a mixture of anionic lipids, cationic lipids and zwitterionic lipids. Similarly, a mixture may comprise both saturated and unsaturated lipids. Exemplary phospholipids include, but are not limited to, phosphatidylethanolamines, phosphatidylcholines, phosphatidylserines, and phosphatidylglycerols. Cationic lipids include, but are not limited to, 1,2-distearyloxy-N,N-dimethyl-3-aminopropane (DSDMA), dioleoyl trimethylammonium propane (DOTAP), 1,2-dioleyloxy-N,Ndimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA). Zwitterionic lipids include, but are not limited to, acyl zwitterionic lipids and ether zwitterionic lipids. Examples of useful zwitterionic lipids include dodecylphosphocholine, DPPC, and DOPC.

The liposomes or LNPs may contain one or more or all of the following: (i) a lipid for encapsulation and for endosomal escape; (ii) a neutral lipid for stabilization; (iii) a helper lipid for stabilization; and (iv) a stealth lipid. See, e.g., Finn et al. (2018) Cell Rep. 22(9):2227-2235 and WO 2017/173054 A1, each of which is herein incorporated by reference in its entirety for all purposes.

In some examples, the liposomes or LNPs comprise cationic lipids. In some examples, the liposomes or LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g., WO 2019/067992, WO 2017/173054, WO 2015/095340, and WO 2014/136086, each of which is herein incorporated by reference in its entirety for all purposes. In some examples, the LNPs comprise molar ratios of a cationic lipid amine to RNA phosphate (N:P) of about 4.5, about 5.0, about 5.5, about 6.0, or about 6.5. In some examples, the terms cationic and ionizable in the context of LNP lipids are interchangeable (e.g., wherein ionizable lipids are cationic depending on the pH).

The lipid for encapsulation and endosomal escape can be a cationic lipid. The lipid can also be a biodegradable lipid, such as a biodegradable ionizable lipid. One example of a suitable lipid is Lipid A or LP01, which is (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235 and WO 2017/173054 A1, each of which is herein incorporated by reference in its entirety for all purposes. Another example of a suitable lipid is Lipid B, which is ((5-((dimethylamino)methyl)-1,3-phenylene)bis(oxy))bis(octane-8,1-diyl)bis(decanoate), also called ((5-((dimethylamino)methyl)-1,3-phenylene)bis(oxy))bis(octane-8,1-diyl)bis(decanoate). Another example of a suitable lipid is Lipid C, which is 2-((4-(((3-(dimethylamino)propoxy)carbonyl)oxy)hexadecanoyl)oxy)propane-1,3-diyl(9Z,9′Z,12Z,12′Z)-bis(octadeca-9,12-dienoate). Another example of a suitable lipid is Lipid D, which is 3-(((3-(dimethylamino)propoxy)carbonyl)oxy)-3-(octanoyloxy)tridecyl 3-octylundecanoate. Other suitable lipids include heptatriaconta-6,9,28,31-tetraen-19-yl 4-(dimethylamino)butanoate (also known as [(6Z,9Z,28Z,31Z)-heptatriaconta-6,9,28,31-tetraen-19-yl]4-(dimethylamino)butanoate or Dlin-MC3-DMA (MC3))).

Additional suitable cationic lipids include, but are not limited to 1,2-DiLinoleyloxy-N,N-dimethylaminopropane (DLinDMA), 1,2-Dilinolenyloxy-N,N-dimethylaminopropane (DLenDMA), dioctadecyldimethylammonium (DODMA), distearyldimethylammonium (DSDMA), N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N(N′,N′-dimethylaminoethane)-carbamoyl)cholesterol (DC-Chol), and N-(1,2-dimyristyloxyprop-3-yl)-N,N-dimethyl-N-hydroxyethyl ammonium bromide (DMRIE). For example, cationic lipids that have a positive charge at below physiological pH include, but are not limited to, DODAP, DODMA, and DMDMA. In some embodiments, the cationic lipids comprise C18 alkyl chains, ether linkages between the head group and alkyl chains, and 0 to 3 double bonds. Such lipids include, e.g., DSDMA, DLinDMA, DLenDMA, and DODMA. The cationic lipids may comprise ether linkages and pH titratable head groups. Such lipids include, e.g., DODMA. Additional cationic lipids are described in U.S. Pat. Nos. 7,745,651; 5,208,036; 5,264,618; 5,279,833; 5,283,185; 5,753,613; and 5,785,992, incorporated herein by reference.

In some embodiments, the cationic lipids may comprise a protonatable tertiary amine head group. Such lipids are referred to herein as ionizable lipids. Ionizable lipids refer to lipid species comprising an ionizable amine head group and typically comprising a pKa of less than about 7. In environments with an acidic pH, the ionizable amine head group is protonated such that the ionizable lipid preferentially interacts with negatively charged molecules (e.g., nucleic acids such as the recombinant polynucleotides described herein) thus facilitating liposome or LNP assembly and encapsulation. Therefore, in some embodiments, ionizable lipids can increase the loading of nucleic acids into liposomes or LNPs. In environments where the pH is greater than about 7 (e.g., physiologic pH of 7.4), the ionizable lipid comprises a neutral charge. When particles comprising ionizable lipids are taken up into the low pH environment of an endosome (e.g., pH<7), the ionizable lipid is again protonated and associates with the anionic endosomal membranes, promoting release of the contents encapsulated by the particle.

In some embodiments, the liposomes or LNPs may comprise one or more non-cationic helper lipids. Exemplary helper lipids include (1,2-dilauroyl-sn-glycero-3-phosphoethanolamine) (DLPE), 1,2-diphytanoyl-sn-glycero-3-phosphoethanolamine (D iPPE), 1,2-distearoyl-sn-glycero-3-phosphocholine (DSPC), 1,2-dipalmitoyl-sn-glycero-3-phosphocholine (DPPC), 1,2-dioleyl-sn-glycero-3-phosphoethanolamine (DOPE), 1,2-dipalmitoyl-sn-glycero-3-phosphoethanolamine (DPPE), 1,2-dimyristoyl-sn-glycero-3-phosphoethanolamine (DMPE), (1,2-dioleoyl-sn-glycero-3-phospho-(1′-rac-glycerol) (DOPG), 1,2-distearoyl-sn-glycero-3-phosphoethanolamine (DSPE), ceramides, sphingomyelins, and cholesterol.

Some such lipids suitable for use in the liposomes or LNPs described herein are biodegradable in vivo. Examples of biodegradable lipids include, but are not limited to, (9Z,12Z)-3-((4,4-bis(octyloxy)butanoyl)oxy)-2-((((3-20 (diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl(9Z,12Z)-octadeca-9,12-dienoate) or another ionizable lipid. See, e.g., PCT Publication Nos. WO2017/173054, WO2015/095340, and WO2014/136086. In some embodiments, the term cationic and ionizable in the context of liposome or LNP lipids is interchangeable, e.g., wherein ionizable lipids are cationic depending on the pH.

Such lipids may be ionizable depending upon the pH of the medium they are in. For example, in a slightly acidic medium, the lipids may be protonated and thus bear a positive charge. Conversely, in a slightly basic medium, such as, for example, blood where pH is approximately 7.35, the lipids may not be protonated and thus bear no charge. In some embodiments, the lipids may be protonated at a pH of at least about 9, 9.5, or 10. The ability of such a lipid to bear a charge is related to its intrinsic pKa. For example, the lipid may, independently, have a pKa in the range of from about 5.8 to about 6.2.

Neutral lipids function to stabilize and improve processing of the liposomes or LNPs. Examples of suitable neutral lipids include a variety of neutral, uncharged or zwitterionic lipids. Examples of neutral phospholipids suitable for use in the present disclosure include, but are not limited to, 5-heptadecylbenzene-1,3-diol (resorcinol), dipalmitoylphosphatidylcholine (DPPC), distearoylphosphatidylcholine or 1,2-distearoyl-sn-glycero-3-phosphocholine (DSPC), phosphocholine (DOPC), dimyristoylphosphatidylcholine (DMPC), phosphatidylcholine (PLPC), 1,2-diarachidonoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-diarachidoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine, 1-stearoyl-2-oleoyl-sn-glycero-3-phosphocholine (SOPC), and combinations thereof. For example, the neutral phospholipid may be selected from the group consisting of distearoylphosphatidylcholine (DSPC) and dimyristoyl phosphatidyl ethanolamine (DMPE).

Helper lipids include lipids that enhance transfection. The mechanism by which the helper lipid enhances transfection can include enhancing particle stability. In certain cases, the helper lipid can enhance membrane fusogenicity. Helper lipids include steroids, sterols, and alkyl resorcinols. Examples of suitable helper lipids suitable include cholesterol, 5-heptadecylresorcinol, and cholesterol hemisuccinate. In one example, the helper lipid may be cholesterol or cholesterol hemisuccinate.

Stealth lipids include lipids that alter the length of time the nanoparticles can exist in vivo. Stealth lipids may assist in the formulation process by, for example, reducing particle aggregation and controlling particle size. Stealth lipids may modulate pharmacokinetic properties of the liposomes or LNPs. Suitable stealth lipids include lipids having a hydrophilic head group linked to a lipid moiety.

The hydrophilic head group of stealth lipid can comprise, for example, a polymer moiety selected from polymers based on PEG (sometimes referred to as poly(ethylene oxide)), poly(oxazoline), poly(vinyl alcohol), poly(glycerol), poly(N-vinylpyrrolidone), polyaminoacids, and poly N-(2-hydroxypropyl)methacrylamide. The term PEG means any polyethylene glycol or other polyalkylene ether polymer. In certain liposome or LNP formulations, the PEG, is a PEG-2K, also termed PEG 2000, which has an average molecular weight of about 2,000 daltons. See, e.g., WO 2017/173054 A1, herein incorporated by reference in its entirety for all purposes.

The lipid moiety of the stealth lipid may be derived, for example, from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. The dialkylglycerol or dialkylglycamide group can further comprise one or more substituted alkyl groups.

As one example, the stealth lipid may be selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000](PEG2k-DMG), 1,2-distearoyl-sn-glycero-3-phosphoethanolamine-N-[methoxy(polyethylene glycol)-2000](PEG2k-DSPE), 1,2-distearoyl-sn-glycerol, methoxypoly ethylene glycol (PEG2k-DSG), poly(ethylene glycol)-2000-dimethacrylate (PEG2k-DMA), and 1,2-distearyloxypropyl-3-amine-N-[methoxy(polyethylene glycol)-2000](PEG2k-DSA). In one particular example, the stealth lipid may be PEG2k-DMG.

In some embodiments, the liposomes or LNPs may further comprise one or more of PEG-modified lipids that comprise a poly(ethylene)glycol chain of up to 5 kDa in length covalently attached to a lipid comprising one or more C6-C20 alkyls. In some embodiments, the liposomes or LNPs further comprise 1,2-Distearoyl-sn-glycero-3-phosphoethanolamine-Poly(ethylene glycol) (DSPE-PEG), or 1,2-distearoyl-sn-glycero-3-phosphoethanolamine-N-[amino(polyethylene glycol)](DSPE-PEG-amine). In some embodiments, the PEG-modified lipid comprises about 0.1% to about 1% of the total lipid content in a lipid nanoparticle. In some embodiments, the PEG-modified lipid comprises about 0.1%, about 0.2% about 0.3%, about 0.4%, about 0.5%, about 0.6%, about 0.7%, about 0.8%, about 0.9%, or about 1.0%, of the total lipid content in the liposome or lipid nanoparticle.

In some embodiments, a liposome or LNP described herein may comprise a conjugated lipid that inhibits aggregation of lipid particles. Such lipid conjugates include, but are not limited to, PEG-lipid conjugates such as, e.g., PEG coupled to dialkyloxypropyls (e.g., PEG-DAA conjugates), PEG coupled to diacylglycerols (e.g., PEG-DAG conjugates), PEG coupled to cholesterol, PEG coupled to phosphatidylethanolamines, and PEG conjugated to ceramides (see, e.g., U.S. Pat. No. 5,885,613), cationic PEG lipids, polyoxazoline (POZ)-lipid conjugates (e.g., POZ-DAA conjugates), polyamide oligomers (e.g., ATTA-lipid conjugates), and mixtures thereof. Additional examples of POZ-lipid conjugates are described in PCT Publication No. WO 2010/006282. PEG or POZ can be conjugated directly to the lipid or may be linked to the lipid via a linker moiety. Any linker moiety suitable for coupling the PEG or the POZ to a lipid can be used including, e.g., non-ester containing linker moieties and ester-containing linker moieties. In certain embodiments, non-ester containing linker moieties, such as amides or carbamates, are used.

The liposomes or LNPs can comprise different respective molar ratios of the component lipids in the formulation. The mol-% of the CCD lipid may be, for example, from about 30 mol-% to about 60 mol-%. The mol-% of the helper lipid may be, for example, from about 30 mol-% to about 60 mol-%. The mol-% of the neutral lipid may be, for example, from about 1 mol-% to about 20 mol-%. The mol-% of the stealth lipid may be, for example, from about 1 mol-% to about 10 mol-%

The liposomes or LNPs can have different ratios between the positively charged amine groups of the biodegradable lipid (N) and the negatively charged phosphate groups (P) of the nucleic acid to be encapsulated. This may be mathematically represented by the equation N/P. For example, the N/P ratio may be from about 0.5 to about 100. The N/P ratio can also be from about 4 to about 6.

In some embodiments, the liposome or LNP can comprise a nuclease agent (e.g., CRISPR/Cas system, ZFN, or TALEN), can comprise a polynucleotide molecule (e.g., guide RNA), can comprise a nucleic acid construct encoding a polypeptide of interest (e.g., antibody or antigen-binding fragment), or can comprise both a nuclease agent (e.g., a CRISPR/Cas system) and a nucleic acid construct encoding a polypeptide of interest (e.g., a donor template for use in gene editing). Regarding CRISPR/Cas systems, the liposomes or LNPs can comprise the Cas protein in any form (e.g., protein, DNA, or mRNA) and/or can comprise the guide RNA(s) in any form (e.g., DNA or RNA). In one example, the liposomes or LNPs comprise the Cas protein in the form of mRNA (e.g., a modified RNA as described herein) and the guide RNA(s) in the form of RNA (e.g., a modified guide RNA as disclosed herein). As another example, the liposomes or LNPs can comprise the Cas protein in the form of protein and the guide RNA(s) in the form of RNA). In one example, the guide RNA and the Cas protein are each introduced in the form of RNA via LNP-mediated delivery in the same LNP. As discussed in more detail elsewhere herein, one or more of the RNAs can be modified. For example, guide RNAs can be modified to comprise one or more stabilizing end modifications at the 5′ end and/or the 3′ end. Such modifications can include, for example, one or more phosphorothioate linkages at the 5′ end and/or the 3′ end and/or one or more 2′-O-methyl modifications at the 5′ end and/or the 3′ end. As another example, Cas mRNA modifications can include substitution with pseudouridine (e.g., fully substituted with pseudouridine), 5′ caps, and polyadenylation. Other modifications are also contemplated as disclosed elsewhere herein. Delivery through such methods can result in transient Cas expression and/or transient presence of the guide RNA, and the biodegradable lipids improve clearance, improve tolerability, and decrease immunogenicity.

In certain liposomes or LNPs, the cargo can include a guide RNA or a nucleic acid encoding a guide RNA. In certain liposomes or LNPs, the cargo can include an mRNA encoding a Cas nuclease, such as Cas9, and a guide RNA or a nucleic acid encoding a guide RNA. In certain liposomes or LNPs, the cargo can include a nucleic acid construct encoding a polypeptide of interest (e.g., antibody or antigen-binding fragment) as described elsewhere herein. In certain liposomes or LNPs, the cargo can include an mRNA encoding a Cas nuclease, such as Cas9, a guide RNA or a nucleic acid encoding a guide RNA, and a nucleic acid construct encoding a polypeptide of interest (e.g., antibody or antigen-binding fragment). In some liposomes or LNPs, the lipid component comprises an amine lipid such as a biodegradable, ionizable lipid. In some instances, the lipid component comprises biodegradable, ionizable lipid, cholesterol, DSPC, and PEG-DMG. For example, Cas9 mRNA and gRNA can be delivered to cells and animals utilizing lipid formulations comprising ionizable lipid ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate), cholesterol, DSPC, and PEG2k-DMG.

In some liposomes or LNPs, the cargo can comprise Cas mRNA (e.g., Cas9 mRNA) and gRNA. The Cas mRNA and gRNAs can be in different ratios. For example, the LNP formulation can include a ratio of Cas mRNA to gRNA nucleic acid ranging from about 25:1 to about 1:25. Alternatively, the liposome or LNP formulation can include a ratio of Cas mRNA to gRNA nucleic acid of from about 2:1 to about 1:2. In specific examples, the ratio of Cas mRNA to gRNA can be about 2:1.

In some liposomes or LNPs, the cargo can comprise a nucleic acid construct encoding a polypeptide of interest (e.g., antibody or antigen-binding fragment) and gRNA. The nucleic acid construct encoding a polypeptide of interest (e.g., antibody or antigen-binding fragment) and gRNAs can be in different ratios. For example, the liposome or LNP formulation can include a ratio of nucleic acid construct to gRNA nucleic acid ranging from about 25:1 to about 1:25.

A specific example of a suitable LNP has a nitrogen-to-phosphate (N/P) ratio of about 4.5 and contains biodegradable cationic lipid, cholesterol, DSPC, and PEG2k-DMG in an about 45:44:9:2 molar ratio (about 45:about 44:about 9:about 2). The biodegradable cationic lipid can be (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate. See, e.g., Finn et al. (2018) Cell Rep. 22(9):2227-2235, herein incorporated by reference in its entirety for all purposes. The Cas9 mRNA can be in an about 1:1 (about 1:about 1) ratio by weight to the guide RNA. Another specific example of a suitable LNP contains Dlin-MC3-DMA (MC3), cholesterol, DSPC, and PEG-DMG in an about 50:38.5:10:1.5 molar ratio (about 50:about 38.5:about 10:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP has a nitrogen-to-phosphate (N/P) ratio of about 6 and contains biodegradable cationic lipid, cholesterol, DSPC, and PEG2k-DMG in an about 50:38:9:3 molar ratio (about 50:about 38:about 9:about 3). The biodegradable cationic lipid can be Lipid A ((9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 (about 2:about 1) ratio by weight to the guide RNA.

Another specific example of a suitable LNP has a nitrogen-to-phosphate (N/P) ratio of about 3 and contains a cationic lipid, a structural lipid, cholesterol (e.g., cholesterol (ovine) (Avanti 700000)), and PEG2k-DMG (e.g., PEG-DMG 2000 (NOF America-SUNBRIGHT® GM-020(DMG-PEG)) in an about 50:10:38.5:1.5 ratio (about 50:about 10:about 38.5:about 1.5) or an about 47:10:42:1 ratio (about 47:about 10:about 42:about 1). The structural lipid can be, for example, DSPC (e.g., DSPC (Avanti 850365)), SOPC, DOPC, or DOPE. The cationic/ionizable lipid can be, for example, Dlin-MC3-DMA (e.g., Dlin-MC3-DMA (Biofine International)). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Another specific example of a suitable LNP contains Dlin-MC3-DMA, DSPC, cholesterol, and a PEG lipid in an about 45:9:44:2 ratio (about 45:about 9:about 44:about 2). Another specific example of a suitable LNP contains Dlin-MC3-DMA, DOPE, cholesterol, and PEG lipid or PEG DMG in an about 50:10:39:1 ratio (about 50:about 10:about 39:about 1). Another specific example of a suitable LNP has Dlin-MC3-DMA, DSPC, cholesterol, and PEG2k-DMG at an about 55:10:32.5:2.5 ratio (about 55:about 10:about 32.5:about 2.5). Another specific example of a suitable LNP has Dlin-MC3-DMA, DSPC, cholesterol, and PEG-DMG in an about 50:10:38.5:1.5 ratio (about 50:about 10:about 38.5:about 1.5). Another specific example of a suitable LNP has Dlin-MC3-DMA, DSPC, cholesterol, and PEG-DMG in an about 50:10:38.5:1.5 ratio (about 50:about 10:about 38.5:about 1.5). The Cas9 mRNA can be in an about 1:2 ratio (about 1:about 2) by weight to the guide RNA. The Cas9 mRNA can be in an about 1:1 ratio (about 1:about 1) by weight to the guide RNA. The Cas9 mRNA can be in an about 2:1 ratio (about 2:about 1) by weight to the guide RNA.

Other examples of suitable LNPs can be found, e.g., in WO 2019/067992, WO 2020/082042, US 2020/0270617, WO 2020/082041, US 2020/0268906, WO 2020/082046 (see, e.g., pp. 85-86), and US 2020/0289628, each of which is herein incorporated by reference in its entirety for all purposes.

Dynamic Light Scattering (“DLS”) can be used to characterize the polydispersity index (“PDI”) and size of the liposomes and LNPs. In some embodiments, the PDI may range from about 0.005 to about 0.75. In some embodiments, the PDI may range from about 0.01 to about 0.5. In some embodiments, the PDI may range from about 0.02 to about 0.4. In some embodiments, the PDI may range from about 0.03 to about 0.35. In some embodiments, the PDI may range from about 0.1 to about 0.35.

The LNPs disclosed herein may have a size of about 1 to about 250 nm. In some embodiments, the LNPs may have a size of about 10 to about 200 nm. In some embodiments, the LNPs may have a size of about 20 to about 150 nm. In some embodiments, the LNPs may have a size of about 50 to about 150 nm. In some embodiments, the LNPs may have a size of about 50 to about 100 nm. In some embodiments, the LNPs may have a size of about 50 to about 120 nm. In some embodiments, the LNPs may have a size of about 75 to about 150 nm. In some embodiments, the LNPs may have a size of about 30 to about 200 nm. In some embodiments, the average sizes (diameters) of the fully formed nanoparticles are measured by dynamic light scattering on a Malvern Zetasizer (e.g., the nanoparticle sample may be diluted in phosphate buffered saline (PBS) so that the count rate is approximately 200-400 kcts, and the data may be presented as a weighted-average of the intensity measure).

In some embodiments, the liposomes or LNPs may be formed with an average encapsulation efficiency ranging from about 50% to about 100%. In some embodiments, the liposomes or LNPs may be formed with an average encapsulation efficiency ranging from about 50% to about 70%. In some embodiments, the liposomes or LNPs may be formed with an average encapsulation efficiency ranging from about 70% to about 90%. In some embodiments, the liposomes or LNPs may be formed with an average encapsulation efficiency ranging from about 90% to about 100%. In some embodiments, the liposomes or LNPs may be formed with an average encapsulation efficiency ranging from about 75% to about 95%.

In addition to liposomes and LNPs, the first and/or second component of the system described herein may be in the form of other carriers for delivery of nucleic acid and/protein molecules. Examples of other suitable carriers include, but are not limited to, lipoids and lipoplexes, particulate or polymeric nanoparticles, inorganic nanoparticles, peptide carriers, nanoparticle mimics, nanotubes, conjugates, immune stimulating complexes (ISCOM), virus-like particles (VLPs), self-assembling proteins, or emulsion delivery systems such as cationic submicron oil-in-water emulsions.

Polymeric microparticles or nanoparticles can also be used to encapsulate or adsorb a polypeptide (e.g., Cas protein) or polynucleotide (e.g., guide RNA). The particles may be substantially non-toxic and biodegradable. The particles useful for delivering a polynucleotide (e.g., guide RNA) may have an optimal size and zeta potential. For example, the microparticles may have a diameter in the range of 0.02 μm to 8 μm. In the instances when the composition has a population of micro- or nanoparticles with different diameters, at least 80%, 85%, 90%, or 95% of those particles ideally have diameters in the range of 0.03-7 μm. The particles may also have a zeta potential of between 40-100 mV, in order to provide maximal adsorption of the polynucleotide (e.g., guide RNA) to the particles.

Non-toxic and biodegradable polymers include, but are not limited to, poly(ahydroxy acids), polyhydroxy butyric acids, polylactones (including polycaprolactones), polydioxanones, polyvalerolactone, polyorthoesters, polyanhydrides, polycyanoacrylates, tyrosine-derived polycarbonates, polyvinyl-pyrrolidinones or polyester-amides, one or more natural polymers such as a polysaccharide, for example pullulan, alginate, inulin, and chitosan, and combinations thereof. In some embodiments, the particles are formed from poly(ahydroxy acids), such as a poly(lactides) (PLA), poly(g-glutamic acid) (g-PGA), poly(ethylene glycol) (PEG), polystyrene, copolymers of lactide and glycolide such as a poly(D,L-lactide-co-glycolide) (PLG), and copolymers of D,L-lactide and caprolactone. Useful PLG polymers can include those having a lactide/glycolide molar ratio ranging, for example, from 20:80 to 80:20 e.g., 25:75, 40:60, 45:55, 55:45, 60:40, 75:25. Useful PLG polymers include those having a molecular weight between, for example, 5,000-200,000 Da e.g., between 10,000-100,000, 20,000-70,000, 40,000-50,000 Da.

The polymeric nanoparticle may also form hydrogel nanoparticles, hydrophilic three-dimensional polymer networks with favorable properties including flexible mesh size, large surface area for multivalent conjugation, high water content, and high loading capacity for antigens. Polymers such as Poly(L-lactic acid) (PLA), PLGA, PEG, and polysaccharides are suitable for forming hydrogel nanoparticles.

For example, the inorganic nanoparticles may be calcium phosphate nanoparticles, silicon nanoparticles or gold nanoparticles. Inorganic nanoparticles typically have a rigid structure and comprise a shell in which a polypeptide or polynucleotide is encapsulated or a core to which the polypeptide or polynucleotide may be covalently attached. The core may comprise one or more atoms such as gold (Au), silver (Ag), copper (Cu) atoms, Au/Ag, Au/Cu, Au/Ag/Cu, Au/Pt, Au/Pd or Au/Ag/Cu/Pd or calcium phosphate (CaP).

Other molecules suitable for complexing with the polypeptides or polynucleotides of the disclosure include cationic molecules, such as, polyamidoamine, dendritic polylysine, polyethylene irinine or polypropylene imine, polylysine, chitosan, DNA-gelatin coarcervates, DEAE dextran, dendrimers, or polyethylenimine (PEI).

In some embodiments, polypeptides or polynucleotides of the present disclosure can be conjugated to nanoparticles. Nanoparticles that may be used for conjugation with antigens and/or antibodies of the present disclosure include but not are limited to chitosan-shelled nanoparticles, carbon nanotubes, PEGylated liposomes, poly(d,l-lactide-co-glycolide)/montmorillonite (PLGA/MMT) nanoparticles, poly(lactide-co-glycolide) (PLGA) nanoparticles, poly-(malic acid)-based nanoparticles, and other inorganic nanoparticles (e.g., nanoparticles made of magnesium-aluminium layered double hydroxides with disuccinimidyl carbonate (DSC), and TiO2 nanoparticles).

Oil-in-water emulsions may also be used for delivering a polypeptide or polynucleotide (e.g., mRNA) to a subject. Examples of oils useful for making the emulsions include animal (e.g., fish) oil or vegetable oil (e.g., nuts, grains and seeds). The oil may be biodegradable and biocompatible. Exemplary oils include, but are not limited to, tocopherols and squalene, a shark liver oil which is a branched, unsaturated terpenoid and combinations thereof. Terpenoids are branched chain oils that are synthesized biochemically in 5-carbon isoprene units.

The aqueous component of the emulsion can be water or can be water in which additional components have been added. For example, it may include salts to form a buffer e.g., citrate or phosphate salts, such as sodium salts. Exemplary buffers include a borate buffer, a citrate buffer, a histidine buffer a phosphate buffer, a Tris buffer, or a succinate buffer.

In some embodiments, the oil-in water emulsions include one or more cationic molecules. For example, a cationic lipid can be included in the emulsion to provide a positively charged droplet surface to which negatively-charged polynucleotide (e.g., mRNA) can attach. Exemplary cationic lipids include, but are not limited to: 1,2-dioleoyloxy-3-(trimethylammonio)propane (DOTAP), 1,2-Dimyristoyl-3-Trimethyl-AmmoniumPropane (DMTAP), 3′-[N-(N′,N′-Dimethylaminoethane)-carbamoyl]Cholesterol (DC Cholesterol), dimethyldioctadecyl-ammonium (DDA e.g., the bromide), dipalmitoyl(C16:0)trimethyl ammonium propane (DPTAP), distearoyltrimethylammonium propane (DSTAP). Other useful cationic lipids include benzalkonium chloride (BAK), benzethonium chloride, cholesterol hemisuccinate choline ester, lipopolyamines (e.g., dioctadecylamidoglycylspermine (DOGS), dipalmitoyl phosphatidylethanol-amidospermine (DPPES)), cetramide, cetylpyridinium chloride (CPC), cetyl trimethylammonium chloride (CTAC), cationic derivatives of cholesterol (e.g., cholesteryl-3.beta.-oxysuccinamidoethylenetrimethylammonium salt, cholesteryl-3.beta.-oxysuccinamidoethylene-dimethylamine, cholesteryl-3.beta.-carboxyamidoethylenetrimethylammonium salt, and cholesteryl-3.beta.-carboxyamidoethylenedimethylamine), N,N′,N′-polyoxyethylene (10)-N-tallow-1,3-diaminopropane, dodecyltrimethylammonium bromide, hexadecyltrimethyl-ammonium bromide, mixed alkyl-trimethyl-ammonium bromide, benzyldimethyldodecylammonium chloride, benzyldimethylhexadecyl-ammonium chloride, benzyltrimethylammonium methoxide, cetyldimethylethylammonium bromide, dimethyldioctadecyl ammonium bromide (DDAB), methylbenzethonium chloride, decamethonium chloride, methyl mixed trialkyl ammonium chloride, methyl trioctylammonium chloride), N,N-dimethyl-N-[2 (2-methyl-4-(1,1,3,3tetramethylbutyl)-phenoxy]-ethoxy)ethyl]-benzenemetha-naminium chloride (DEBDA), cholesteryl (4′-trimethylammonio) butanoate), N-alkyl pyridinium salts (e.g., cetylpyridinium bromide and cetylpyridinium chloride), N-alkylpiperidinium salts, dicationic bolaform electrolytes (C12Me6; C12BU6), dialkylglycetylphosphorylcholine, lysolecithin, L-alpha.dioleoylphosphatidylethanolamine, lipopoly-L (or D)-lysine (LPLL, LPDL), poly(L (or D)-lysine conjugated to N-glutarylphosphatidylethanolamine, dialkyldimethylammonium salts, [1-(2,3-dioleyloxy)-propyl]-N,N,N,trimethylammonium chloride, 1,2-diacyl-3-(trimethylammonio) propane (acyl group can be dimyristoyl, dipalmitoyl, distearoyl, or dioleoyl), 1,2-diacyl-3 (dimethylammonio)propane (acyl group can be dimyristoyl, dipalmitoyl, distearoyl, or dioleoyl), 1,2-dioleoyl-3-(4′-trimethyl-ammonio)butanoyl-sn-glycerol, 1,2-dioleoyl 3-succinyl-sn-glycerol choline ester, didodecyl glutamate ester with pendant amino group (C GluPhCnN), and ditetradecyl glutamate ester with pendant amino group (C14GluCnN+).

In some embodiments, in addition to the oil and cationic lipid, an emulsion can also include a non-ionic surfactant and/or a zwitterionic surfactant. Examples of useful surfactants include, but are not limited to: the polyoxyethylene sorbitan esters surfactants, e.g., polysorbate 20 and polysorbate 80; copolymers of ethylene oxide, propylene oxide, and/or butylene oxide, linear block copolymers; phospholipids, e.g., phosphatidylcholine; polyoxyethylene fatty ethers derived from lauryl, cetyl, stearyl and oleyl alcohols; polyoxyethylene-9-lauryl ether; octoxynols; (octylphenoxy)polyethoxyethanol; and sorbitan esters.

In some embodiments, a polynucleotide described herein may be incorporated into polynucleotide complexes, such as, but not limited to, nanoparticles (e.g., polynucleotide self-assembled nanoparticles, polymer-based self-assembled nanoparticles, inorganic nanoparticles, lipid nanoparticles, semiconductive/metallic nanoparticles), gels and hydrogels, polynucleotide complexes with cations and anions, microparticles, and any combination thereof.

In some embodiments, the polynucleotides disclosed herein may be formulated as self-assembled nanoparticles. As a non-limiting example, polynucleotides may be used to make nanoparticles which may be used in a delivery system for the polynucleotides (See e.g., PCT Publication No. WO2012/125987). In some embodiments, the polynucleotide self-assembled nanoparticles may comprise a core of the polynucleotides disclosed herein and a polymer shell. The polymer shell may be any of the polymers described herein and are known in the art. In an additional embodiment, the polymer shell may be used to protect the polynucleotides in the core.

In some embodiments, self-assembled nanoparticles may be microsponges formed of long polymers of polynucleotide hairpins which form into crystalline “pleated” sheets before self-assembling into microsponges. These microsponges are densely-packed sponge like microparticles which may function as an efficient carrier and may be able to deliver cargo to a cell. The microsponges may be from 1 μm to 300 nm in diameter. The microsponges may be complexed with other agents known in the art to form larger microsponges. As a non-limiting example, the microsponge may be complexed with an agent to form an outer layer to promote cellular uptake such as polycation polyethyleneime (PEI). This complex can form a 250-nm diameter particle that can remain stable at high temperatures (150° C.) (Grabow and Jaegar, Nature Materials 2012, 11:269-269). Additionally, these microsponges may be able to exhibit an extraordinary degree of protection from degradation by ribonucleases. In an embodiment, the polymer-based self-assembled nanoparticles such as, but not limited to, microsponges, may be fully programmable nanoparticles. The geometry, size and stoichiometry of the nanoparticle may be precisely controlled to create the optimal nanoparticle for delivery of cargo such as, but not limited to, polynucleotides.

In some embodiments, a polynucleotide disclosed herein may be formulated in inorganic nanoparticles (see U.S. Pat. No. 8,257,745). The inorganic nanoparticles may include, but are not limited to, clay substances that are water swellable. As a non-limiting example, the inorganic nanoparticle may include synthetic smectite clays which are made from simple silicates (See U.S. Pat. Nos. 5,585,108 and 8,257,745).

In some embodiments, a polynucleotide disclosed herein may be formulated in water-dispersible nanoparticle comprising a semiconductive or metallic material (U.S. Patent Application Publication No. 2012/0228565; herein incorporated by reference in its entirety) or formed in a magnetic nanoparticle (U.S. Patent Application Publication No. 2012/0265001 and 2012/0283503). The water-dispersible nanoparticles may be hydrophobic nanoparticles or hydrophilic nanoparticles.

In some embodiments, the polynucleotides disclosed herein may be encapsulated into any hydrogel known in the art which may form a gel when injected into a subject. Hydrogels are a network of polymer chains that are hydrophilic, and are sometimes found as a colloidal gel in which water is the dispersion medium. Hydrogels are highly absorbent (they can contain over 99% water) natural or synthetic polymers. Hydrogels also possess a degree of flexibility very similar to natural tissue, due to their significant water content. The hydrogel described herein may be used to encapsulate lipid nanoparticles which are biocompatible, biodegradable and/or porous.

As a non-limiting example, the hydrogel may be an aptamer-functionalized hydrogel. The aptamer-functionalized hydrogel may be programmed to release one or more polynucleotides using polynucleotide hybridization. (Battig et al., J. Am. Chem. Society. 2012 134:12410-12413). In some embodiments, the polynucleotide may be encapsulated in a lipid nanoparticle and then the lipid nanoparticle may be encapsulated into a hydrogel.

In some embodiments, the polynucleotides disclosed herein may be encapsulated into a fibrin gel, fibrin hydrogel or fibrin glue. In another embodiment, the polynucleotides may be formulated in a lipid nanoparticle or a rapidly eliminated lipid nanoparticle prior to being encapsulated into a fibrin gel, fibrin hydrogel or a fibrin glue. In yet another embodiment, the polynucleotides may be formulated as a lipoplex prior to being encapsulated into a fibrin gel, hydrogel or a fibrin glue. Fibrin gels, hydrogels and glues comprise two components, a fibrinogen solution and a thrombin solution which is rich in calcium (See e.g., Spicer and Mikos, Journal of Controlled Release 2010. 148: 49-55; Kidd et al. Journal of Controlled Release 2012. 157:80-85). The concentration of the components of the fibrin gel, hydrogel and/or glue can be altered to change the characteristics, the network mesh size, and/or the degradation characteristics of the gel, hydrogel and/or glue such as, but not limited to changing the release characteristics of the fibrin gel, hydrogel and/or glue. (See e.g., Spicer and Mikos, Journal of Controlled Release 2010. 148: 49-55; Kidd et al. Journal of Controlled Release 2012. 157:80-85; Catelas et al. Tissue Engineering 2008. 14:119-128). This feature may be advantageous when used to deliver the polynucleotide disclosed herein. (See e.g., Kidd et al. Journal of Controlled Release 2012. 157:80-85; Catelas et al. Tissue Engineering 2008. 14:119-128).

In some embodiments, a polynucleotide disclosed herein may include cations or anions. In one embodiment, the formulations include metal cations such as, but not limited to, Zn²⁻, Ca²⁺, Cu²⁺, Mg²⁺ and combinations thereof. As a non-limiting example, formulations may include polymers and a polynucleotide complexed with a metal cation (See U.S. Pat. Nos. 6,265,389 and 6,555,525).

In some embodiments, a polynucleotide may be formulated in nanoparticles and/or microparticles. These nanoparticles and/or microparticles may be molded into any size shape and chemistry. As an example, the nanoparticles and/or microparticles may be made using the PRINT® technology by LIQUIDA TECHNOLOGIES (Morrisville, N.C.) (See e.g., International Pub. Publication No. WO2007/024323).

In some embodiments, the polynucleotides disclosed herein may be formulated in NanoJackets and NanoLiposomes by Keystone Nano (State College, Pa.). NanoJackets are made of compounds that are naturally found in the body including calcium, phosphate and may also include a small amount of silicates. Nanojackets may range in size from 5 to 50 nm and may be used to deliver hydrophilic and hydrophobic compounds such as, but not limited to, polynucleotides, primary constructs and/or polynucleotide. NanoLiposomes are made of lipids such as, but not limited to, lipids which naturally occur in the body. NanoLiposomes may range in size from 60-80 nm and may be used to deliver hydrophilic and hydrophobic compounds such as, but not limited to, polynucleotides, primary constructs and/or polynucleotide. In one aspect, the polynucleotides disclosed herein are formulated in a NanoLiposome such as, but not limited to, Ceramide NanoLiposomes.

### Gene Editing Systems

In one aspect, a system or a composition described herein that is capable of introducing a gene-editing system (e.g., the CRISPR/Cas system) into a target cell (e.g., a B cell or HSC). The system comprises in one component a gene editing molecule or a polynucleotide molecule comprising a sequence encoding the gene editing molecule.

In some embodiments, at least one component of the system described herein may further comprise a guide RNA (gRNA) molecule or a sequence encoding said gRNA molecule.

In one embodiment, the system or a composition of the present disclosure comprises a recombinant viral particle comprising a gene-editing molecule and a second recombinant viral particle comprising a guide RNA (gRNA) and a sequence encoding an antibody or a fragment thereof. In certain embodiments, the gene-editing molecule is a functional fragment or derivative thereof.

A “gene-editing molecule” is a molecule (e.g., a protein or a polynucleotide molecule (e.g., mRNA) encoding such protein) used for modifying a genomic locus of interest (i.e., target) in a cell (e.g., eukaryotic, mammalian, human, or non-human cell). Such modifications include, but are not limited to a disruption, deletion, repair, mutation, addition, alteration, or modification of a gene sequence at a target locus in a gene. Examples of gene-editing molecules include, but are not limited to, endonucleases. Endonucleases are enzymes that cleave the phosphodiester bond within a polynucleotide chain, but they only break internal phosphodiester bonds. Examples of gene-editing endonucleases useful in the compositions and methods of the present disclosure include, but are not limited to, zinc finger nucleases (ZFNs), transcription activator-like effector nucleases (TALENs), meganucleases, restriction endonucleases, recombinases, and Clustered Regularly Interspersed Short Palindromic Repeats, (CRISPR)/CRISPR-associated (Cas) proteins.

### A. Cas Fusion Molecule

The methods and compositions disclosed herein can utilize the Clustered Regularly Interspersed Short Palindromic Repeats (CRISPR)/CRISPR-associated (Cas) systems or components of such systems to modify a genome within a cell. CRISPR/Cas systems include transcripts and other elements involved in the expression of, or directing the activity of, Cas genes. A CRISPR/Cas system can be, for example, a type I, a type 11, or a type III system. Alternatively, a CRISPR/Cas system can be a type V system (e.g., subtype V-A or subtype V-B). The methods and compositions disclosed herein can employ CRISPR/Cas systems by utilizing CRISPR complexes (comprising a guide RNA (gRNA) complexed with a Cas protein) for site-directed cleavage of nucleic acids.

CRISPR/Cas systems used in the compositions and methods disclosed herein can be non-naturally occurring. A “non-naturally occurring” system includes anything indicating the involvement of the hand of man, such as one or more components of the system being altered or mutated from their naturally occurring state, being at least substantially free from at least one other component with which they are naturally associated in nature, or being associated with at least one other component with which they are not naturally associated. For example, some CRISPR/Cas systems employ non-naturally occurring CRISPR complexes comprising a gRNA and a Cas protein that do not naturally occur together, employ a Cas protein that does not occur naturally, or employ a gRNA that does not occur naturally.

### (i) Cas Molecule

“Cas molecules”, “Cas proteins” or “Cas nucleases” useful in the compositions and methods of the invention generally comprise at least one RNA recognition or binding domain that can interact with guide RNAs (gRNAs, described in more detail below). Cas proteins can also comprise nuclease domains (e.g., DNase or RNase domains), DNA binding domains, helicase domains, protein-protein interaction domains, dimerization domains, and other domains. A nuclease domain possesses catalytic activity for nucleic acid cleavage, which includes the breakage of the covalent bonds of a nucleic acid molecule. Cleavage can produce blunt ends or staggered ends, and it can be single-stranded or double-stranded. For example, a wild type Cas9 protein will typically create a blunt cleavage product. Alternatively, a wild type Cpf1 protein (e.g., FnCpf1) can result in a cleavage product with a 5-nucleotide 5′ overhang, with the cleavage occurring after the 18th base pair from the PAM sequence on the non-targeted strand and after the 23rd base on the targeted strand. A Cas protein can have full cleavage activity to create a double-strand break at a target genomic locus (e.g., a double-strand break with blunt ends), or it can be a nickase that creates a single-strand break at a target genomic locus.

Examples of Cas proteins useful in the compositions and methods of the invention include Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof.

An exemplary Cas protein is a Cas9 protein or a protein derived from Cas9 from a type II CRISPR/Cas system. Cas9 proteins are from a type II CRISPR/Cas system and typically share four key motifs with a conserved architecture. Motifs 1, 2, and 4 are RuvC-like motifs, and motif 3 is an HNH motif Exemplary Cas9 proteins are from Streptococcus pyogenes, Streptococcus thermophilus, Streptococcus sp., Staphylococcus aureus, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, Neisseria meningitidis, or Campylobacter jejuni. Additional examples of the Cas9 family members are described in WO 2014/131833, herein incorporated by reference in its entirety for all purposes. Cas9 from S. pyogenes (SpCas9) (assigned SwissProt accession number Q99ZW2) is an exemplary Cas9 protein. Cas9 from S. aureus (SaCas9) (assigned UniProt accession number J7RUA5) is another exemplary Cas9 protein. Cas9 from Campylobacter jejuni (CjCas9) (assigned UniProt accession number Q0P897) is another exemplary Cas9 protein. See, e.g., Kim et al. (2017) Nat. Comm. 8:14500, herein incorporated by reference in its entirety for all purposes. SaCas9 is smaller than SpCas9, and CjCas9 is smaller than both SaCas9 and SpCas9.

Another example of a Cas protein is a Cpf1 (CRISPR from Prevotella and Francisella 1) protein. Cpf1 is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in Cas9 proteins, and the RuvC-like domain is contiguous in the Cpf1 sequence, in contrast to Cas9 where it contains long inserts including the HNH domain. See, e.g., Zetsche et al. (2015) Cell 163(3):759-771, herein incorporated by reference in its entirety for all purposes. Exemplary Cpf1 proteins are from Francisella tularensis 1, Francisella tularensis subsp. novicida, Prevotella albensis, Lachnospiraceae bacterium MC2017 1, Butyrivibrio proteoclasticus, Peregrinibacteria bacterium GW2011 GWA2 33 10, Parcubacteria bacterium GW2011 GWC2 44 17, Smithella sp. SCADC, Acidaminococcus sp. BV3L6, Lachnospiraceae bacterium MA2020, Candidatus Methanoplasma termitum, Eubacterium eligens, Moraxella bovoculi 237, Leptospira inadai, Lachnospiraceae bacterium ND2006, Porphyromonas crevioricanis 3, Prevotella disiens, and Porphyromonas macacae. Cpf1 from Francisella novicida U112 (FnCpf1; assigned UniProt accession number A0Q7Q2) is an exemplary Cpf1 protein.

Cas proteins can be wild type proteins (i.e., those that occur in nature), modified Cas proteins (i.e., Cas protein variants), or fragments of wild type or modified Cas proteins. Cas proteins can also be active variants or fragments with respect to catalytic activity of wild type or modified Cas proteins. Active variants or fragments with respect to catalytic activity can comprise at least 80%, 85%, 90%, 91%, 92%, 93%, 94%, 95%, 96%, 97%, 98%, 99% or more sequence identity to the wild type or modified Cas protein or a portion thereof, wherein the active variants retain the ability to cut at a desired cleavage site and hence retain nick-inducing or double-strand-break-inducing activity. Assays for nick-inducing or double-strand-break-inducing activity are known and generally measure the overall activity and specificity of the Cas protein on DNA substrates containing the cleavage site.

One example of a modified Cas protein is the modified SpCas9-HF1 protein, which is a high-fidelity variant of Streptococcus pyogenes Cas9 harboring alterations (N497A/R661A/Q695A/Q926A) designed to reduce non-specific DNA contacts. See, e.g., Kleinstiver et al. (2016) Nature 529(7587):490-495, herein incorporated by reference in its entirety for all purposes. Another example of a modified Cas protein is the modified eSpCas9 variant (K848A/K1003A/R1060A) designed to reduce off-target effects. See, e.g., Slaymaker et al. (2016) Science 351(6268):84-88, herein incorporated by reference in its entirety for all purposes. Other SpCas9 variants include K855A and K810A/K1003A/R1060A.

Cas proteins can be modified to increase or decrease one or more of nucleic acid binding affinity, nucleic acid binding specificity, and enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

Cas proteins can comprise at least one nuclease domain, such as a DNase domain. For example, a wild type Cpf1 protein generally comprises a RuvC-like domain that cleaves both strands of target DNA, perhaps in a dimeric configuration. Cas proteins can also comprise at least two nuclease domains, such as DNase domains. For example, a wild type Cas9 protein generally comprises a RuvC-like nuclease domain and an HNH-like nuclease domain. The RuvC and HNH domains can each cut a different strand of double-stranded DNA to make a double-stranded break in the DNA. See, e.g., Jinek et al. (2012) Science 337:816-821, herein incorporated by reference in its entirety for all purposes.

One or more of the nuclease domains can be deleted or mutated so that they are no longer functional or have reduced nuclease activity. For example, if one of the nuclease domains is deleted or mutated in a Cas9 protein, the resulting Cas9 protein can be referred to as a nickase and can generate a single-strand break at a guide RNA recognition sequence within a double-stranded DNA but not a double-strand break (i.e., it can cleave the complementary strand or the non-complementary strand, but not both). If both of the nuclease domains are deleted or mutated, the resulting Cas protein (e.g., Cas9) will have a reduced ability to cleave both strands of a double-stranded DNA (e.g., a nuclease-null or nuclease-inactive Cas protein, or a catalytically dead Cas protein (dCas)). An example of a mutation that converts Cas9 into a nickase is a D10A (aspartate to alanine at position 10 of Cas9) mutation in the RuvC domain of Cas9 from S. pyogenes. Likewise, H939A (histidine to alanine at amino acid position 839), H840A (histidine to alanine at amino acid position 840), or N863A (asparagine to alanine at amino acid position N863) in the HNH domain of Cas9 from S. pyogenes can convert the Cas9 into a nickase. Other examples of mutations that convert Cas9 into a nickase include the corresponding mutations to Cas9 from S. thermophilus. See, e.g., Sapranauskas et al. (2011) Nucleic Acids Research 39:9275-9282 and WO 2013/141680, each of which is herein incorporated by reference in its entirety for all purposes. Such mutations can be generated using methods such as site-directed mutagenesis, PCR-mediated mutagenesis, or total gene synthesis. Examples of other mutations creating nickases can be found, for example, in WO 2013/176772 and WO 2013/142578, each of which is herein incorporated by reference in its entirety for all purposes. If all of the nuclease domains are deleted or mutated in a Cas protein (e.g., both of the nuclease domains are deleted or mutated in a Cas9 protein), the resulting Cas protein (e.g., Cas9) will have a reduced ability to cleave both strands of a double-stranded DNA (e.g., a nuclease-null or nuclease-inactive Cas protein). One specific example is a D10A/H840A S. pyogenes Cas9 double mutant or a corresponding double mutant in a Cas9 from another species when optimally aligned with S. pyogenes Cas9. Another specific example is a D10A/N863A S. pyogenes Cas9 double mutant or a corresponding double mutant in a Cas9 from another species when optimally aligned with S. pyogenes Cas9.

Examples of inactivating mutations in the catalytic domains of Staphylococcus aureus Cas9 proteins are also known. For example, the Staphylococcus aureus Cas9 enzyme (SaCas9) may comprise a substitution at position N580 (e.g., N580A substitution) and a substitution at position D10 (e.g., D10A substitution) to generate a nuclease-inactive Cas protein. See, e.g., WO 2016/106236, herein incorporated by reference in its entirety for all purposes.

Examples of inactivating mutations in the catalytic domains of Cpf1 proteins are also known. With reference to Cpf1 proteins from Francisella novicida U112 (FnCpf1), Acidaminococcus sp. BV3L6 (AsCpf1), Lachnospiraceae bacterium ND2006 (LbCpf1), and Moraxella bovoculi 237 (MbCpf1 Cpf1), such mutations can include mutations at positions 908, 993, or 1263 of AsCpf1 or corresponding positions in Cpf1 orthologs, or positions 832, 925, 947, or 1180 of LbCpf1 or corresponding positions in Cpf1 orthologs. Such mutations can include, for example one or more of mutations D908A, E993A, and D1263A of AsCpf1 or corresponding mutations in Cpf1 orthologs, or D832A, E925A, D947A, and D1180A of LbCpf1 or corresponding mutations in Cpf1 orthologs. See, e.g., US 2016/0208243, herein incorporated by reference in its entirety for all purposes.

Cas fusion proteins can also be tethered to labeled nucleic acids. Such tethering (i.e., physical linking) can be achieved through covalent interactions or noncovalent interactions, and the tethering can be direct (e.g., through direct fusion or chemical conjugation, which can be achieved by modification of cysteine or lysine residues on the protein or intein modification), or can be achieved through one or more intervening linkers or adapter molecules such as streptavidin or aptamers. See, e.g., Pierce et al. (2005) Mini Rev. Med. Chem. 5(1):41-55; Duckworth et al. (2007) Angew. Chem. Int. Ed Engl. 46(46):8819-8822; Schaeffer and Dixon (2009) Australian J. Chem. 62(10):1328-1332; Goodman et al. (2009) Chembiochem. 10(9):1551-1557; and Khatwani et al. (2012) Bioorg. Med. Chem. 20(14):4532-4539, each of which is herein incorporated by reference in its entirety for all purposes. Noncovalent strategies for synthesizing protein-nucleic acid conjugates include biotin-streptavidin and nickel-histidine methods. Covalent protein-nucleic acid conjugates can be synthesized by connecting appropriately functionalized nucleic acids and proteins using a wide variety of chemistries. Some of these chemistries involve direct attachment of the oligonucleotide to an amino acid residue on the protein surface (e.g., a lysine amine or a cysteine thiol), while other more complex schemes require post-translational modification of the protein or the involvement of a catalytic or reactive protein domain. Methods for covalent attachment of proteins to nucleic acids can include, for example, chemical cross-linking of oligonucleotides to protein lysine or cysteine residues, expressed protein-ligation, chemoenzymatic methods, and the use of photoaptamers. The labeled nucleic acid can be tethered to the C-terminus, the N-terminus, or to an internal region within the Cas protein. Preferably, the labeled nucleic acid is tethered to the C-terminus or the N-terminus of the Cas protein. Likewise, the Cas protein can be tethered to the 5′ end, the 3′ end, or to an internal region within the labeled nucleic acid. That is, the labeled nucleic acid can be tethered in any orientation and polarity. Preferably, the Cas protein is tethered to the 5′ end or the 3′ end of the labeled nucleic acid.

In some embodiments, the nucleic acids encoding the Cas proteins of the invention, or functional fragments or derivatives thereof, can be codon optimized for efficient translation into protein in a particular cell or organism. For example, the nucleic acid encoding a Cas protein, or functional fragment or derivative thereof, can be modified to substitute codons having a higher frequency of usage in a bacterial cell, a yeast cell, a human cell, a non-human cell, a mammalian cell, a rodent cell, a mouse cell, a rat cell, or any other host (e.g. packaging) and/or target cell of interest. When a fusion RNA encoding a Cas protein, or a functional fragment or derivative thereof, is introduced into the cell, the Cas protein, or functional fragment or derivative thereof, can be transiently or conditionally expressed in the cell.

In certain embodiments, the Cas molecule is a Cas9 molecule, or a functional fragment or derivative thereof. In certain embodiments, the Cas9 can be wild type Cas9, a Cas9 nickase, a dead Cas9 (dCas9) a split Cas9, and a Cas9 fusion protein. In certain embodiments, the Cas9 is a Streptococcus pyogenes or Staphylococcus aureus Cas9. In certain embodiments, the sequence of the Cas9 mRNA is codon optimized for expression in a eukaryotic cell.

Optionally, the Cas mRNA can be codon optimized for efficient translation into the Cas protein, or functional fragment or derivative thereof, in a particular cell or organism. For example, the nucleic acid sequence encoding the Cas protein, or a functional fragment or derivative thereof, can be modified to substitute codons having a higher frequency of usage in a bacterial cell, a yeast cell, a human cell, a non-human cell, a mammalian cell, a rodent cell, a mouse cell, a rat cell, or any other host (e.g., packaging) and/or target cell of interest, as compared to the naturally occurring polynucleotide sequence.

In certain embodiments, the Cas protein is Cas9, or functional fragment or derivative thereof. In certain embodiments, the Cas9 is selected from the group consisting of wild type Cas9, a Cas9 nickase, a dead Cas9 (dCas9), a split Cas9, an inducible Cas9, and a Cas9 fusion protein. In certain embodiments, the Cas9 is a Streptococcus pyogenes or Staphylococcus aureus Cas9. In certain embodiments, the sequence of the Cas9 mRNA is codon optimized for expression in a eukaryotic cell.

Cas proteins, or functional fragment or derivative thereof, also be operably linked to other heterologous polypeptides as fusion proteins. For example, a Cas protein, or functional fragment or derivative thereof, can be fused to a cleavage domain, an epigenetic modification domain, a transcriptional activation domain, or a transcriptional repressor domain. See WO 2014/089290, herein incorporated by reference in its entirety for all purposes. Examples of transcriptional activation domains include a herpes simplex virus VP16 activation domain, VP64 (which is a tetrameric derivative of VP16), a NFκB p65 activation domain, p53 activation domains 1 and 2, a CREB (cAMP response element binding protein) activation domain, an E2A activation domain, and an NFAT (nuclear factor of activated T-cells) activation domain. Other examples include activation domains from Oct1, Oct-2A, SP1, AP-2, CTF1, P300, CBP, PCAF, SRC1, PvALF, ERF-2, OsGAI, HALF-1, C1, AP1, ARF-5, ARF-6, ARF-7, ARF-8, CPRF1, CPRF4, MYC-RP/GP, TRABIPC4, and HSF1. See, e.g., US 2016/0237456, EP3045537, and WO 2011/145121, each of which is incorporated by reference in its entirety for all purposes. In some cases, a transcriptional activation system can be used comprising a dCas9-VP64 fusion protein paired with MS2-p65-HSF1. Guide RNAs in such systems can be designed with aptamer sequences appended to sgRNA tetraloop and stem-loop 2 designed to bind dimerized MS2 bacteriophage coat proteins. See, e.g., Konermann et al. (2015) Nature 517(7536):583-588, herein incorporated by reference in its entirety for all purposes. Examples of transcriptional repressor domains include inducible cAMP early repressor (ICER) domains, Kruppel-associated box A (KRAB-A) repressor domains, YY1 glycine rich repressor domains, Sp1-like repressors, E(spl) repressors, IκB repressor, and MeCP2. Other examples include transcriptional repressor domains from A/B, KOX, TGF-beta-inducible early gene (TIEG), v-erbA, SID, SID4X, MBD2, MBD3, DNMT1, DNMG3A, DNMT3B, Rb, ROM2, See, e.g., EP3045537 and WO 2011/145121, each of which is incorporated by reference in its entirety for all purposes. Cas proteins can also be fused to a heterologous polypeptide providing increased or decreased stability. The fused domain or heterologous polypeptide can be located at the N-terminus, the C-terminus, or internally within the Cas protein, or functional fragment or derivative thereof.

As one example, a Cas protein, or functional fragment or derivative thereof, can be fused to one or more heterologous polypeptides that provide for subcellular localization. Such heterologous polypeptides can include, for example, one or more nuclear localization signals (NLS) such as the SV40 NLS and/or an alpha-importin NLS for targeting to the nucleus, a mitochondrial localization signal for targeting to the mitochondria, an ER retention signal, and the like. See, e.g., Lange et al. (2007). J. Biol. Chem. 282:5101-5105, herein incorporated by reference in its entirety for all purposes. Such subcellular localization signals can be located at the N-terminus, the C-terminus, or anywhere within the Cas protein, or functional fragment or derivative thereof. An NLS can comprise a stretch of basic amino acids, and can be a monopartite sequence or a bipartite sequence. Optionally, the Cas protein, or functional fragment or derivative thereof, comprises two or more NLSs, including an NLS (e.g., an alpha-importin NLS) at the N-terminus and/or an NLS (e.g., an SV40 NLS) at the C-terminus.

Cas proteins, or functional fragment or derivative thereof, can also be operably linked to a heterologous polypeptide for ease of tracking or purification, such as a fluorescent protein, a purification tag, or an epitope tag. Examples of fluorescent proteins include green fluorescent proteins (e.g., GFP, GFP-2, tagGFP, turboGFP, eGFP, Emerald, Azami Green, Monomeric Azami Green, CopGFP, AceGFP, ZsGreenl), yellow fluorescent proteins (e.g., YFP, eYFP, Citrine, Venus, YPet, PhiYFP, ZsYellowl), blue fluorescent proteins (e.g. eBFP, eBFP2, Azurite, mKalamal, GFPuv, Sapphire, T-sapphire), cyan fluorescent proteins (e.g. eCFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRedl, AsRed2, eqFP611, mRaspberry, mStrawberry, Jred), orange fluorescent proteins (mOrange, mKO, Kusabira-Orange, Monomeric Kusabira-Orange, mTangerine, tdTomato), and any other suitable fluorescent protein. Examples of tags include glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, hemagglutinin (HA), nus, Softag 1, Softag 3, Strep, SBP, Glu-Glu, HSV, KT3, S, S1, T7, V5, VSV-G, histidine (His), biotin carboxyl carrier protein (BCCP), and calmodulin.

The Cas fusion protein can be produced using routine molecular biology techniques, such as those described above or in He et al., supra. Alternatively, Cas fusion protein can be prepared by various other methods.

In certain embodiments, the nucleic acid encoding the Cas fusion protein comprises a regulatory element, including for example, a promoter, an enhancer, or a transcriptional repressor-binding element. Exemplary expression control sequences are known in the art and described in, for example, Goeddel, (1990) Gene Expression Technology: Methods in Enzymology, Vol. 185, Academic Press, San Diego, Calif, incorporated by reference in its entirety for all purposes.

### B. Transcription Activator-Like Effector Nucleases, Zinc Figure Nucleases, Meganucleases, and Restriction Endonucleases

In certain embodiments, the gene-editing molecule can be zinc finger nucleases (ZFns), transcription activator-like effector nucleases (TALENs), meganucleases, and/or restriction endonucleases. Fusion RNA and fusion protein molecules using these gene-editing molecules, or functional fragment or derivative thereof, for use in the compositions and methods of the invention can be made in the same fashion and structure as that disclosed above for Cas molecules, or functional fragment or derivative thereof.

Transcription activator-like effector nucleases (TALEN) are restriction enzymes that can be engineered to cut target sequences of DNA. They are made by fusing a TAL effector DNA-binding domain to a DNA cleavage domain (a nuclease which cuts DNA strands). TAL effector nucleases are a class of sequence-specific nucleases that can be used to make double-strand breaks at specific target sequences in the genome of a prokaryotic or eukaryotic organism. TAL effector nucleases are created by fusing a native or engineered transcription activator-like (TAL) effector, or functional part thereof, to the catalytic domain of an endonuclease, such as, for example, FokI. The unique, modular TAL effector DNA binding domain allows for the design of proteins with potentially any given DNA recognition specificity. Thus, the DNA binding domains of the TAL effector nucleases can be engineered to recognize specific DNA target sites and thus, used to make double-strand breaks at desired target sequences. See, WO 2010/079430; Morbitzer et al. (2010) PNAS 10.1073/pnas.1013133107; Scholze & Boch (2010) Virulence 1:428-432; Christian et al. Genetics (2010) 186:757-761; Li et al. (2010) Nuc. Acids Res. doi: 10.1093/nar/gkg704; and Miller et al. (2011) Nature Biotechnology 29:143-148; all of which are herein incorporated by reference in their entirety and for all purposes.

Examples of suitable TAL nucleases, and methods for preparing suitable TAL nucleases, are disclosed, e.g., in US Patent Application No. 2011/0239315 A1, 2011/0269234 A1, 2011/0145940 A1, 2003/0232410 A1, 2005/0208489 A1, 2005/0026157 A1, 2005/0064474 A1, 2006/0188987 A1, and 2006/0063231 A1 (each hereby incorporated by reference in their entirety and for all purposes). In various embodiments, TAL effector nucleases are engineered that cut in or near a target nucleic acid sequence in, e.g., a genomic locus of interest, wherein the target nucleic acid sequence is at or near a sequence to be modified by a targeting vector. The TAL nucleases suitable for use with the various methods and compositions provided herein include those that are specifically designed to bind at or near target nucleic acid sequences to be modified by targeting vectors.

In one embodiment, each monomer of the TALEN comprises 12-25 TAL repeats, wherein each TAL repeat binds a 1 bp subsite. In certain embodiments, the gene-editing molecule is a chimeric protein comprising a TAL repeat-based DNA binding domain operably linked to an independent nuclease. In certain embodiments, the independent nuclease is a FokI endonuclease. In one embodiment, the gene-editing molecule comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by about 6 bp to about 40 bp cleavage site, and wherein the FokI nucleases dimerize and make a double strand break at a target sequence.

In certain embodiments, the gene-editing molecule comprises a first TAL-repeat-based DNA binding domain and a second TAL-repeat-based DNA binding domain, wherein each of the first and the second TAL-repeat-based DNA binding domain is operably linked to a FokI nuclease, wherein the first and the second TAL-repeat-based DNA binding domain recognize two contiguous target DNA sequences in each strand of the target DNA sequence separated by a 5 bp or 6 bp cleavage site, and wherein the FokI nucleases dimerize and make a double strand break.

The gene-editing molecule employed in the various methods and compositions disclosed herein can further comprise a zinc-finger nuclease (ZFN). Zinc finger nucleases (ZFNs) are a class of engineered DNA-binding proteins that assist targeted editing of the genome by creating double strand breaks (DSBs) in DNA at targeted locations. ZFNs comprise two functional domains: i) a DNA-binding domain comprising a chain of two-finger modules (each recognizing a unique hexamer (6 bp) sequence of DNA—two-finger modules are stitched together to form a Zinc Finger Protein, each with specificity of ≥24 bp) and ii) a DNA-cleaving domain comprising a nuclease domain of Fok I. When the DNA-binding and -cleaving domains are fused together, a highly-specific pair of “genomic scissors” are created.

In certain embodiments, each monomer of the ZFN comprises 3 or more zinc finger-based DNA binding domains, wherein each zinc finger-based DNA binding domain binds to a 3 bp subsite. In other embodiments, the ZFN is a chimeric protein comprising a zinc finger-based DNA binding domain operably linked to an independent nuclease. In certain embodiments, the independent endonuclease is a FokI endonuclease. In certain embodiments, the gene-editing molecule comprises a first ZFN and a second ZFN, wherein each of the first ZFN and the second ZFN is operably linked to a FokI nuclease, wherein the first and the second ZFN recognize two contiguous target DNA sequences in each strand of the target DNA sequence separated by about 6 bp to about 40 bp cleavage site or about a 5 bp to about 6 bp cleavage site, and wherein the FokI nucleases dimerize and make a double strand break. See, e.g., US20060246567; US20080182332; US20020081614; US20030021776; WO/2002/057308A2; US20130123484; US20100291048; and, WO/2011/017293A2, each of which is herein incorporated by reference in their entirety for all purposes.

In certain embodiments of the compositions and methods provided herein, the gene-editing molecule comprises (a) a chimeric protein comprising a zinc finger-based DNA binding domain fused to a FokI endonuclease; or (b) a chimeric protein comprising a Transcription Activator-Like Effector Nuclease (TALEN) fused to a FokI endonuclease.

In still another embodiment, the gene-editing molecule is a meganuclease. Meganucleases have been classified into four families based on conserved sequence motifs, the families are the LAGLIDADG, GIY-YIG, H-N-H, and His-Cys box families. These motifs participate in the coordination of metal ions and hydrolysis of phosphodiester bonds. HEases are notable for their long recognition sites, and for tolerating some sequence polymorphisms in their DNA substrates. Meganuclease domains, structure and function are known, see e.g., Guhan and Muniyappa (2003) Crit Rev Biochem Mol Biol 38:199-248; Lucas et al., (2001) Nucleic Acids Res 29:960-9; Jurica and Stoddard, (1999) Cell Mol Life Sci 55:1304-26; Stoddard, (2006) Q Rev Biophys 38:49-95; and Moure et al., (2002) Nat Struct Biol 9:764. In some examples a naturally occurring variant, and/or engineered derivative meganuclease is used. Methods for modifying the kinetics, cofactor interactions, expression, optimal conditions, and/or recognition site specificity, and screening for activity are known, see e.g., Epinat et al., (2003) Nucleic Acids Res 31:2952-62; Chevalier et al., (2002) Mol Cell 10:895-905; Gimble et al., (2003) Mol Biol 334:993-1008; Seligman et al., (2002) Nucleic Acids Res 30:3870-9; Sussman et al., (2004) J Mol Biol 342:31-41; Rosen et al., (2006) Nucleic Acids Res 34:4791-800; Chames et al., (2005) Nucleic Acids Res 33:e178; Smith et al., (2006) Nucleic Acids Res 34:e149; Gruen et al., (2002) Nucleic Acids Res 30:e29; Chen and Zhao, (2005) Nucleic Acids Res 33:e154; WO2005105989; WO2003078619; WO2006097854; WO2006097853; WO2006097784; and WO2004031346.

Any meganuclease can be used herein, including, but not limited to, I-SceI, I-SceII, I-SceIlI, I-SceIV, I-SceV, I-SceVI, I-SceVII, I-CeuI, I-CeuAIIP, I-Crel, I-CrepsbIP, I-CrepsbIlP, I-CrepsbIIIP, I-CrepsbIVP, I-TliI, I-PpoI, PI-PspI, F-SceI, F-SceII, F-SuvI, F-TevI, F-TevII, I-Aural, I-Anil, I-Chul, I-CmoeI, I-CpaI, I-CpaII, I-CsmI, I-CvuI, I-CvuAIP, I-DdiI, I-DdiII, I-DirI, I-DmoI, I-HmuI, I-HmuII, I-HsNIP, I-LlaI, I-MsoI, I-NaaI, I-NanI, I-NcIIP, I-NgrIP, I-NitI, I-NjaI, I-Nsp236IP, I-PakI, I-PboIP, I-PcuIP, I-PcuAI, I-PcuVI, I-PgrIP, I-PobIP, I-PorI, I-PorIIP, I-PbpIP, I-SpBetaIP, I-ScaI, I-SexIP, I-SneIP, I-SpomI, I-SpomCP, I-SpomIP, I-SpomIIP, I-SquIP, I-Ssp6803I, I-SthPhiJP, I-SthPhiST3P, I-SthPhiSTe3bP, I-TdeIP, I-TevI, I-TevII, I-TevIII, I-UarAP, I-UarHGPAIP, I-UarHGPA13P, I-VinIP, I-ZbiIP, PI-MtuI, PI-MtuHIP PI-MtuHIIP, PI-PfuI, PI-PfulI, PI-Pkol, PI-Pkoll, PI-Rma438121P, PI-SpBetalP, PI-SceI, PI-Tful, PI-TfuII, PI-ThyI, PI-TliI, PI-TliII, or any active variants or fragments thereof.

In one embodiment, the meganuclease recognizes double-stranded DNA sequences of 12 to 40 base pairs. In one embodiment, the meganuclease recognizes one perfectly matched target sequence in the genome. In one embodiment, the meganuclease is a homing nuclease. In one embodiment, the homing nuclease is a LAGLIDADG family of homing nuclease. In one embodiment, the LAGLIDADG family of homing nuclease is selected from I-SceI, I-CreI, and I-DmoI.

Gene-editing molecules can further comprise restriction endonucleases, which include Type I, Type II, Type III, and Type IV endonucleases. Type I and Type III restriction endonucleases recognize specific recognition sites, but typically cleave at a variable position from the nuclease binding site, which can be hundreds of base pairs away from the cleavage site (recognition site). In Type II systems the restriction activity is independent of any methylase activity, and cleavage typically occurs at specific sites within or near to the binding site. Most Type II enzymes cut palindromic sequences, however Type IIa enzymes recognize non-palindromic recognition sites and cleave outside of the recognition site, Type IIb enzymes cut sequences twice with both sites outside of the recognition site, and Type Its enzymes recognize an asymmetric recognition site and cleave on one side and at a defined distance of about 1-20 nucleotides from the recognition site. Type IV restriction enzymes target methylated DNA. Restriction enzymes are further described and classified, for example in the REBASE database (webpage at rebase.neb.com; Roberts et al., (2003) Nucleic Acids Res 31:418-20), Roberts et al., (2003) Nucleic Acids Res 31:1805-12, and Belfort et al., (2002) in Mobile DNA II, pp. 761-783, Eds. Craigie et al., (ASM Press, Washington, D.C.).

ZFNs and TALENs introduce DSBs in a target genomic sequence and activate non-homologous end-joining (NHEJ)-mediated DNA repair, which generates a mutant allele comprising an insertion or a deletion of a nucleic acid sequence at the genomic locus of interest and thereby causes disruption of the genomic locus of interest in a cell. DSBs also stimulate homology-directed repair (HDR) by homologous recombination if a repair template is provided. HDR can result in a perfect repair that restores the original sequence at the broken site, or it can be used to direct a designed modification, such as a deletion, insertion, or replacement of the sequence at the site of the double strand break.

### C. Guide RNAs

In one aspect, the system described herein comprises a guide RNA (gRNA).

A “guide RNA” or “gRNA” is an RNA molecule that binds to a Cas protein (e.g., Cas9 protein), or functional fragment or derivative thereof, and targets the Cas protein to a specific location within a target DNA. Guide RNAs can comprise two segments: a “DNA-targeting segment” and a “protein-binding segment”. “Segment” includes a section or region of a molecule, such as a contiguous stretch of nucleotides in an RNA. Some gRNAs, such as those for Cas9, can comprise two separate RNA molecules: an “activator-RNA” (e.g., tracrRNA) and a “targeter-RNA” (e.g., CRISPR RNA or crRNA). Other gRNAs are a single RNA molecule (single RNA polynucleotide), which can also be called a “single-molecule gRNA,” a “single-guide RNA,” or an “sgRNA”. See, e.g., WO 2013/176772, WO 2014/065596, WO 2014/089290, WO 2014/093622, WO 2014/099750, WO 2013/142578, and WO 2014/131833, each of which is herein incorporated by reference in its entirety for all purposes. For Cas9, for example, a single-guide RNA can comprise a crRNA fused to a tracrRNA (e.g., via a linker). For Cpf1, for example, only a crRNA is needed to achieve binding to a target sequence. The terms “guide RNA” and “gRNA” include both double-molecule (i.e., modular) gRNAs and single-molecule gRNAs.

An exemplary two-molecule gRNA comprises a crRNA-like (“CRISPR RNA” or “targeter-RNA” or “crRNA” or “crRNA repeat”) molecule and a corresponding tracrRNA-like (“trans-acting CRISPR RNA” or “activator-RNA” or “tracrRNA”) molecule. A crRNA comprises both the DNA-targeting segment (single-stranded) of the gRNA and a stretch of nucleotides that forms one half of the dsRNA duplex of the protein-binding segment of the gRNA.

A corresponding tracrRNA (activator-RNA) comprises a stretch of nucleotides that forms the other half of the dsRNA duplex of the protein-binding segment of the gRNA. A stretch of nucleotides of a crRNA are complementary to and hybridize with a stretch of nucleotides of a tracrRNA to form the dsRNA duplex of the protein-binding domain of the gRNA. As such, each crRNA can be said to have a corresponding tracrRNA.

In systems in which both a crRNA and a tracrRNA are needed, the crRNA and the corresponding tracrRNA hybridize to form a gRNA. In systems in which only a crRNA is needed, the crRNA can be the gRNA. The crRNA additionally provides the single-stranded DNA-targeting segment that hybridizes to a guide RNA recognition sequence. If used for modification within a cell, the exact sequence of a given crRNA or tracrRNA molecule can be designed to be specific to the species in which the RNA molecules will be used. See, e.g., Mali et al. (2013) Science 339:823-826; Jinek et al. (2012) Science 337:816-821; Hwang et al. (2013) Nat. Biotechnol. 31:227-229; Jiang et al. (2013) Nat. Biotechnol. 31:233-239; and Cong et al. (2013) Science 339:819-823, each of which is herein incorporated by reference in its entirety for all purposes.

The DNA-targeting segment (crRNA) of a given gRNA comprises a nucleotide sequence that is complementary to a sequence (i.e., the guide RNA recognition sequence) in a target DNA. The DNA-targeting segment of a gRNA interacts with a target DNA in a sequence-specific manner via hybridization (i.e., base pairing). As such, the nucleotide sequence of the DNA-targeting segment may vary and determines the location within the target DNA with which the gRNA and the target DNA will interact. The DNA-targeting segment of a subject gRNA can be modified to hybridize to any desired sequence within a target DNA. Naturally occurring crRNAs differ depending on the CRISPR/Cas system and organism but often contain a targeting segment of between 21 to 72 nucleotides length, flanked by two direct repeats (DR) of a length of between 21 to 46 nucleotides (see, e.g., WO 2014/131833, herein incorporated by reference in its entirety for all purposes). In the case of S. pyogenes, the DRs are 36 nucleotides long and the targeting segment is 30 nucleotides long. The 3′ located DR is complementary to and hybridizes with the corresponding tracrRNA, which in turn binds to the Cas protein.

The DNA-targeting segment can have a length of at least about 12 nucleotides, at least about 15 nucleotides, at least about 17 nucleotides, at least about 18 nucleotides, at least about 19 nucleotides, at least about 20 nucleotides, at least about 25 nucleotides, at least about 30 nucleotides, at least about 35 nucleotides, or at least about 40 nucleotides. Such DNA-targeting segments can have a length from about 12 nucleotides to about 100 nucleotides, from about 12 nucleotides to about 80 nucleotides, from about 12 nucleotides to about 50 nucleotides, from about 12 nucleotides to about 40 nucleotides, from about 12 nucleotides to about 30 nucleotides, from about 12 nucleotides to about 25 nucleotides, or from about 12 nucleotides to about 20 nucleotides. For example, the DNA targeting segment can be from about 15 nucleotides to about 25 nucleotides (e.g., from about 17 nucleotides to about 20 nucleotides, or about 17 nucleotides, about 18 nucleotides, about 19 nucleotides, or about 20 nucleotides). See, e.g., US 2016/0024523, herein incorporated by reference in its entirety for all purposes. For Cas9 from S. pyogenes, a typical DNA-targeting segment is between 16 and 20 nucleotides in length or between 17 and 20 nucleotides in length. For Cas9 from S. aureus, a typical DNA-targeting segment is between 21 and 23 nucleotides in length. For Cpf1, a typical DNA-targeting segment is at least 16 nucleotides in length or at least 18 nucleotides in length.

TracrRNAs can be in any form (e.g., full-length tracrRNAs or active partial tracrRNAs) and of varying lengths. They can include primary transcripts or processed forms. For example, tracrRNAs (as part of a single-guide RNA or as a separate molecule as part of a two-molecule gRNA) may comprise or consist of all or a portion of a wild type tracrRNA sequence (e.g., about or more than about 20, 26, 32, 45, 48, 54, 63, 67, 85, or more nucleotides of a wild type tracrRNA sequence). Examples of wild type tracrRNA sequences from S. pyogenes include 171-nucleotide, 89-nucleotide, 75-nucleotide, and 65-nucleotide versions. See, e.g., Deltcheva et al. (2011) Nature 471:602-607; WO 2014/093661, each of which is herein incorporated by reference in its entirety for all purposes. Examples of tracrRNAs within single-guide RNAs (sgRNAs) include the tracrRNA segments found within +48, +54, +67, and +85 versions of sgRNAs, where “+n” indicates that up to the +n nucleotide of wild type tracrRNA is included in the sgRNA. See U.S. Pat. No. 8,697,359, herein incorporated by reference in its entirety for all purposes.

The percent complementarity between the DNA-targeting sequence and the guide RNA recognition sequence within the target DNA can be at least 60% (e.g., at least 65%, at least 70%, at least 75%, at least 80%, at least 85%, at least 90%, at least 95%, at least 97%, at least 98%, at least 99%, or 100%). The percent complementarity between the DNA-targeting sequence and the guide RNA recognition sequence within the target DNA can be at least 60% over about 20 contiguous nucleotides. As an example, the percent complementarity between the DNA-targeting sequence and the guide RNA recognition sequence within the target DNA is 100% over the 14 contiguous nucleotides at the 5′ end of the guide RNA recognition sequence within the complementary strand of the target DNA and as low as 0% over the remainder. In such a case, the DNA-targeting sequence can be considered to be 14 nucleotides in length. As another example, the percent complementarity between the DNA-targeting sequence and the guide RNA recognition sequence within the target DNA is 100% over the seven contiguous nucleotides at the 5′ end of the guide RNA recognition sequence within the complementary strand of the target DNA and as low as 0% over the remainder. In such a case, the DNA-targeting sequence can be considered to be 7 nucleotides in length. In some guide RNAs, at least 17 nucleotides within the DNA-target sequence are complementary to the target DNA. For example, the DNA-targeting sequence can be 20 nucleotides in length and can comprise 1, 2, or 3 mismatches with the target DNA (the guide RNA recognition sequence). Preferably, the mismatches are not adjacent to a protospacer adjacent motif (PAM) sequence (e.g., the mismatches are in the 5′ end of the DNA-targeting sequence, or the mismatches are at least 2, 3, 4, 5, 6, 7, 8, 9, 10, 11, 12, 13, 14, 15, 16, 17, 18, or 19 base pairs away from the PAM sequence).

The protein-binding segment of a gRNA can comprise two stretches of nucleotides that are complementary to one another. The complementary nucleotides of the protein-binding segment hybridize to form a double-stranded RNA duplex (dsRNA). The protein-binding segment of a subject gRNA interacts with a Cas protein, or functional fragment or derivative thereof, and the gRNA directs the bound Cas protein, or functional fragment or derivative thereof, to a specific nucleic acid sequence within target DNA via the DNA-targeting segment.

Guide RNAs can include modifications or sequences that provide for additional desirable features (e.g., modified or regulated stability; subcellular targeting; tracking with a fluorescent label; a binding site for a protein or protein complex; and the like). Examples of such modifications include, for example, a 5′ cap (e.g., a 7-methylguanylate cap (m7G)); a 3′ polyadenylated tail (i.e., a 3′ poly(A) tail); a riboswitch sequence (e.g., to allow for regulated stability and/or regulated accessibility by proteins and/or protein complexes); a stability control sequence; a sequence that forms a dsRNA duplex (i.e., a hairpin); a modification or sequence that targets the RNA to a subcellular location (e.g., nucleus, mitochondria, chloroplasts, and the like); a modification or sequence that provides for tracking (e.g., direct conjugation to a fluorescent molecule, conjugation to a moiety that facilitates fluorescent detection, a sequence that allows for fluorescent detection, and so forth); a modification or sequence that provides a binding site for proteins (e.g., proteins that act on DNA, including transcriptional activators, transcriptional repressors, DNA methyltransferases, DNA demethylases, histone acetyltransferases, histone deacetylases, and the like); and combinations thereof. Other examples of modifications include engineered stem loop duplex structures, engineered bulge regions, engineered hairpins 3′ of the stem loop duplex structure, or any combination thereof. See, e.g., US 2015/0376586, herein incorporated by reference in its entirety for all purposes. A bulge can be an unpaired region of nucleic acids within the duplex made up of the crRNA-like region and the minimum tracrRNA-like region. A bulge can comprise, on one side of the duplex, an unpaired 5′-XXXY-3′ where X is any purine and Y can be a nucleotide that can form a wobble pair with a nucleotide on the opposite strand, and an unpaired nucleotide region on the other side of the duplex.

In some cases, a transcriptional activation system can be used comprising a dCas9-VP64 fusion protein paired with MS2-p65-HSF1. Guide RNAs in such systems can be designed with aptamer sequences appended to sgRNA tetraloop and stem-loop 2 designed to bind dimerized MS2 bacteriophage coat proteins. See, e.g., Konermann et al. (2015) Nature 517(7536):583-588, herein incorporated by reference in its entirety for all purposes.

Guide RNAs can be provided in any form. For example, the gRNA can be provided in the form of RNA, either as two molecules (separate crRNA and tracrRNA) or as one molecule (sgRNA). The gRNA can also be provided in the form of DNA encoding the gRNA. The DNA encoding the gRNA can encode a single RNA molecule (sgRNA) or separate RNA molecules (e.g., separate crRNA and tracrRNA). In the latter case, the DNA encoding the gRNA can be provided as one DNA molecule or as separate DNA molecules encoding the crRNA and tracrRNA, respectively.

When a gRNA is provided in the form of DNA, the gRNA can be transiently, conditionally, or constitutively expressed in the cell. DNAs encoding gRNAs can be stably integrated into the genome of the cell and operably linked to a promoter active in the cell. Alternatively, DNAs encoding gRNAs can be operably linked to a promoter in an expression construct. For example, the DNA encoding the gRNA can be in a vector comprising a heterologous nucleic acid. Promoters that can be used in such expression constructs include promoters active, for example, in one or more of a eukaryotic cell, a human cell, a non-human cell, a mammalian cell, a non-human mammalian cell, a rodent cell, a mouse cell, a rat cell, a hamster cell, a rabbit cell, a pluripotent cell, an embryonic stem (ES) cell, an adult stem cell, a developmentally restricted progenitor cell, an induced pluripotent stem (iPS) cell, or a one-cell stage embryo. Such promoters can be, for example, conditional promoters, inducible promoters, constitutive promoters, or tissue-specific promoters. Such promoters can also be, for example, bidirectional promoters. In certain embodiments, an RNA Pol III promoter can be operatively linked to a gRNA sequence (if included in the lentivirus vector) to control expression of such sequence. RNA Pol III promoters are frequently used to express small RNAs, such as small interfering RNA (siRNA)/short hairpin RNA (shRNA) and guide RNA sequences used in CRISPR-Cas9 systems. Examples of RNA Pol III promoters that can be used in the invention include, but are not limited to, the human U6 promoter, a rat U6 polymerase III promoter, or a mouse U6 polymerase III promoter, and the H1 promoter, which are described in, for example Goomer and Kunkel, Nucl. Acids Res., 20 (18): 4903-4912 (1992), and Myslinski et al., Nucleic Acids Res., 29(12): 2502-9 (2001).

### D. Guide RNA Recognition Sequences

The term “guide RNA recognition sequence” includes nucleic acid sequences present in a target DNA to which a DNA-targeting segment of a gRNA will bind, provided sufficient conditions for binding exist. For example, gRNA recognition sequences include sequences to which a gRNA is designed to have complementarity, where hybridization between a guide RNA recognition sequence and a DNA targeting sequence promotes the formation of a CRISPR complex. Full complementarity is not necessarily required, provided that there is sufficient complementarity to cause hybridization and promote formation of a CRISPR complex. Guide RNA recognition sequences also include cleavage sites for Cas proteins, described in more detail below. A gRNA recognition sequence can comprise any polynucleotide, which can be located, for example, in the nucleus or cytoplasm of a cell or within an organelle of a cell, such as a mitochondrion or chloroplast.

The gRNA recognition sequence within a target DNA can be targeted by (i.e., be bound by, or hybridize with, or be complementary to) a Cas protein or a gRNA. Suitable DNA/RNA binding conditions include physiological conditions normally present in a cell. Other suitable DNA/RNA binding conditions (e.g., conditions in a cell-free system) are known in the art (see, e.g., Molecular Cloning: A Laboratory Manual, 3rd Ed. (Sambrook et al., Harbor Laboratory Press 2001), herein incorporated by reference in its entirety for all purposes). The strand of the target DNA that is complementary to and hybridizes with the Cas protein or gRNA can be called the “complementary strand,” and the strand of the target DNA that is complementary to the “complementary strand” (and is therefore not complementary to the Cas protein or gRNA) can be called “noncomplementary strand” or “template strand.”

The Cas protein can cleave the nucleic acid at a site within or outside of the nucleic acid sequence present in the target DNA to which the DNA-targeting segment of a gRNA will bind. The “cleavage site” includes the position of a nucleic acid at which a Cas protein produces a single-strand break or a double-strand break. For example, formation of a CRISPR complex (comprising a gRNA hybridized to a guide RNA recognition sequence and complexed with a Cas protein) can result in cleavage of one or both strands in or near (e.g., within 1, 2, 3, 4, 5, 6, 7, 8, 9, 10, 20, 50, or more base pairs from) the nucleic acid sequence present in a target DNA to which a DNA-targeting segment of a gRNA will bind. If the cleavage site is outside of the nucleic acid sequence to which the DNA-targeting segment of the gRNA will bind, the cleavage site is still considered to be within the “guide RNA recognition sequence.” The cleavage site can be on only one strand or on both strands of a nucleic acid. Cleavage sites can be at the same position on both strands of the nucleic acid (producing blunt ends) or can be at different sites on each strand (producing staggered ends (i.e., overhangs)). Staggered ends can be produced, for example, by using two Cas proteins, each of which produces a single-strand break at a different cleavage site on a different strand, thereby producing a double-strand break. For example, a first nickase can create a single-strand break on the first strand of double-stranded DNA (dsDNA), and a second nickase can create a single-strand break on the second strand of dsDNA such that overhanging sequences are created. In some cases, the guide RNA recognition sequence of the nickase on the first strand is separated from the guide RNA recognition sequence of the nickase on the second strand by at least 2, 3, 4, 5, 6, 7, 8, 9, 10, 15, 20, 25, 30, 40, 50, 75, 100, 250, 500, or 1,000 base pairs.

Site-specific binding and cleavage of target DNA by Cas proteins can occur at locations determined by both (i) base-pairing complementarity between the gRNA and the target DNA and (ii) a short motif, called the protospacer adjacent motif (PAM), in the target DNA. The PAM can flank the guide RNA recognition sequence. Optionally, the guide RNA recognition sequence can be flanked on the 3′ end by the PAM. Alternatively, the guide RNA recognition sequence can be flanked on the 5′ end by the PAM. For example, the cleavage site of Cas proteins can be about 1 to about 10 or about 2 to about 5 base pairs (e.g., 3 base pairs) upstream or downstream of the PAM sequence. In some cases (e.g., when Cas9 from S. pyogenes or a closely related Cas9 is used), the PAM sequence of the non-complementary strand can be 5′-N₁GG-3′, where N₁ is any DNA nucleotide and is immediately 3′ of the guide RNA recognition sequence of the non-complementary strand of the target DNA. As such, the PAM sequence of the complementary strand would be 5′-CCN₂-3′, where N₂ is any DNA nucleotide and is immediately 5′ of the guide RNA recognition sequence of the complementary strand of the target DNA. In some such cases, N₁ and N₂ can be complementary and the N₁-N₂ base pair can be any base pair (e.g., N₁=C and N₂=G; N₁=G and N₂=C; N₁=A and N₂=T; or N₁=T, and N₂=A). In the case of Cas9 from S. aureus, the PAM can be NNGRRT or NNGRR, where N can A, G, C, or T, and R can be G or A. In the case of Cas9 from C. jejuni, the PAM can be, for example, NNNNACAC or NNNNRYAC, where N can be A, G, C, or T, and R can be G or A. In some cases (e.g., for FnCpf1), the PAM sequence can be upstream of the 5′ end and have the sequence 5′-TTN-3′.

Examples of gRNA recognition sequences include a DNA sequence complementary to the DNA-targeting segment of a gRNA, or such a DNA sequence in addition to a PAM sequence. For example, the target motif can be a 20-nucleotide DNA sequence immediately preceding an NGG motif recognized by a Cas9 protein, such as GN₁₉NGG or N₂₀NGG. See, e.g., WO 2014/165825, herein incorporated by reference in its entirety for all purposes. The guanine at the 5′ end can facilitate transcription by RNA polymerase in cells. Other examples of guide RNA recognition sequences can include two guanine nucleotides at the 5′ end (e.g., GGN₂₀NGG) to facilitate efficient transcription by T7 polymerase in vitro. See, e.g., WO 2014/065596, herein incorporated by reference in its entirety for all purposes. Other guide RNA recognition sequences can have between 4-22 nucleotides in length, including the 5′ G or GG and the 3′ GG or NGG. Yet other guide RNA recognition sequences can have between 14 and 20 nucleotides in length.

In various embodiments, the gRNA is complimentary to a sequence at the IgH locus, J Chain locus, or Ig Kappa locus in a target cell (e.g., B cell or HSC). In some embodiments, the gRNA is complimentary to a sequence at the J Chain locus. In one embodiment, the gRNA is complimentary to a sequence in the 4th exon of the J Chain locus. In one embodiment, the gRNA is complimentary to a sequence in the 1st intron of the J Chain locus.

As a non-limiting example, nucleotide sequences encoding antibodies, antigen-binding fragments, and antibody-like molecules (e.g., single chain antibody-like molecules) can be inserted into the IgH locus. The expressed antibodies, antigen-binding fragments, and antibody-like molecules (e.g., single chain antibody-like molecules) can function as surface BCR capable of switching to secreted antibody forms as per natural BCRs. As another non-limiting example, sequences coding for antibodies, antigen-binding fragments, or antibody-like proteins (e.g., single chain antibody-like molecules), and non-antibody proteins can be inserted into J chain and/or Ig kappa locus and expressed as a secreted product.

### E. Repair Template

In one aspect, the system described herein comprises a sequence corresponding to a repair template.

As used herein, the terms “repair template”, “RT”, “recombination template”, “donor nucleic acid molecule” or “donor polynucleotide”, which can be used interchangeably, refer to a segment of DNA that one desires to integrate at the target locus. In certain embodiments, the repair template comprises one or more polynucleotides of interest. In other embodiments, the repair template can comprise one or more expression cassettes. A given expression cassette can comprise a polynucleotide of interest, a polynucleotide encoding a selection marker and/or a reporter gene along with the various regulatory components that influence expression.

In certain embodiments, the repair template can comprise a segment of genomic DNA, a cDNA, a regulatory region, or any portion or combination thereof. In certain embodiments, the repair template can comprise a nucleic acid from a eukaryote, a mammal, a human, a non-human mammal, a rodent, a rat, a non-rat rodent, a mouse, a hamster, a rabbit, a pig, a bovine, a deer, a sheep, a goat, a chicken, a cat, a dog, a ferret, a primate (e.g., marmoset, rhesus monkey), a domesticated mammal, or an agricultural mammal or any other organism of interest.

In certain embodiments, the repair template comprises a knock-in allele of at least one exon of an endogenous gene. In certain embodiments, the repair template comprises a knock-in allele of the entire endogenous gene (i.e., “gene-swap knock-in”).

In further embodiments, the repair template comprises a conditional allele. In certain embodiments, the conditional allele is a multifunctional allele, as described in US 2011/0104799, which is incorporated by reference in its entirety. In certain embodiments, the conditional allele comprises: (a) an actuating sequence in sense orientation with respect to transcription of a target gene, and a drug selection cassette in sense or antisense orientation; (b) in antisense orientation a nucleotide sequence of interest (NSI) and a conditional by inversion module (COIN, which utilizes an exon-splitting intron and an invertible genetrap-like module; see, for example, US 2011/0104799, which is incorporated by reference in its entirety); and (c) recombinable units that recombine upon exposure to a first recombinase to form a conditional allele that (i) lacks the actuating sequence and the DSC, and (ii) contains the NSI in sense orientation and the COIN in antisense orientation.

In certain embodiments, the repair template is under 10 kb in size.

In certain embodiments, the repair template comprises a deletion of, for example, a eukaryotic cell, a mammalian cell, a human cell, or a non-human mammalian cell genomic DNA sequence.

In certain embodiments, the repair template comprises an insertion or a replacement of a eukaryotic, a mammalian, a human, or a non-human mammalian nucleic acid sequence with a homologous or orthologous human nucleic acid sequence. In certain embodiments, the repair template comprises an insertion or replacement of a DNA sequence with a homologous or orthologous human nucleic acid sequence at an endogenous locus that comprises the corresponding DNA sequence.

In certain embodiments, the genetic modification is an addition of a nucleic acid sequence.

In further embodiments, the repair template results in the replacement of a portion of the mammalian, human cell, or non-human mammalian target locus (e.g., Ig locus) from another organism.

Still in other embodiments, the repair template comprises a polynucleotide sharing across its full length at least 80%, 85%, 90%, 91%, 92%, 93%, 94%, 95%, 96%, 97%, 98%, 99% to a portion of the locus it is replacing (e.g., an Ig locus).

The given repair template and the corresponding region of the mammalian, human cell, or non-human mammalian locus being replaced can be a coding region, an intron, an exon, an untranslated region, a regulatory region, a promoter, or an enhancer or any combination thereof. Moreover, the given repair template and/or the region of the mammalian, human cell, or non-human mammalian locus being deleted can be of any desired length, including for example, between 10-100 nucleotides in length, 100-500 nucleotides in length, 500-1 kb nucleotides in length, 1 Kb to 1.5 kb nucleotides in length, 1.5 kb to 2 kb nucleotides in length, 2 kb to 2.5 kb nucleotides in length, 2.5 kb to 3 kb nucleotides in length, 3 kb to 5 kb nucleotides in length, 5 kb to 8 kb nucleotides in length, 8 kb to 10 kb nucleotides in length or more. In other instances, the size of the insertion or replacement is from about 5 kb to about 10 kb, from about 10 kb to about 20 kb, from about 20 kb to about 40 kb, from about 40 kb to about 60 kb, from about 60 kb to about 80 kb, from about 80 kb to about 100 kb, from about 100 kb to about 150 kb, from about 150 kb to about 200 kb, from about 200 kb to about 250 kb, from about 250 kb to about 300 kb, from about 300 kb to about 350 kb, from about 350 kb to about 400 kb, from about 400 kb to about 800 kb, from about 800 kb to 1 Mb, from about 1 Mb to about 1.5 Mb, from about 1.5 Mb to about 2 Mb, from about 2 Mb, to about 2.5 Mb, from about 2.5 Mb to about 2.8 Mb, from about 2.8 Mb to about 3 Mb. In other embodiments, the given repair template and/or the region of the mammalian, human cell, or non-human mammalian locus being deleted is at least 100, 200, 300, 400, 500, 600, 700, 800, or 900 nucleotides or at least 1 kb, 2 kb, 3 kb, 4 kb, 5 kb, 6 kb, 7 kb, 8 kb, 9 kb, 10 kb, 11 kb, 12 kb, 13 kb, 14 kb, 15 kb, 16 kb or greater.

DNA of the repair template can be stably integrated into the genome of the cell

In certain embodiments, the promoter is a tissue-specific promoter. In certain embodiments, the promoter is an immune cell-specific promoter. In certain embodiments, the immune cell promoter is a B cell promoter. In certain embodiments, the immune cell promoter is a HSC promoter.

In certain embodiments, the promoter is a developmentally-regulated promoter. In certain embodiments, the developmentally-regulated promoter is active only during an embryonic stage of development. In certain embodiments, the developmentally-regulated promoter is active only in an adult cell.

In specific embodiments, the promoter may be selected based on the cell type. Thus the various promoters find use in a eukaryotic cell, a non-rat eukaryotic cell, a mammalian cell, a non-human mammalian cell, a pluripotent cell, a non-pluripotent cell, a non-human pluripotent cell, a human pluripotent cell, a human ES cell, a human adult stem cell, a developmentally-restricted human progenitor cell, a human iPS cell, a human cell, a rodent cell, a non-rat rodent cell, a rat cell, a mouse cell, a hamster cell, a fibroblast or a CHO cell.

In some embodiments, the repair template comprises a nucleic acid flanked with site-specific recombination target sequences. It is recognized the while the entire nucleic acid can be flanked by such site-specific recombination target sequences, any region or individual polynucleotide of interest within the insert nucleic acid can also be flanked by such sites. The site-specific recombinase can be introduced into the cell by any means, including by introducing the recombinase polypeptide into the cell or by introducing a polynucleotide encoding the site-specific recombinase into the target cell. The polynucleotide encoding the site-specific recombinase can be located within the repair template or within a separate polynucleotide. The site-specific recombinase can be operably linked to a promoter active in the cell including, for example, an inducible promoter, a promoter that is endogenous to the cell, a promoter that is heterologous to the cell, a cell-specific promoter, a tissue-specific promoter, or a developmental stage-specific promoter. Site-specific recombination target sequences, which can flank the nucleic acid or any polynucleotide of interest in the nucleic acid can include, but are not limited to, loxP, lox511, lox2272, lox66, lox71, loxM2, lox5171, FRT, FRT11, FRT71, attp, att, FRT, rox, and a combination thereof.

In certain embodiments, the site-specific recombination sites flank a polynucleotide encoding a selection marker and/or a reporter gene contained within the repair template. In such instances following integration of the repair template the targeted locus the sequences between the site-specific recombination sites can be removed.

In certain embodiments, the repair template comprises a polynucleotide encoding a selection marker. The selection marker can be contained in a selection cassette. Such selection markers include, but are not limited, to neomycin phosphotransferase (neor), hygromycin B phosphotransferase (hygr), puromycin-N-acetyltransferase (puror), blasticidin S deaminase (bsrr), xanthine/guanine phosphoribosyl transferase (gpt), or herpes simplex virus thymidine kinase (HSV-k), or a combination thereof. In certain embodiments, the polynucleotide encoding the selection marker is operably linked to a promoter active in the cell, rat cell, pluripotent rat cell, the ES rat cell, a eukaryotic cell, a non-rat eukaryotic cell, a pluripotent cell, a non-pluripotent cell, a non-human pluripotent cell, a human pluripotent cell, a human ES cell, a human adult stem cell, a developmentally-restricted human progenitor cell, a human iPS cell, a mammalian cell, a non-human mammalian cell, a human cell, a rodent cell, a non-rat rodent cell, a mouse cell, a hamster cell, a fibroblast, or a CHO cell. When serially tiling polynucleotides of interest into a targeted locus, the selection marker can comprise a recognition site for a gene-editing molecule, as outlined above. In certain embodiments, the polynucleotide encoding the selection marker is flanked with a site-specific recombination target sequences.

The repair template can further comprise a reporter gene operably linked to a promoter, wherein the reporter gene encodes a reporter protein selected from the group consisting of or comprising LacZ, mPlum, mCherry, tdTomato, mStrawberry, J-Red, DsRed, mOrange, mKO, mCitrine, Venus, YPet, enhanced yellow fluorescent protein (eYFP), Emerald, enhanced green fluorescent protein (EGFP), CyPet, cyan fluorescent protein (CFP), Cerulean, T-Sapphire, luciferase, alkaline phosphatase, and/or a combination thereof. Such reporter genes can be operably linked to a promoter active in the cell. Such promoters can be an inducible promoter, a promoter that is endogenous to the reporter gene or the cell, a promoter that is heterologous to the reporter gene or to the cell, a cell-specific promoter, a tissue-specific promoter, or a developmental stage-specific promoter.

In certain embodiments, the genomic locus comprises a mouse genomic DNA sequence, a rat genomic DNA sequence, eukaryotic genomic DNA sequence, a non-rat eukaryotic genomic DNA sequence, a mammalian genomic DNA sequence, a human genomic DNA sequence, or non-human DNA sequence mammalian, or a combination thereof. In certain embodiments, the genomic locus comprises, in any order, rat and human genomic DNA sequences. In certain embodiments, the genomic locus comprises, in any order, mouse and human genomic DNA sequences. In certain embodiments, the genomic locus comprises, in any order, mouse and rat genomic DNA sequences. In certain embodiments, the genomic locus comprises, in any order, rat, mouse, and human genomic DNA sequences.

In certain embodiments, the repair template comprises a selection cassette. In certain embodiments, the selection cassette comprises a nucleic acid sequence encoding a selective marker, wherein the nucleic acid sequence is operably linked to a promoter active in rat ES cells. In certain embodiments, the selective marker is selected from or comprises a hygromycin resistance gene or a neomycin resistance gene.

In certain embodiments, the nucleic acid comprises a genomic locus that encodes a protein expressed in a B cell. In certain embodiments, the nucleic acid comprises a genomic locus that encodes a protein expressed in a primary B cell. In certain embodiments, the nucleic acid comprises a genomic locus that encodes a protein expressed in an immature B cell. In certain embodiments, the nucleic acid comprises a genomic locus that encodes a protein expressed in a mature B cell.

In certain embodiments, the repair template comprises a regulatory element. In certain embodiments, the regulatory element is a promoter. In certain embodiments, the regulatory element is an enhancer. In certain embodiments, the regulatory element is a transcriptional repressor-binding element.

In certain embodiments, the genetic modification comprises a deletion of a non-protein-coding sequence, but does not comprise a deletion of a protein-coding sequence. In certain embodiments, the deletion of the non-protein-coding sequence comprises a deletion of a regulatory element. In certain embodiments, the genetic modification comprises a deletion of a regulatory element. In certain embodiments, the genetic modification comprises an addition of a promoter or a regulatory element. In certain embodiments, the genetic modification comprises a replacement of a promoter or a regulatory element.

In one aspect, provided herein are non-limiting exemplary templates for insertion into a locus of interest in the target cell (e.g., B cell or HSC).

In one embodiment, an exemplary template for insertion into the IgH locus comprises: a 5′ IgH homology region, a splice acceptor, a 2A sequence with 5′ furin cleavage sequence, a nucleotide sequence encoding a light chain variable region, a nucleotide sequence encoding a light chain constant region, a 2A sequence with 5′ furin cleavage sequence, a nucleotide sequence encoding a heavy chain variable region, a splice donor sequence, and/or a 3′ IgH homology region. The heavy chain and light chain sequences can be in either order.

In one embodiment, an exemplary template for insertion into the J chain exon 4 locus comprises: 5′ J Chain exon 4 homology region, a 2A sequence with 5′ furin cleavage sequence, a nucleotide sequence encoding a light chain variable region, a nucleotide sequence encoding a light chain constant, a 2A sequence with 5′ furin cleavage sequence, a nucleotide sequence encoding a heavy chain variable region, a nucleotide sequence encoding a heavy chain constant region, and/or a 3′ J Chain exon 4 homology region. The heavy chain and light chain sequences can be in either order.

In one embodiment, an exemplary template for insertion into the J chain exon 4 locus comprises: 5′ J Chain exon 4 homology region, a 2A sequence with 5′ furin cleavage sequence, Gene of Interest, and/or a 3′ J Chain exon 4 homology region.

In one embodiment, an exemplary template for insertion into the ROSA/safe harbor site comprises: 5′ ROSA locus homology region, a promoter, a nucleotide sequence encoding a light chain variable region, a nucleotide sequence encoding a light chain constant, a 2A sequence with 5′ furin cleavage sequence, a nucleotide sequence encoding a heavy chain variable, a nucleotide sequence encoding a heavy chain constant region, a poly A sequence, and/or 3′ ROSA locus homology region. The heavy chain and light chain sequences can be in either order.

In one embodiment, an exemplary template for insertion into the ROSA/safe harbor site comprises: 5′ ROSA locus homology region, a promoter, Gene of Interest, a poly A sequence, and/or 3′ ROSA locus homology region.

### Methods of Use and Making

A further embodiment of the recombinant viral capsid proteins described herein is their use for delivering a nucleotide of interest to a target cell. The target cell may be a B cell and/or a hematopoietic stem cell (HSC).

In some embodiments, a nucleotide of interest may be a transfer plasmid, which may generally comprise 5′ and 3′ inverted terminal repeat (ITR) sequences flanking the reporter gene(s) or therapeutic gene(s) (which may be under the control of a viral or non-viral promoter, when encompassed within an AAV vector. In one embodiment, a nucleotide of interest is a transfer plasmid comprising from 5′ to 3′: a 5′ ITR, a promoter, a gene (e.g., a reporter and/or therapeutic gene) and a 3′ITR.

Non-limiting examples of useful promoters include, e.g., cytomegalovirus (CMV)-promoter, the spleen focus forming virus (SFFV)-promoter, the elongation factor 1 alpha (EF1a)-promoter (the 1.2 kb EF1a-promoter or the 0.2 kb EF1a-promoter), the chimeric EF 1 a/IF4-promoter, and the phospho-glycerate kinase (PGK)-promoter. An internal enhancer may also be present in the viral construct to increase expression of the gene of interest. For example, the CMV enhancer (Karasuyama et al. 1989. J. Exp. Med. 169:13, which is incorporated herein by reference in its entirety) may be used. In some embodiments, the CMV enhancer can be used in combination with the chicken 13-actin promoter.

A variety of reporter genes (or detectable moieties) can be encapsulated in a multimeric structure comprising the recombinant viral capsid proteins described herein. Exemplary reporter genes include, for example, β-galactosidase (encoded lacZ gene), Green Fluorescent Protein (GFP), enhanced Green Fluorescent Protein (eGFP), MmGFP, blue fluorescent protein (BFP), enhanced blue fluorescent protein (eBFP), mPlum, mCherry, tdTomato, mStrawberry, J-Red, DsRed, mOrange, mKO, mCitrine, Venus, YPet, yellow fluorescent protein (YFP), enhanced yellow fluorescent protein (eYFP), Emerald, CyPet, cyan fluorescent protein (CFP), Cerulean, T-Sapphire, luciferase, alkaline phosphatase, or a combination thereof. The methods described herein demonstrate the construction of targeting vectors that employ the use of a reporter gene that encodes green fluorescent protein, however, persons of skill upon reading this disclosure will understand that non-human animals described herein can be generated in the absence of a reporter gene or with any reporter gene known in the art.

A variety of therapeutic genes (encoding an antibody or antigen-binding fragments) can also be encapsulated in the can be encapsulated in a multimeric structure comprising the recombinant viral capsid proteins described herein, e.g., as part of a transfer vector.

A further embodiment of the present invention is a process for the preparation of a recombinant capsid protein, the method comprising the steps of:
a) expressing a nucleic acid encoding the recombinant capsid protein under suitable conditions, and
b) isolating the expressed capsid protein of step a).

In some embodiments, a viral particle as described herein comprises a mosaic capsid, e.g., a capsid comprising capsid proteins genetically modified as described herein (in the absence or presence of a covalent bond with a targeting ligand) in a certain ratio with reference capsid proteins. Mosaic capsid and methods of making such mosaic viral particles may be find in, for example, WO2020242984, the content of which is incorporated herein by reference in its entirety, and in the Examples sections below. An exemplary method for making such a mosaic viral particle comprises
a) expressing a nucleic acid encoding the recombinant capsid protein and a nucleotide encoding a reference capsid protein at a ratio (wt/wt) of 1:1 and 10:1 under suitable conditions, and
b) isolating the expressed capsid protein of step a).

Generally speaking, a mosaic capsid formed according to the method will be considered to have a modified capsid protein:reference capsid protein ratio similar to the ratio (wt:wt) of nucleic acids encoding same used to produce the mosaic capsid. Accordingly, in some embodiments, a composition described herein comprises, or a method described herein combines, a recombinant viral capsid protein and a reference capsid protein (or combination of reference capsid proteins) at a ratio that ranges from 1:1 to 1:15. In some embodiments, the ratio is 1:2. In some embodiments, the ratio is 1:3. In some embodiments, the ratio is 1:4. In some embodiments, the ratio is 1:5. In some embodiments, the ratio is 1:6. In some embodiments, the ratio is 1:7. In some embodiments, the ratio is 1:8. In some embodiments, the ratio is 1:9. In some embodiments, the ratio is 1:10. In some embodiments, the ratio is 1:11. In some embodiments, the ratio is 1:12. In some embodiments, the ratio is 1:13. In some embodiments, the ratio is 1:14. In some embodiments, the ratio is 1:15.

In some embodiments, a viral particle as described herein comprises a non-primate animal AAV. In some AAV capsid protein embodiments of the invention, the non-primate animal AAV is a non-primate AAV listed in Table 2 of WO2020242984, the content of which is incorporated herein by reference in its entirety. In some embodiments, the non-primate AAV is an avian AAV (AAAV), a sea lion AAV or a bearded dragon AAV. In some embodiments, the non-primate animal AAV is an AAAV, and optionally an amino acid sequence of an AAAV capsid protein comprises a modification is at position 1444 or 1580 of a VP1 capsid protein of AAAV. In some embodiments, the non-primate animal AAV is a squamate AAV, e.g., a bearded dragon AAV, and optionally an amino acid sequence of a bearded dragon AAV comprises a modification is at position 1573 or 1436 of a VP1 capsid protein of a bearded dragon AAV. In some embodiments, the non-primate animal AAV is a mammalian AAV, e.g., a sea lion AAV, and optionally an amino acid sequence of a seal lion AAV comprises a modification at position selected from the group consisting of 1429, 1430, 1431, 1432, 1433, 1434, 1436, 1437, and A565 of a VP1 capsid protein of a sea lion AAV.

Further embodiments of the present invention include a method for altering the tropism of a virus, the method comprising the steps of: (a) inserting a nucleic acid encoding a heterologous epitope into a nucleic acid sequence encoding an viral capsid protein to form a nucleotide sequence encoding a genetically modified capsid protein comprising the heterologous epitope and/or (b) culturing a packaging cell in conditions sufficient for the production of viral vectors, wherein the packaging cell comprises the nucleotide sequence. A further embodiment of the present invention is a method for displaying a heterologous epitope on the surface of a capsid protein, the method comprising the steps of: a) expressing the nucleic acid according to this invention under suitable conditions, and b) isolating the expressed capsid protein of step a).

In some embodiments, the packaging cell further comprises a helper plasmid and/or a transfer plasmid comprising a nucleotide of interest. In some embodiments, the methods further comprise isolating self-complementary adeno-associated viral vectors from culture supernatant. In some embodiments, the methods further comprise lysing the packaging cell and isolating single-stranded adeno-associated viral vectors from the cell lysate. In some embodiments, the methods further comprise (a) clearing cell debris, (b) treating the supernatant containing viral vectors with DNase I and MgCl₂, (c) concentrating viral vectors, (d) purifying the viral vectors, and (e) any combination of (a)-(d).

Packaging cells useful for production of the viral vectors described herein include, e.g., animal cells permissive for the virus, or cells modified so as to be permissive for the virus; or the packaging cell construct, for example, with the use of a transformation agent such as calcium phosphate. Non-limiting examples of packaging cell lines useful for producing viral vectors described herein include, e.g., human embryonic kidney 293 (HEK-293) cells (e.g., American Type Culture Collection [ATCC] No. CRL-1573), HEK-293 cells that contain the SV40 Large T-antigen (HEK-293T or 293T), HEK293T/17 cells, human sarcoma cell line HT-1080 (CCL-121), lymphoblast-like cell line Raj i (CCL-86), glioblastoma-astrocytoma epithelial-like cell line U87-MG (HTB-14), T-lymphoma cell line HuT78 (TIB-161), NIH/3T3 cells, Chinese Hamster Ovary cells (CHO) (e.g., ATCC Nos. CRL9618, CCL61, CRL9096), HeLa cells (e.g., ATCC No. CCL-2), Vero cells, NITH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, CAP cells, CAP-T cells, and the like.

L929 cells, the FLY viral packaging cell system outlined in Cosset et al (1995) J Virol 69, 7430-7436, NS0 (murine myeloma) cells, human amniocytic cells (e.g., CAP, CAP-T), yeast cells (including, but not limited to, S. cerevisiae, Pichia pastoris), plant cells (including, but not limited to, Tobacco NTl, BY-2), insect cells (including but not limited to SF9, S2, SF21, Tni (e.g. High 5)) or bacterial cells (including, but not limited to, E. coli).

For additional packaging cells and systems, packaging techniques and vectors for packaging the nucleic acid genome into the pseudotyped viral vector see, for example, Polo, et al, Proc Natl Acad Sci USA, (1999) 96:4598-4603. Methods of packaging include using packaging cells that permanently express the viral components, or by transiently transfecting cells with plasmids.

### Pharmaceutical Compositions, Dosage Forms and Administration

A further embodiment provides a medicament comprising at least one component of the system (e.g., a polynucleotide molecule comprises a sequence encoding the antibody or antigen-binding fragment thereof; a gene editing molecule or a polynucleotide molecule comprising a sequence encoding said gene editing molecule) described herein. In some embodiments, the medicament comprises a recombinant viral capsid protein and appropriate binding molecule according to this disclosure. Preferably such medicament is useful as a gene transfer vector.

Also disclosed herein are pharmaceutical compositions comprising a viral vectors described herein and a pharmaceutically acceptable carrier and/or excipient. In addition, disclosed herein are pharmaceutical dosage forms comprising the viral vector described herein.

As discussed herein, the compositions including viral vectors described herein can be used for various therapeutic applications (in vivo and ex vivo) and as research tools.

Pharmaceutical compositions based on the viral vectors disclosed herein can be formulated in any conventional manner using one or more physiologically acceptable carriers and/or excipients. The viral vector may be formulated for administration by, for example, injection, inhalation or insulation (either through the mouth or the nose) or by oral, buccal, parenteral or rectal administration, or by administration directly to a tumor.

The pharmaceutical compositions can be formulated for a variety of modes of administration, including systemic, topical or localized administration. Techniques and formulations can be found in, for example, Remington's Pharmaceutical Sciences, Meade Publishing Co., Easton, Pa. For systemic administration, injection is preferred, including intramuscular, intravenous, intraperitoneal, and subcutaneous. For the purposes of injection, the pharmaceutical compositions can be formulated in liquid solutions, preferably in physiologically compatible buffers, such as Hank's solution or Ringer's solution. In addition, the pharmaceutical compositions may be formulated in solid form and redissolved or suspended immediately prior to use. Lyophilized forms of the pharmaceutical composition are also suitable.

For oral administration, the pharmaceutical compositions may take the form of, for example, tablets or capsules prepared by conventional means with pharmaceutically acceptable excipients such as binding agents (e.g. pregelatinized maize starch, polyvinylpyrrolidone or hydroxypropyl methylcellulose); fillers (e.g. lactose, microcrystalline cellulose or calcium hydrogen phosphate); lubricants (e.g. magnesium stearate, talc or silica); disintegrants (e.g. potato starch or sodium starch glycolate); or wetting agents (e.g. sodium lauryl sulfate). The tablets can also be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g. sorbitol syrup, cellulose derivatives or hydrogenated edible fats); emulsifying agents (e.g. lecithin or acacia); non-aqueous vehicles (e.g. ationd oil, oily esters, ethyl alcohol or fractionated vegetable oils); and preservatives (e.g. methyl or propyl-p-hydroxybenzoates or sorbic acid). The preparations can also contain buffer salts, flavoring, coloring and sweetening agents as appropriate.

The pharmaceutical compositions can be formulated for parenteral administration by injection, e.g. by bolus injection or continuous infusion. Formulations for injection can be presented in a unit dosage form, e.g. in ampoules or in multi-dose containers, with an optionally added preservative. The pharmaceutical compositions can further be formulated as suspensions, solutions or emulsions in oily or aqueous vehicles, and may contain other agents including suspending, stabilizing and/or dispersing agents.

Additionally, the pharmaceutical compositions can also be formulated as a depot preparation. These long acting formulations can be administered by implantation (e.g. subcutaneously or intramuscularly) or by intramuscular injection. Thus, for example, the compounds may be formulated with suitable polymeric or hydrophobic materials (e.g. as an emulsion in an acceptable oil) or ion exchange resins, or as sparingly soluble derivatives, for example, as a sparingly soluble salt. Other suitable delivery systems include microspheres, which offer the possibility of local noninvasive delivery of drugs over an extended period of time. This technology can include microspheres having a precapillary size, which can be injected via a coronary catheter into any selected part of an organ without causing inflammation or ischemia. The administered therapeutic is men slowly released from the microspheres and absorbed by the surrounding cells present in the selected tissue.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, bile salts, and fusidic acid derivatives. In addition, detergents may be used to facilitate permeation. Transmucosal administration can occur using nasal sprays or suppositories. For topical administration, the viral vector described herein can be formulated into ointments, salves, gels, or creams as generally known in the art. A wash solution can also be used locally to treat an injury or inflammation in order to accelerate healing.

Pharmaceutical forms suitable for injectable use can include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid. It must be stable under the conditions of manufacture and certain storage parameters (e.g. refrigeration and freezing) and must be preserved against the contaminating action of microorganisms, such as bacteria and fungi.

If formulations disclosed herein are used as a therapeutic to boost an immune response in a subject, a therapeutic agent can be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required viral vector size in the case of dispersion and by the use of surfactants. The prevention of the action of microorganisms can be brought about by various antibacterial and antifungal agents known in the art. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions can be prepared by incorporating the active compounds or constructs in the required amount in the appropriate solvent with various of the other ingredients enumerated above, as required, followed by filtered sterilization.

Upon formulation, solutions can be administered in a manner compatible with the dosage formulation and in such amount as is therapeutically effective. The formulations are easily administered in a variety of dosage forms, such as the type of injectable solutions described above, but slow release capsules or microparticles and microspheres and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intratumorally, intramuscular, subcutaneous and intraperitoneal administration. In this context, sterile aqueous media that can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion.

The person responsible for administration will, in any event, determine the appropriate dose for the individual subject. For example, a subject may be administered viral vectors described herein on a daily or weekly basis for a time period or on a monthly, bi-yearly or yearly basis depending on need or exposure to a pathogenic organism or to a condition in the subject (e.g. cancer).

In addition to the compounds formulated for parenteral administration, such as intravenous, intratumorally, intradermal or intramuscular injection, other pharmaceutically acceptable forms include, e.g., tablets or other solids for oral administration; liposomal formulations; time release capsules; biodegradable and any other form currently used.

One may also use intranasal or inhalable solutions or sprays, aerosols or inhalants. Nasal solutions can be aqueous solutions designed to be administered to the nasal passages in drops or sprays. Nasal solutions can be prepared so that they are similar in many respects to nasal secretions. Thus, the aqueous nasal solutions usually are isotonic and slightly buffered to maintain a pH of 5.5 to 7.5. In addition, antimicrobial preservatives, similar to those used in ophthalmic preparations, and appropriate drug stabilizers, if required, may be included in the formulation. Various commercial nasal preparations are known and can include, for example, antibiotics and antihistamines and are used for asthma prophylaxis.

Oral formulations can include excipients as, for example, pharmaceutical grades of mannitol, lactose, starch, magnesium stearate, sodium saccharine, cellulose, magnesium carbonate and the like. These compositions take the form of solutions, suspensions, tablets, pills, capsules, sustained release formulations or powders. In certain defined embodiments, oral pharmaceutical compositions will include an inert diluent or assimilable edible carrier, or they may be enclosed in hard or soft-shell gelatin capsule, or they may be compressed into tablets, or they may be incorporated directly with the food of the diet. For oral therapeutic administration, the active compounds may be incorporated with excipients and used in the form of ingestible tablets, buccal tablets, troches, capsules, elixirs, suspensions, syrups, wafers, and the like.

The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup of elixir may contain the active compounds sucrose as a sweetening agent methyl and propylparabens as preservatives, a dye and flavoring, such as cherry or orange flavor.

Further embodiments disclosed herein can concern kits for use with methods and compositions. Kits can also include a suitable container, for example, vials, tubes, mini- or microfuge tubes, test tube, flask, bottle, syringe or other container. Where an additional component or agent is provided, the kit can contain one or more additional containers into which this agent or component may be placed. Kits herein will also typically include a means for containing the viral vectors and any other reagent containers in close confinement for commercial sale. Such containers may include injection or blow-molded plastic containers into which the desired vials are retained. Optionally, one or more additional active agents such as, e.g., anti-inflammatory agents, anti-viral agents, anti-fungal or anti-bacterial agents or anti-tumor agents may be needed for compositions described.

Dose ranges and frequency of administration can vary depending on the nature of the viral vectors and the medical condition as well as parameters of a specific patient and the route of administration used. In some embodiments, viral vector compositions can be administered to a subject at a dose ranging from about 1×10⁵ plaque forming units (pfu) to about 1×10¹⁵ pfu, depending on mode of administration, the route of administration, the nature of the disease and condition of the subject. In some cases, the viral vector compositions can be administered at a dose ranging from about 1×10⁸ pfu to about 1×10¹⁵ pfu, or from about 1×10¹⁰ pfu to about 1×10¹⁵ pfu, or from about 1×10⁸ pfu to about 1×10¹² pfu. A more accurate dose can also depend on the subject in which it is being administered. For example, a lower dose may be required if the subject is juvenile, and a higher dose may be required if the subject is an adult human subject. In certain embodiments, a more accurate dose can depend on the weight of the subject. In certain embodiments, for example, a juvenile human subject can receive from about 1×108 pfu to about 1×10¹⁰ pfu, while an adult human subject can receive a dose from about 1×1010 pfu to about 1×10¹² pfu.

Compositions disclosed herein may be administered by any means known in the art. For example, compositions may include administration to a subject intravenously, intratumorally, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intrathecally, subcutaneously, subconjunctival, intravesicularlly, mucosally, intrapericardially, intraumbilically, intraocularly, orally, locally, by inhalation, by injection, by infusion, by continuous infusion, by localized perfusion, via a catheter, via a lavage, in a cream, or in a lipid composition.

Any method known to one skilled in the art maybe used for large scale production of viral vectors, packaging cells and vector constructs described herein. For example, master and working seed stocks may be prepared under GMP conditions in qualified primary CEFs or by other methods. Packaging cells may be plated on large surface area flasks, grown to near confluence and viral vectors purified. Cells may be harvested and viral vectors released into the culture media isolated and purified, or intracellular viral vectors released by mechanical disruption (cell debris can be removed by large-pore depth filtration and host cell DNA digested with endonuclease). Virus viral vectors may be subsequently purified and concentrated by tangential-flow filtration, followed by diafiltration. The resulting concentrated bulk maybe formulated by dilution with a buffer containing stabilizers, filled into vials, and lyophilized. Compositions and formulations may be stored for later use. For use, lyophilized viral vectors may be reconstituted by addition of diluent.

Certain additional agents used in the combination therapies can be formulated and administered by any means known in the art.

### EXAMPLES

The following examples are provided for illustrative purposes only and are not intended to limit the scope of the invention.

### Example 1: Anti-PcrV mAb Expression by AAV in P. Aeruginosa Challenge

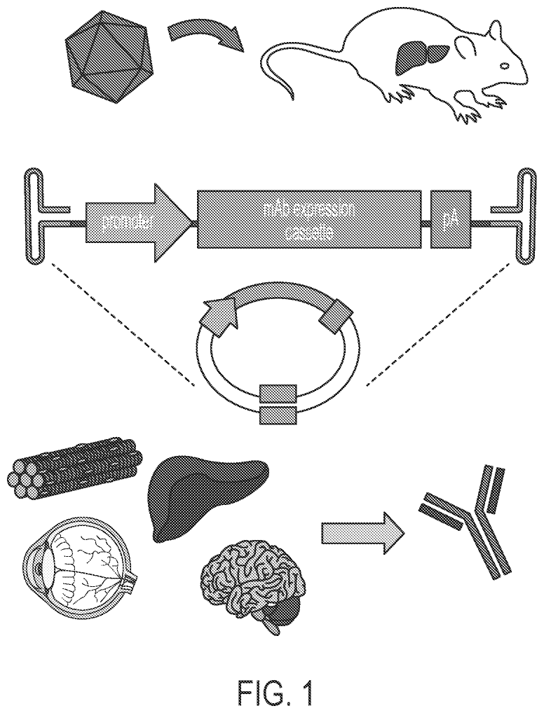

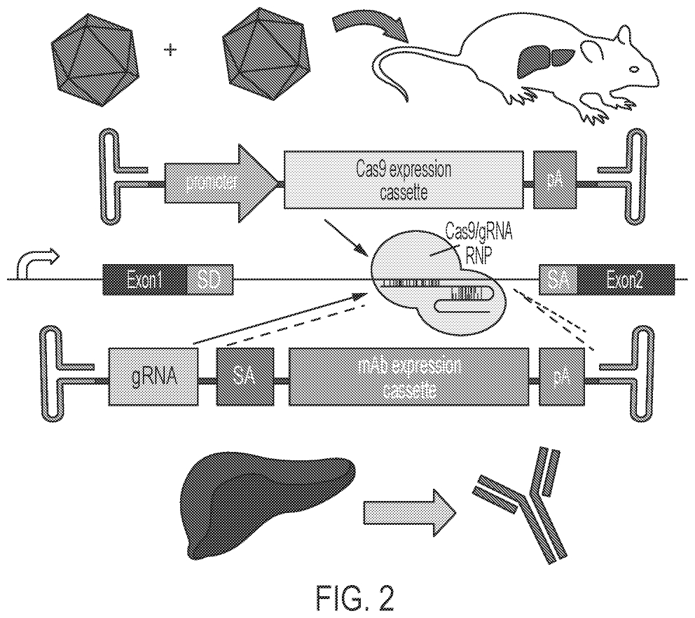

Several approaches to express monoclonal antibodies in vivo via AAV delivery have been pursued. These include AAV-mediated delivery of transgene for episome expression (FIG. 1) and AAV+Cas9/gRNA-mediated insertion of transgene into genomic locus in the liver (FIG. 2). As a proof-of-concept study, anti-PcrV mAb expressed by AAV protected mice from P. aeruginosa challenge in an acute pneumonia model. P. aeruginosa is an opportunistic bacterial pathogen that causes a wide variety of infections, including pneumonia. mAb that targets PcrV, a protein expressed on the bacterial surface that promotes delivery of cytotoxins into the host during infection, was selected for the study.

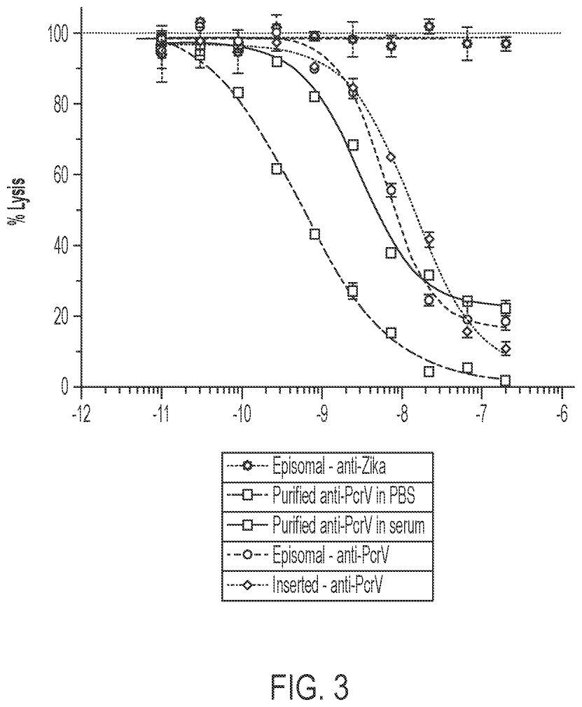

Results were successful. In vitro neutralization with episomal and liver-inserted anti-PcrB mAbs from mouse serum was within 2-5 fold of CHO-purified mAb (FIG. 3). Further, the in vivo challenge model with P. aeruginosa demonstrated protection from lethal infection with episomal and liver-inserted anti-PcrV mAbs (FIG. 4).

### Example 2: BCR Editing of Peripheral Mouse B Cells Ex Vivo

Success of immunization depends on the ability of the host to respond to a given immunogen and generate the appropriate response. In certain populations (e.g., young children, elderly, immunocompromized, etc.), vaccines sometimes fail to properly elicit the desired response. For several infectious agents, design of an immunogen to generate a sufficiently broad and potent immune response has not been successful even in normal healthy populations. Additionally, for some pathogens (e.g., Dengue), vaccination may actually result in enhancement of infection (ADE) rather than protection depending on individual vaccine responses such as the isotype of antibodies elicited. Monoclonal antibodies can either be selected or designed to overcome many of these issues, but compared to vaccines, passively delivered antibodies are short lived, and life-long immunity would require frequent re-administration.

In the present Example, CRISPR was used to engineer B cells ex vivo to express a specific BCR from the Ig locus and to reintroduce those modified B cells back into the host to become part of the immune repertoire. The engineered B cells should deliver near-term protection to a pathogen and long term adaptive humoral immunity capable of rapid recall and affinity maturation. Furthermore, this approach can be used to express antibodies with tailored Fc fit for purpose, such as decreased FcR binding to avoid ADE. The present engineering solution was based exclusively on viral vector delivery.

An additional challenge to the project success is the long-term engraftment of ex vivo engineered B cells back into the host to establish memory B and plasma cells derived from the adoptively transferred cells. It is proposed herein to investigate prime/boost technologies to facilitate engraftment and characterize Ag-specific responses over time. One approach is the engagement of the engineered BCR with a costimulatory receptor on B cells to selectively expand engineered B cells without generation of endogenous B cells to the specific antigen.

To engineer the specificity of the B cell receptor (BCR) in peripheral mouse B cells, a CRISPR-Cas9-mediated insertion of a BCR cassette into the heavy chain antibody locus was used. Expression of an inserted antibody gene that utilizes the endogenous heavy chain constant region was achieved by inserting a cassette containing a full-length light chain antibody sequence and a heavy chain variable sequence in the genomic region downstream of the final J gene but upstream of the Eu enhancer. The BCR cassette did not contain a promoter and was designed to use a splice acceptor site to capture transcription of the endogenously rearranged heavy chain variable gene. This has the advantage of placing the inserted BCR cassette under the transcriptional control of the endogenous heavy chain promoter, preventing expression in cells without a productively rearranged BCR gene. This strategy also involves simultaneous disruption of the Kappa light chain constant region in order to avoid mispairing of the inserted heavy chain with an endogenous Kappa light chain.

### Methods

### B Cell Isolation and Culture

Spleens from mice were harvested in B cell isolation buffer and processed into single-cell suspensions. Splenocytes were washed once in B cell isolation buffer, after which B cells were enriched using the EasySep Mouse B Cell Isolation Kit (STEMCELL Technologies) according to the manufacturer's specifications. Isolated B cells were spun and re-suspended in B cell media containing the indicated stimulation factors at 5×10⁵ cells/ml, then placed in an incubator at 37° C. for 24 hours before editing.

### RNP Nucleofection and AAV Infection

For each nucleofection of 3×10⁶ B cells, RNPs were generated by combining 150 pmol Truecut Cas9 Protein v2 (Invitrogen) with 400 pmol of sgRNA (200 pmol V_{H} gRNA1 and 200 pmol mIgK gRNA7) (IDT) in Mouse B cell nucleofection Buffer (Lonza) at a total volume of 20 ul. RNPs were incubated for 15-20 minutes before addition of B cells to allow for complex formation. B cells were collected and counted, then washed 1 time in PBS. 3×10⁶ B cells were resuspended in Mouse B cell nucleofection buffer, mixed with the complexed RNP, and transferred to a nucleofection cuvette. Cells were electroporated with program Z-001 on a Lonza Nucleofector 2b device. Immediately after nucleofection, 400 μl of B cell media lacking serum but containing stimulation factors was added to the cuvette. Cells were brought to 1×10⁶ cells/ml in B cell media lacking serum but containing growth factors and transferred to wells of culture plates, after which AAV was added. After 2 hours at 37° C., an equal volume of B cell media containing stimulation factors and 2× serum was added to wells to bring final serum concentration to 10%, after which cells were returned to the incubator.

### Homology Repair Template

To insert the BCR construct of interest, AAV plasmids containing templates for homology-based repair in B cells were produced. The 5′ ITR in the AAV genome was immediately followed by a 970-bp homology arm. After the homology arm is a 70-bp splice acceptor site derived from the mouse Ighg1 gene, followed by the first two bases of mouse IgM exon 1, a Gly-Ser-Gly linker, and a T2A peptide. Next is the full-length light chain variable region of interest and the relevant light chain antibody constant region, then a furin cleavage site, a second Gly-Ser-Gly linker, a P2A peptide, and the relevant heavy chain variable region. Finally, a 60-bp splice donor region from mouse Ighj1 was followed by a 859-bp homology region and the 3′ ITR.

AAV serotype 1 containing the homology repair template was produced by the Regeneron Viral Vectors Technology Core Facility. AAV was used to infect B cells at approximately 1×10⁵ viral genomes per cell.

### Flow Cytometry

48 hours after editing, B cells were harvested and transferred to wells of a 96-well round bottom plate. Cells were stained with a viability dye (Invitrogen), then with a biotinylated form of the relevant antigen followed by a fluorescently-labeled streptavidin and surface markers. Stained cells were analyzed by flow cytometry on a BD FACSymphony A3 device to assess the frequency of antigen-binding cells.

### Cell Transfer and Immunization

48 hours after editing, cells were collected, spun, and rested for 3 hours in B cell media lacking stimulation factors. Cells were then washed once with warm PBS, resuspended in PBS, and intravenously injected into recipient mice. 24 hours after cell transfer, recipient mice were immunized intraperitoneally with 25 ug antigen in AdjuPhos adjuvant (Invivogen).

### Buffers and Media

### B Cell Isolation Buffer:

1×PBS without Ca²⁺ and Mg²⁺
2% Fetal Bovine Serum (Gibco)
2 mM EDTA (Gibco)
1× Penicillin-Streptomycin-Glutamine (Gibco)

### B Cell Media:

RPMI-1640
10% Fetal Bovine Serum (Gibco)
1× Penicillin-Streptomycin-Glutamine (Gibco)
10 mM Hepes (Gibco)
55 nM 2-Mercaptoethanol (Gibco)

### B Cell Stimulation Conditions

1. CD40L-HA (100 ng/ml) (R&D)
   Anti-HA antibody (100 ng/ml) (R&D)
   Recombinant mouse IL-4 (4 ng/ml) (Peprotech)
2. CD40L-HA (100 ng/ml) (R&D)
   Anti-HA antibody (100 ng/ml) (R&D)
   Anti-CD180 antibody (2 μg/ml) (Biolegend)
      gRNA Target Sequences

(SEQ ID NO: 1)
Vh gRNA1: TGCTAAAACAATCCTATGGC
(SEQ ID NO: 2)
mIgK gRNA7: TGGTGCAGCATCAGCCCCTG

### Culture Reagents

recombinant mouse IL-4
recombinant mouse CD40L-HA tag
anti-HA antibody
anti-CD180 (Biolegend)
EasySep Mouse B cell isolation kit (STEMCELL Technologies)
Mouse B cell nucleofector Kit (Lonza)
TrueCut Cas9 Protein v2 (Invitrogen)
Penicillin-Streptomycin-Glutamine (Gibco)
2-Mercaptoethanol (Gibco)
HEPES (Gibco)
Fetal Bovine Serum, certified, heat inactivated (Gibco)

### Flow Cytometry Reagents are Shown in Table 1, Below:

TABLE 1
Flow Cytometry Reagents

| Target | Fluorophore | Supplier |
|---|---|---|
| B220 | APC | Biolegend |
| CD19 | APC/Cy7 | Biolegend |
| IgM | PE/Cy7 | Biolegend |
| mIgK | BV421 | BD Biosciences |
| SA-FITC | FITC | eBioscience |
| Viability--Blue LIVE/DEAD | BUV496 channel | Thermo |
| TruStain FcX ™ (anti-mouse | N/A | Biolegend |
| CD16/32) | | |
| BCMA-FITC | FITC | Acro Biosystems |
| IgG1 | BV421 | BD |
| IgG2a | A-647 | Southern Biotech |
| IgD | PerCP-Cy5.5 | BioLegend |
| CD19 | APC-Cy7 | Biolegend |
| mIgL | PE | Southern Biotech |
| FAS | BV650 | BD |
| mIgK | A-700 | BD |

### Results

Mouse splenic B cells were cultured in stimulation condition #2 and edited to express a BCR of interest as described. 48 hours after editing, cells were washed and stained for viability, surface markers, and antigen binding. The percentage of B cells that express the introduced BCR and bind relevant antigen is mocked control (0.28), RNP control (0.13), and RNP+AAV1 (13.7) as shown in FIG. 6.

### Example 3: Optimizing BCR Editing Technologies for mAb Expression in B Cells

While many techniques to reprogram B cell antigen specificity have been tried, the following engineered specificity was optimized to increase ex vivo targeting efficiency and, ultimately, in vivo efficacy as well. An overview of the general process used is shown in FIGS. 7A-7C, where Cas9/gRNA RNP and AAV delivery of repair template is used to insert antibody gene into the heavy chain locus of B cells in vivo.

### ULC-Pairing and Full Length BCR Insertion

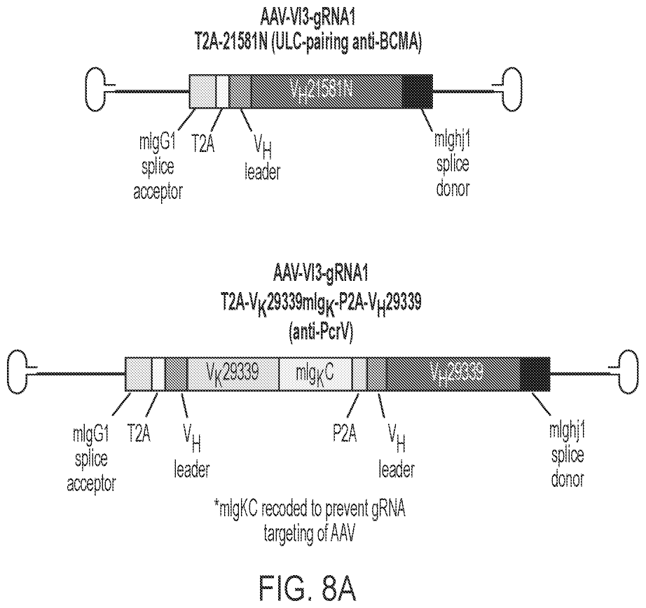

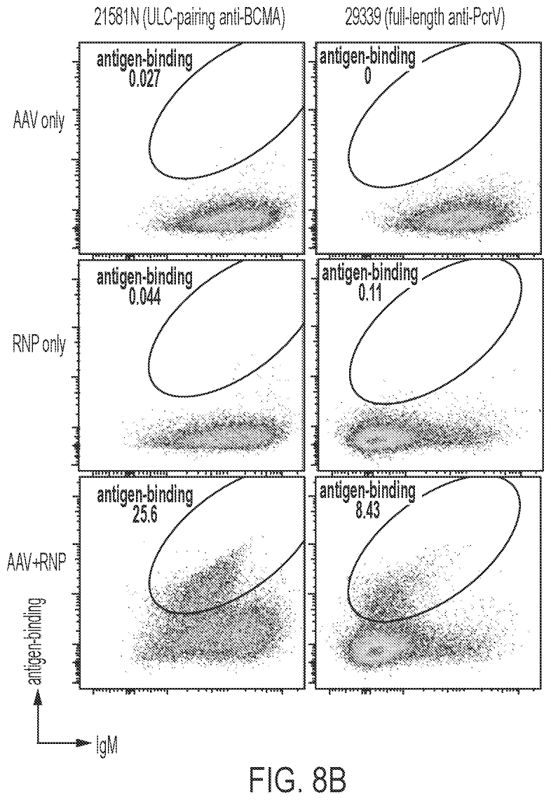

AAV-V13-gRNA1 T2A-21581N (ULC-pairing anti-BCMA) or AAV-V13-gRNA1 T2A-VK29339mIgK-P2A-VH29339 (anti-PcrV) were inserted into mouse splenic B cells cultured with CD40L-HA, anti-HA, and IL-4 (FIG. 8A). Three millions cells were nucleofected at 24 hrs with 150 pmol Cas9 and 400-500 pmol sgRNA, including 400 pmol VH gRNA1 for ULC-pairing; 250 pmol VH gRNA1, and 125 pmol each of IgK gRNA4+IgK gRNA6 for full-length. Results were 500,000 cells infected with AAV1 at 1e5 vg/cell, with substantial antigen binding only in the AAV-RNP condition (FIG. 8B).

Testing Template Designs-mCherry Insertion into V13 ULC B Cells

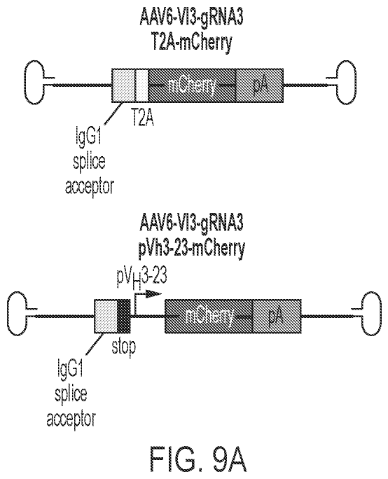

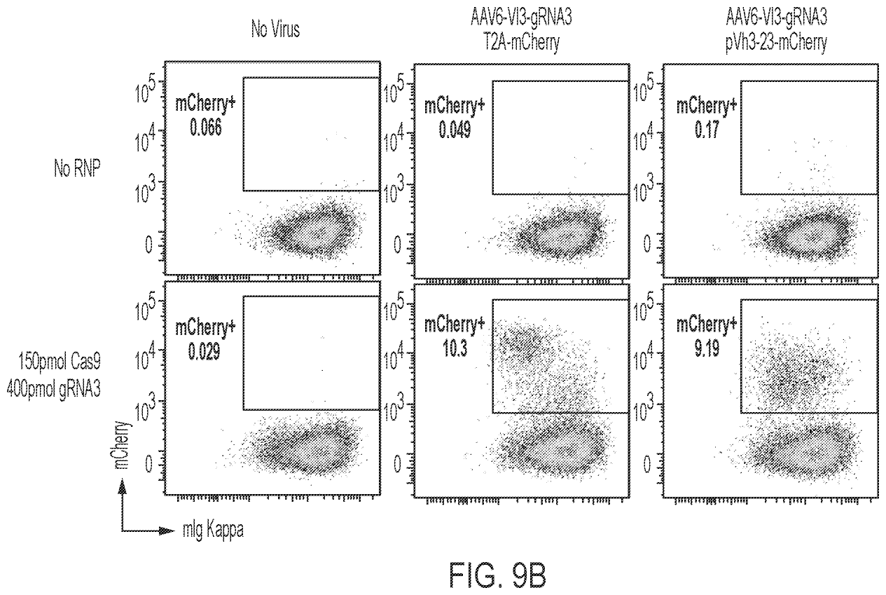

A promoter-less AAV6-VI3-gRNA3 T2A-mCherry or AAV6-VI3-gRNA3 pVh3-23-mCherry was inserted into VI3-ULC B-cells cultured as above (FIG. 9A). Results were 600,000 cells infected with AAV at 5e5 vg/cell, with both inserts showing around 10% mCherry expression (10.3% for T2A-mCherry and 9.19% for pVh3-23-mCherry) (FIG. 9B). Thus, the promoter-less T2A strategy seemed to produce as much mCherry as the additional promoter variant.

Alternate Sets of gRNAs and Homology Arms for BCR Insertion

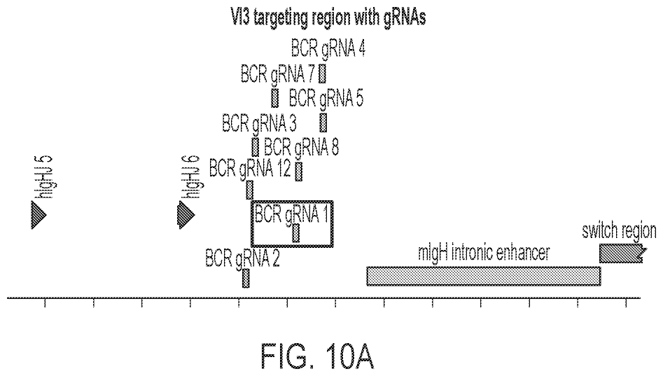

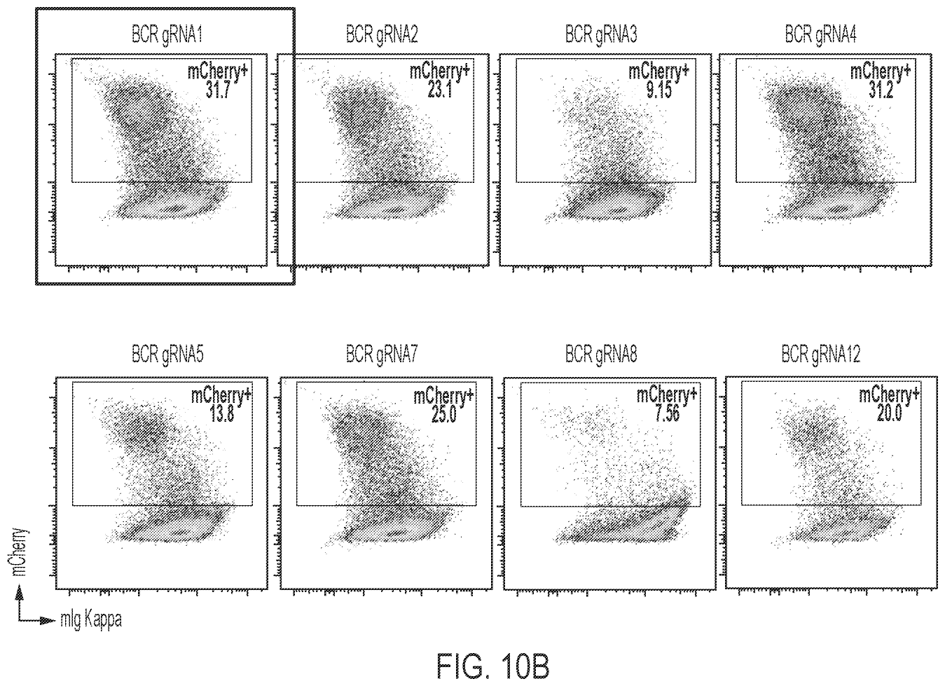

Multiple gRNA targeting sites are available for heavy chain locus VI3 BCR insertion. Using the same promoter-less V13-gRNA3 T2A-mCherry insert as above (FIG. 9A) but switching out gRNAs, eight different gRNAs were used to compare for greatest mCherry expression. (FIG. 10A). Results show that BCR gRNA 1 had the best expression at 31.7%, with BCR gRNA4 at 31.2%. All results shown in FIG. 10B.

### Ig Kappa Deletion for Full Length Antibody Insertion

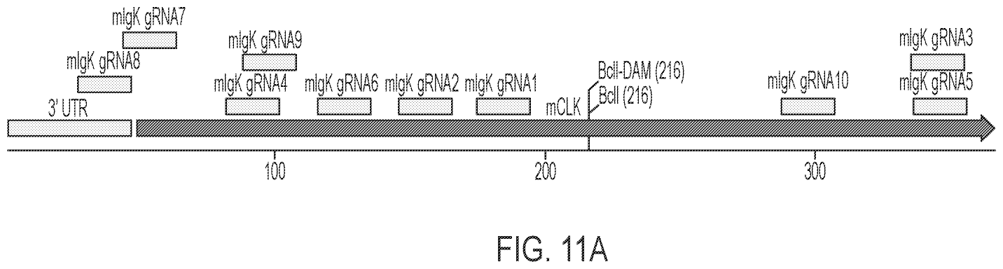

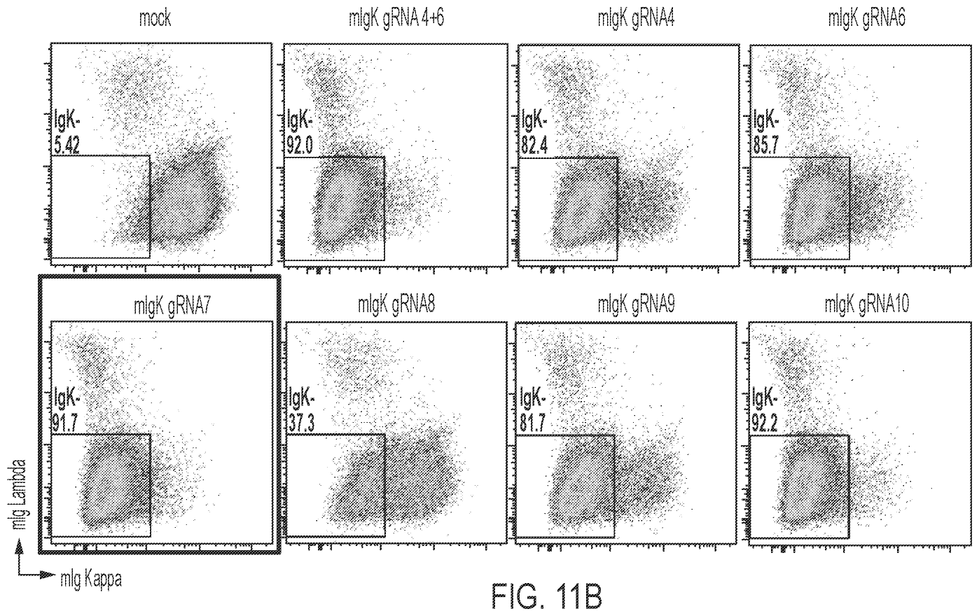

Multiple gRNA targeting sites are also available in the Ig Kappa locus to disrupt expression of the endogenous light chain and support a full-length antibody insertion. 7 different gRNA were used for comparison, gRNA4, gRNA6, gRNA7, gRNA8, gRNA9, gRNA10, and gRNA4+6, all of which are shown in FIG. 11A. Again, mouse splenic B cells were cultured with CD40L-HA, anti-HA, and IL-4. 3 million cells were nucleofected at 24 hrs with 150 pmol Cas9 and 400 pmol gRNA, with the results analyzed 2 days post-nucleofection. Results show that gRNA7, which cuts at the splice acceptor site and doesn't require recoding of Kappa constant in the AAV template, has the lowest mlg Lambda and mlg Kappa expression at 91.7%, followed by gRNA10. All results are shown in FIG. 11B.

mCherry Insertion into V13 B Cells—Effect of Stimulation

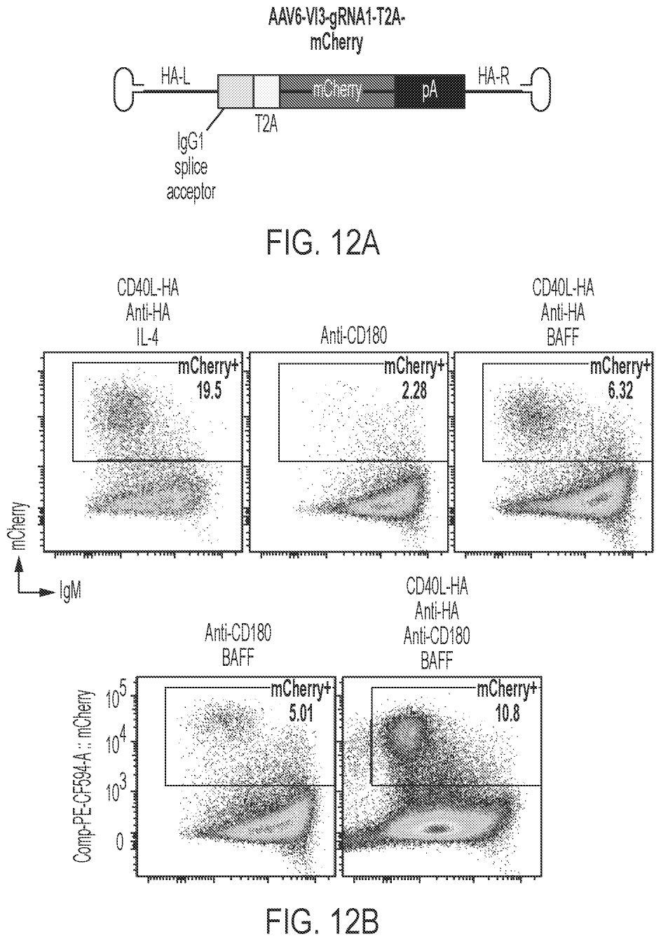

Mouse splenic B cells were cultured with the following growth factors: 1) CD40L-HA, Anti-HA, and IL-4 (as previous), 2) anti-CD180, 3) CD40L-HA, anti-HA, and BAFF, 4) anti-CD180 and BAFF, and 5) CD40L-HA, anti-HA, anti-CD180, and BAFF. 3 million cells were nucleofected at 24 hrs with 150 pmol Cas9, 400 pmol gRNA, and AAV6-VI3-gRNA1-T2A-mCherry (FIG. 12A). 500,000 cells were infected with AAV6 at 2.5e5 vg/cell and analyzed 3 days post-infection. mCherry expression was strongest in conditions 1 (19.5) and 5 (10.8) as shown in FIG. 12B.

### BCR Insertion in Cells Grown in Growth Conditions

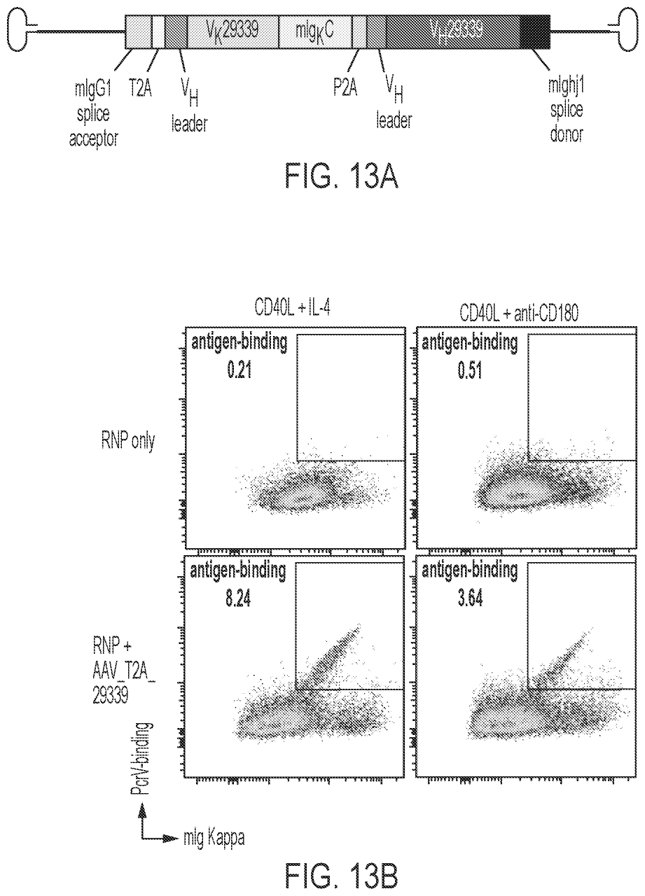

To test more stimulation conditions, mouse splenic B cells were cultured with either 1) CD40L-HA, anti-HA, and IL-4, or 2) CD40L-HA, anti-HA, and anti-CD180. 3 million cells were nucleofected with 150 pmol Cas9, 400 pmol total gRNA (BCR-gRNA1 and mlgK-gRNA7), and a full-length H1H29338 antibody (FIG. 13A). 500,000 cells were infected with AAV1 at 2e5 vg/cell and analyzed 2 days post-infection. Both conditions worked, with condition 1 at 8.24 and condition 2 at 3.64% respectively as shown in FIG. 13B.

Transfer and Immunization Experiment with Anti-PcrV Edited B Cells

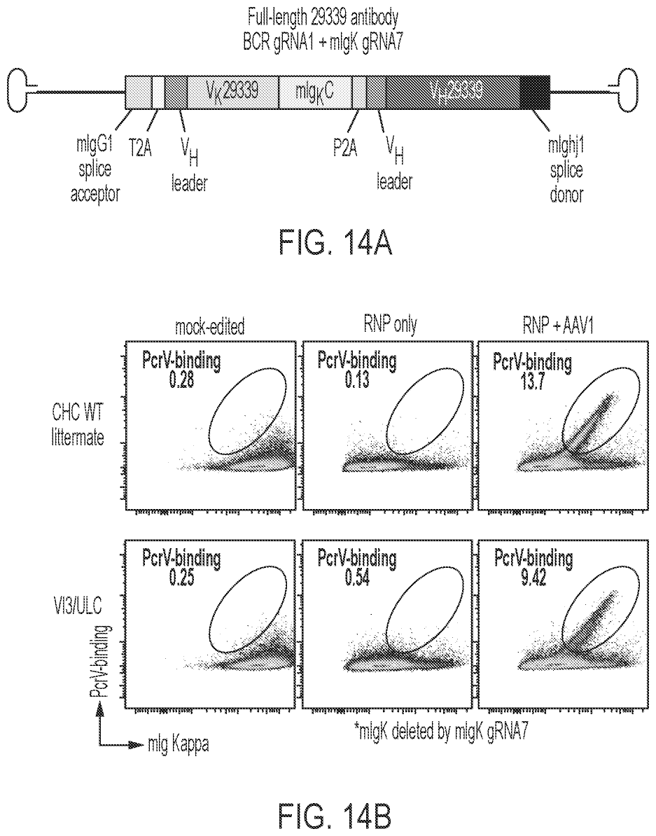

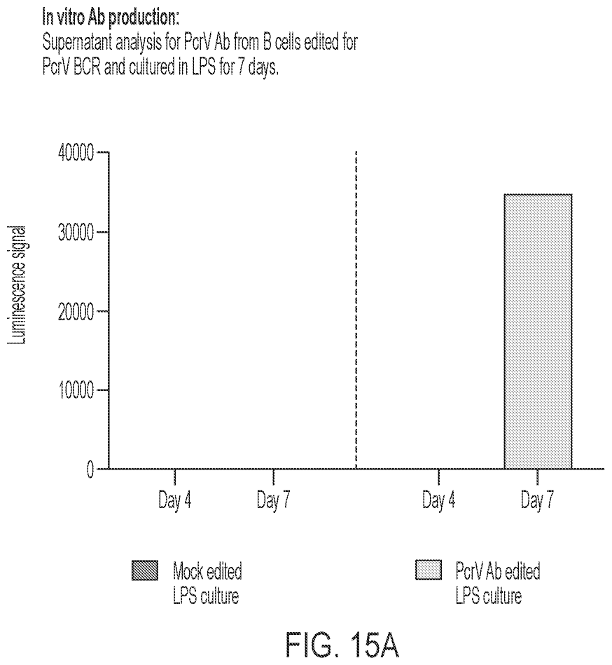

For the following experiments, B cells from a CHC WT mouse were grown in CD40L-HA, anti-HA, and anti-CD180 before RNP nucleofection and AAV1 infection with h1h29339 anti-PcrV full length antibody (FIG. 14A) after 24 hours. 24 hours post editing, 3×10⁶ cells per mouse were transferred to CHC anti-flu mouse littermates (all B cells have a pre-rearranged BCR specific for flu HA) with partial B cell depletion with anti-CD20 to create niche space for the new cells. Additionally, 72 hours post editing, 6 million cells per mouse were transferred to additional littermates. 7 to 8 days after immunization, the mice serum is extracted to test for anti-PcrV antibodies. Compared to the standard non-insertion VI3/ULC, the antibodies from the CHC WT littermates performed well (13.7 to 9.42 respectively, full results shown in FIG. 14B).

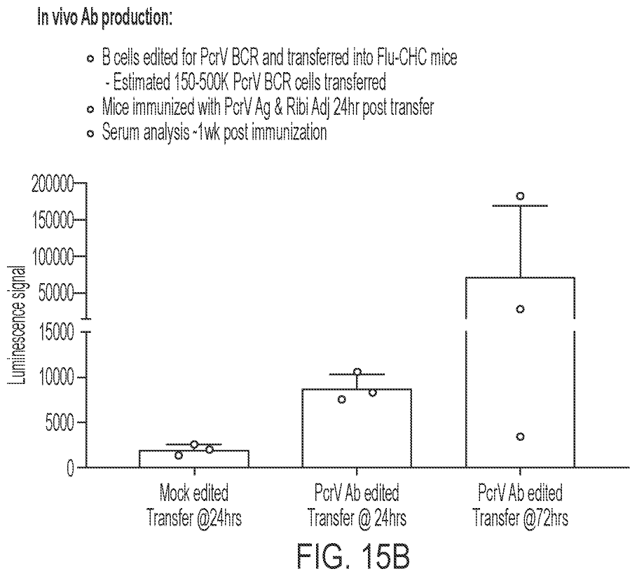

Further, B cells edited to express anti-PcrV BCR can mature to produce anti-PcrV antibodies both in vitro and in vivo after the adoptive transfer and immunization into mice. In vitro, supernatant analysis for PcrV antibodies from B cells edited for PcrV BCR and cultured in LPS for 7 days showed antibody production (FIG. 15A). In vivo, B cells were edited for PcrV BCR and transferred into Flu-CHC mice as previously described, with the serum analysis about 1 week post immunization, with the mice serum again producing antibodies (FIG. 15B).

### Successful Short-Term In Vivo Expansion of Cultured Activated Donor B Cells

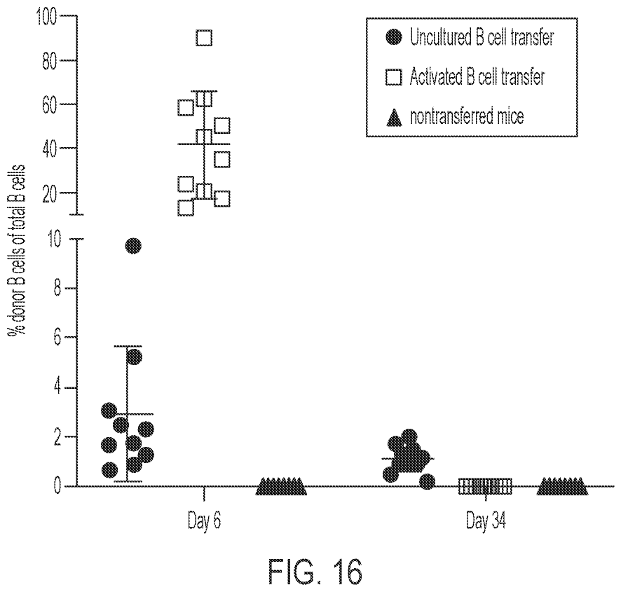

Two factors that significantly influence the ability of donor B cells to engraft are the available space to receive cells in the B cell compartment and the activation/maturation state of the donor cell. Making space is accomplished by conditioning the recipient via partial cell depletion to make space for the incoming B cells. In vitro, the B cell activation conditions have to be modulated, because while strong activating conditions make for great in vitro expansion, the cells may be short lived post injection. For example, in an adoptive transfer of donor B cells from HA antigen immune mice into a CD20 cell-depleted naïve mouse recipient, the donor in vitro activated B cells appear greatly expanded one week post transfer but fail to persist 1 month later (FIG. 16). However, donor non-activated B cells fail to significantly expand one week post transfer, but are still present 1 month later.

### Example 4: Driving Hematopoietic Expression of Cas9 Using AAV

Adeno-associated virus (AAV) based gene therapy vectors are the current gold standard for in vivo delivery of transgenes (1). The DNA packaging capacity of AAV is limited by the capsid and corresponds approximately to the wild-type AAV genome size: ˜4.7 kb. The coding sequence of many therapeutic modalities, such as the programmable nuclease Cas9, are approaching this limit. Together with other features encoded in recombinant AAV vectors that are necessary for transgene delivery and expression (e.g., terminal repeats, termination signals and others), this leaves little space for regulatory sequences such as promoters and enhancers. Both commonly used viral and endogenous human regulatory elements exceed these size limitations and can therefore not be used for AAV-mediated delivery of large transgenes.

The present inventors designed a short transcriptional regulatory sequence for the expression of large transgenes within an AAV context by identifying a short (80 bp), ubiquitous enhancer element that drives high expression of a reporter gene in several cell types when coupled to a minimal promoter.

The present inventors reasoned that enhancer sequences that are active in a wide range of cell types might contain clusters of transcription factor binding sites responsible for activity in a subset of cell types. Such a partial sequence would lose activity in some cells, while retaining activity in a relevant subset of cells. To test this hypothesis, the enhancer of spleen focus forming virus (SFFV, 408 bp) was chosen. SFFV has been shown to be active in hematopoietic cells. The present inventors first identified clusters of potentially cis-regulatory transcription factor binding sites by mapping JASPAR transcription factor binding site profiles. A minimal core-promoter within SFFV, necessary to initiate transcription, was then identified. Based on these predicted elements the present inventors removed the core-promoter and selected and introduced 5 partial SFFV sequences individually into an AAV construct, coupled to the adenovirus major late minimal promoter. Among these sequences, SFFV-4 displayed strong activity in murine B cells, approaching the activity levels of full-length SFFV.

### Transcription Factor Binding Site Mapping

Transcription factor binding sites were mapped within the SFFV sequence using fimo (version 5.1.1, http://meme-suite.org/tools/fimo). The JASPAR CORE database of transcription factor binding motifs Ojaspar.genereg.net) in MEME format was used as input for fimo, together with the full-length sequence of SFFV (see Table 2, below).

TABLE 2
SFFV sequences

| ID | SEQUENCE |
|---|---|
| SFFV | GTAACGCCATTTTGCAAGGCATGGAAAAATACCAAACCAAGA |
| | ATAGAGAAGTTCAGATCAAGGGCGGGTACATGAAAATAGCTA |
| | ACGTTGGGCCAAACAGGATATCTGCGGTGAGCAGTTTCGGCC |
| | CCGGCCCGGGGCCAAGAACAGATGGTCACCGCAGTTTCGGCC |
| | CCGGCCCGAGGCCAAGAACAGATGGTCCCCAGATATGGCCCA |
| | ACCCTCAGCAGTTTCTTAAGACCCATCAGATGTTTCCAGGCT |
| | CCCCCAAGGACCTGAAATGACCCTGCGCCTTATTTGAATTAA |
| | CCAATCAGCCTGCTTCTCGCTTCTGTTCGCGCGCTTCTGCTT |
| | CCCGAGCTCTATAAAAGAGCTCACAACCCCTCACTCGGCGCG |
| | CCAGTCCTCCGACAGACTGAGTCGCCCGGG |
| | (SEQ ID NO: 3) |
| SFFV-4 | GCTCCCCCAAGGACCTGAAATGACCCTGCGCCTTATTTGAAT |
| | TAACCAATCAGCCTGCTTCTCGCTTCTGTTCGCGCGCT |
| | (SEQ ID NO: 4) |
| MLP | GGGGGGCTATAAAAGGGGGTGGGGGCGTTCGTCCTCACTCT |
| | (SEQ ID NO: 5) |

### Selection of Sub-Sequences of SFFV

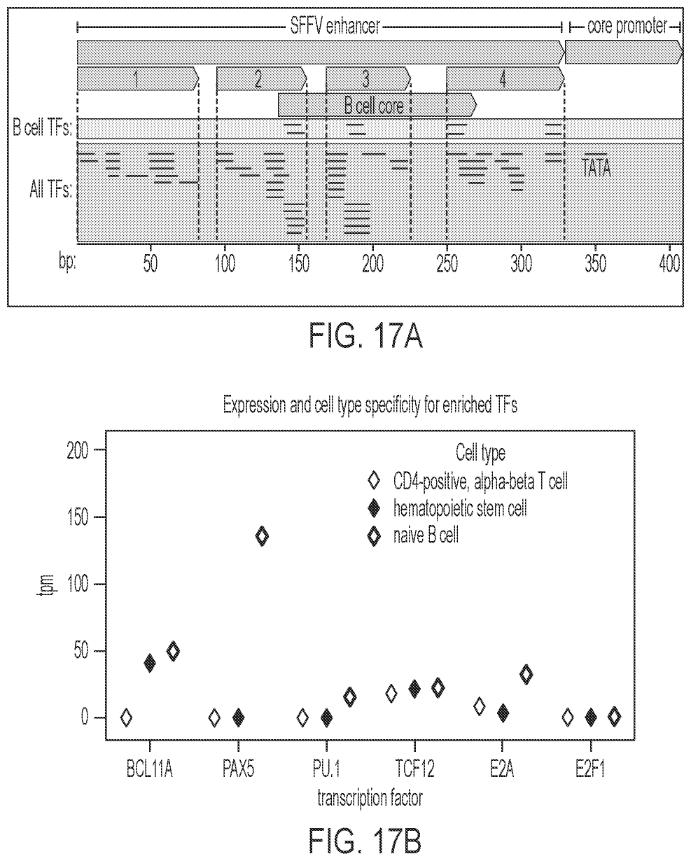

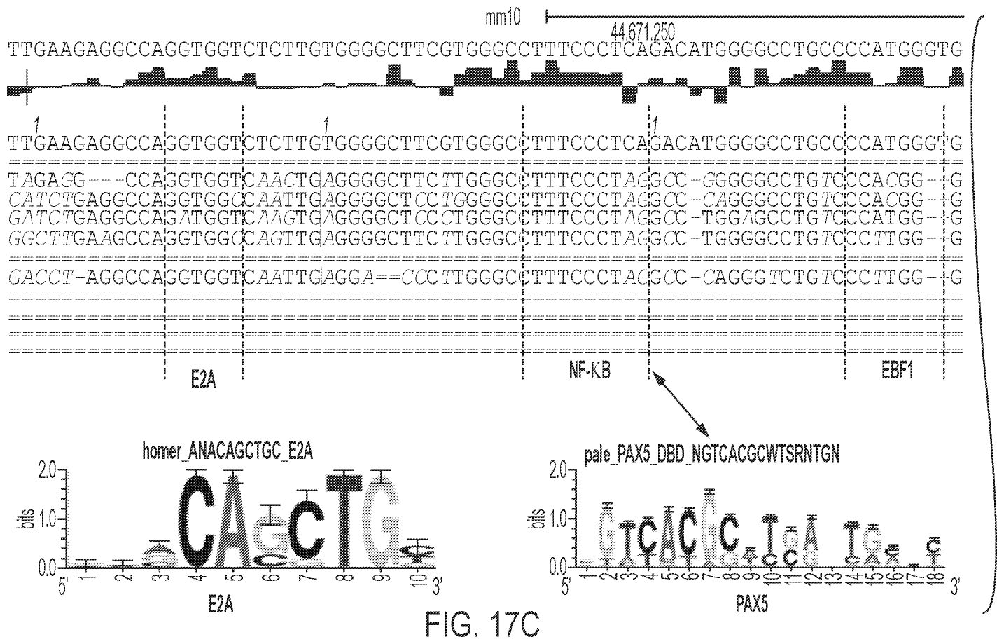

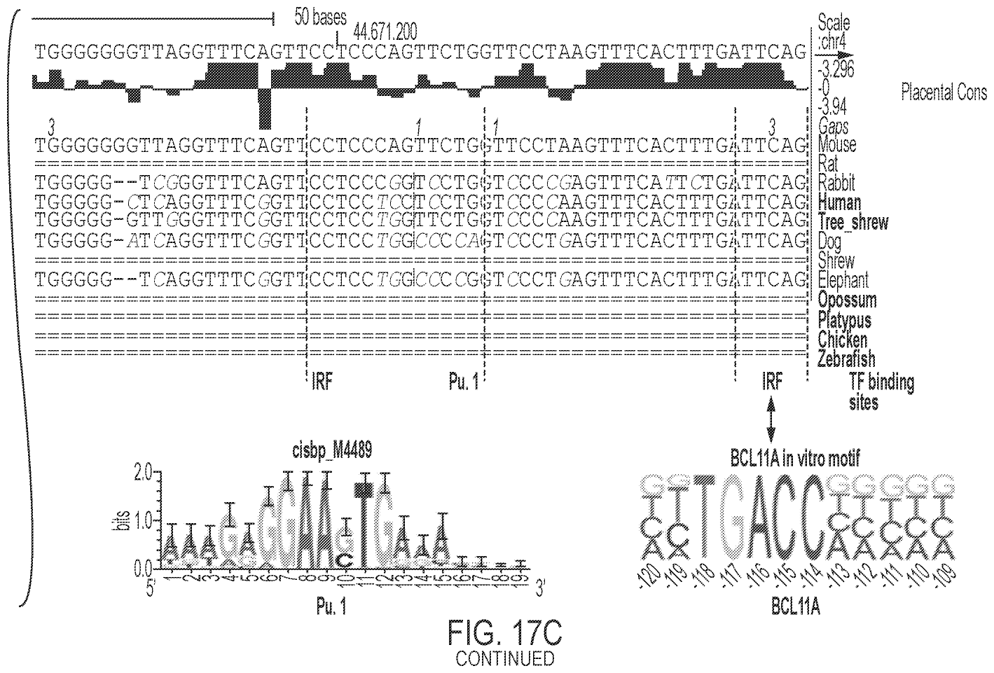

In order to select sub-sequences of SFFV that would potentially be active in hematopoietic cells, short contiguous stretches of sequence within SFFV were selected that did not contain DNA sequence without predicted transcription factor binding sites (FIG. 17A). 4 such sub-sequences were selected based on all transcription factor binding sites and one ‘B cell core’ sub-sequence, selected based on B cell specific transcription factor binding sites (FIGS. 17B and 17C). A putative core promoter was also identified within SFFV based on the location of a TATA-box, which is frequently found ˜30 bp upstream of the transcription start site. When testing transcriptional activity of these sub-sequences, this putative core promoter sequence was replaced with the adenovirus major late core promoter (MLP).

### Generation of Reporter Constructs and Infection of Primary Murine B Cells

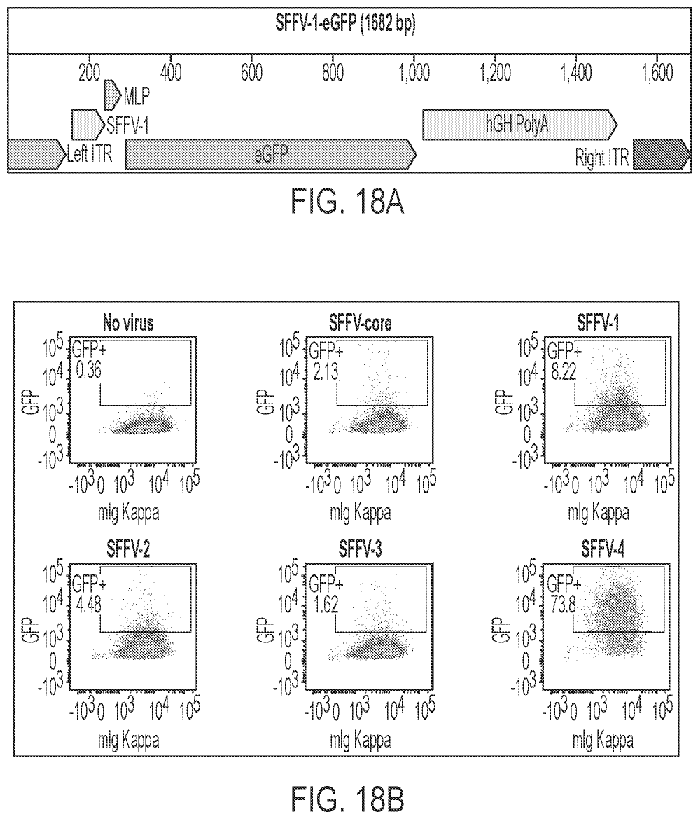

The SFFV sub-sequences were ordered as double-stranded DNA (gBlock, IDT) and diluted to 10 ng/μl in water. To facilitate cloning, MluI and EcoRI restriction sites were added to the 5′ and 3′ end, respectively. All candidates were ligated into the recipient pAAV-GFP vector using restriction digest followed by T4 DNA ligation, resulting in 5 candidate AAV plasmids expressing GFP under the control of the SFFV sub-sequence, paired with MLP (FIG. 18A). Primary mouse B cells were infected with purified AAV and the number of GFP positive cells (based on a negative control) was measured using fluorescent activated cell sorting (FACS). 5e5 vg/cell of AAV6 crude viral prep, cells cultured with CD40L-HA, anti-HA, and IL-4, 3 days post-infection. Results shown in FIG. 18B.

B Cell Line Test: R AMOS vs. HEK293-HZ

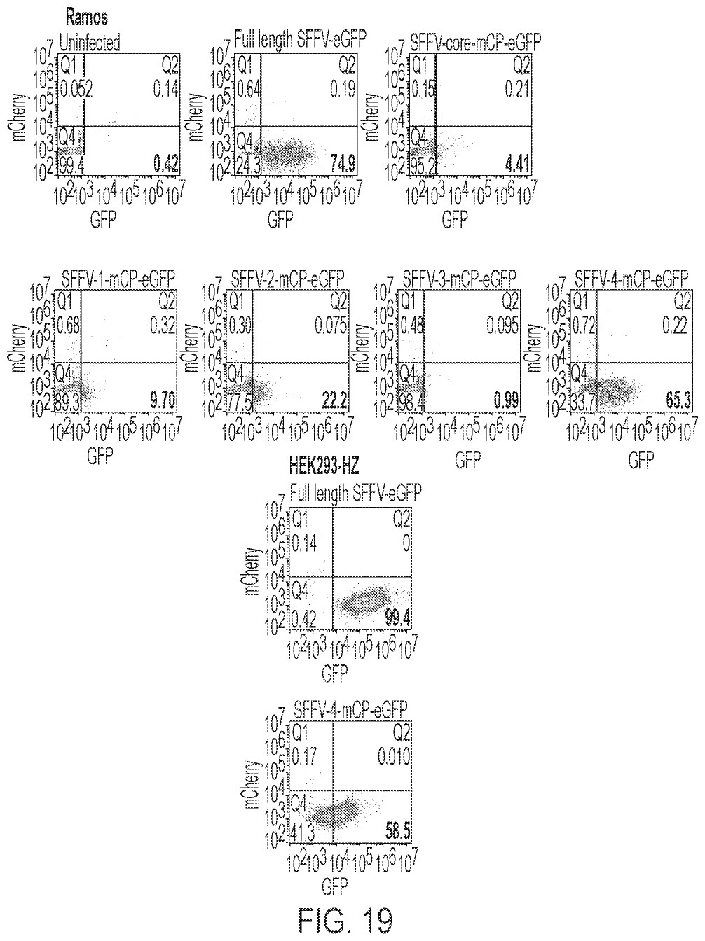

Full length SFFV-eGFP and variants, including SFFV-core-mCP-GFP, SFFV1-mCP-eGFP, SFFV2-mCP-eGFP, SFFV3-mCP-eGFP, and SFFV4-mCP-eGFP, were transfected into Ramos and HEK293-HZ cells. SFFV4 showed activity in Ramos and HEK cells at 121 bp in length (FIG. 19).

### Use of HS-B Pax5 Enhancer

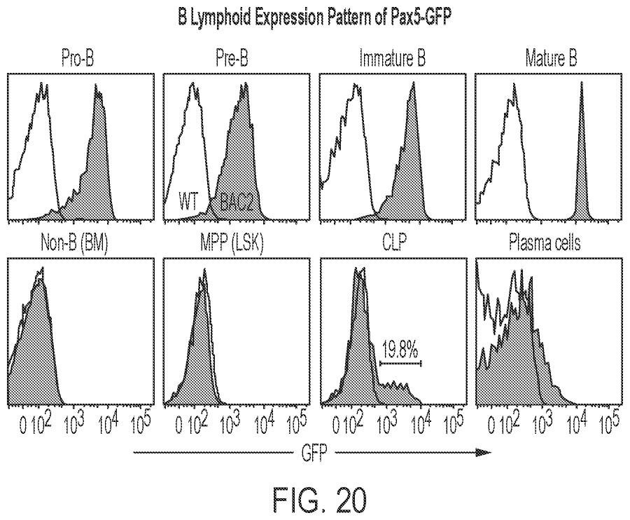

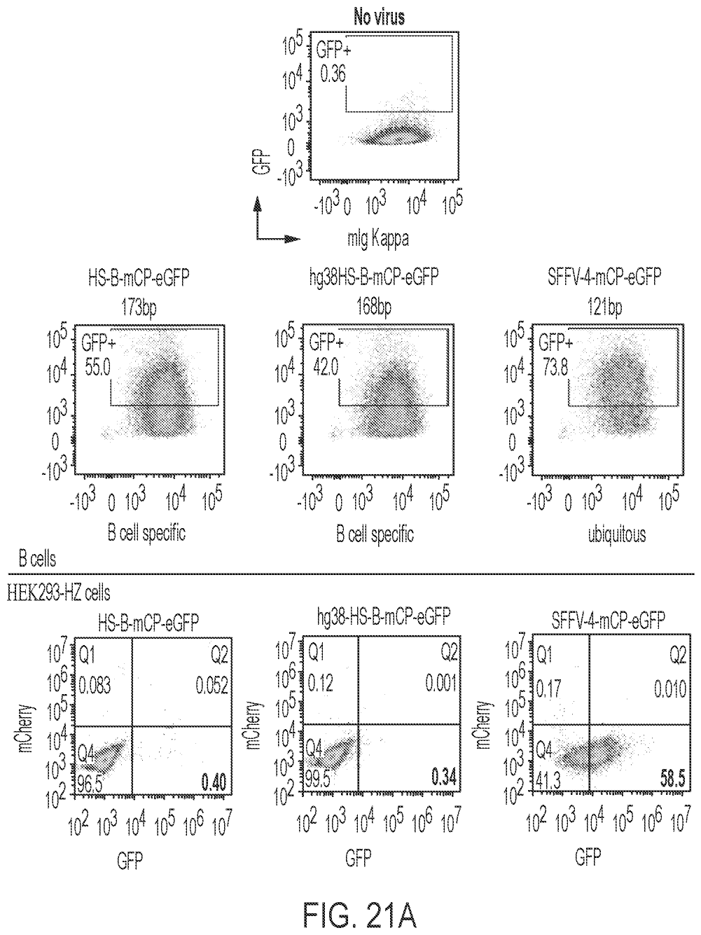

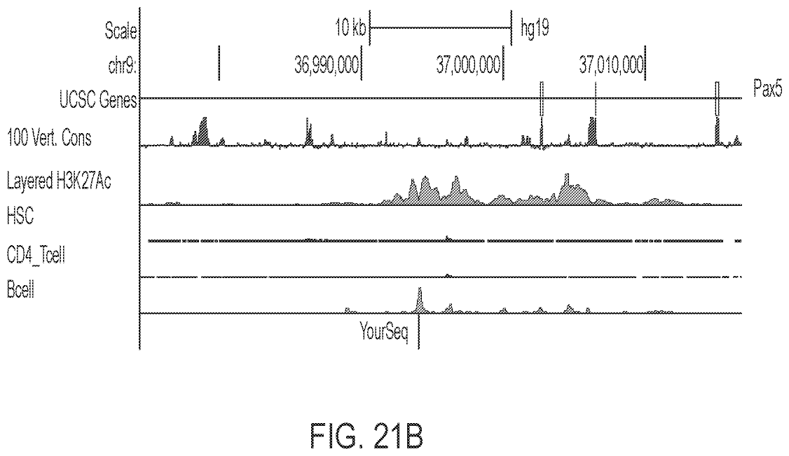

HS-B is a 180 bp B cell specific Pax5 enhancer as shown in luciferase expression in mouse B cell line (FIG. 20). For AAV-GFP tests of 120-170 base pair promoters in primary B cells and HEK293-HZ cells, three promoters were used with mCP-eGFP: 1) HS-B, 2) hg38HS-B, and 3) SFFV4. The cells were cultured and transfected with 5e5 vg per cell of AAV6 crude viral prep and CD40L-HA, anti-HA, and IL-4. Results are shown in FIG. 21A.

### Example 5: AAV Retargeting to CD20 for Targeted B Cell Transduction

Adeno-associated virus (AAV) is currently one of the leading viral vectors used in gene therapy to treat human diseases. Although AAV has many advantages as a gene therapy vector, one of the drawbacks to its use in systemic gene delivery is the relatively broad tropism of the virus and its tendency to preferentially target the liver. For many potential gene therapy applications, limiting infection to specific tissues or cell types would be advantageous. This Example relates to developing a cell type-specific AAV vector for in vivo delivery of therapeutic transgenes. AAV vectors were rationally engineered to target specific cell types by genetically abolishing the natural tropism of the virus and then redirecting the virus to target specific cells using monoclonal antibodies. Two parallel platforms for antibody-mediated AAV retargeting were developed: one is an affinity-based approach and the other relies on covalent coupling of antibodies to AAV particles. Both of these approaches were used to retarget AAV to specific cell types in vitro, in mice, and in non-human primates (NHPs).

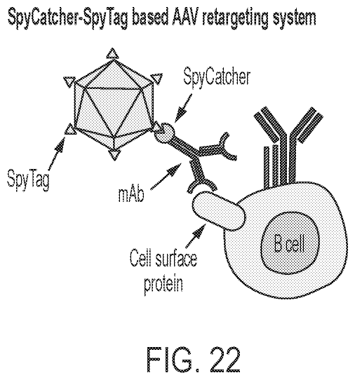

CD20 is a B cell marker expressed in healthy and malignant B cells, and is involved in calcium signaling. CD20 is expressed early in B cell development, but expression is lost during differentiation into plasma cells. CD20 is the target of many antibody drugs such as Ofatumamab (HuMax) and Rituximab (Rituxan) which bind to different epitopes on the extracellular loops. Initial AAV retargeting experiments were performed with HuMax. Since AAV variable loops can tolerate foreign peptide insertions, SpyTag:SpyCatcher binding system was used to attach mAb to the surface of the AAV capsid for retargeting purposes (FIG. 22).

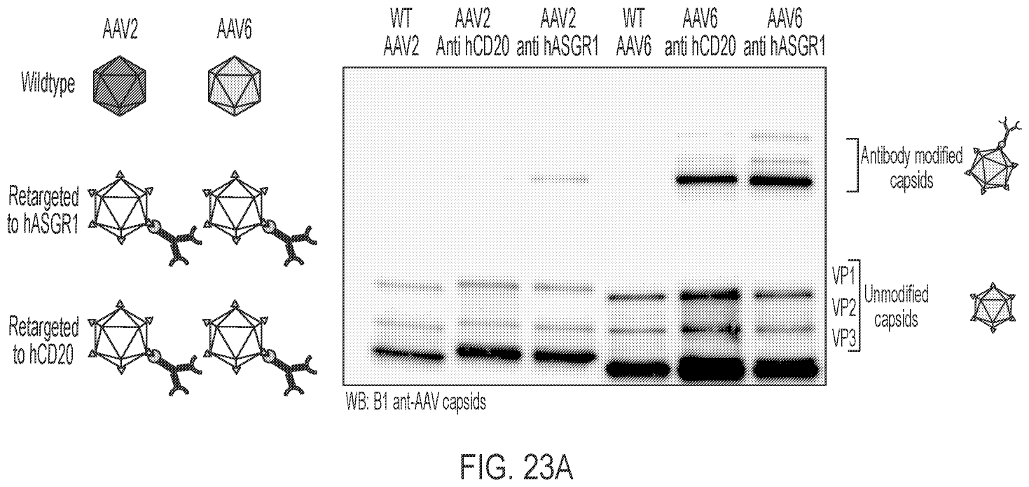

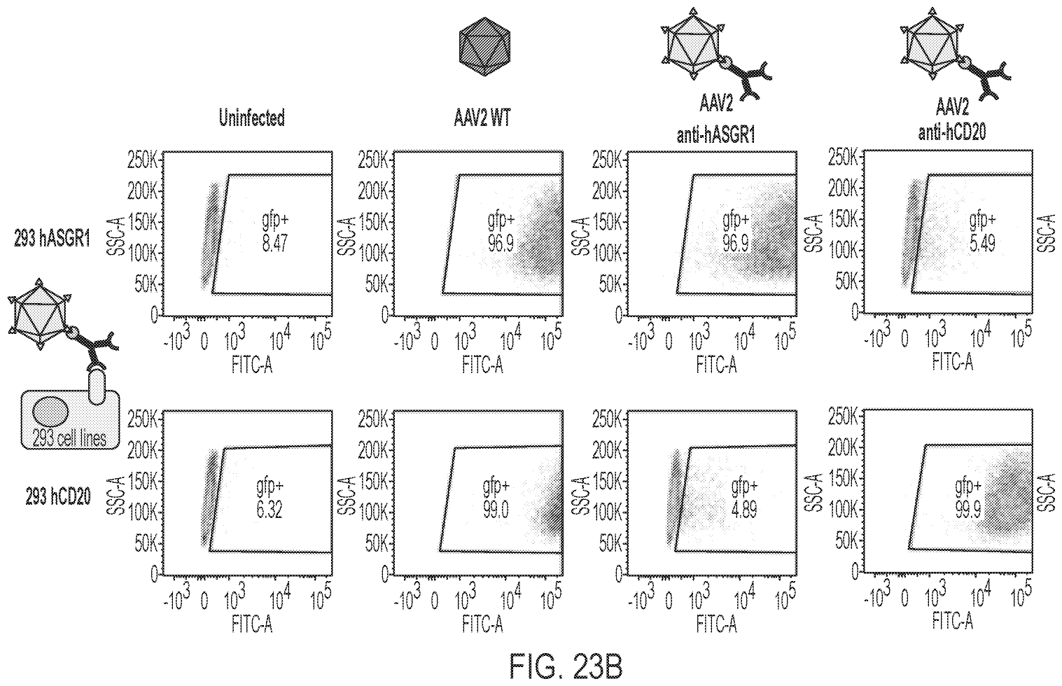

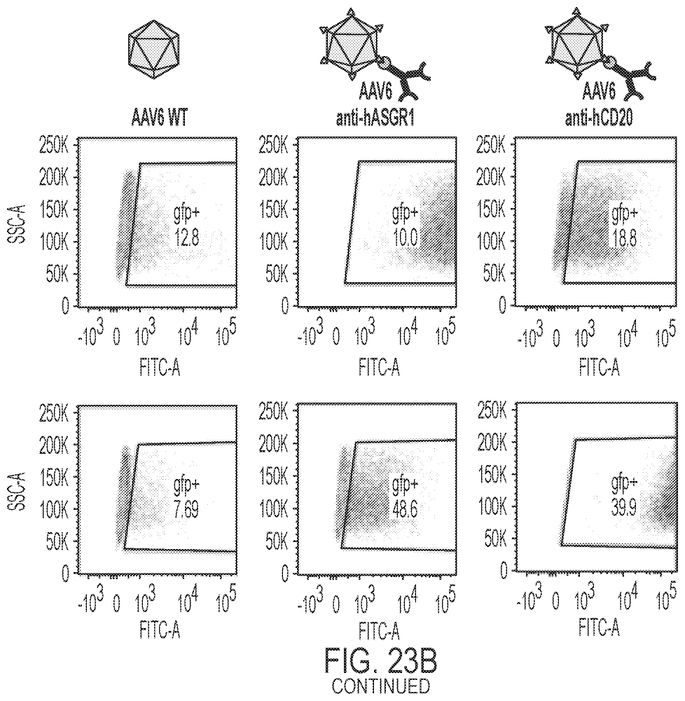

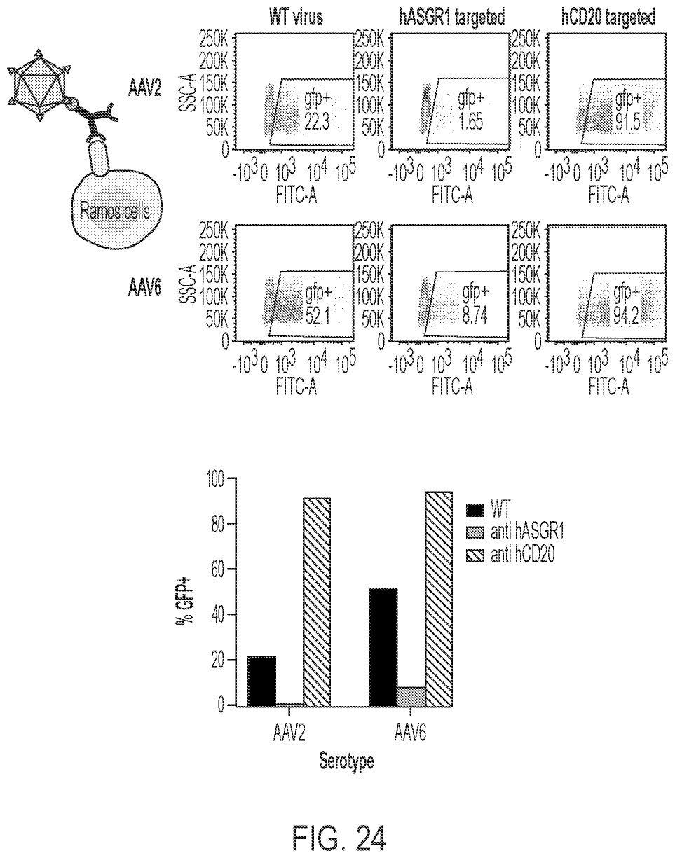

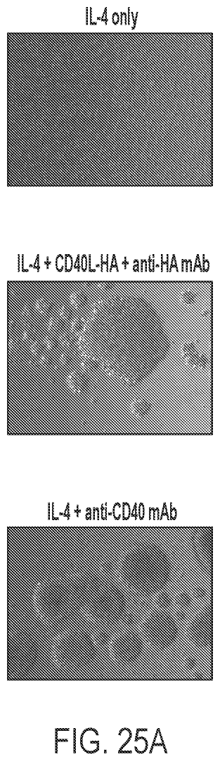

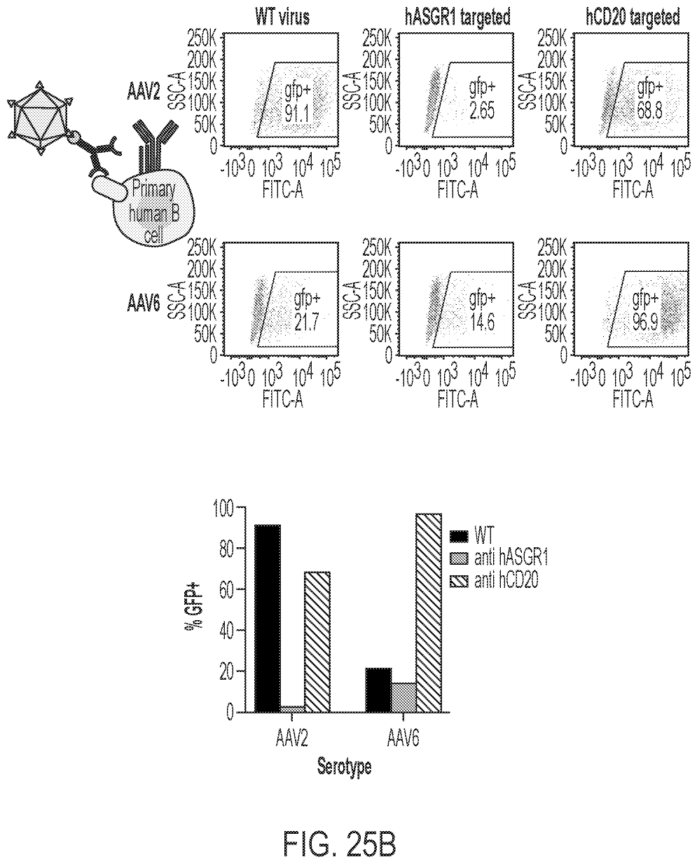

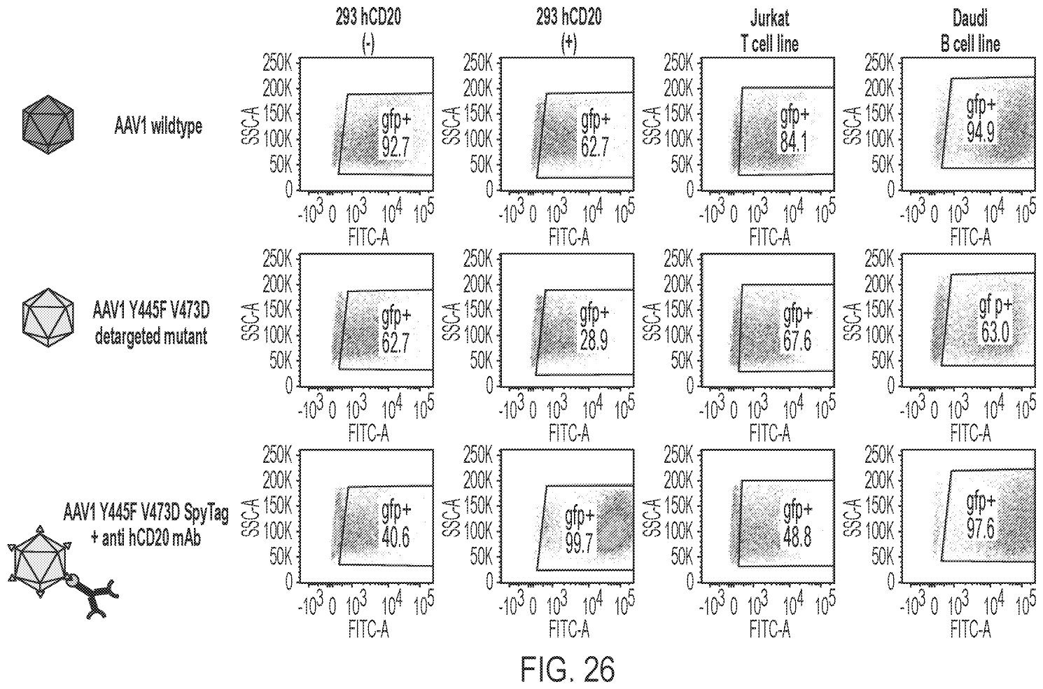

With regards to CD20, both AAV2 and AAV6 were targeted to CD20-expressing cells, including HEK-293 (FIG. 23B) and Ramos (FIG. 24) cell lines. While AAV2/CD20 was highly accurate, AAV6/CD20 appeared not fully de-targeted and displayed some off-target transduction. To determine if this system can be used to retarget AAV to primary human B cells, CD19+ B cells were isolated from human peripheral blood, and cultured under various stimulation conditions: 1) IL-4 only, 2) IL-4, CD40L-HA, and anti-HA mAb, and 3) IL-4 and anti-CD40 mAb. The cells were infected with AAV2/CD20 or AAV6/CD20, and virus-delivered eGFP was measured by flow cytometry on day 4 post-infection. The results showed that while both AAV2 and AAV6 can be targeted to primary human B cells via CD20, AAV6/CD20 demonstrates a dramatic enhancement in transduction (FIG. 25B).

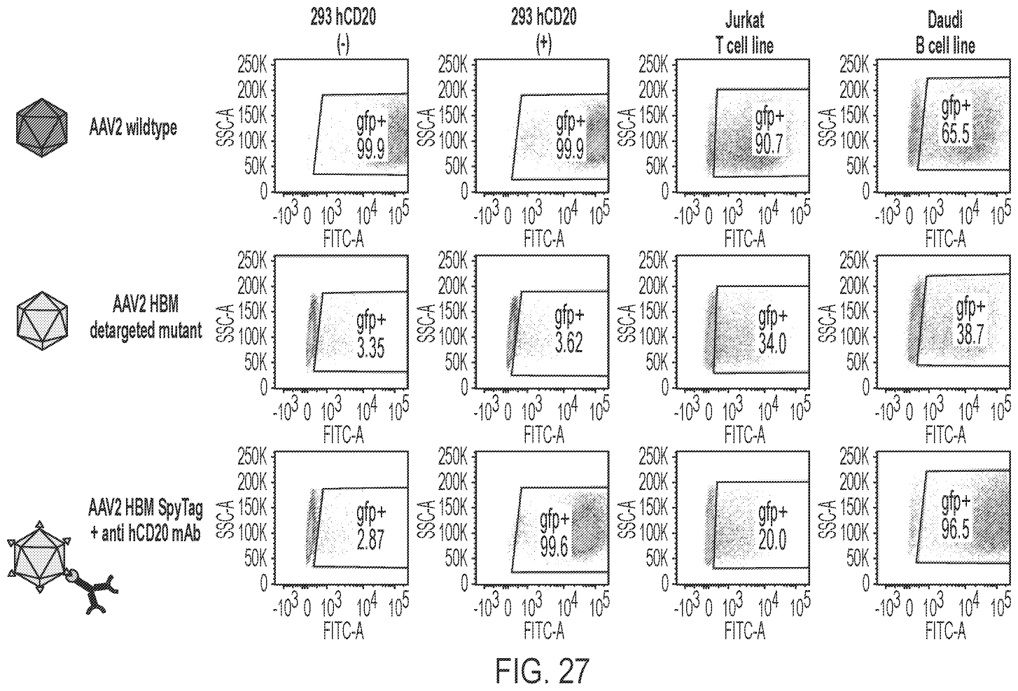

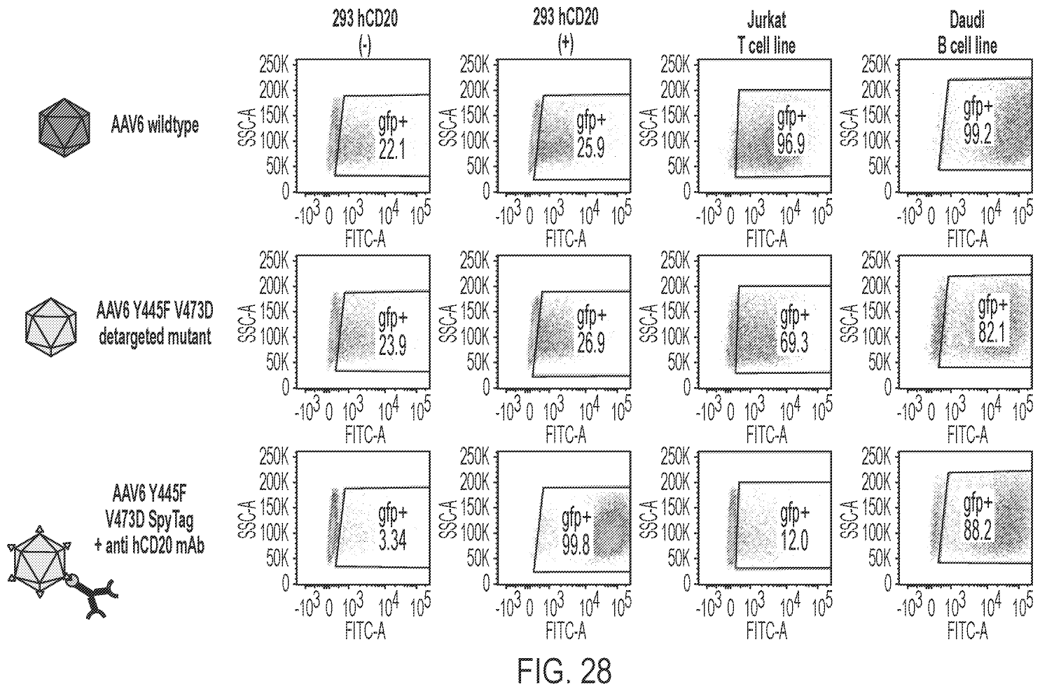

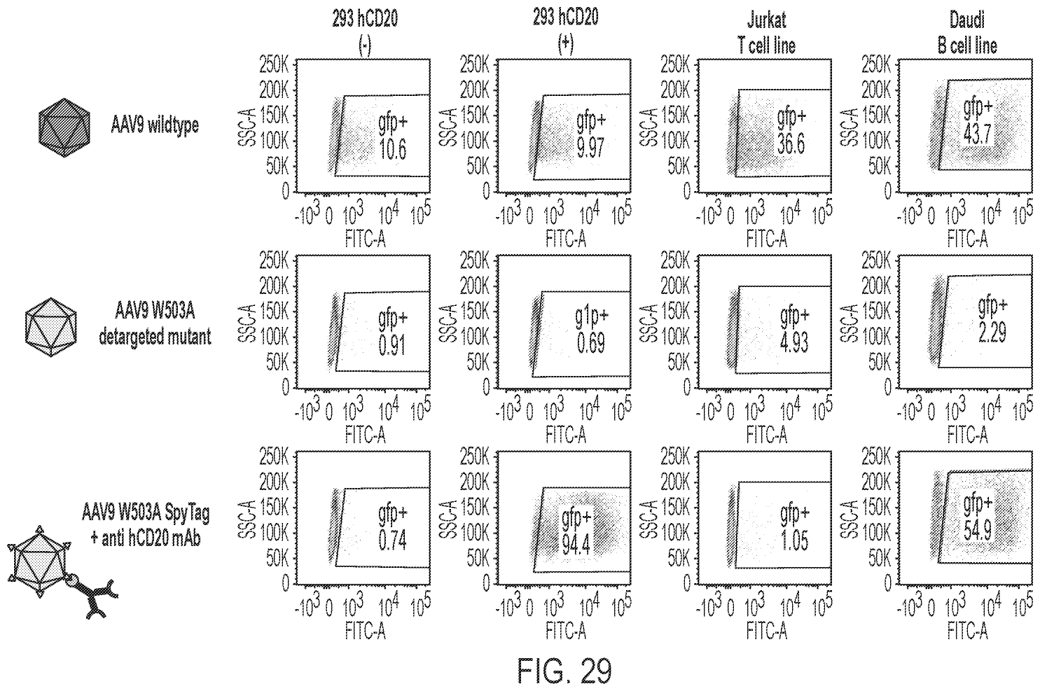

Additional testing was performed for the retargeting of AAV1, AAV2, AAV6 and AAV9 against 1) HEK 293 hCD20(−), 2) HEK 293 hCD20(+), 3) Jurkat T cell, and 4) Daudi B cell lines. Three types of each virus were used: wild-type (WT), a detargeted mutant, and the detargeted mutant attached to a CD20 antibody with SpyTag:SpyCatcher. Each virus delivered SFFV-eGFP with GFP signal measured by flow cytometry. AAV1 results showed that the AAV1 detargeted mutant still transduced and antibody conjugation slightly decreased off-target transduction, while the retargeted virus was comparable to WT in the Daudi cell line (FIG. 26). AAV2 results showed that AAV2-CD20 shows gain-of-function on the Daudi cell line (FIG. 27). AAV6 results showed that the AAV6 retargeted mutant was not completely detargeted, non-binding mAb decreased off-target transduction, and AAV6-CD20 showed a gain-of-function in the 293 hCD20(+) cell line (FIG. 28). Finally, the AAV9 results showed that AAV9-CD20 exhibited a gain-of-function in the hCD20(+) cell line and low off-target transduction (FIG. 29).

### Example 6: In Vivo Targeting of AAV to Human B Cells for BCR Editing and Antibody Production

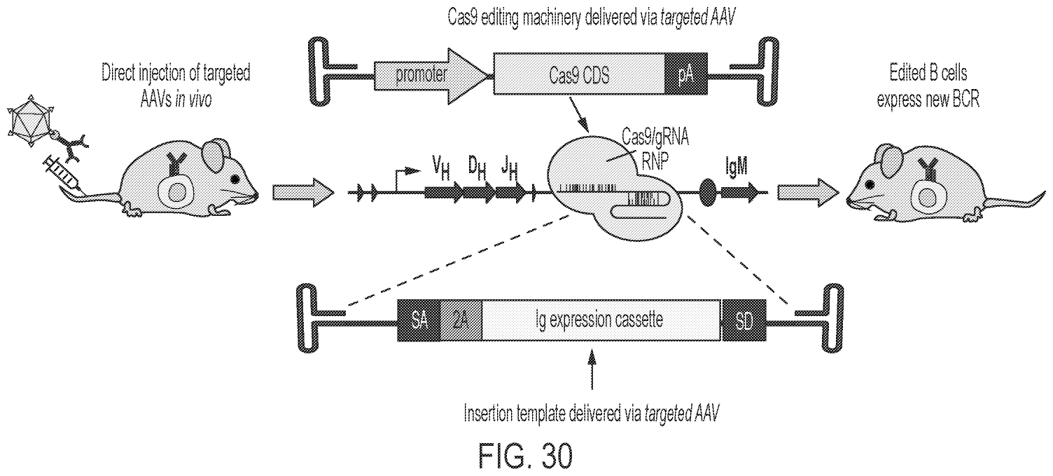

While an ex vivo cell engineered approach to vaccination may be feasible for an individual, it is unrealistic for treating a population. To meet this need, cell editing methods that rely solely on injectables must be developed. The present inventors propose to translate the ex vivo B cell targeting and editing technology to in vivo application by delivering the viral vectors in vivo to mediate BCR insertion (FIG. 30). Inefficiencies in in vivo B cell editing may be overcome through in vivo highly selective expansion of the engineered B cells using methods developed.

Effective Targeting of Human B Cells In Vivo with AAV

Primary human B cells are retargeted ex vivo with several types of AAVs conjugated to CD20 antibodies (as shown in Example 5). Next, the specificity and efficiency of CD20-retargeted viruses is evaluated in a mixed culture setting (human PBMCs) ex vivo. Additionally, other CD20 antibodies with different binding properties and affinities are tested, for example, antibodies and comparable such as Rituximab. Then, the targeting arms are expanded to CD22, CD79, and CD180 antibodies to determine which AAV-antibody combination is the most successful. Finally, AAV retargeting in in vivo humanized mice is performed, first with CD20, and then with other antibodies if necessary. The goal is to identify the best AAV-targeting antibody combination to successfully retarget primary human B cells in vivo with little to no off-target effects.

Effective Cas9 Expression from AAV

Expressing full-length Cas9 from an AAV genome is challenging because most endogenous promoters are too large, and even viral promoters are typically >400 bps. However, for the proposed system to work, full Cas9 needs to be expressed. Much work has already been performed on shortening existing strong promoters (SFFV), and strengthening existing short and cell-type specific promoters (as shown in Example 4). Additional work is performed to shorten existing strong promoters, strengthen existing short and cell-type specific promoters, and identify novel cell-type specific promoters to allow for robust, full length Cas9 in vivo expression post AAV infection of B cells.

### Effective, Dual-AAV Vector Mediated BCR Editing of B Cells In Vivo in Mice and Non-Human Primates

Once the most effective and on-target AAV vector/antibody combination is identified, and once effective Cas9 expression is shown, the final step is to perform and optimize in vivo BCR editing of B cells. The ratio between Cas9-expressing AAV and the gRNA and insertion template-expressing AAV is varied to optimize in vivo BCR insertion efficiencies, first in mice, then in non-human primates. Frequencies of BCR expression are assessed by flow cytometry, both with and without B cell expansion methods. Finally, antibody responses over time and in response to antigen challenges are characterized.

### Example 7: Viral Vector Targeting of Human Stem Cells (HSC)

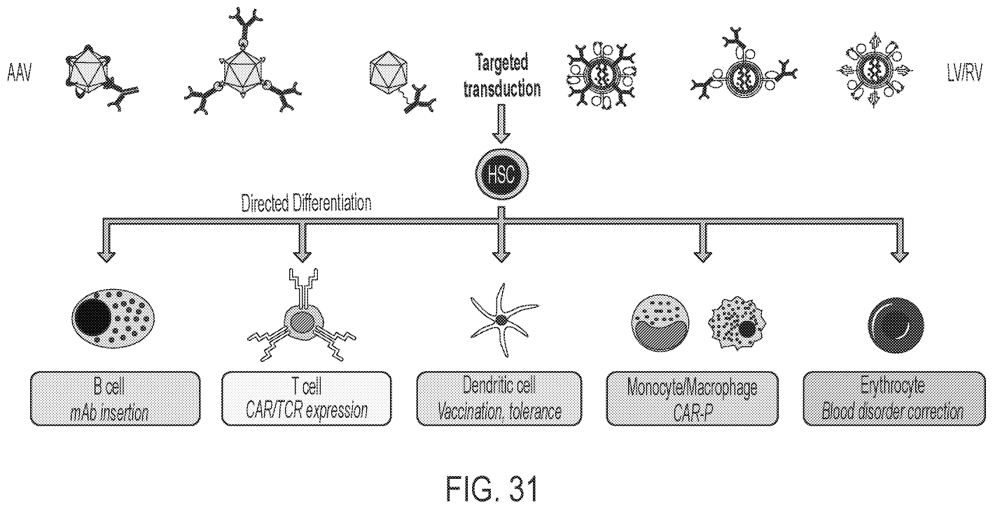

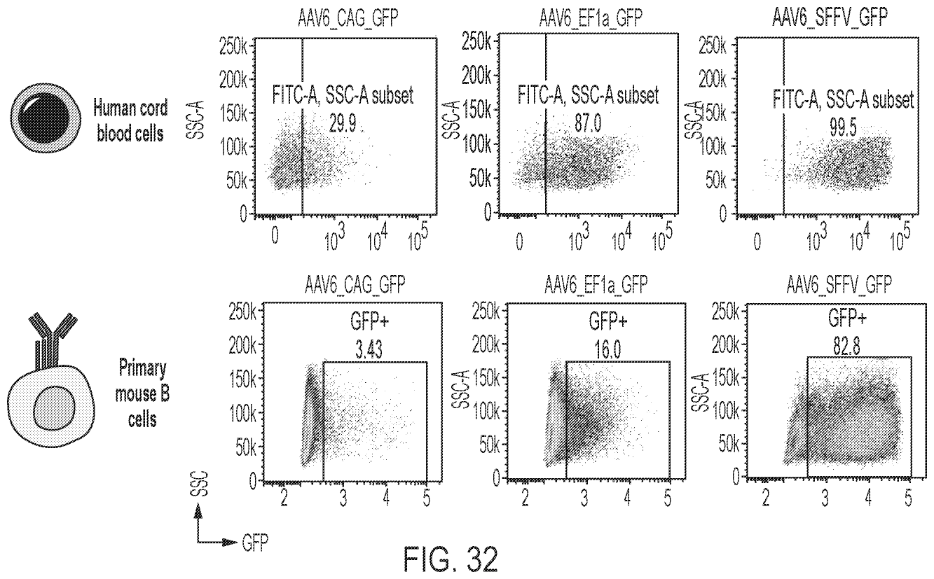

While targeting viral vectors to B cells has been discussed, human stem cells are upstream of immune cells and represent a target for transduction by a range of viruses (FIG. 31). The main marker of long-term hematopoietic stem cells (LT-HSC) in the human hematopoietic system. To test retargeting of AAV with antibodies to HSCs, AAV2, AAV6, and AAV9 were attached to an anti-hCD34 (My10) antibody via the SpyTag:SpyCatcher system. First, AAV6-hCD34-GFP, with three different promotors, were used to infect human cord blood cells and primary mouse B cells, which showed that SFFV was the preferred promoter (FIG. 32).

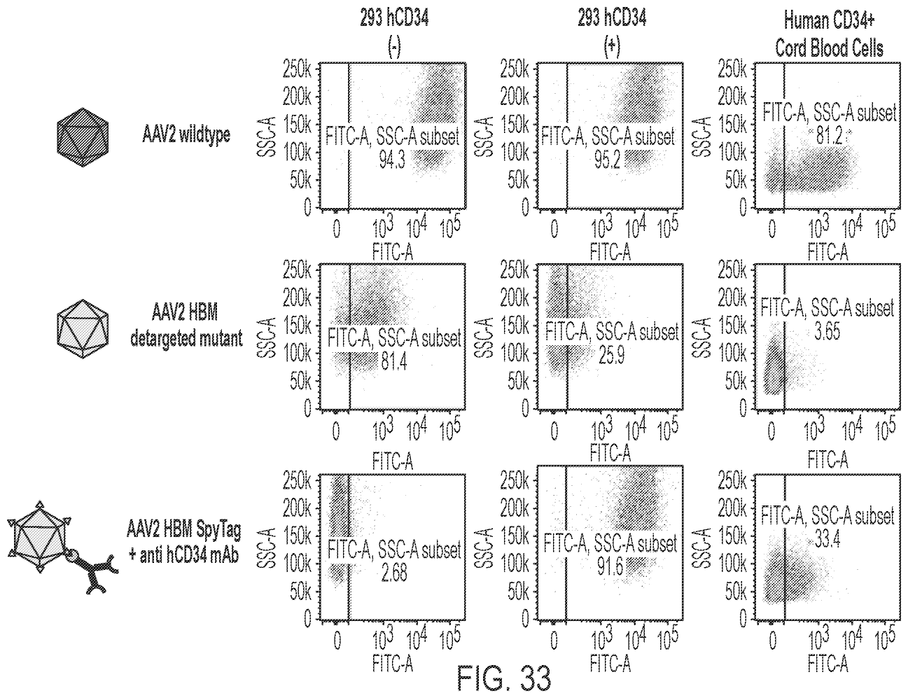

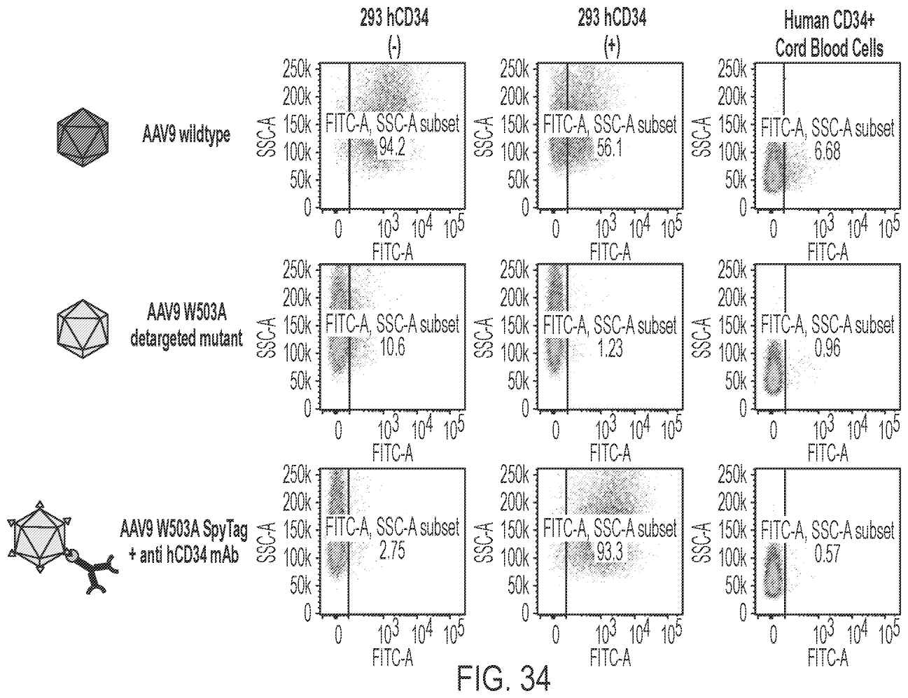

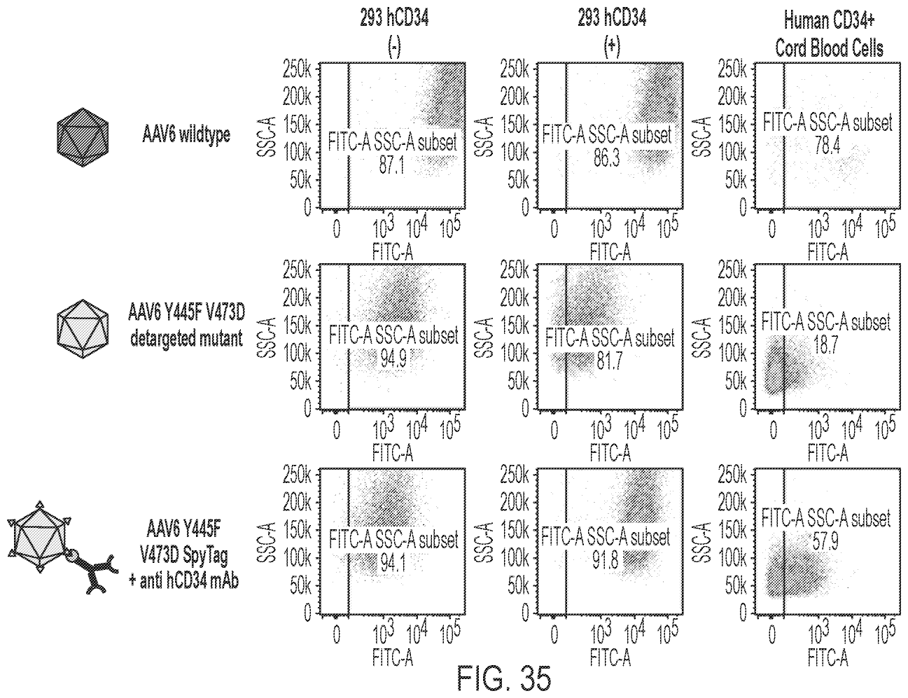

Next, AAV2-hCD34, packed with SFFV-eGFP, was retargeted to HSCs. Results indicate that natural tropism overrides retargeting antibody on human cord blood cells, while non-binding mAb decreases off-target transduction and anti-CD34 mAb can retarget AAV2 HBM mutant in 203/hCD34 and human cord blood cells (FIG. 33). In a similar experiment, replacing AAV2 with AAV9, the results showed a gain of function on 293 hCD34+ cell line in the presence of CD34 antibody; low off-target transduction; and poor transduction of human cord blood cells with AAV9+/− anti-hCD34 antibody (FIG. 34). Likewise, when AAV6 was used, natural tropism overrode retargeting antibody on human cord blood cells, but anti-CD34 mAbs robustly retargeted AAV6 HBM mutants in 293/hCD34 cells and moderately retargeted in human cord blood cells (FIG. 35).

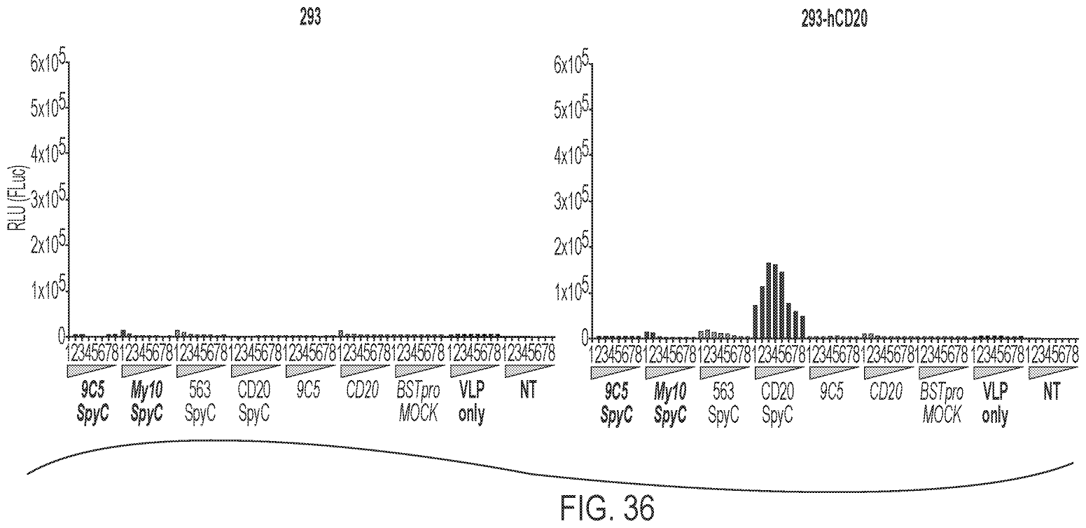

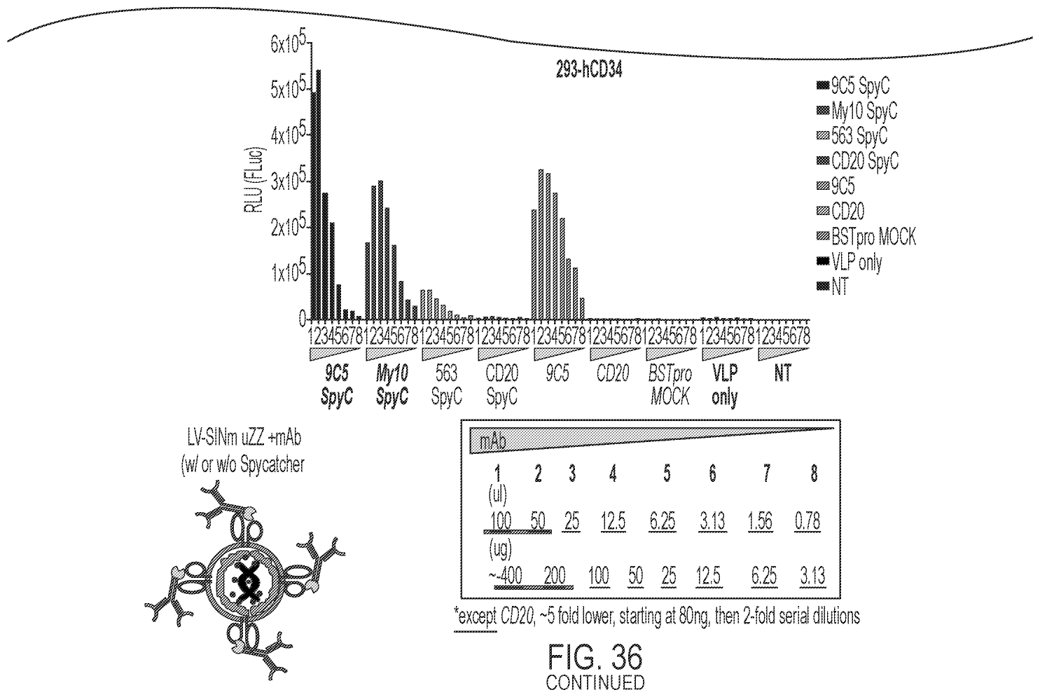

Other virus types besides AAV were used. Lentiviral vectors conjugated to anti-CD34 comparator mAbs were specifically retargeted to CD34-expressing cells, with a mAb-dependent transduction efficacy (FIG. 36). Here, 10,000 cells were seeded per plate (96-well plate), and 2E+08 VG of LV-SINmuZZ EF1a-FLuc was mixed with 2-fold serial diluted CHOt supe in DMEM (starting at 100 ul). After 30 min Incubation at 37° C., LV-CHOt mix was added to cells and incubated at 37° C. Fluc readout was performed 4 days after transduction. Results are shown with 9 conditions: 1) 9C5 (CD34)-SpyC, 2) My1C (CD34)-SpyC, 3) 563 (CD34)-SpyC, 4) CD20-SpyC, 5) 9C5, 6) CD20, 7) BSTpro MOCK, 8) VLP only, and 9) NT. The experiment was repeated for HEK293 cells, 293-hCD20 cells, and 293-hCD34 cells.

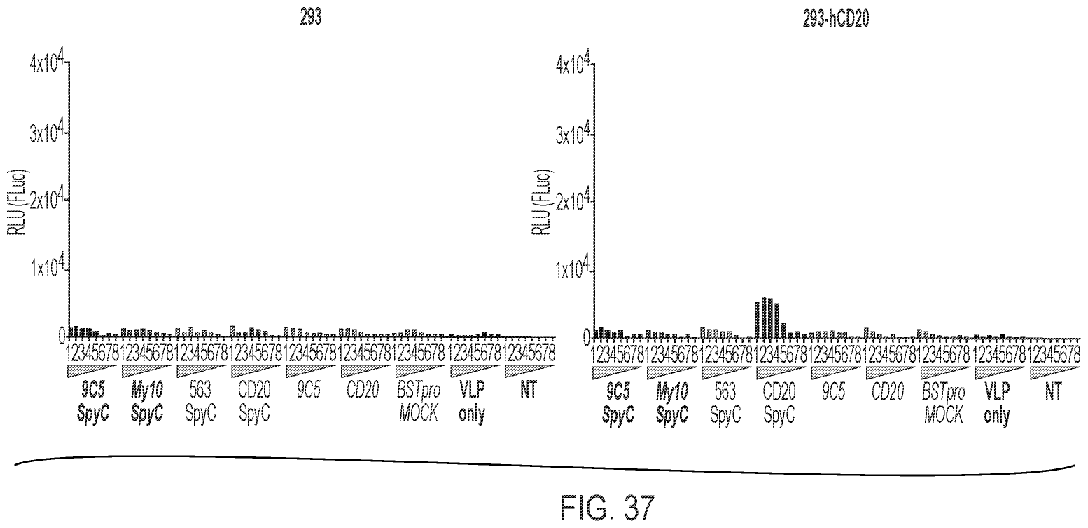

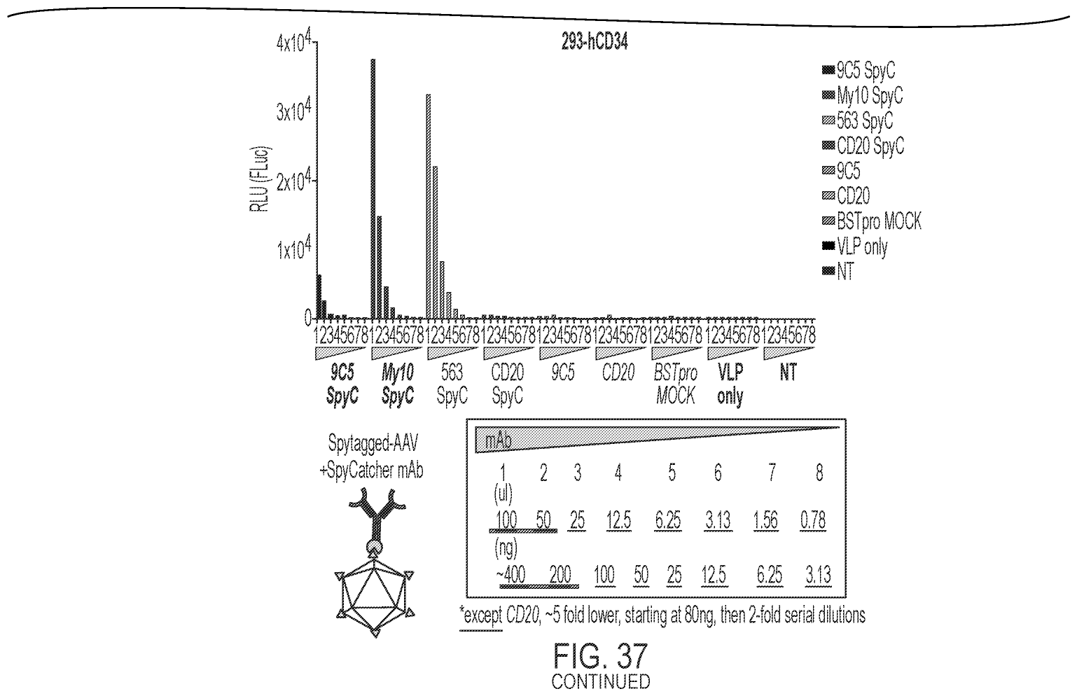

With similar conditions as above, SpyTagged AAV2 conjugated to anti-CD34-SpyCatcher comparator mAbs was specifically retargeted to CD34-expressing cells, with a mAb-dependent transduction efficacy (FIG. 37). Results are shown with 9 conditions: 1) 9C5 (CD34)-SpyC, 2) My1C (CD34)-SpyC, 3) 563 (CD34)-SpyC, 4) CD20-SpyC, 5) 9C5, 6) CD20, 7) BSTpro MOCK, 8) VLP only, and 9) NT. The experiment was repeated for HEK293 cells, 293-hCD20 cells, and 293-hCD34 cells.

In summary, both lentivirus displaying the ZZ domain of protein A on their surface and SpyTagged AAV2 can be conjugated to anti-CD34 comparator antibodies fused to SpyCatcher protein. Anti-CD34-conjugated lentivirus and AAV2 were specifically retargeted to CD34-expressing HEK 293T cells with low background. In those cells transduction efficacy showed variability that was dependent of the anti-CD34 mAb clone and viral vector platform:
LV: 9C5>My10>563
AAV2:My10>5639C5
   Additionally, SFFV promoter drove strong transgene expression in primary human HSCs. Mosaic SpyTagged AAV2, AAV6 and AAV9 conjugated to the My10 anti-CD34 comparator mAb showed retargeting to HEK 293T/hCD34 human primary HSC in a serotype-dependent manner. AAV9 demonstrated a gain-of-function on 293/hCD34 cell line in presence of CD34 antibody with low off-target transduction, but did not transduce primary HSC with our without retargeting antibody. Retargeted AAV2 and AAV6 with CD34 antibody demonstrated the same transduction level in 293 hCD34+ cell line than the WT serotypes. Retargeted AAV2 and AAV6 with CD34 antibody showed increased transduction compared to the de-targeted serotype in both HEK 293T hCD34+ cell line and primary HSC, but in all cases the retargeted virus did not transduce better than the WT serotypes. It is important to find new CD34 high affinity binders, driving both a specific and efficient transduction to human HSC.

### Example 8: CD34 Immunization to Retarget Viral Vectors to HSPCs

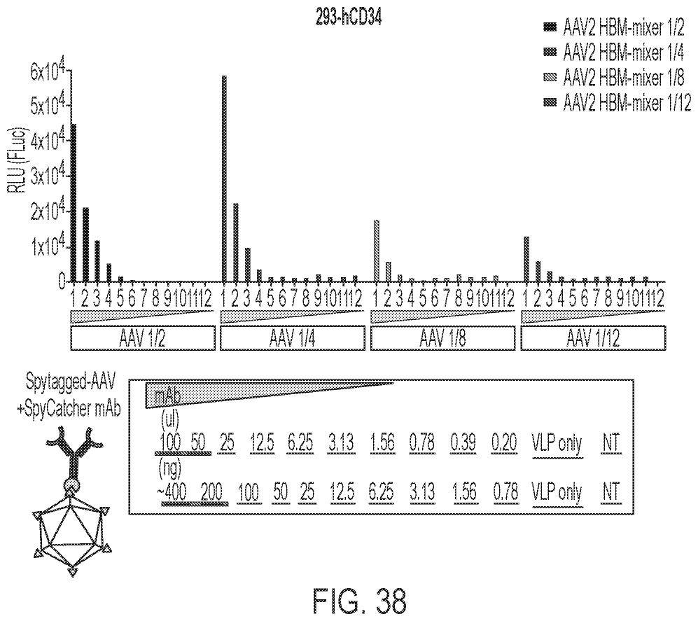

Optimization of mosaicism ratio revealed that AAV2 HBM-mixer 14 led to higher transduction in HEK293T/hCD34 cell line. Similar to Example 7, screening for platform gene delivery against CD34 was started by seeding 10,000 cells per well in 96-well black wall clear bottom plate in with three cell types (293, 293-hCD20, 293-hCD34). Next, mix 5E+09 VG of AAV2 ⅛ SpyTag/HBM SFFV-FLuc was 2-fold serially diluted CHOt supe in DMEM (starting at 100 μl) and incubated at 37 C for 1.5 hr. Then, AAV2-CHOt mix was added to cells and incubated at 37° C. In three days, the cells were collected for flow cytometry analysis. The results are shown in FIG. 38 for different types of HBM mixers. AAV2 HBM-mixer 14 had the highest transduction in the 293-CD34 cells.

### Example 9: Gene Transfer in Mouse HSPCs Using Anti-CD117 and Anti-SCA-1 mAbs

Human and mouse long-term HSPCs do not express the same makers, as CD34 is only a marker of LT-HSPCs in humans. To continue testing in mice, lentiviral vectors were retargeted with anti-CD117 (the c-kit proto-oncogene product) and anti-Sca-1 antibodies.

Lentiviral Vectors Retargeted with Anti-CD117 and Anti-Sca-1 Transduce Efficiently Cell Lines Expressing the Respective Target Antigen Receptor In Vitro

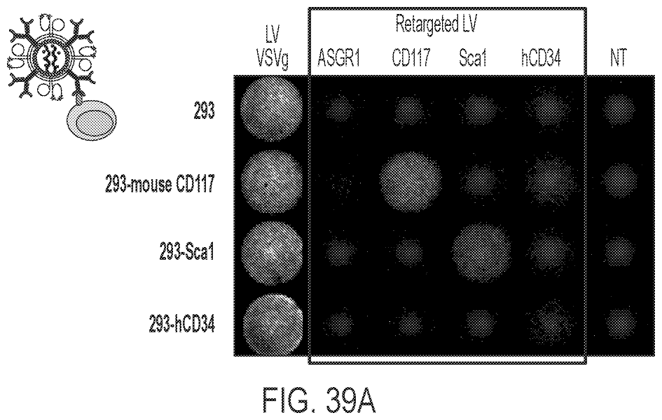

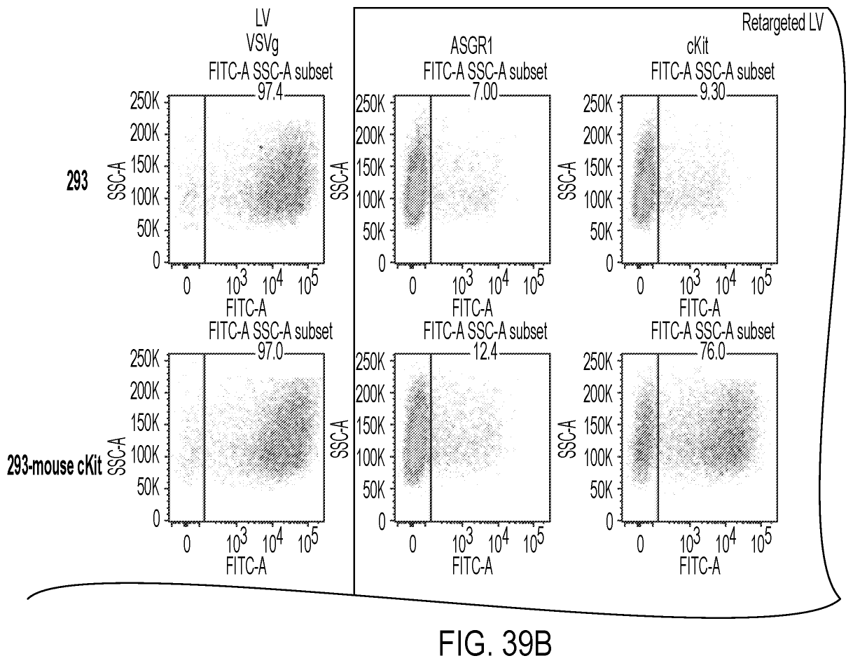

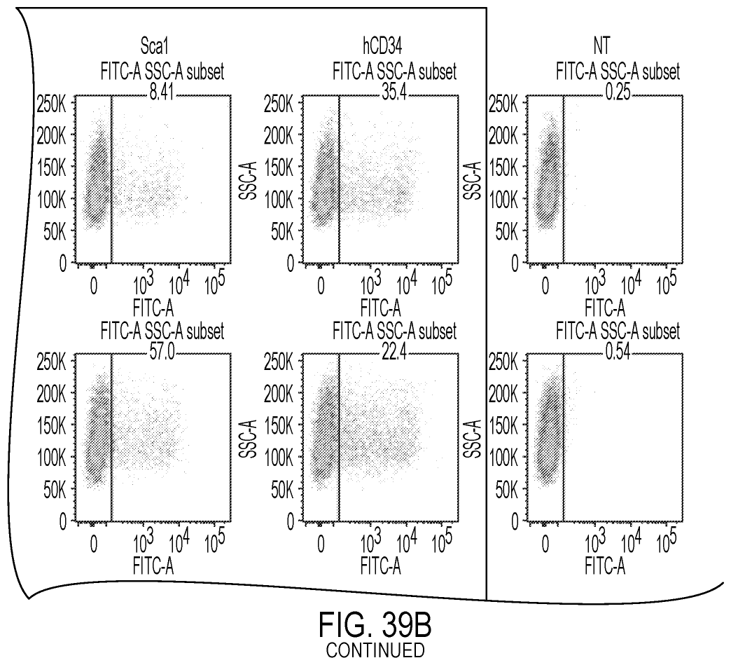

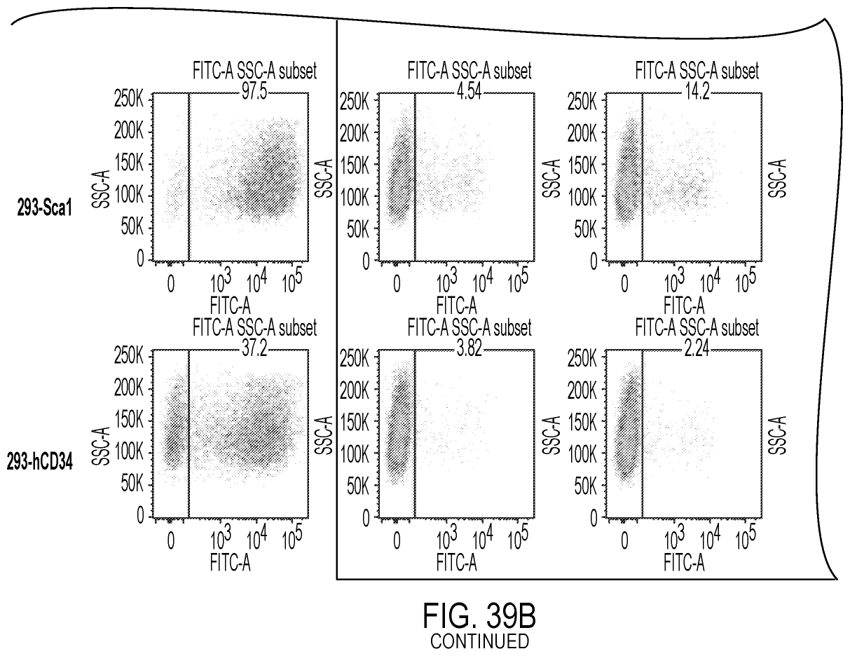

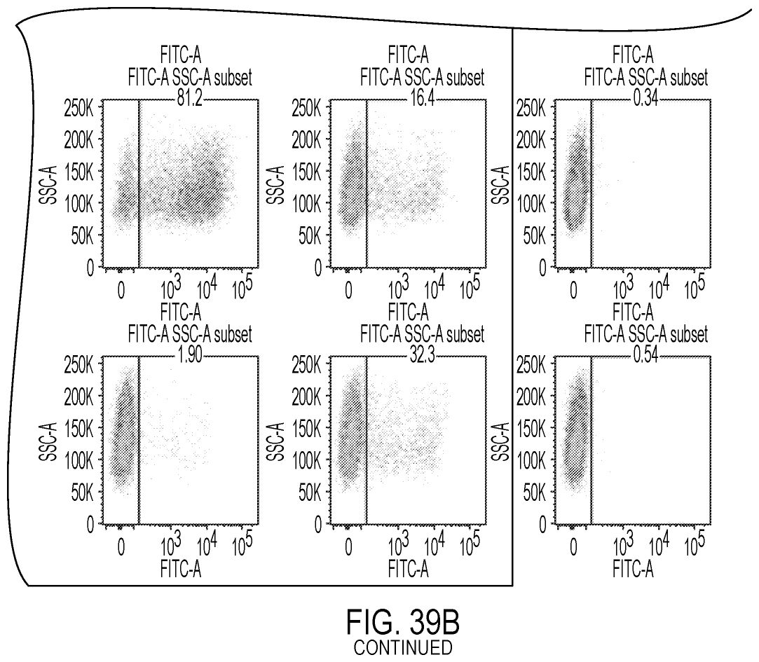

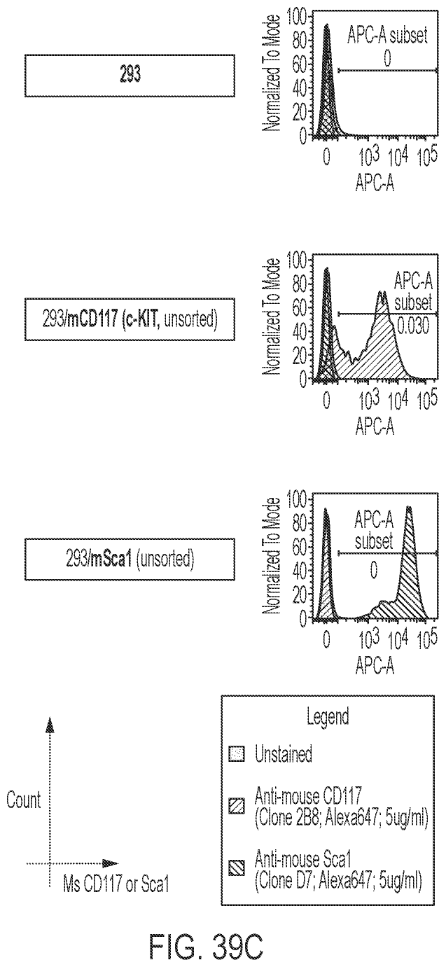

1E+04 cells were seeded in a 96-well black well clear bottom plate in 100 μl DCM with 4 g/ml polybrene. The cells were transduced with 2E+04 VG per cell in 100 μl DCM aliquots with 4 μg/ml polybrene. After 2 days, fluorescent imaging and analysis of GFP was performed by flow cytometry. Results showed that the vectors successfully retargeted the cell lines expressing their respective target antigens (FIGS. 39A-39C).

Surface Expression of CD117 (c-KIT) and Sca-1 are Detected on Mouse HSPCs (Two Days Post Expansion)

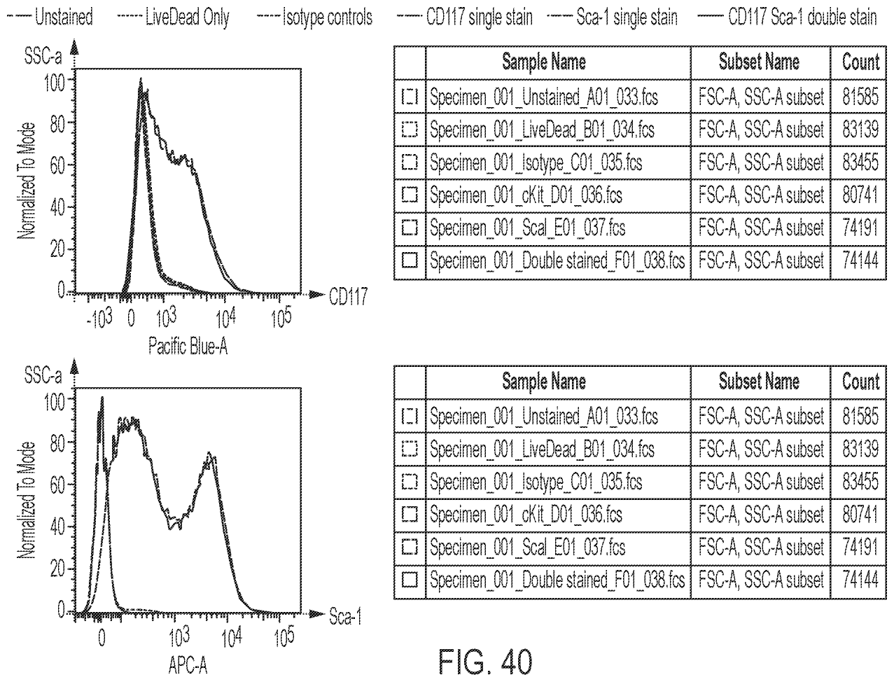

At day zero, mouse HSPCs were isolated from collected bone marrow. Cells were cultured in a progenitor medium of SFEM+SCF (100 ng/mL), TPO (100 ng/mL), Flt3L (100 ng/mL), IL-6 (50 ng/mL), and IL-3 (30 ng/mL). Two days post isolation, the cells were stared for CD117 and Sca-1, then transduced with pseudoparticles with a SFFV-GFP reporter. Two days after transduction (day 4 post isolation) the readout of GFP expression is performed via FACS. The results showed surface expression of CD117 and Sca-1 on the murine HSPCs (FIG. 40).

Mouse HSPCs are Transduced with Lentiviral Vector Pseudotyped with Anti-Mouse CD117 mAb and SINmu

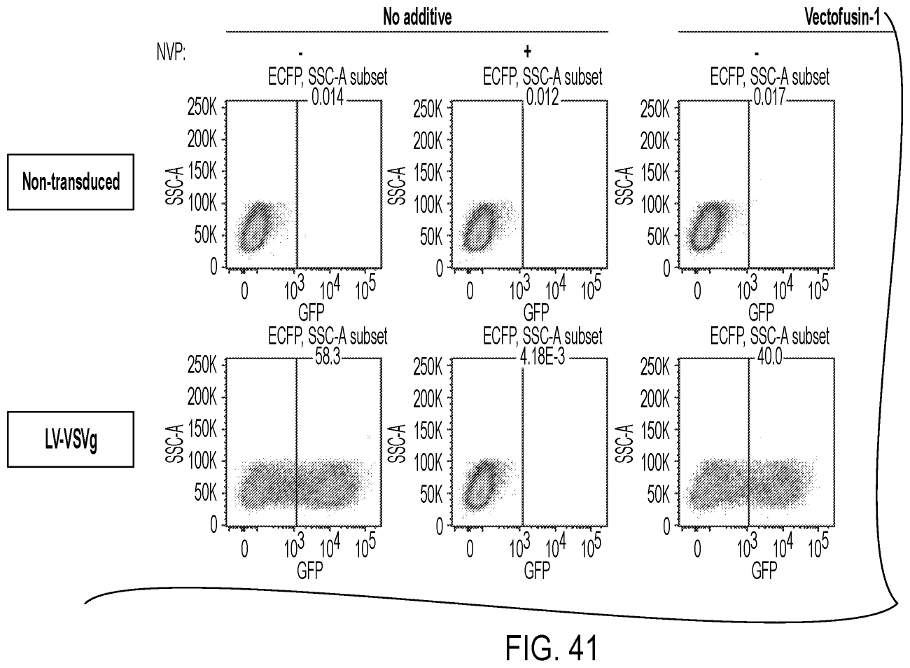

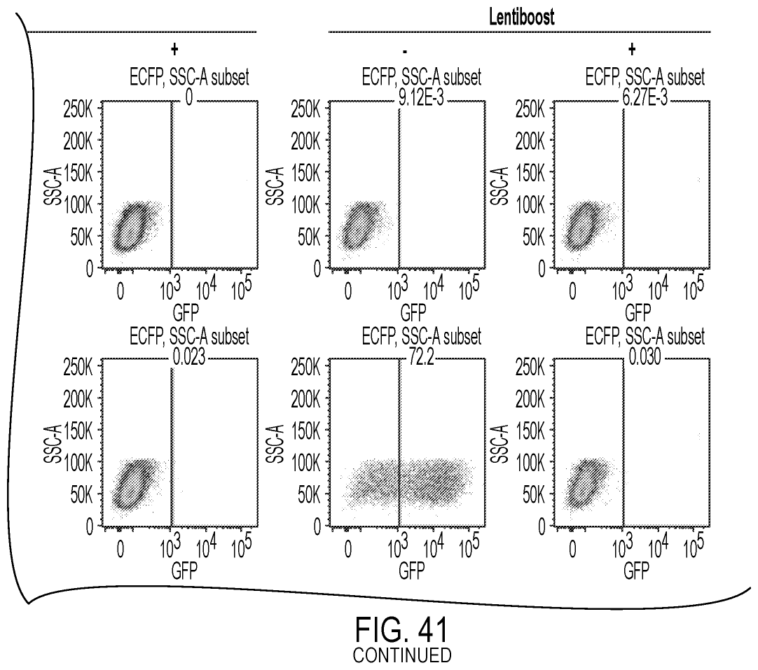

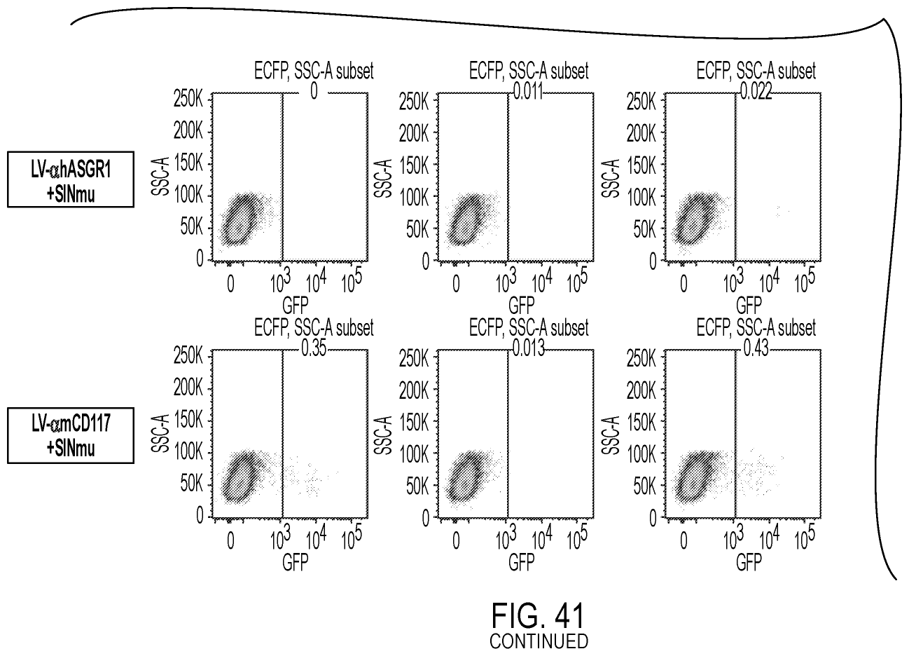

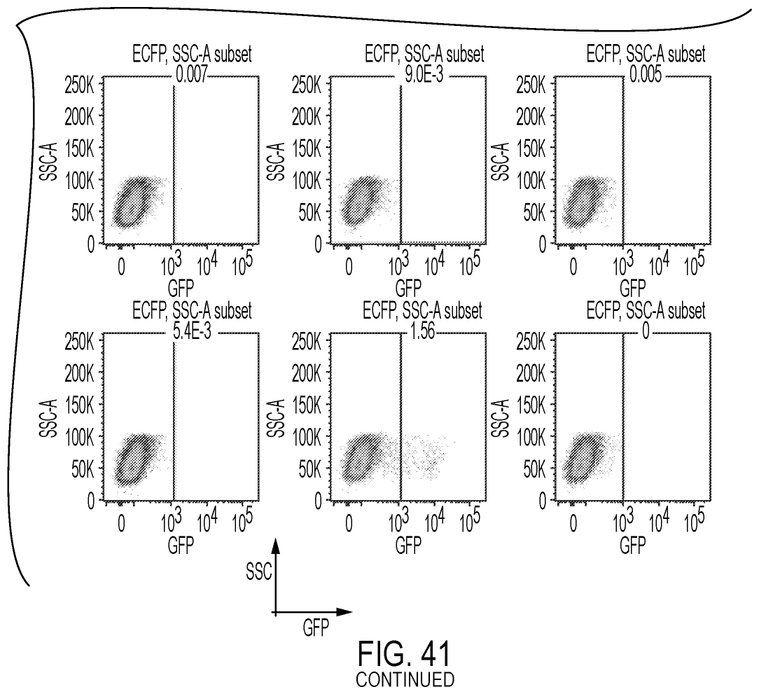

LV pseudotyped with α-CD117+SINmu and α-Sca1+SINmu were functional as transduction was observed in cell lines (FIG. 41). LV pseudotyped with α-CD 117+SINmu can transduce expanded mouse primary HSPCs with very low efficiency. This was an entry issue as LV pseudotyped with VSVg was able to transduce expanded mouse HSPCs efficiently.

### Example 10: Retarget SpyTagged AAV2 Conjugated with CD117/Sca1 Plus SpyCatcher Antibody in Engineered Cell Lines

As described previously, AAV modified with SpyTags can be conjugated to a corresponding antibody modified with SpyCatcher. Here, AAV2 was conjugated with either CD117 or Sca-1 mAbs via the SpyCatcher system.

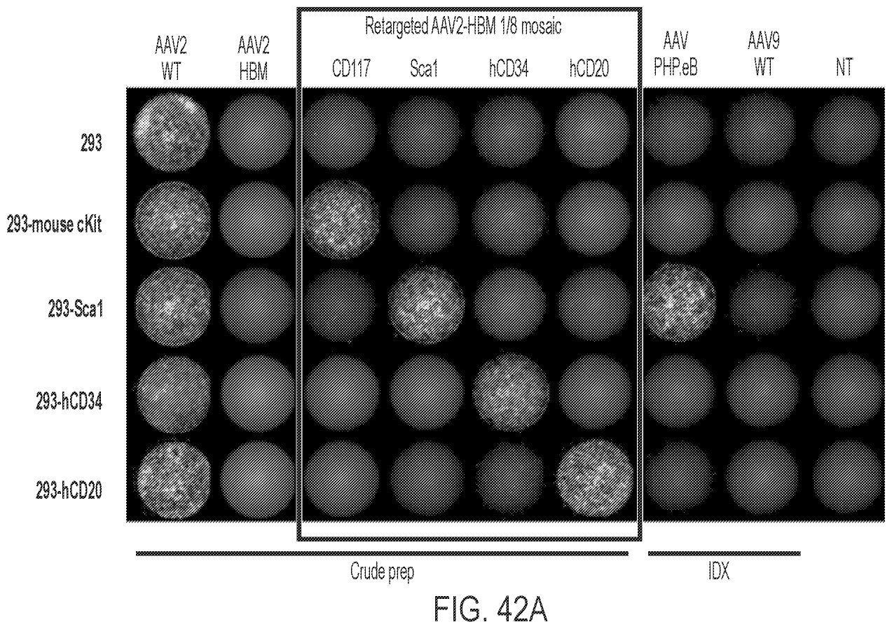

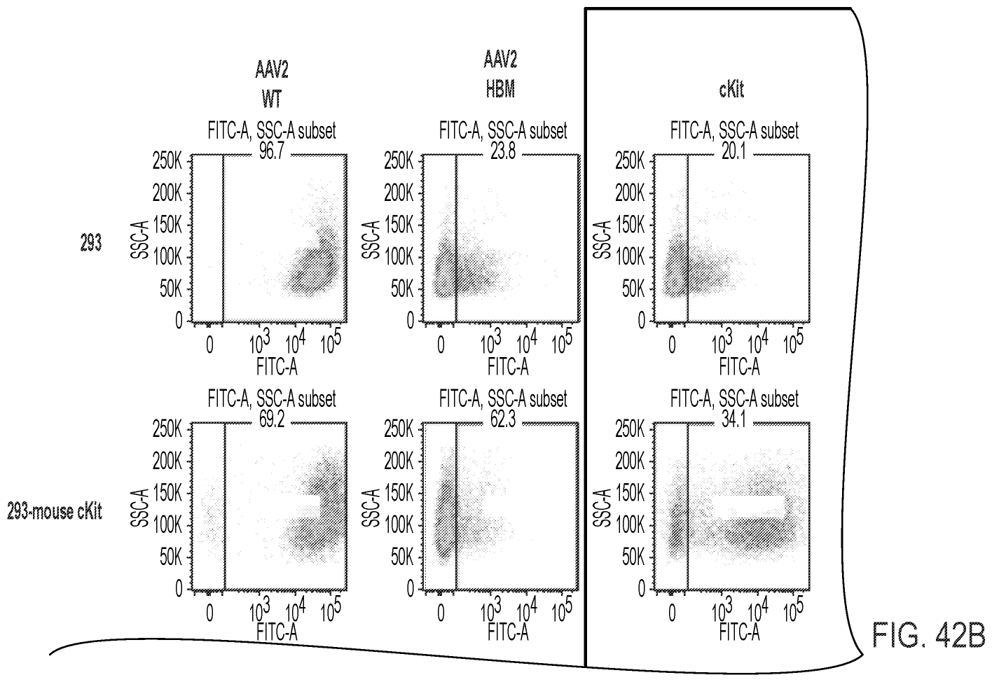

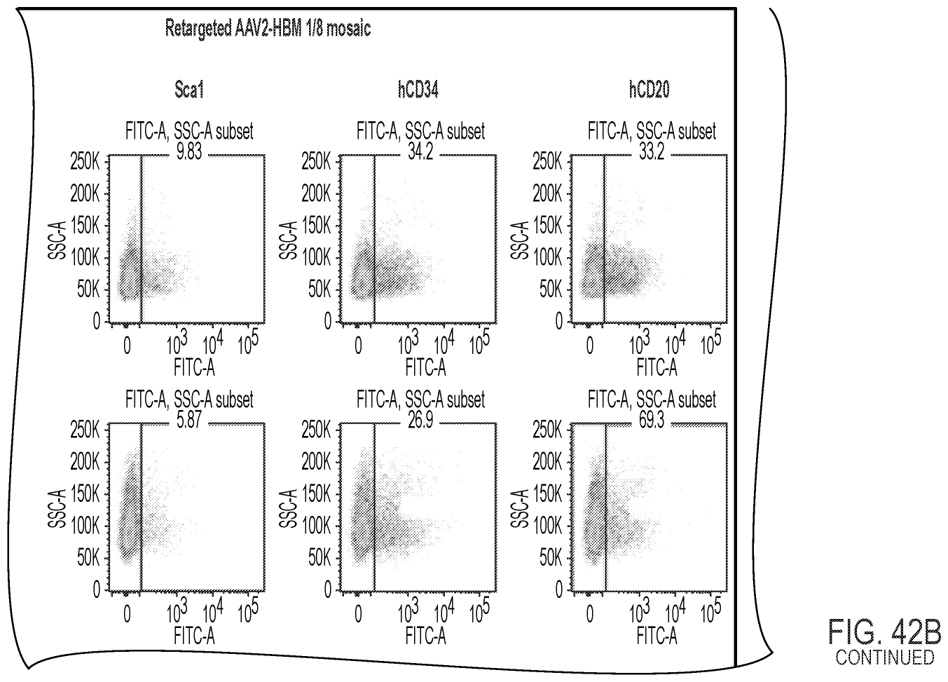

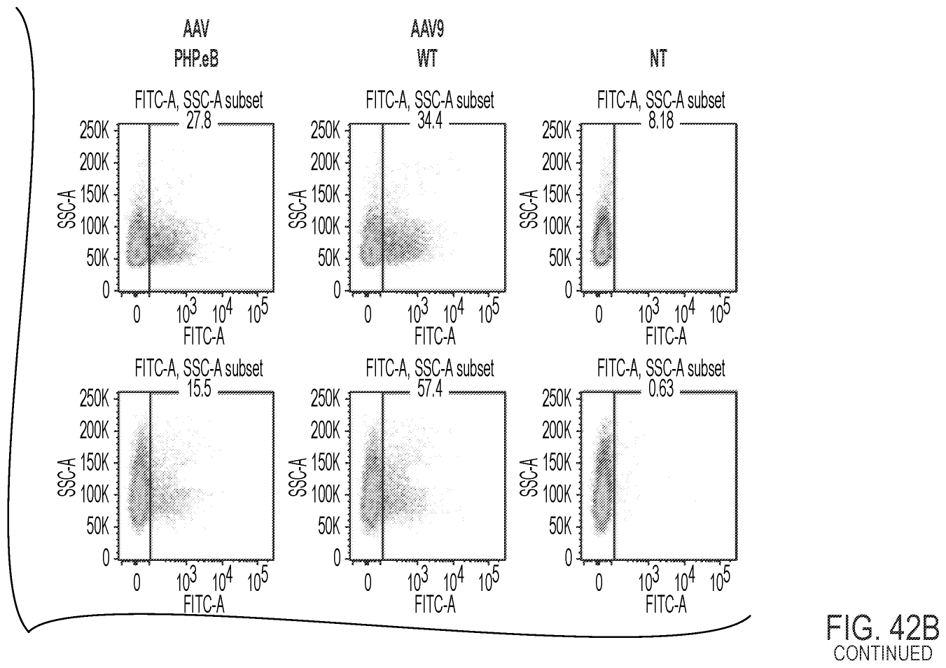

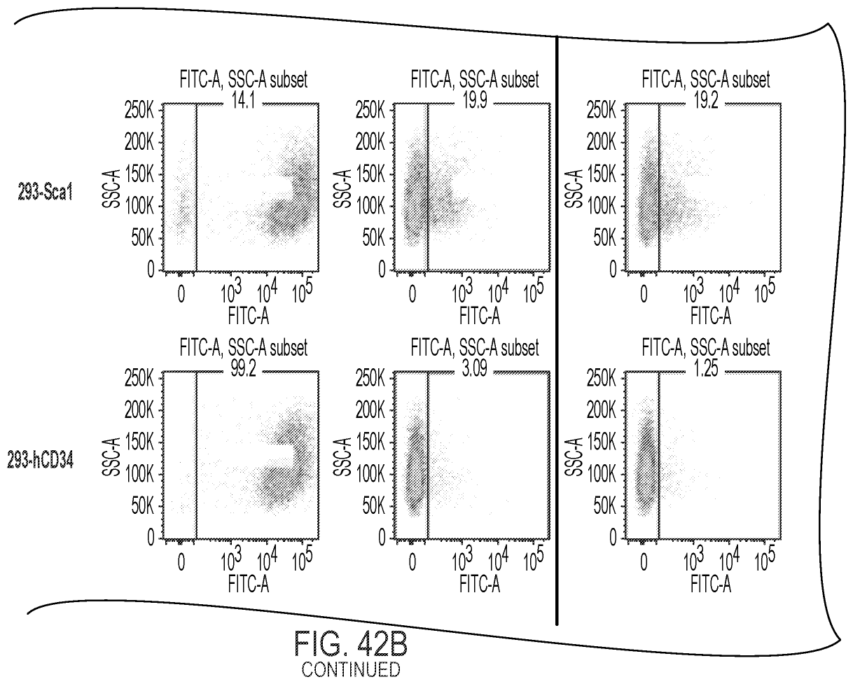

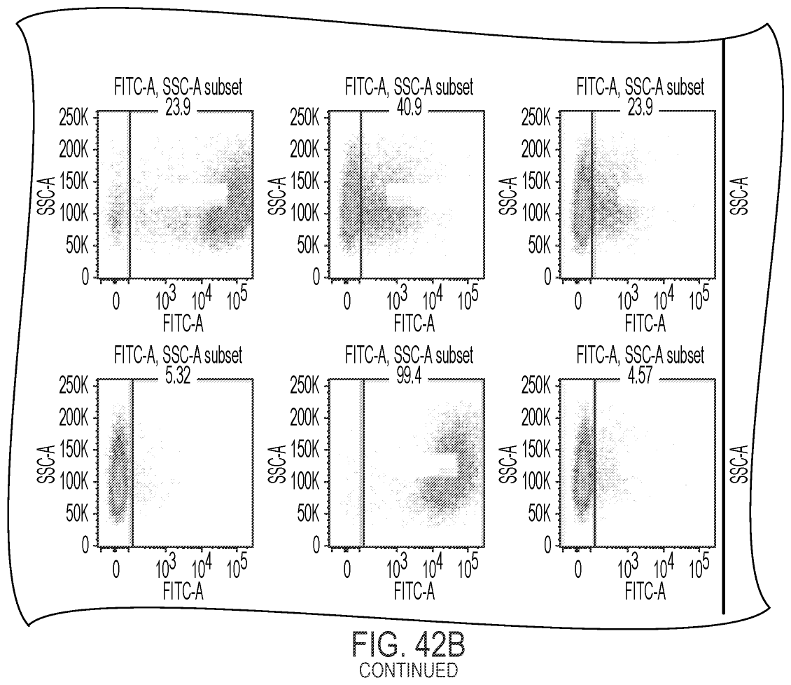

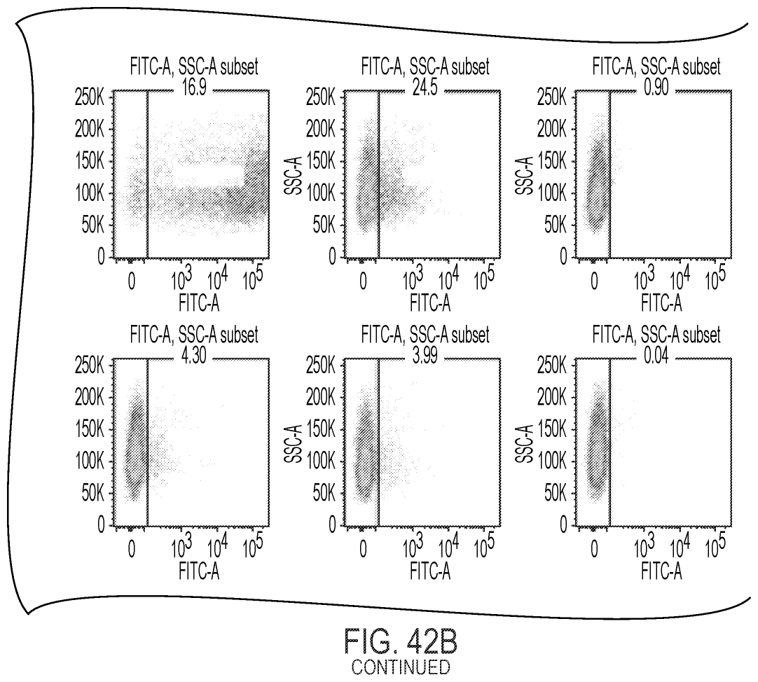

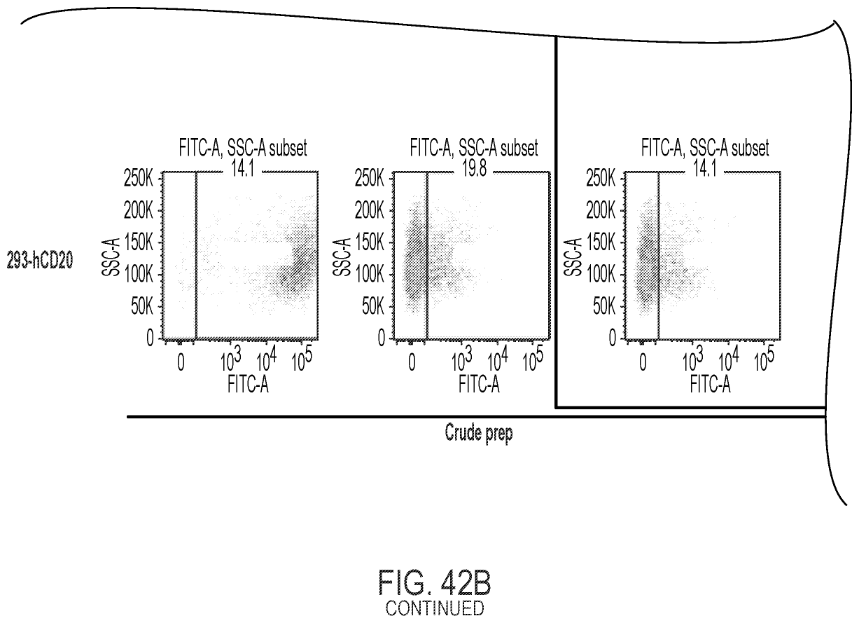

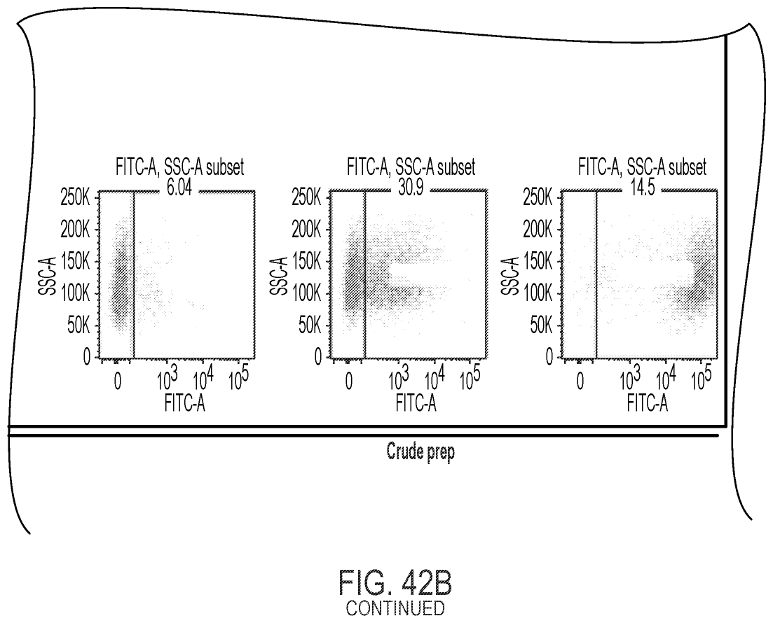

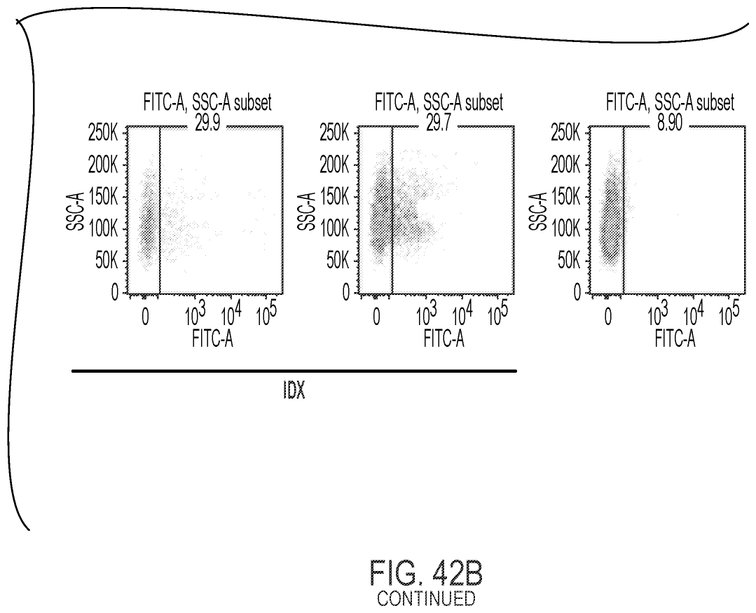

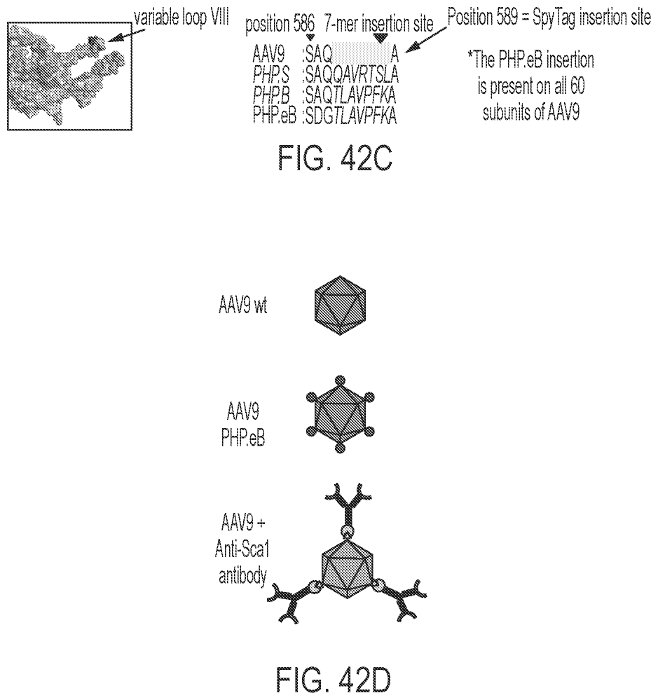

Sca-1 (LY6A) is known to drive AAB-PHP.B transport across the blood-brain barrier (BBB). The Ly6a gene encoding Sca-1 is associated with high AAB-PHP.B transduction across the BBB. AAV-PHP.B binds to LY6A (SCA-1) protein. PHP.eB is a peptide insertion library variant of AAV9 that directly binds Sca1 and crosses the BBB in mice (FIG. 42C). As the demonstration that the interaction of PHP.eB with Sca-1 can be mimicked with an anti-Sca-1 antibody (FIG. 42D), SpyTagged AAV2 were efficiently retargeted to cell lines expressing CD117 or Sca-1 in vitro. Retargeted AAV2-HBM ⅛ mosaic with either CD117, Sca-1, hCD34, of hCD20 successfully retargeted to HEK293 cell lines expressing those markers (FIGS. 42A and 42B).

Mosaic AAV2 HBM-SpyCatcher conjugated to SpyTagged anti-CD117 and anti-Sca-1 transduced very efficiently HEK293T cells over-expressing the respective target surface antigen. Anti-Sca-1 AAV2 transduced with the same efficacy Sca-1 expressing cells than AAV-PH.eB. This provides a demonstration that an antibody conjugation can substitute for a peptide insertion on an engineered capsid.

### Example 11: In Vivo Targeting of AAV and Lentivirus to Human Stem Cells for BCR Editing and Antibody Production (Prophetic)

While an ex vivo cell engineered approach to vaccination may be feasible for an individual, it is unrealistic for treating a population. To meet this need, cell editing methods that rely solely on injectables must be developed. The present inventors propose to translate the ex vivo stem cell targeting and editing technology to in vivo application by delivering the viral vectors in vivo to mediate BCR insertion. The primary challenge to success of this approach is the optimization of in vivo stem cell transduction efficiency with the co-delivered viral vectors. Inefficiencies in in vivo stem cell editing may be overcome through in vivo highly selective expansion of the engineered stem cells using methods developed herein.

Effective Targeting of Human Stem Cells In Vivo with AAV or Lentivirus

Human stem cells can be successfully retargeted ex vivo with several types of AAVs and lentivirus conjugated to CD34 antibodies (as shown in Example 7). Next, the specificity and efficiency of CD34-retargeted viruses is evaluated in a mixed culture setting (human PBMCs) ex vivo. Additionally, other CD34 antibodies with different binding properties and affinities are tested. Then, the targeting arms are expanded to other potential antibodies to determine which virus-antibody combination is the most successful. Finally, viral retargeting in in vivo humanized mice is performed, first in CD34, and then with other antibodies. The goal is to identify the best viral-targeting antibody combination to successfully retarget human stem cells in vivo with little to no off-target effects.

Effective Cas9 Expression from AAV or Lentivirus

Expressing full length Cas9 from an AAV genome is challenging because most endogenous promoters are too large, and even viral promoter are typically >400 bps. However, for the proposed system to work, full Cas9 will need to be expressed. Much work has already been performed on shortening existing strong promoters (SFFV) and strengthening existing short and cell-type specific promoters (as shown in Example 4). Additional work is performed to shorten existing strong promoters, strengthen existing short and cell-type specific promoters, and identify novel cell-type specific promoters to allow for robust, full length Cas9 in vivo expression post AAV infection of stem cells. Lentivirus options are also pursued.

### Effective, Dual-Viral Vector Mediated BCR Editing of Stem Cells In Vivo in Mice and Non-Human Primates

Once the most effective and on-target viral vector/antibody combination is identified, and once effective Cas9 expression is shown, the final step is to perform and optimize in vivo BCR editing of stem cells. The ratio between Cas9-expressing virus (whether AAV or lentivirus) and the insertion template-expressing virus (whether AAV or lentivirus) is varied to optimize in vivo BCR insertion efficiencies, first in mice, then in non-human primates. Frequencies of BCR expression is assessed by flow cytometry, both with and without stem cell expansion methods. Finally, antibody responses over time and in response to antigen challenges are characterized.

### Example 12. Directed Evolution of B Cell Targeting Viruses

1. In vitro iterative selection of AAV mutants that selectively infect primary human B cells ex vivo
a. Libraries of AAV mutants are generated either by random peptide insertion into surface-exposed loops or by a shuffling/error prone PCR approach.
b. Candidate mutants with desired properties are selected by iteratively producing virus, infecting cells, isolating nuclear viral genomes from cells of interest, and re-cloning the isolated viral genomes for the next round of virus production and selection.
c. Libraries are first selected on purified primary human B cells, then selected for specific transduction of B cells within a mixed population of human PBMCs.
2. In vivo iterative selection of AAV mutants that selectively infect B cells in NHPs
a. Libraries generated as described above are injected systemically into NHPs.
b. Multiple organs are sampled; candidate mutant capsid sequences are isolated from B cells from peripheral blood, spleen, and bone marrow.
c. Each round of selection is performed in 2 individual animals; 3 rounds of selection require 6 NHPs.
3. Combinatorial iterative selection of AAV mutants that efficiently infect B cells when combined with antibody-mediated retargeting
a. Libraries are produced on the backbone of capsids that contain modifications required for antibody-mediated retargeting (i.e., SpyTag or Myc epitope tag) and screened as described above to identify mutants that enhance transduction efficiency in the context of antibody retargeting.

### Example 13. Exploring B1 B Cells as Candidates for Life-Long Expression of Engineered Antibodies

B1 B cells (B220lo CD5+ CD23− CD43+ IgMhi IgDlo) are long-lived, self-renewing innate-like B cells that predominantly inhabit the peritoneal and pleural cavities and are generated during development from the yolk sac and fetal liver. The principal function unique to B1 B cells is spontaneous, constitutive secretion of “natural” IgM serum antibody against non-protein antigens. These cells function to rapidly clean up apoptotic cell debris via immune complexes and opsonization. B1 cells' BCRs are restricted, with preferential usage of JH proximal VH gene segments during V(D)J recombination, with fewer N-region insertions and a lower rate of somatic mutations. Lack of TdT during B cell develop early in life when these cells are mostly generated. See, e.g., Montecino-Rodriguez and Dorshkind, Immunity, 2012, 36(1):13-21.

B2 B cells are positively selected using pre-BCR with SLC (Surrogate Light Chain), negatively selected with mature BCR. Pre-BCR signaling is required to signal and initiate light chain rearrangement.

B1 B cells tend to have heavy chains 1) that bind poorly to SLC, and 2) associate with limited number of LC. Reduced IL-7R/STAT5 levels in fetal liver promote immunoglobulin kappa gene recombination at the early pro-B cell stage. Differentiating B cells directly generate a mature B cell receptor (BCR)-bypass requirement for pre-BCR pairing with SLC. This ‘alternative’ development positively selects for B cells with self-reactive, skewed specificity receptors. B1 B cells rapidly produce antibodies in response to TLR agonists in the “absence” of BCR stimulation. See, e.g., Genestier et al., J Immunol, 2007, 178:7779-7786.

TLR stimulation appears to cause migration of B1 cells into sites of inflammation and produce “natural” IgM regardless of antigen specificity. Upon flu challenge, B1 cells are primary early IgM producers in airways, whereas B2 are primary producers in serum. See, e.g., Yang et al., PNAS, 2012 109(14):5388-5393.

The present inventors hypothesized that B1 B cells may be good candidates for life-long expression of engineered antibodies due to their long life with self renewing capacity, potential for constitutive steady state antibody production, and rapid production of high levels of IgM upon TLR stimulation (within 2 days). Also, B1 B cells are primary early IgM producers at sites of infection.

While B2 antibody engineered cells should produce antibodies in response to antigen and get better with repeat exposures (i.e., adaptive immunity), B1 antibody engineered cells should express constitutive low level of antibodies in the steady state and temporarily increase levels upon TLR agonist treatment.

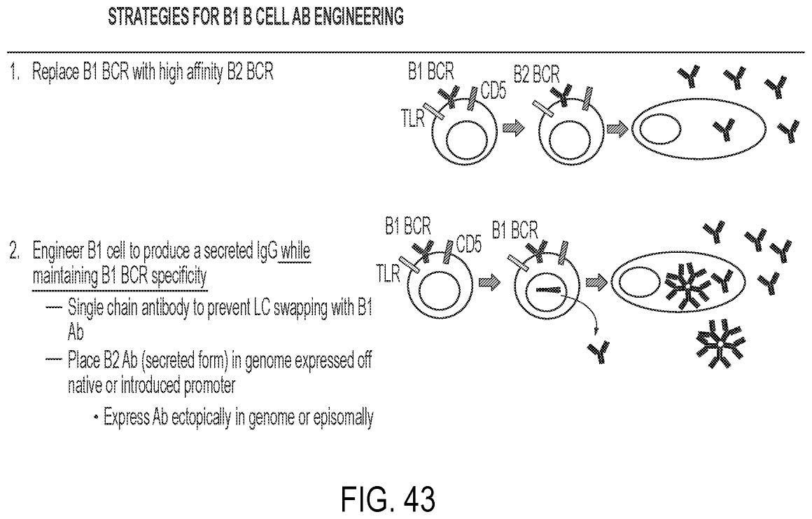

Proposed strategies for B1 B cell antibody engineering are shown in FIG. 43. Strategy 1: replace B1 BCR with high affinity B2 BCR. Strategy 2: engineer B1 cell to produce a secreted IgG while maintaining B1 BCR specificity (single chain antibody to prevent LC swapping with B1 antibody, place B2 antibody (secreted form) in genome expressed off native or introduced promoter, express antibodies ectopically in genome or episomally).

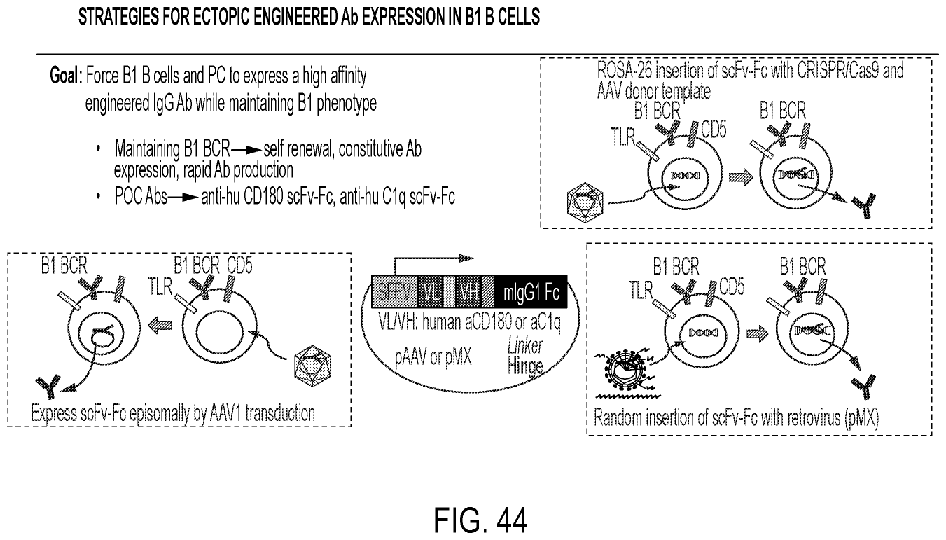

FIG. 44 shows proposed strategies for ectopic engineered antibody expression in B1 B cells. The goal is to force B1 B cells and PC to express a high affinity engineered IgG antibodies, while maintaining B1 phenotype. Transgene expression via random integration into the genome is subjected to position effects and silencing. In addition, random gene insertion might interrupt or activate the neighboring genes. Genomic safe harbor sites are transcriptionally active, therefore allowing robust and stable gene expression. Furthermore, a transgene insertion at genomic safe harbors does not have adverse effect on the host cell genome. CRISPR technology can be utilized to do targeted gene insertion at these genomic loci. For mouse cells, ROSA26 is proved to be a genomic safe harbor locus. ROSA26 (also known as ROSAβgeo26 locus) in the mouse genome is first found in chromosome 6. Inserted transgene expressed at high levels uniformly in nearly all tissues. This locus expresses one coding transcript and two noncoding transcripts, and only the non-coding transcripts are disrupted by the insertion.

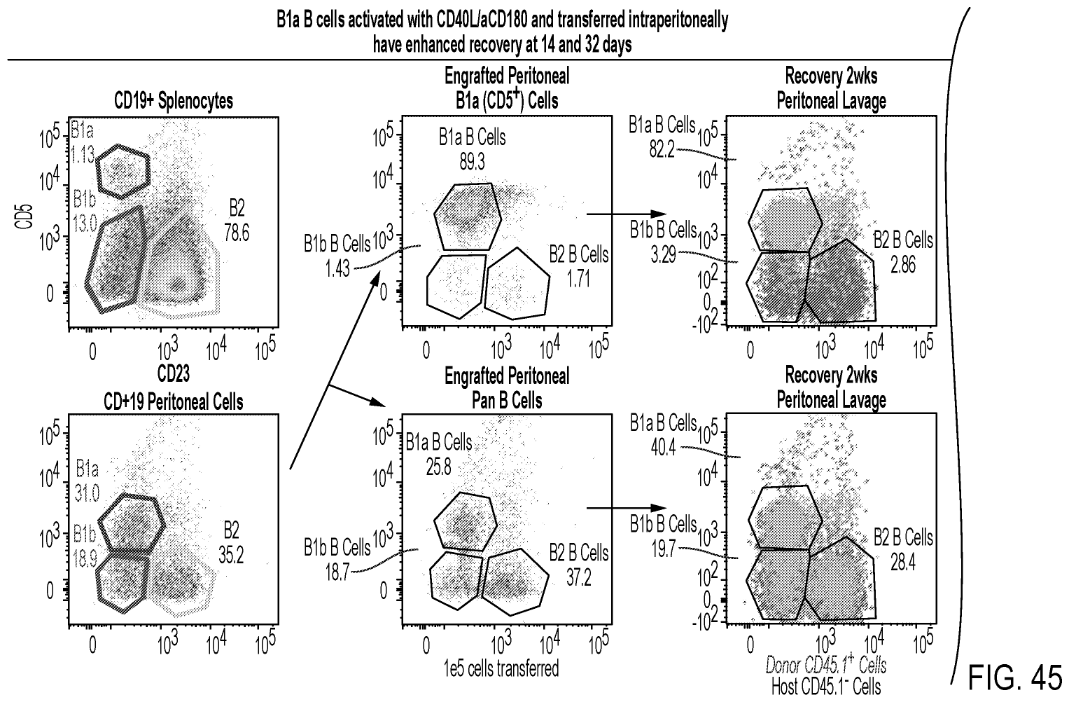

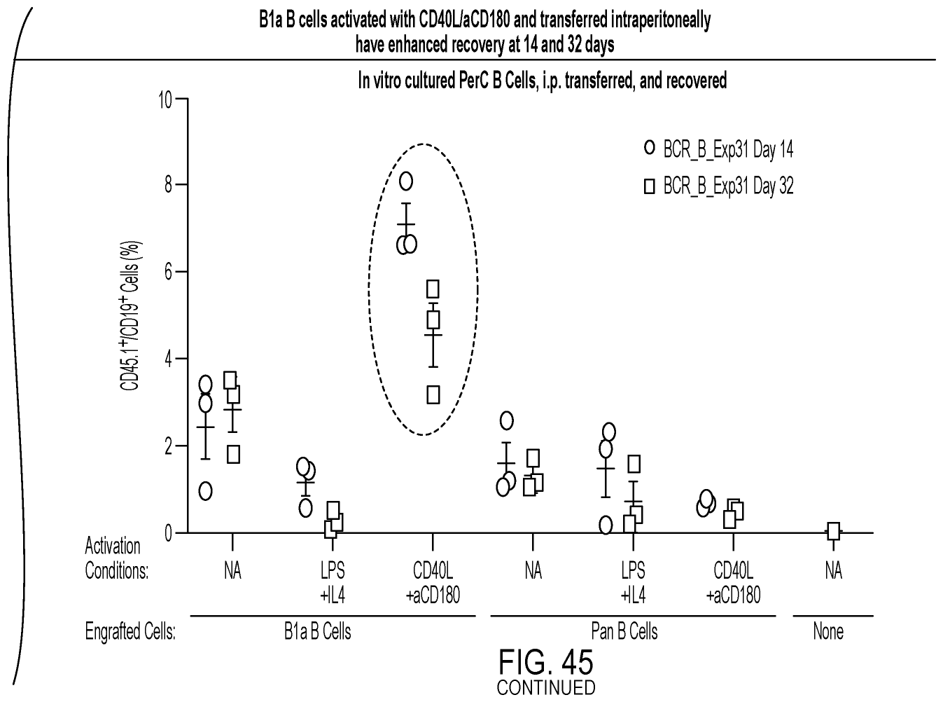

FIG. 45 shows that B1a B cells activated with CD40L/aCD180 and transferred intraperitoneally have enhanced recovery at 14 and 32 days.

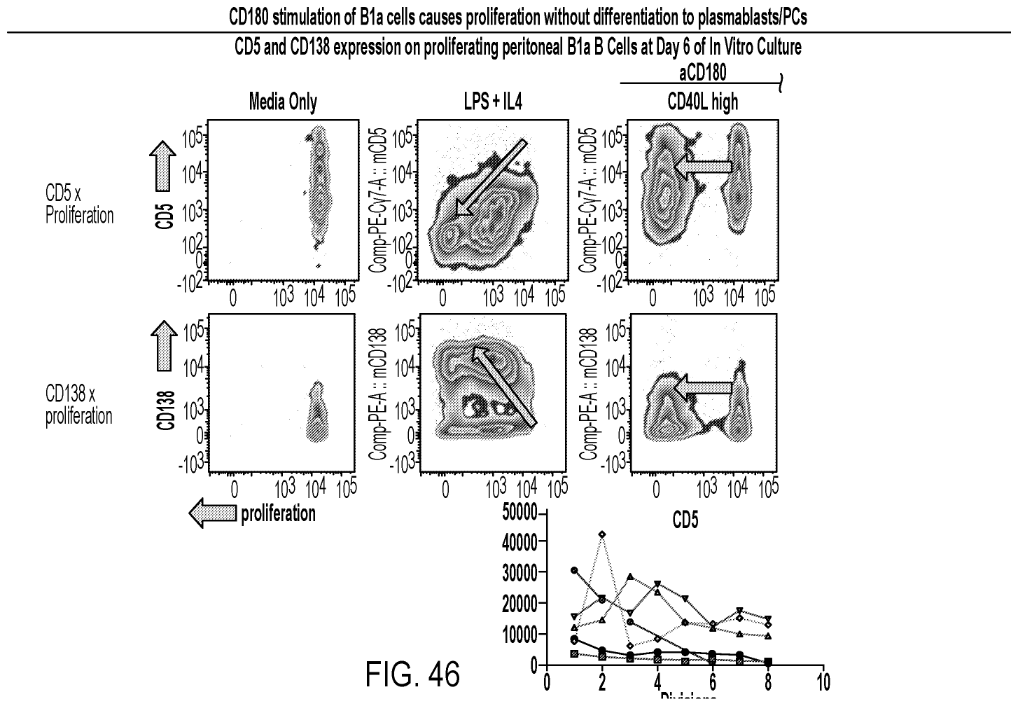

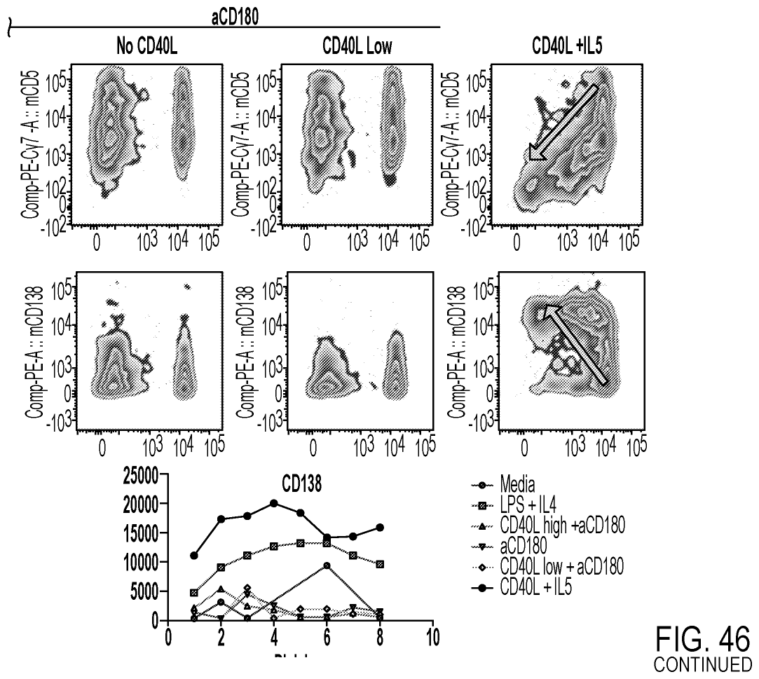

FIG. 46 shows that CD180 stimulation of B1a cells causes proliferation without differentiation to plasmablasts/PCs.

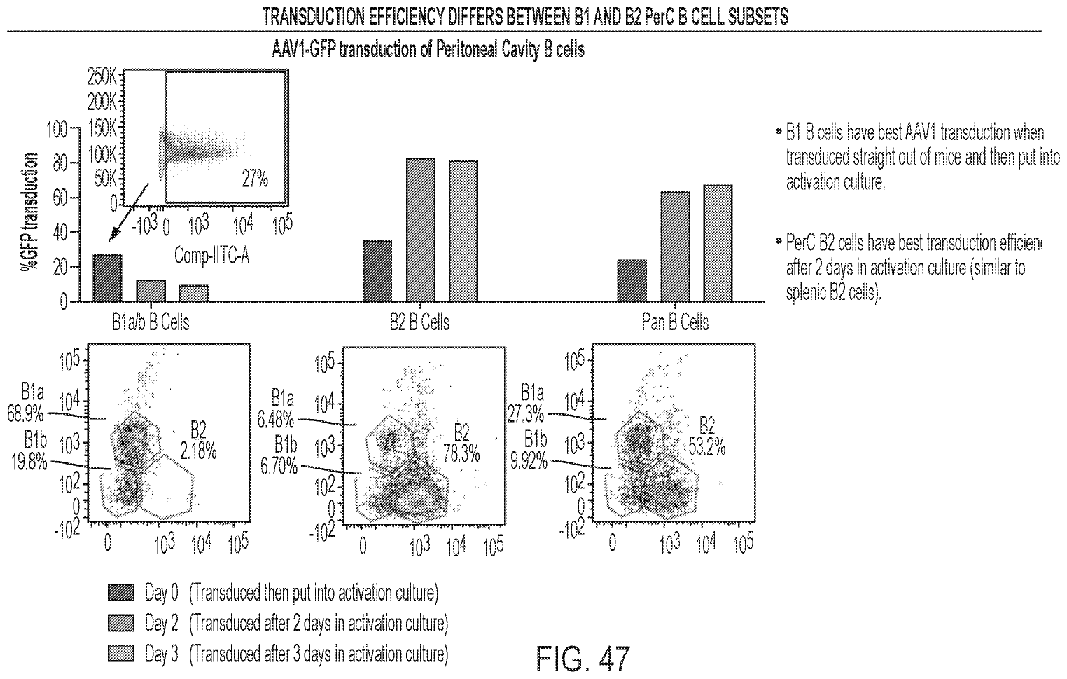

FIG. 47 demonstrates that transduction efficiency differs between B1 and B2 peritoneal cavity (PerC) B cell subsets. B1 B cells have best AAV1 transduction when transduced straight out of mice and then put into activation culture. PerC B2 cells have best transduction efficiency after 2 days in activation culture (similar to splenic B2 cells). This experiment demonstrates the ability to transduce and engineer both B1 and B2 peritoneal cavity B cells.

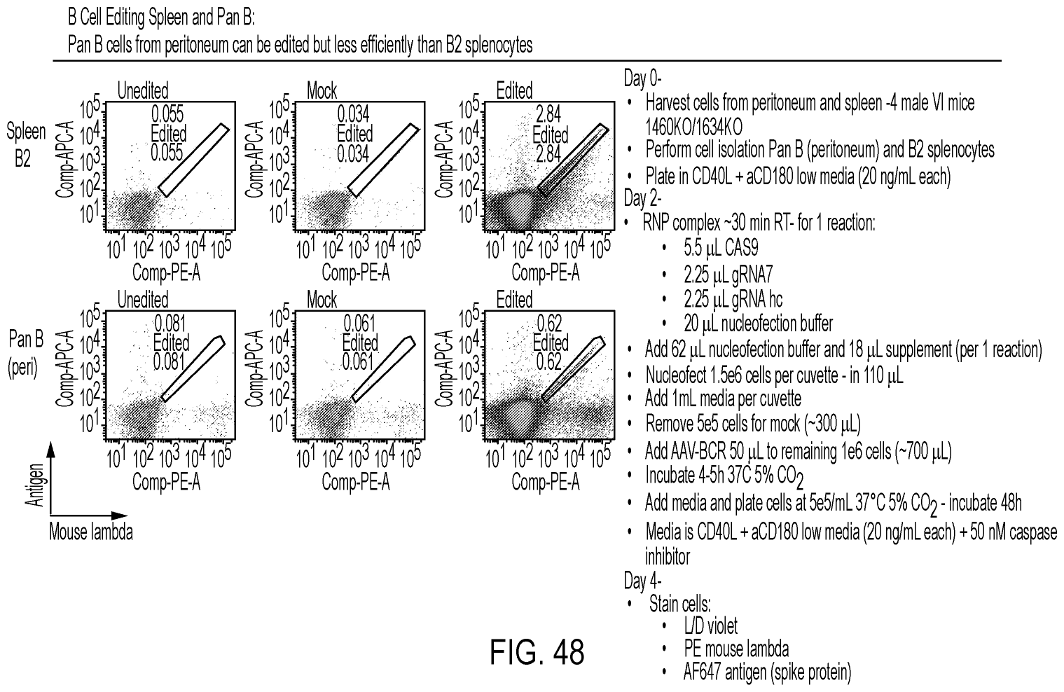

FIG. 48 shows that Pan B cells from peritoneum can be edited but less efficiently than B2 splenocytes. The protocol of the study was as follows:
Day 0—
Harvest cells from peritoneum and spleen—4 male VI mice 1460KO/1634KO
Perform cell isolation Pan B (peritoneum) and B2 splenocytes
Plate in CD40L+aCD180 low media (20 ng/mL each)
Day 2—
RNP complex ˜30 min RT—for 1 reaction:
5.5 μL CAS9
2.25 μL gRNA7
2.25 μL gRNA he
20 μL nucleofection buffer
Add 62 μL nucleofection buffer and 18 μL supplement (per 1 reaction)
Nucleofect 1.5e6 cells per cuvette—in 110 μL
Add 1 mL media per cuvette
Remove 5e5 cells for mock (˜300 μL)
Add AAV-BCR 50 μL to remaining 1e6 cells (˜700 μL)
Incubate 4-5 h 37 C 5% CO₂
Add media and plate cells at 5e5/mL 37° C. 5% CO₂—incubate 48 h
Media is CD40L+aCD180 low media (20 ng/mL each)+50 nM caspase inhibitor
Day 4—
Stain cells:
L/D violet
PE mouse lambda
AF647 antigen (spike protein)

Taken together, this Example demonstrates the following:
(1) successful isolation, transfer and recovery of multiple subsets of peritoneal cavity B cells in donor/recipient experiments;
(2) in vitro CD40L/aCD180 stimulation of B1a cells enhanced engraftment in recipients;
(3) B1 B cells have better AAV transduction when transduced just prior to activation culture;
(4) B2 B cells from peritoneal cavity have better transduction when transduced after activation culture (similar to splenic B2 cells).

### Example 14: Identification of In Vitro Culture Conditions that Favor Re-Engraftment of Ex Vivo Cultured Mouse B Cells

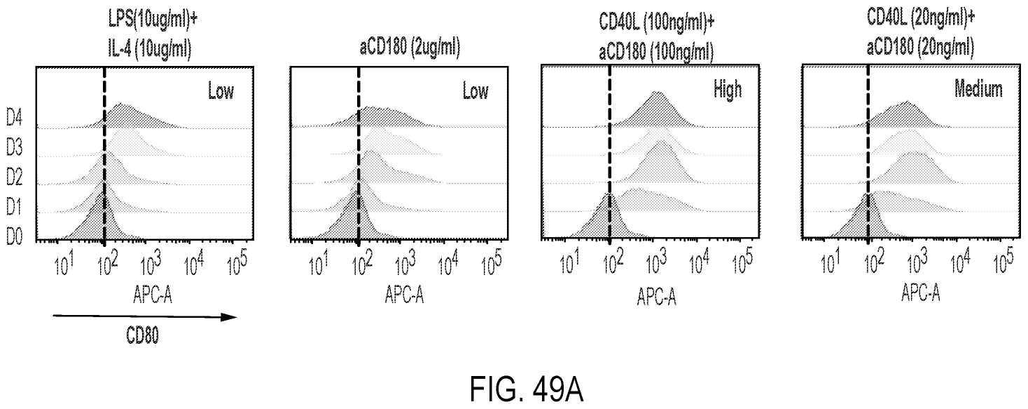

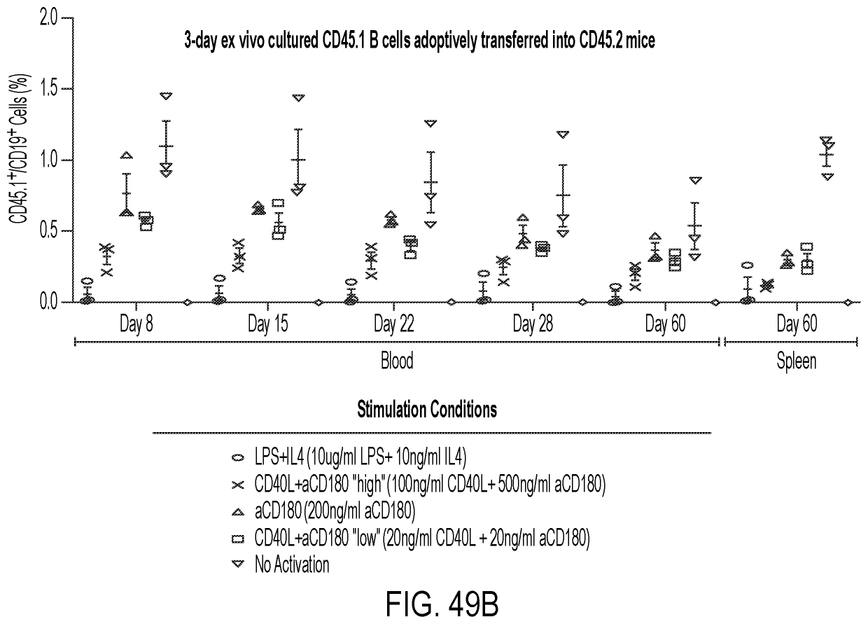

Murine B cells were isolated from spleens using EasySep Mouse B Cell Isolation kit and placed into culture containing the indicated amounts of B cell activation molecules. Cultures were analyzed by flow cytometry over the first four days for expression of CD80 (FIG. 49A).

Murine CD45.1 B cells were isolated from spleens using EasySep Mouse B Cell Isolation kit and placed into culture for 3 days with indicated amounts of B cell activation molecules. Cultured B cells were then adoptively transferred into congenic CD45.2 recipient mice. The frequency of donor CD45.1 cells were determined by flow cytometry in blood and spleen samples at the indicated times (FIG. 49B).

### Example 15: Ex Vivo AAV Transduction/Editing and Transfer of Cultured Non-Differentiated Cas9 Mouse B Cells into SRG Mice

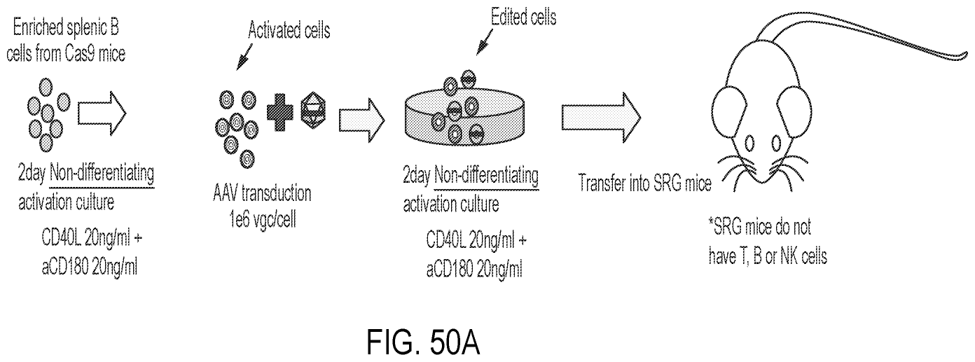

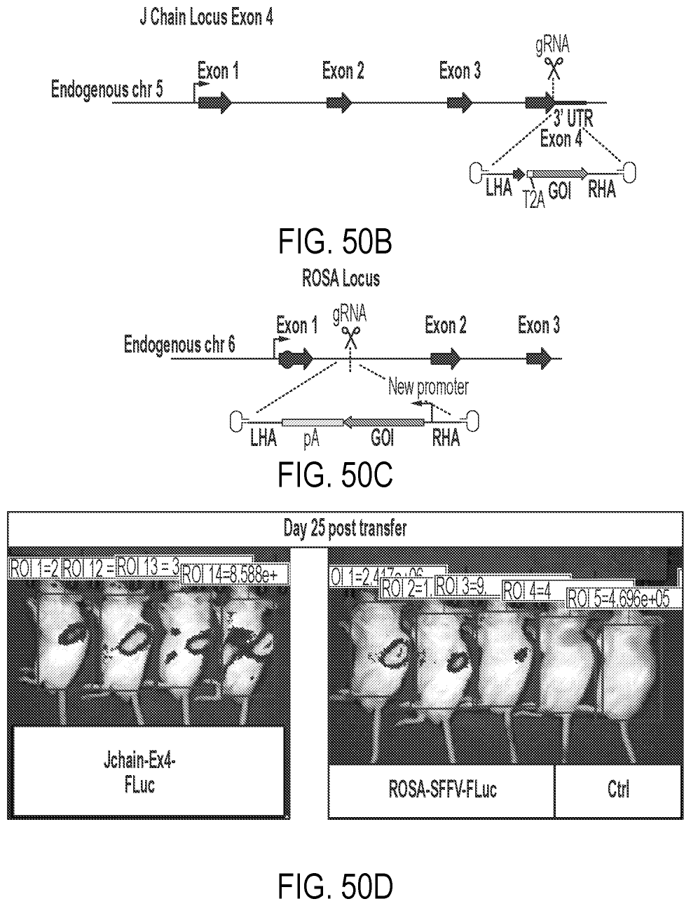

Enriched murine B cells from spleens of Cas9 ready mice were stimulated in culture using the indicated low amounts of CD40L and aCD180 for 2 days. These culture conditions activate B cells to proliferate without differentiation. After 2 days the B cells are transduced with AAV coding for gRNAs and homology templates. The homology templates code for luciferase expression from either J chain locus using endogenous J Chain promoter or from the ROSA locus using a synthetic B cell specific promoter Hg38-mCP. Cells were cultured for 2 more days in low activation conditions and then transferred into SRG mice where luciferase signal was measured in vivo overtime using IVIS technology (FIGS. 50A-50C).

### Example 16: Editing Strategies into Different Murine Loci for Different Modalities

This Example illustrates gene editing strategies used for mouse IgH locus insertion and ROSA locus insertion and the predicted protein products generated from the edited loci.

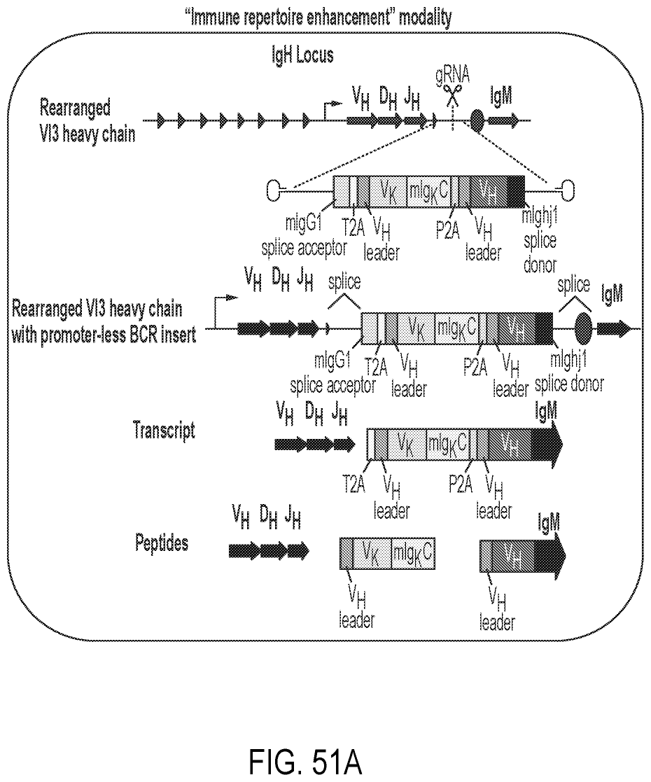

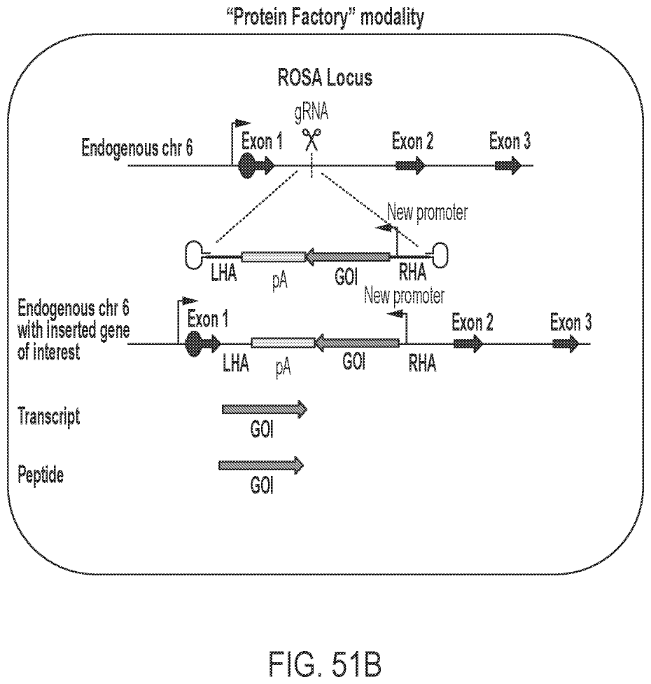

Editing into IgH locus is used for engineering a new BCR into the B cell via RNA splice “highjacking” of the endogenous VH RNA transcript to encode the new full Light Chain and Heavy Chain VDJ that splices back to the endogenous heavy chain constant region used by the cell (FIG. 51A).

The ROSA locus is commonly used in the mouse as a safe harbor for gene insertion. A construct can be edited into the ROSA locus that brings in promoters for various purposes (e.g. ubiquitous promoter, B cell specific promoter, etc.) (FIG. 51B).

### Example 17: Mouse J Chain Locus Insertion Strategies to Highly Express Proteins of Interest in Plasma Cells

This Example illustrates gene editing strategies used for mouse J locus insertion and the predicted protein products generated from the edited locus.

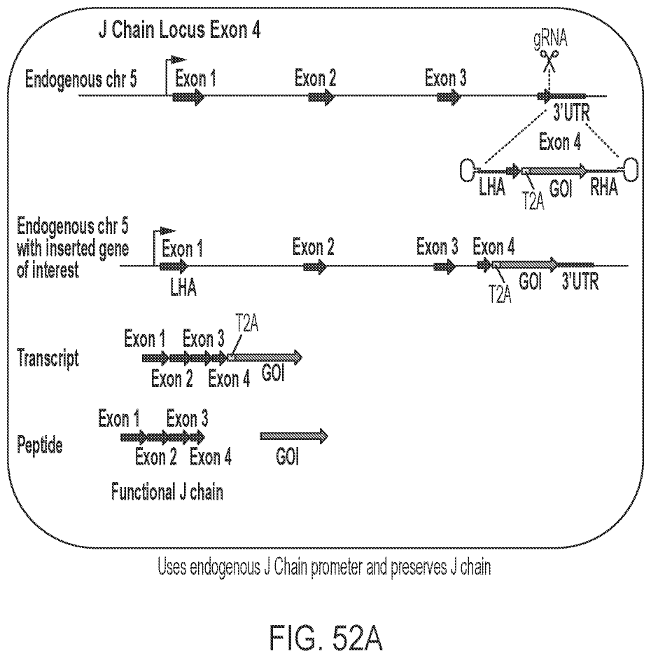

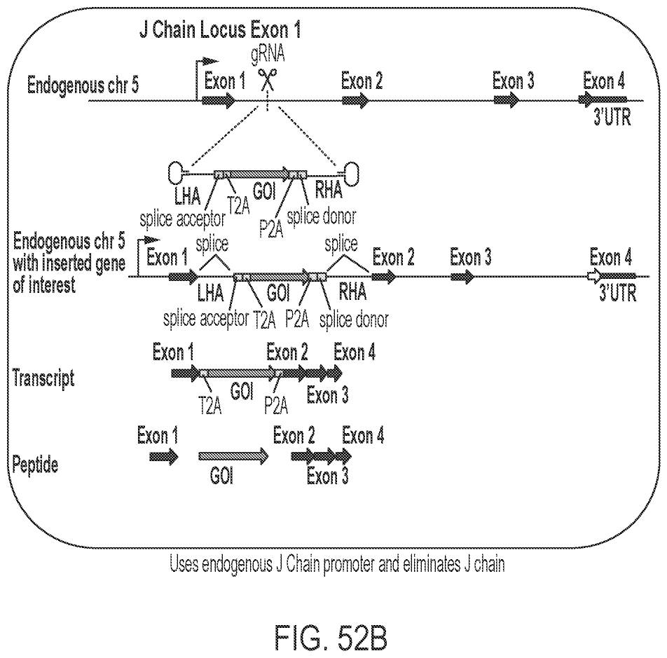

Editing into the 4th exon of mouse J Chain locus can be used for engineering the high expression of gene of interest from plasmablasts and plasma cells while retaining J Chain expression. The gene of interest is fused in frame to the last exon of the endogenous J Chain and processed via T2A technology (FIG. 52A).

Editing into the 1st intron of mouse J Chain locus can be used for engineering the high expression of gene of interest from plasmablasts and plasma cells while eliminating J Chain expression. The gene of interest is expressed off the endogenous J Chain promoter using an RNA splicing “highjack” method and is predicted to replace J Chain expression with expression of gene of interest (FIG. 52B).

### Example 18: Generation of Memory B Cells is Key to Success of In Vivo B Cell Editing for Both Adaptive Antibody and Protein Factory Modalities

Generation of edited B cell memory that can naturally expand is key to in vivo B cell engineering success regardless of desired modality (e.g., BCR swapping, protein factory, etc.).

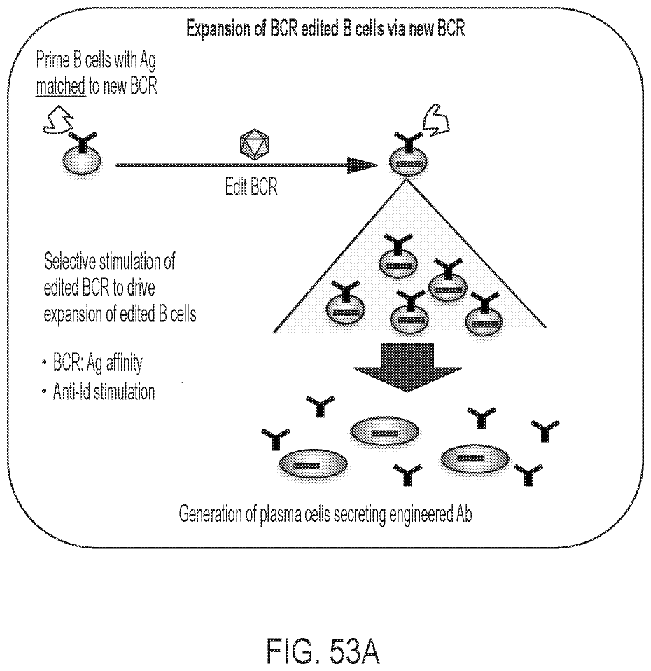

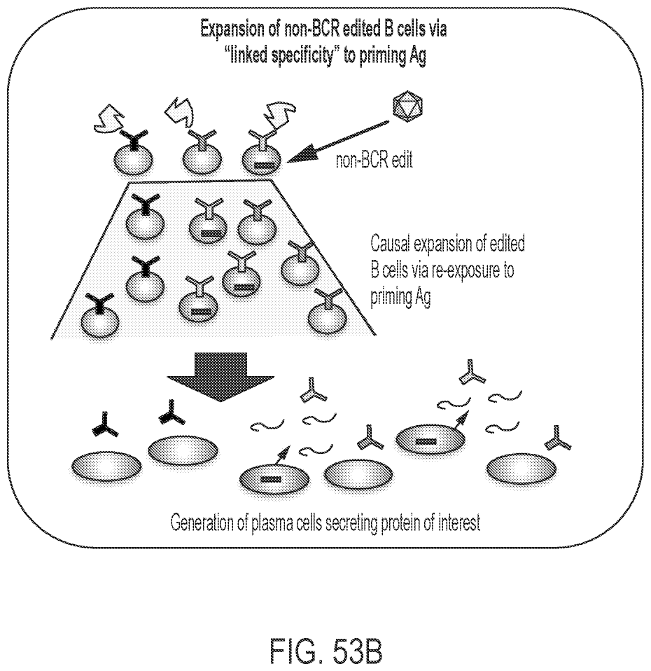

BCR edited B cells can be intentionally expanded in vivo by recruiting them into an immune response using antigen that is cognate for the BCR. This sets up an immune reaction that naturally expands the cells, generates memory B cells from the edited cells and can differentiate the edited B cells into plasma cells that secrete engineered Ab (FIG. 53A).

Editing a gene of interest into a locus other than BCR locus (e.g., IgH) requires linking the editing event to a known antigen in order to intentionally expand edited B cells where the BCR has not been edited. “Linked specificity” is achieved by priming the mice prior to editing by using a defined antigen immunization. B cells recruited into the immune response are preferentially edited by AAV and develop to become memory cells linked to the priming antigen. A boosting strategy using the same antigen as the prime Ag can be employed to further expand the edited cells to achieve higher levels of the protein of interest (FIG. 53B).

### Example 19: Modulation of “Pan B-Cell” Stimulation of Cas9 Mice Enables AAV Editing of B Cells and Ab Production

CD40 and CD180 receptors can be stimulated in vivo to activate B cells to be receptive to AAV editing in Cas9Ready mice. Adjusting the dosing of CD40 and CD180 agonists (e.g. antibody) during priming modulates the level of editing and/or engineered antibody initially produced.

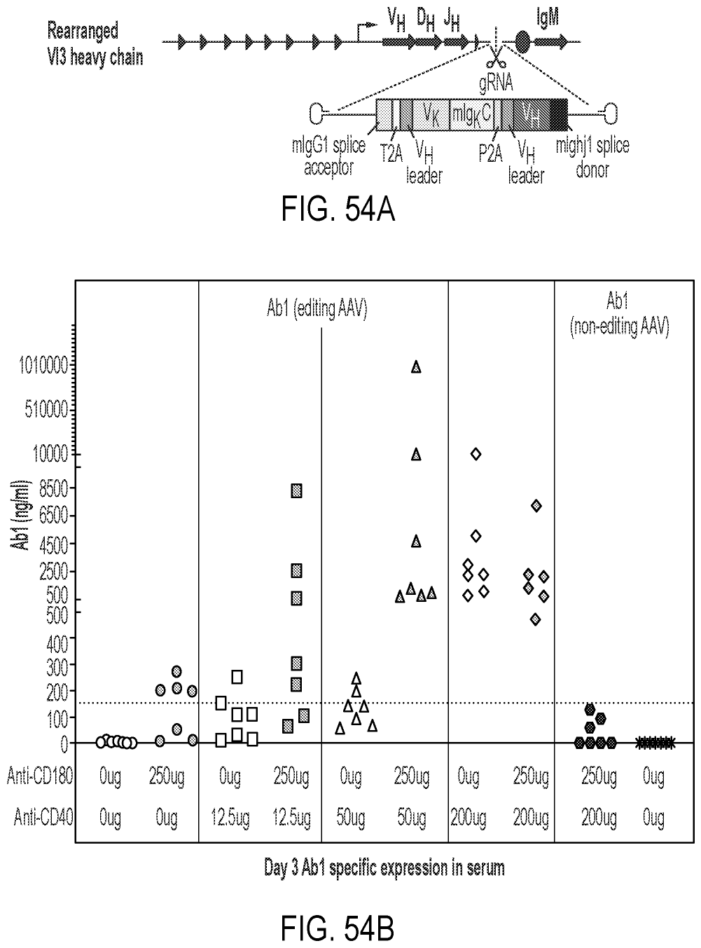

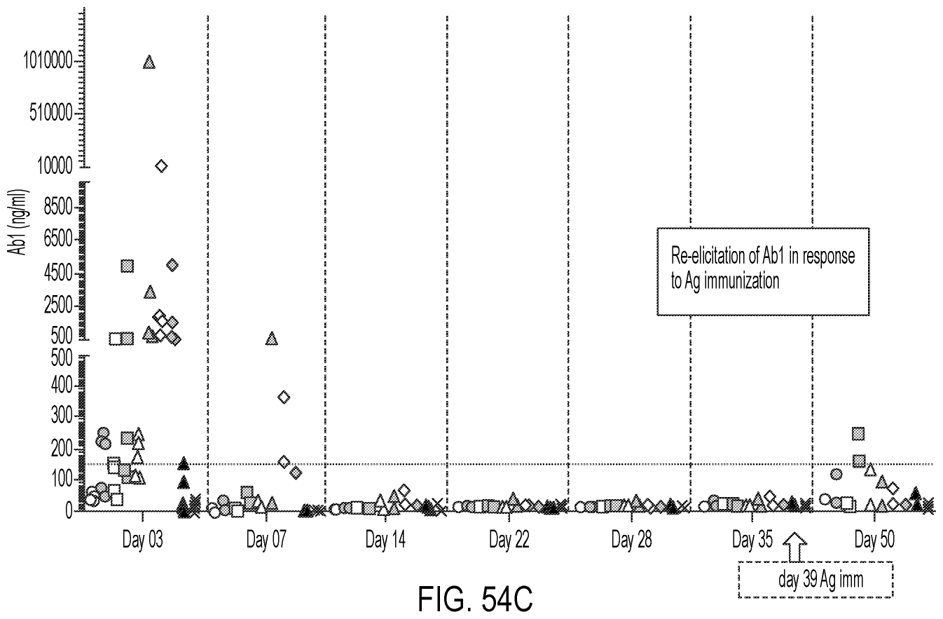

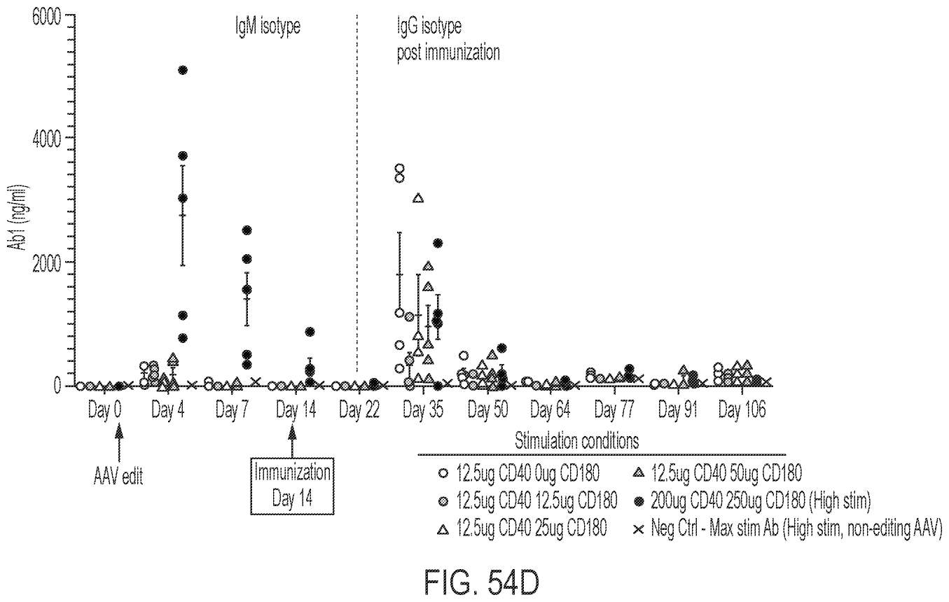

Cas9Ready mice were primed with different amounts of anti-CD40 and anti-CD180 Ab and transduced 3 days later with AAV encoding Ab1 BCR into the IgH locus. Ab1 Ab was detected in the sera of edited mice using an anti-idiotype ELISA method that specifically detects Ab1. CD40 and CD180 pathways synergized to activate B cells, facilitated editing, and elicited antibody production. High levels of anti-CD40 and anti-CD180 enabled B cell editing and rapid production of Ab1 by day 3. Lower levels of anti-CD40 and anti-CD180 enabled B cell editing and resulted in lower levels of Ab1 production at day 3 (FIG. 54B).

Ab1 Ab levels in mice from FIG. 54B were followed over time. Mice were boosted with Ag specific for Ab1 at day 42 post editing. Ab1 was detected in immunized mice from each AAV edited group that received activation priming indicating that edited cells were generated in every group that received priming regardless of strength of priming stimulus and whether that resulted in early Ab1 expression. Negative control was an AAV containing the BCR homology template but lacking the gRNA required for editing (FIG. 54C).

Cas9 mice primed with low dose combinations of anti-CD40 and anti-CD180 were edited as described in FIG. 54A and Ab1 serum Ab was assessed over time. Mice primed and edited with high dose priming elicited early robust short-lived expression of Ab1. This was found to be predominantly IgM. Low dose primed mice expressed little to no early Ab1. Mice were immunized with cognate Ag at day 14 post editing and Ab1 expression was assessed at time points post immunization. Immunization with Ag elicited Ab1 expression from all groups of mice that were primed and edited. Negative control is non-editing AAV lacking gRNA for editing (FIG. 54D). Ab1 Ab expressed post immunization was found to be primarily IgG isotype.

### Example 20: Prime & Boost with Suboptimal BCR:Ag Interaction Encourages Ab1 memB Cells Over Ab Producing PC

Cas9Ready mice primed with an antigen that suboptimally interacts with the editing Ab1 BCR promoted editing without Ab production. Boosting with high affinity antigen induced Ab1 expression from edited B cells.

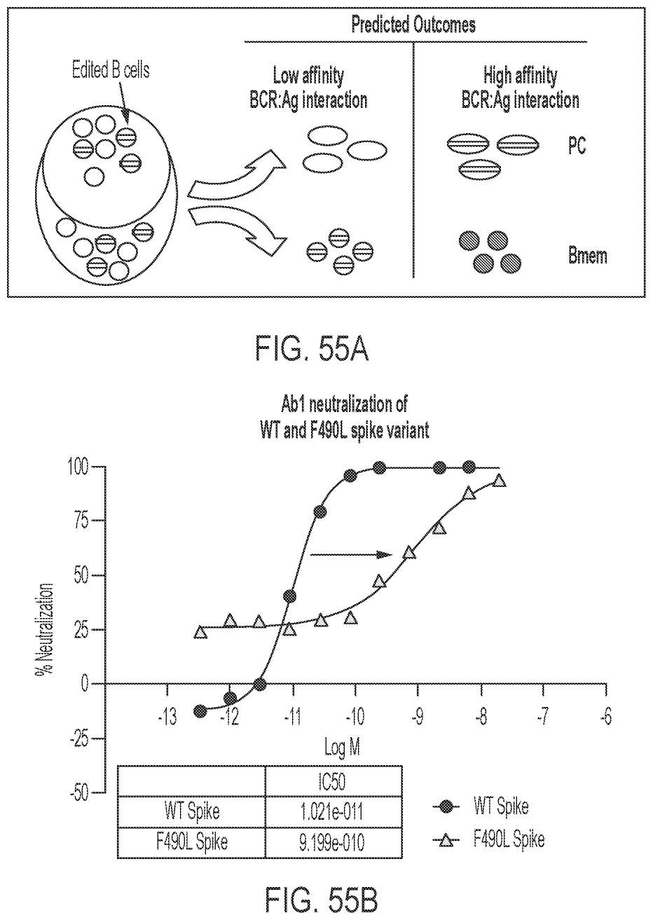

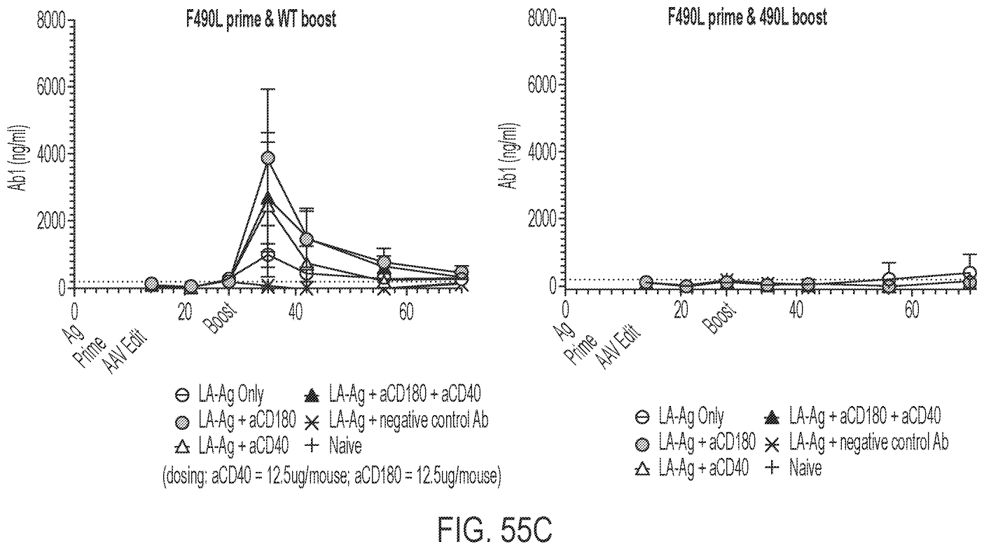

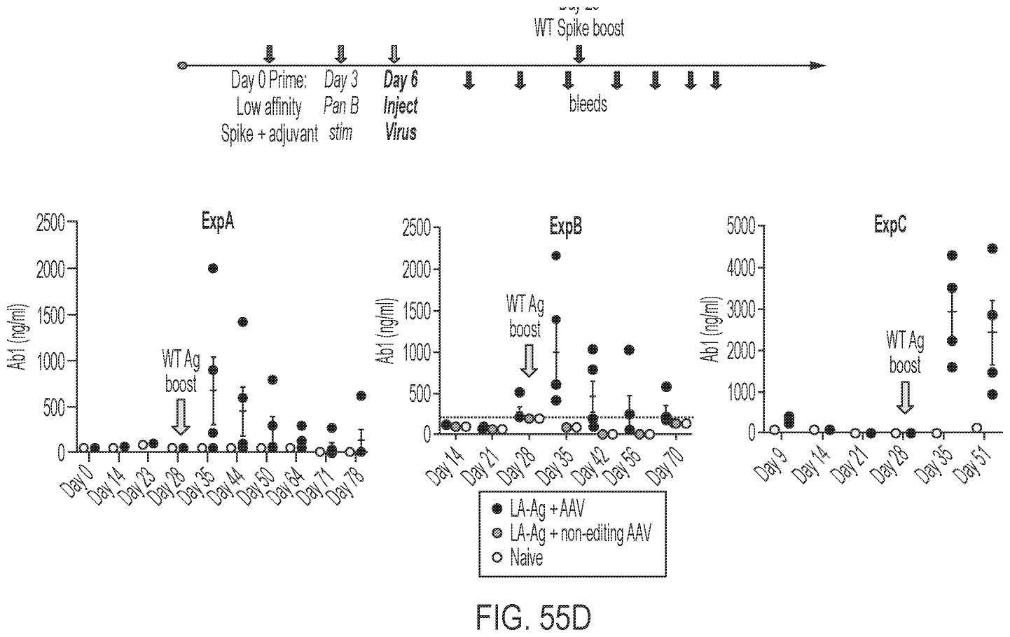

Ab1 exhibits a 90-fold decrease in ability to neutralize F490L spike variant pseudovirus compared to WT spike pseudovirus and represents a “suboptimal” antigen for the Ab1 BCR: interaction (FIG. 55B).

Cas9Ready mice primed with F490L spike Ag in alhydrogel (IP) were edited with Ab1 BCR AAV 6 days post prime and mice were then boosted with protein only of either “high affinity” WT spike Ag or F490L “low affinity” Ag at day 28 post prime. Negative control for editing was REGV157, an AAV lacking gRNA for editing. Ab1 serum Ab titers were assessed over time. In all mice, little to no Ab1 Ab was expressed prior to day 28 Ag boost. Only mice boosted with WT Ag expressed appreciable levels of Ab1 Ab indicating the presence of edited B cells capable of responding to Ag challenge. Mice boosted with suboptimal F490L Ag failed to induce expression of Ab1, indicating importance of high affinity BCR:Ag interactions in stimulating Ab production from edited cells (FIG. 54C).

Three experiments demonstrating editing the Ab1 BCR into Cas9Ready mice primed with “low affinity” Ag (LA-Ag) resulted in reproducible induction of Ab expression following d28 boost with WT “high affinity” Ag (WT-Ag) (FIG. 55D).

### Example 21: Demonstration of Recall to Ag from BCR Edited B Cells. The Addition of aCD180 to Ag Prime Increased Number of Edited B Cells Able to be Recalled One Month and 3 Months Post Editing

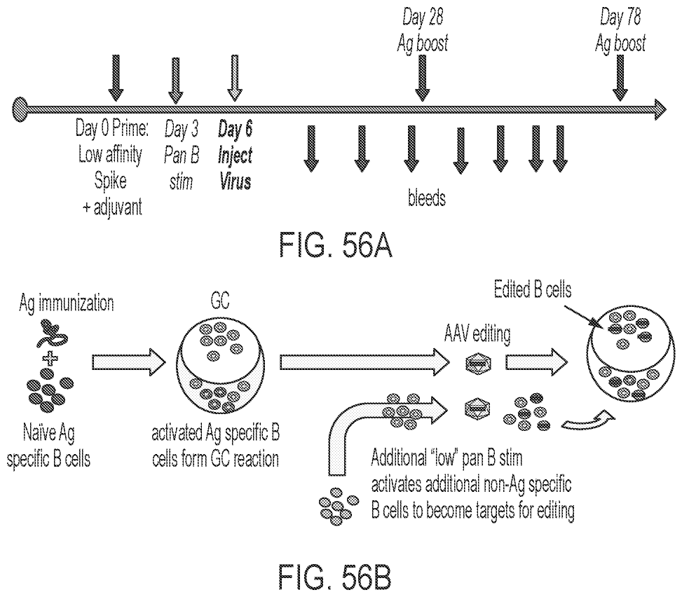

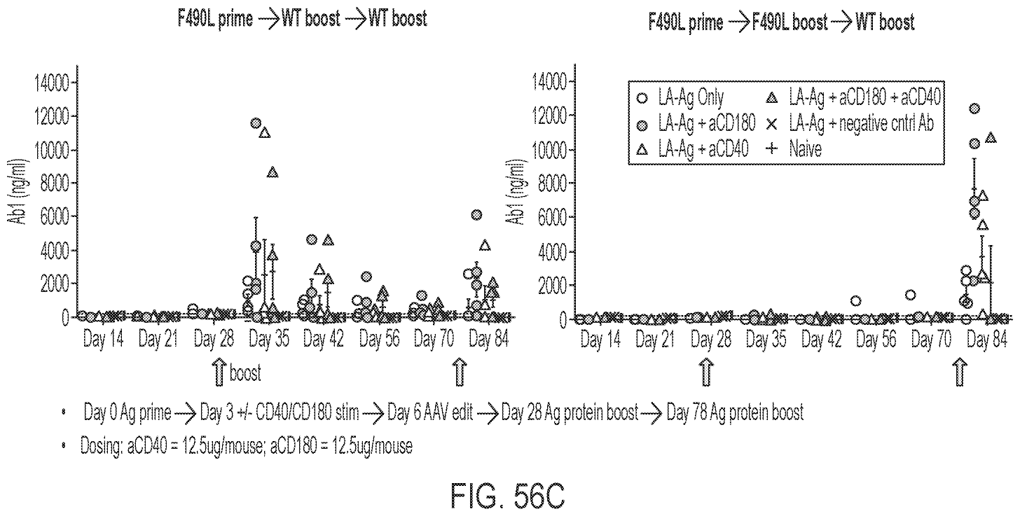

Cas9Ready mice were Ag “low affinity” primed +/−anti-CD180/anti-CD40 and then edited with Ab1 BCR AAV. Mice were boosted as indicated with either “high affinity” WT Ag or “low affinity” F490L Ag at day 28 and day 78 post prime. Ab1 serum levels assessed over time. The concept of combining “pan B” stimulation with the Ag priming was that Ag priming alone stimulates a limited number of B cells to be edited and that additional broader B cell activation using low dose anti-CD180 and/or anti-CD40 will increase the number of B cells capable of being edited (FIGS. 56A-56B).

Memory recall to “high affinity” WT Ag was observed in all groups of mice edited with Ab1 BCR as noted by induction of new Ab1 Ab in response to d78 Ag boost. Notably, this response to WT Ag was also observed in mice that failed to express Ab1 in response to “low affinity” boost at day 28, indicating that Ab1 memory B cells had been generated and survived for 3 months waiting for recall. Furthermore, combining “pan-B” stimulation with the Ag prime elicited higher levels of Ab1 Ab upon WT Ag boost indicating that more edited B cells were present at boost compared to mice primed with Ag alone (FIG. 56C).

### Example 22: Demonstration of Long-Term Persistence of In Vivo Edited B Cells (Non-IgH Locus) in Ag-Primed Mice

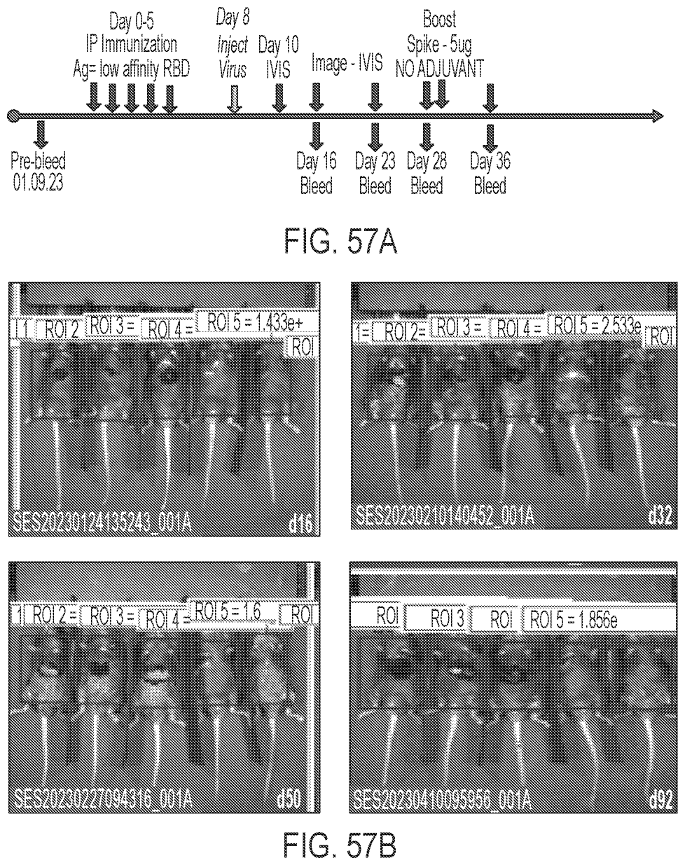

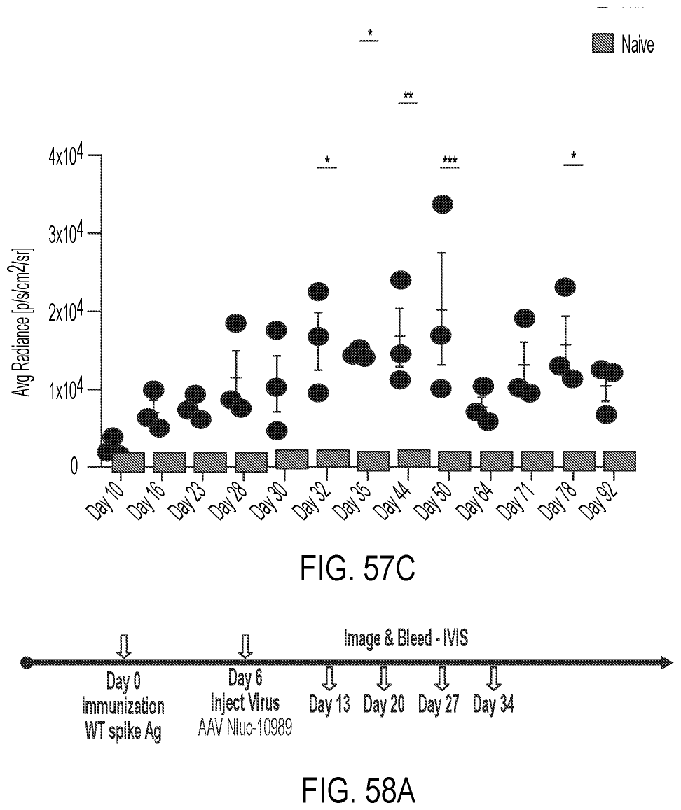

Cas9Ready mice were primed with antigen and edited with AAV that inserts luciferase into the ROSA locus driven by a B cell-specific synthetic Hg38-mCP promoter. Mice showed long term persistence of luciferase signal in draining lymph nodes (DLN) of peritoneal cavity (FIGS. 57A-57C).

IVIS imaging of luciferase signal from mice edited to express luciferase from a B cell specific promoter edited into the ROSA locus (FIG. 57B). The signal persisted in draining lymph nodes of the peritoneal cavity and is distinct from liver or spleen expression.

Longitudinal analysis of luciferase signal indicating durability of in vivo edited B cells in mice is shown in FIG. 57C.

### Example 23: AAV “Nluc-Ab1” Editing into IgH Locus Enables In Vivo Tracking of BCR Edited Cells Over Time

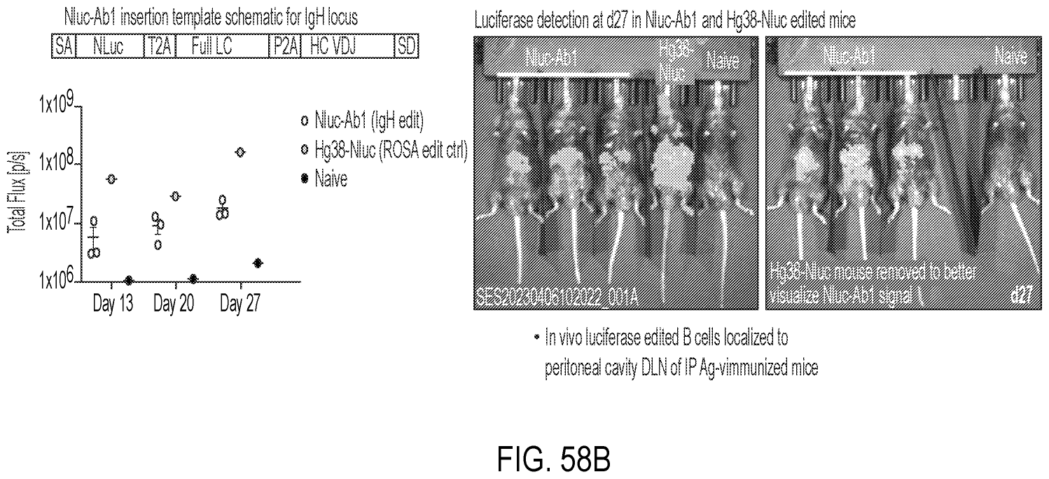

Cas9Ready mice were primed with antigen and edited with AAV that expressed luciferase and Ab1 BCR from the IgH locus (FIG. 58A).

An AAV template that modified the IgH locus to express both luciferase and Ab1 BCR shows localization of BCR edited B cells to the draining lymph nodes in the peritoneal cavity of mice primed with Ag (IP delivery). The luciferase signal from nLuc-Ab1 edited mice was compared to mice edited with ROSA locus AAV Hg-38-nLuc (FIG. 58B).

### Example 24: Peritoneal Cavity B Cell Editing Achieved Via IP Delivery of AAV into Unprimed Cas9Ready Mice

The tonic activation state of peritoneal cavity innate B cells, unlike conventional B cells, enables editing in absence of a priming stimulus. It was hypothesized herein that the unusual properties of innate B cells (i.e., self renewing, constitutive Ab expression, rapid plasma cell differentiation) make these cells an attractive target for expressing therapeutic proteins of interest.

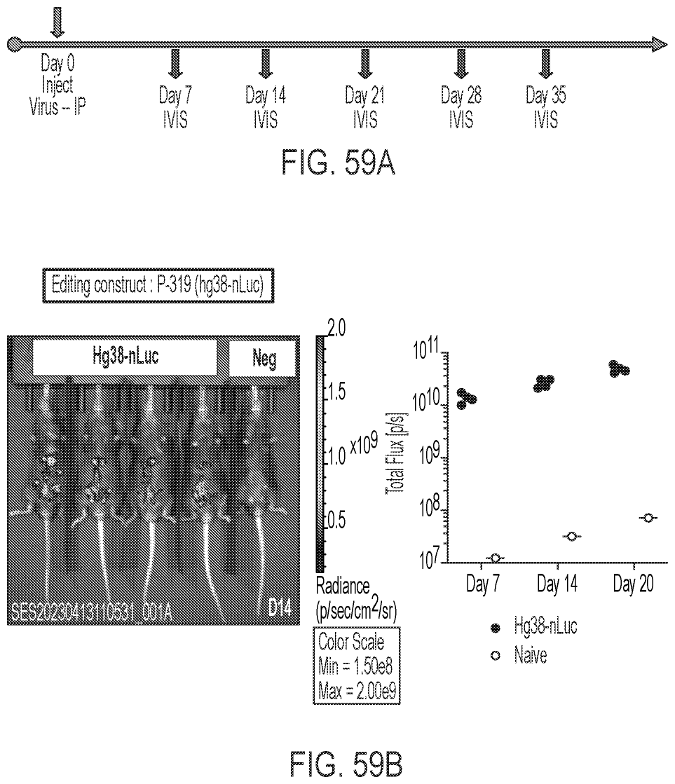

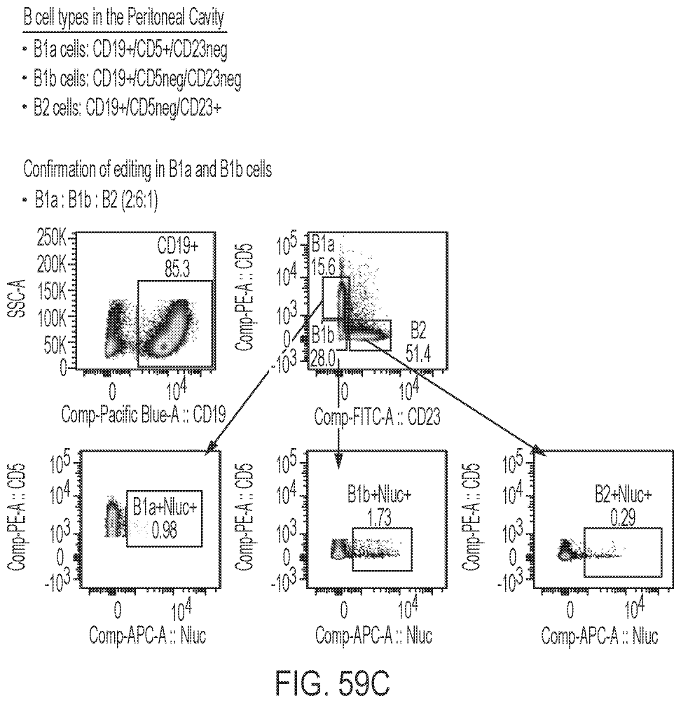

Unstimulated Cas9Ready mice were injected IP with AAV Hg-38-nLuc expressing B cell specific luciferase from the ROSA locus. The luciferase signal was readily observed in all the draining lymph nodes of the peritoneal cavity of Cas9Ready mice edited with B cell specific luciferase (FIG. 59A).

Flowcytometric analysis of B cells isolated from the peritoneal cavity of IP AVV edited Cas9Ready mice. Phenotyping of B cells for CD19, CD5, CD23 as indicated to define populations of B1a and B1b “innate” B cells and B2 “conventional” B cells. nLuc positive signal was predominantly observed in B1b and B1a B cells of the peritoneal cavity (FIG. 59B).

The present invention is not to be limited in scope by the specific embodiments described herein. Indeed, various modifications of the invention in addition to those described herein will become apparent to those skilled in the art from the foregoing description. Such modifications are intended to fall within the scope of the appended claims. It is further to be understood that all values are approximate and are provided for description.

All patents, patent applications, publications, test methods, product descriptions, literature, and other materials cited herein are hereby incorporated by reference in their entirety for all purposes as if physically present in this specification.

1. A system for producing an antibody or an antigen-binding fragment thereof in a subject, comprising:
a) a first component comprising a polynucleotide molecule, wherein the polynucleotide molecule comprises a sequence encoding the antibody or antigen-binding fragment thereof, and
b) a second component comprising a gene editing molecule or a polynucleotide molecule comprising a sequence encoding said gene editing molecule.

2. The system of claim 1, wherein an administration of the first and second components to the subject results in an integration of the sequence encoding the antibody or the antigen-binding fragment thereof into the DNA of a B cell and/or a hematopoietic stem cell (HSC) of the subject, causing the production of the antibody or the antigen-binding fragment in the subject.

3. The system of claim 1, wherein an administration of the first and second components to a B cell and/or a hematopoietic stem cell (HSC) ex vivo results in an integration of the sequence encoding the antibody or antigen-binding fragment thereof into the DNA of said cell to produce a modified B cell or a modified HSC, causing the production of the antibody or antigen-binding fragment thereof in the subject upon administration of said modified B cell or HSC to the subject.

4. The system of any one of claims 1-3, wherein the antibody or antigen-binding fragment thereof binds an antigen associated with a disease or disorder.

5. The system of claim 4, wherein said disease or disorder is an infection, cancer, autoimmune disease, cardiovascular disease, musculoskeletal disorder, or neurodegenerative disease.

6. The system of claim 5, wherein the infection is a viral infection, a bacterial infection, a fungal infection, or a parasite infection.

7. The system of any one of claims 4-6, wherein the antigen is a viral antigen, a bacterial antigen, a fungal antigen, a parasite antigen, or a tumor-associated antigen (TAA).

8. The system of any one of claims 1-7, wherein the gene editing molecule is a Cas nuclease.

9. The system of claim 8, wherein the Cas nuclease is a Cas9 nuclease.

10. The system of any one of claims 1-9, wherein the first or second component further comprises a guide RNA (gRNA) molecule or a sequence encoding said gRNA molecule.

11. The system of claim 10, wherein the first component comprises the polynucleotide molecule comprising the sequence encoding the antibody or antigen-binding fragment thereof and the sequence encoding the gRNA.

12. The system of claim 10, wherein the first component comprises (i) a first polynucleotide molecule comprising the sequence encoding the antibody or antigen-binding fragment thereof, and (ii) a second polynucleotide molecule comprising the sequence encoding the gRNA.

13. The system of claim 10, wherein the first component comprises (i) a first polynucleotide molecule comprising the sequence encoding the antibody or antigen-binding fragment thereof, and (ii) the gRNA molecule.

14. The system of claim 10, wherein the second component comprises the gRNA molecule or the sequence encoding said gRNA molecule.

15. The system of any one of claims 10-14, wherein the gRNA is complimentary to a sequence at the IgH locus, J Chain locus, or Ig Kappa locus.

16. The system of claim 15, wherein the gRNA is complimentary to a sequence in the 4th exon of the J Chain locus.

17. The system of claim 15, wherein the gRNA is complimentary to a sequence in the 1st intron of the J Chain locus.

18. The system of any one of claims 1-17, wherein the sequence encoding the antibody or antigen-binding fragment thereof comprises a sequence encoding the light chain variable region and optionally the light chain constant region of said antibody.

19. The system of any one of claims 1-18, wherein the sequence encoding the antibody or antigen-binding fragment thereof comprises a sequence encoding the heavy chain variable region of said antibody.

20. The system of any one of claims 1-20, wherein the sequence encoding the antibody or antigen-binding fragment thereof is integrated at the IgH locus in the genomic region downstream of the final J gene but upstream of the Eμ enhancer.

21. The system of any one of claims 1-20, wherein the integration of the sequence encoding the antibody or antigen-binding fragment thereof into the DNA of the B cell or HSC results in the disruption of the Kappa light chain constant region.

22. The system of any one of claims 1-21, wherein the polynucleotide molecule comprising the sequence encoding the antibody or antigen-binding fragment thereof comprises from 5′ to 3′ a 5′ IgH homology region, splice acceptor, a 2A sequence with 5′ furin cleavage sequence, a sequence encoding the light chain variable region of said antibody, a sequence encoding the light chain constant region of said antibody, a 2A sequence with 5′ furin cleavage sequence, a sequence encoding the heavy chain variable region of said antibody, splice donor sequence, and 3′ IgH homology region, wherein the heavy chain and light chain sequences can be placed in either order.

23. The system of any one of claims 1-21, wherein the polynucleotide molecule comprising the sequence encoding the antibody or antigen-binding fragment thereof comprises from 5′ to 3′ 5′ J Chain exon 4 homology region, a 2A sequence with 5′ furin cleavage sequence, a sequence encoding the light chain variable region of said antibody, a sequence encoding the light chain constant region of said antibody, a 2A sequence with 5′ furin cleavage sequence, a sequence encoding the heavy chain variable region of said antibody, a sequence encoding the heavy chain constant region of said antibody, 3′ J Chain exon 4 homology region, wherein the heavy chain and light chain sequences can be placed in either order.

24. The system of any one of claims 1-23, wherein the sequence encoding the antibody or antigen-binding fragment thereof does not comprise a promoter sequence.

25. The system of claim 24, wherein, upon integration of the sequence encoding the antibody or antigen-binding fragment thereof into the DNA of the B cell or HSC, said sequence is under the transcriptional control of an endogenous heavy chain promoter in the B cell or HSC.

26. The system of claim 24, wherein, upon integration of the sequence encoding the antibody or antigen-binding fragment thereof into the DNA of the B cell or HSC, said sequence is under the transcriptional control of an endogenous J Chain promoter in the B cell or HSC.

27. The system of any one of claims 1-23, wherein the sequence encoding the antibody or antigen-binding fragment thereof comprises a promoter sequence.

28. The system of claim 27, wherein the promoter is a B cell specific promoter or HSC specific promoter.

29. The system of claim 27, wherein the promoter is Hg38-mCP promoter.

30. The system of claim 27, wherein the promoter is the spleen focus forming virus (SFFV) promoter or a fragment thereof.

31. The system of any one of claims 1-30, wherein the first component and/or the second component are independently selected from a viral vector, a virus-like particle (VLP), a lipid nanoparticle (LNP), a liposome, and a ribonuclear protein (RNP) complex.

32. The system of claim 31, wherein the first component and the second component are both viral vectors.

33. The system of claim 32, wherein the viral vectors are derived from the same viral species.

34. The system of claim 32, wherein the viral vectors are derived from different viral species.

35. The system of any one of claims 31-34, wherein the viral vector is an adeno-associated virus (AAV) vector.

36. The system of claim 35, wherein the AAV vector is derived from AAV1, AAV2, AAV6, AAV9, or AAV9.PHP.

37. The system of claim 35 or claim 36, wherein the AAV vector capsid comprises one or more mutations, wherein said one or more mutations abolish a natural tropism of the AAV vector.

38. The system of any one of claims 31-34, wherein the viral vector is a retroviral vector.

39. The system of claim 38, wherein the retroviral vector is a lentiviral vector.

40. The system of any one of claims 31-39, wherein the viral vector further comprises a targeting moiety.

41. The system of claim 40, wherein the viral vector is an AAV vector and the targeting moiety is inserted into a protein forming the viral capsid or is covalently or non-covalently attached to the protein forming the viral capsid.

42. The system of claim 41, wherein the targeting moiety is attached to the viral capsid via a first member and a second member of a binding pair, wherein said first member and said second member form an isopeptide bond.

43. The system of claim 40, wherein the viral vector is a lentiviral vector and the targeting moiety is covalently or non-covalently attached to a fusogen.

44. The system of any one of claims 40-43, wherein the targeting moiety is a targeting antibody or an antigen-binding fragment thereof.

45. The system of claim 44, wherein the targeting antibody or antigen-binding fragment thereof binds to CD5, CD19, CD20, CD22, CD34, CD38, CD40, CD 117, CD79, CD180, B cell receptor (BCR), B-cell activating factor (BAFF), or Sca-1.

46. The system of any one of claims 1-45, wherein the subject is human.

47. The system of any one of claims 1-45, wherein the subject is an experimental animal.

48. A modified B cell or a modified hematopoietic stem cell (HSC) comprising the system of any one of claims 1-47.

49. A pharmaceutical composition comprising the system of any one of claims 1-47 and a pharmaceutically acceptable carrier or excipient.

50. A kit comprising (i) the system of any one of claims 1-47 and optionally (ii) a container and/or instructions for use.

51. A method for generating a modified B cell or a modified hematopoietic stem cell (HSC) producing an antibody or antigen-binding fragment thereof, comprising transducing ex vivo a B cell or HSC with an effective amount of the system of any one of claims 1-47, wherein the first component and the second component of the system are administered to said cell either simultaneously or sequentially in any order, and wherein the administration of the first and second components results in an integration of the sequence encoding the antibody or antigen-binding fragment thereof into the DNA of said cell, wherein said cell becomes a modified cell.

52. The method of claim 51, wherein the first component and the second component of the system are administered to said cell simultaneously as two separate compositions.

53. The method of claim 51, wherein the first component and the second component of the system are administered to said cell simultaneously as one composition.

54. The method of any one of claims 51-53, wherein said B cell or HSC is present in a heterogeneous cell population during the transduction.

55. The method of any one of claims 51-53, wherein the B cell has been isolated from spleen, peritoneum, or peripheral blood.

56. The method of any one of claims 51-55, wherein the B cell is a primary B cell.

57. The method of any one of claims 51-55, wherein the B cell is a B2 B cell.

58. The method of any one of claims 51-55, wherein the B cell is a B1 B cell.

59. The method of any one of claims 51-58, wherein the B cell is cultured under stimulation conditions prior to and/or after the transduction.

60. The method of claim 59, wherein the stimulation conditions promote B cell activation without differentiation.

61. The method of claim 59 or claim 60, wherein the B cell is cultured in the presence of an agonist of CD40 and/or an agonist of CD180 prior to and/or after the transduction.

62. The method of claim 61, wherein the agonist of CD40 is CD40L or an anti-CD40 antibody.

63. The method of claim 61 or claim 62, wherein the agonist of CD180 is an anti-CD180 antibody.

64. The method of claim 63, wherein the B cell is cultured in the presence of CD40L and/or the anti-CD180 antibody prior to and/or after the transduction.

65. The method of claim 64, wherein the B cell is cultured in the presence of CD40L and the anti-CD180 antibody prior to and/or after the transduction.

66. The method of claim 64 or claim 65, wherein the B cell is cultured in the presence of about 20 ng/ml or less of CD40L and/or about 100 ng/ml or less anti-CD180 antibody prior to and/or after the transduction.

67. The method of claim 66, wherein the B cell is cultured in the presence of about 20 ng/ml CD40L and about 20 ng/ml anti-CD180 antibody prior to and/or after the transduction.

68. The method of any one of claims 64-67, wherein the B cell is cultured in the presence of CD40L and/or anti-CD180 antibody for 4 days or less prior to the transduction.

69. The method of claim 68, wherein the B cell is cultured in the presence of CD40L and/or anti-CD180 antibody for about 2 days prior to the transduction.

70. The method of any one of claims 51-69, further comprising culturing the modified B cell or modified HSC under differentiating conditions to promote differentiation of said modified B cell or modified HSC into a modified plasma cell.

71. The method of any one of claims 51-70, further comprising introducing the modified B cell or the modified HSC or the modified plasma cell into a subject.

72. The method of claim 71, wherein the modified cell is introduced into the subject intraperitoneally.

73. The method of claim 71 or claim 72, wherein the subject has been depleted of CD20+ cells prior to introducing the modified cell.

74. The method of any one of claims 71-73, wherein after introducing the modified cell into the subject, said modified cell is expanded in vivo by administering to the subject an antigen that is recognized by the antibody or antigen-binding fragment thereof which is produced by said modified cell.

75. The method of any one of claims 71-74, wherein the subject is autologous to the modified cell.

76. The method of any one of claims 71-74, wherein the subject is allogeneic to the modified cell.

77. The method of any one of claims 71-76, wherein the subject is human.

78. The method of any one of claims 71-76, wherein the subject is an experimental animal.

79. A modified B cell or modified hematopoietic stem cell (HSC) produced by the method of any one of claims 51-69.

80. A modified plasma cell produced by the method of claim 70.

81. A method for producing an antibody or antigen-binding fragment thereof in vivo in a subject in need thereof, comprising administering to the subject an effective amount of the system of any one of claims 1-47, wherein the first component and the second component of the system are administered either simultaneously or sequentially in any order, and wherein the administration of the first and second components to the subject results in an integration of the sequence encoding the antibody or antigen-binding fragment thereof into the DNA of B cells and/or hematopoietic stem cells (HSCs) of the subject, causing a production of the antibody or antigen-binding fragment thereof in the subject.

82. The method of claim 81, wherein the first component and the second component of the system are administered to the subject simultaneously as two separate compositions.

83. The method of claim 81, wherein the first component and the second component of the system are administered to the subject simultaneously as one composition.

84. The method of any one of claims 81-83, wherein the first component and/or the second component of the system is administered to the subject intraperitoneally.

85. The method of any one of claims 81-84, wherein the method further comprises administering to the subject an effective amount of an agonist of CD40 and/or an agonist of CD180 prior to the administration of the system to the subject.

86. The method of claim 85, wherein the agonist of CD40 is CD40L or an anti-CD40 antibody.

87. The method of claim 85 or claim 86, wherein the agonist of CD180 is an anti-CD180 antibody.

88. The method of claim 87, wherein the method comprises administering to the subject an effective amount of an anti-CD180 antibody and/or an anti-CD40 antibody prior to the administration of the system to the subject.

89. The method of claim 88, wherein the method comprises administering to the subject an effective amount of the anti-CD180 antibody and anti-CD40 antibody prior to the administration of the system to the subject.

90. The method of claim 88 or claim 89, wherein the method comprises administering to the subject about 8.5 mg/kg or less of the anti-CD180 antibody and/or about 1.8 mg/kg or less of the anti-CD40 antibody prior to the administration of the system to the subject.

91. The method of any one of claims 88-90, wherein the method comprises administering to the subject about 0.4 mg/kg of the anti-CD180 antibody.

92. The method of any one of claims 88-91, wherein the method comprises administering to the subject the anti-CD180 antibody and/or anti-CD40 antibody about 7 days or less prior to the administration of the system to the subject.

93. The method of claim 92, wherein the method comprises administering to the subject the anti-CD180 antibody and/or anti-CD40 antibody about 2-3 days prior to the administration of the system to the subject.

94. The method of any one of claims 81-93, wherein the method further comprises administering to the subject an effective amount of an antigen which is recognized by the antibody or antigen-binding fragment thereof, wherein said antigen is administered before and/or after administering the first and/or second component of the system.

95. The method of claim 94, wherein said antigen has a low affinity for the antibody or antigen-binding fragment thereof.

96. The method of claim 94, wherein said antigen has a high affinity for the antibody or antigen-binding fragment thereof.

97. The method of any one of claims 94-96, wherein the method comprises administering to the subject an effective amount of a first antigen, wherein said first antigen has a low affinity for the antibody or antigen-binding fragment thereof and wherein said first antigen is administered prior to administering the first and second components of the system, and administering to the subject an effective amount of a second antigen, wherein said second antigen has a high affinity for the antibody or antigen-binding fragment thereof and wherein said second antigen is administered after administering the first and second components of the system.

98. The method of any one of claims 81-97, wherein the subject is human.

99. The method of any one of claims 81-97, wherein the subject is an experimental animal.

100. A method for treating or reducing the likelihood of a disease or disorder in a subject in need thereof, comprising performing the method of any one of claims 71-78 or the method of any one of claims 81-99, wherein the method results in a production in the subject of an effective amount of the antibody or antigen-binding fragment thereof.

101. The method of claim 100, wherein the disease or disorder is an infection, cancer, autoimmune disease, cardiovascular disease, musculoskeletal disorder, or neurodegenerative disease.

102. The method of claim 101, wherein the infection is a viral infection, a bacterial infection, a fungal infection, or a parasite infection.

103. The method of any one of claims 100-102, wherein the subject is human.
